SEQUENCE LISTING (62 sequences)

SEQ ID NO: 1 - DNA, 20 bp, Artificial Sequence
  Description of Artificial Sequence Synthetic primer
gcagcagtcccaggtagaag

SEQ ID NO: 2 - DNA, 26 bp, Artificial Sequence
  Description of Artificial Sequence Synthetic primer
atttttcatcttacttctgtggcatc

SEQ ID NO: 3 - DNA, 25 bp, Artificial Sequence
  Description of Artificial Sequence Synthetic primer
gcaagttgaataaccggaaatggtt

SEQ ID NO: 4 - DNA, 25 bp, Artificial Sequence
  Description of Artificial Sequence Synthetic primer
agggtgttataagcaatccccagaa

SEQ ID NO: 5 - DNA, 39 bp, Artificial Sequence
  Description of Artificial Sequence Synthetic probe
ctaaaggaaaagtgacacactgttttacaccttggtttt

SEQ ID NO: 6 - DNA, 39 bp, Artificial Sequence
  Description of Artificial Sequence Synthetic probe
aaaaccaaggtgtaaaacagtgtgtcacttttcctttag

SEQ ID NO: 7 - DNA, 39 bp, Artificial Sequence
  Description of Artificial Sequence Synthetic probe
  modified_base (18)..(19): a, c, t, g, unknown or other
ctaaaggaaaagtgacannctgttttacaccttggtttt

SEQ ID NO: 8 - DNA, 39 bp, Artificial Sequence
  Description of Artificial Sequence Synthetic probe
  modified_base (21)..(21): a, c, t, g, unknown or other
aaaaccaaggtgtaaaacagngtgtcacttttcctttag

SEQ ID NO: 9 - DNA, 38 bp, Artificial Sequence
  Description of Artificial Sequence Synthetic probe
tttcaaactagtattgtccgctttatagaaatataagc

SEQ ID NO: 10 - DNA, 38 bp, Artificial Sequence
  Description of Artificial Sequence Synthetic probe
gcttatatttctataaagcggacaatactagtttgaaa

SEQ ID NO: 11 - DNA, 38 bp, Artificial Sequence
  Description of Artificial Sequence Synthetic probe
  modified_base (19)..(20): a, c, t, g, unknown or other
tttcaaactagtattgtcnnctttatagaaatataagc

SEQ ID NO: 12 - DNA, 38 bp, Artificial Sequence
  Description of Artificial Sequence Synthetic probe
  modified_base (19)..(19): a, c, t, g, unknown or other
gcttatatttctataaagnggacaatactagtttgaaa

SEQ ID NO: 13 - DNA, 20 bp, Artificial Sequence
  Description of Artificial Sequence Synthetic primer
gctgtaggggtctcagtgct

SEQ ID NO: 14 - DNA, 20 bp, Artificial Sequence
  Description of Artificial Sequence Synthetic primer
ggccaaatagatgcattgct

SEQ ID NO: 15 - DNA, 21 bp, Artificial Sequence
  Description of Artificial Sequence Synthetic primer
cctctacctggtcatgcaact

SEQ ID NO: 16 - DNA, 22 bp, Artificial Sequence
  Description of Artificial Sequence Synthetic primer
tgtgtaaaaccccttctccact

SEQ ID NO: 17 - DNA, 19 bp, Artificial Sequence
  Description of Artificial Sequence Synthetic primer
ctggtgcttgggttgaatg

SEQ ID NO: 18 - DNA, 18 bp, Artificial Sequence
  Description of Artificial Sequence Synthetic primer
taactcctggggctgcac

SEQ ID NO: 19 - DNA, 21 bp, Artificial Sequence
  Description of Artificial Sequence Synthetic primer
ctgctcgctgaaacagttgta

SEQ ID NO: 20 - DNA, 21 bp, Artificial Sequence
  Description of Artificial Sequence Synthetic primer
ggcattgttcagtgaatgtcc

SEQ ID NO: 21 - DNA, 19 bp, Artificial Sequence
  Description of Artificial Sequence Synthetic primer
agggaagcctacgcagatg

SEQ ID NO: 22 - DNA, 20 bp, Artificial Sequence
  Description of Artificial Sequence Synthetic primer
tggacagtgggcgattttat

SEQ ID NO: 23 - DNA, 22 bp, Artificial Sequence
  Description of Artificial Sequence Synthetic primer
ttacagtgaagctgatgccagt

SEQ ID NO: 24 - DNA, 20 bp, Artificial Sequence
  Description of Artificial Sequence Synthetic primer
ttaggtcccgatggactacg

SEQ ID NO: 25 - DNA, 22 bp, Artificial Sequence
  Description of Artificial Sequence Synthetic primer
tctgacctaccagaccatcctc

SEQ ID NO: 26 - DNA, 22 bp, Artificial Sequence
  Description of Artificial Sequence Synthetic primer
agtagagcgatcaaacacgttg

SEQ ID NO: 27 - DNA, 20 bp, Artificial Sequence
  Description of Artificial Sequence Synthetic primer
ggaagagaagaggcgaggag

SEQ ID NO: 28 - DNA, 20 bp, Artificial Sequence
  Description of Artificial Sequence Synthetic primer
tggctgtgctggtaacagtg

SEQ ID NO: 29 - DNA, 20 bp, Artificial Sequence
  Description of Artificial Sequence Synthetic primer
gcgtgtgaagcactgaagtc

SEQ ID NO: 30 - DNA, 19 bp, Artificial Sequence
  Description of Artificial Sequence Synthetic primer
ggttcagttccgacccaac

SEQ ID NO: 31 - DNA, 20 bp, Artificial Sequence
  Description of Artificial Sequence Synthetic primer
acaggctgttagtccccaga

SEQ ID NO: 32 - DNA, 21 bp, Artificial Sequence
  Description of Artificial Sequence Synthetic primer
gccgtctcagctttattagca

SEQ ID NO: 33 - DNA, 20 bp, Artificial Sequence
  Description of Artificial Sequence Synthetic primer
tgcacttgctttttgacagg

SEQ ID NO: 34 - DNA, 18 bp, Artificial Sequence
  Description of Artificial Sequence Synthetic primer
ttggcaggcgtacaaagc

SEQ ID NO: 35 - DNA, 20 bp, Artificial Sequence
  Description of Artificial Sequence Synthetic primer
ccaggatggaggagtgaagt

SEQ ID NO: 36 - DNA, 20 bp, Artificial Sequence
  Description of Artificial Sequence Synthetic primer
cgcaagggtaaacattcctg

SEQ ID NO: 37 - DNA, 22 bp, Artificial Sequence
  Description of Artificial Sequence Synthetic primer
catcaatgctgctcttatggat

SEQ ID NO: 38 - DNA, 20 bp, Artificial Sequence
  Description of Artificial Sequence Synthetic primer
gcagtgggtattgtgtgacg

SEQ ID NO: 39 - DNA, 20 bp, Artificial Sequence
  Description of Artificial Sequence Synthetic primer
ccacccatctacaaacagca

SEQ ID NO: 40 - DNA, 21 bp, Artificial Sequence
  Description of Artificial Sequence Synthetic primer
ttggtatctcgttgggatctg

SEQ ID NO: 41 - DNA, 20 bp, Artificial Sequence
  Description of Artificial Sequence Synthetic primer
gggaggcttgcttctacctt

SEQ ID NO: 42 - DNA, 18 bp, Artificial Sequence
  Description of Artificial Sequence Synthetic primer
ttccggaacaccttgtgc

SEQ ID NO: 43 - DNA, 21 bp, Artificial Sequence
  Description of Artificial Sequence Synthetic primer
cctttgtggagacaggaatca

SEQ ID NO: 44 - DNA, 20 bp, Artificial Sequence
  Description of Artificial Sequence Synthetic primer
aaggggaaacagtgccatta

SEQ ID NO: 45 - DNA, 24 bp, Artificial Sequence
  Description of Artificial Sequence Synthetic primer
tctgtagtgaattcaagtgggaaa

SEQ ID NO: 46 - DNA, 26 bp, Artificial Sequence
  Description of Artificial Sequence Synthetic primer
ttactgaattgtctgcaaaatacaaa

SEQ ID NO: 47 - DNA, 19 bp, Artificial Sequence
  Description of Artificial Sequence Synthetic primer
ccagtccctggctgaaaat

SEQ ID NO: 48 - DNA, 19 bp, Artificial Sequence
  Description of Artificial Sequence Synthetic primer
gtccccgaagtcgtctagg

SEQ ID NO: 49 - DNA, 26 bp, Artificial Sequence
  Description of Artificial Sequence Synthetic primer
acgagacacttacttacttgaaccaa

SEQ ID NO: 50 - DNA, 20 bp, Artificial Sequence
  Description of Artificial Sequence Synthetic primer
tgtctcccattttgcgatta

SEQ ID NO: 51 - DNA, 18 bp, Artificial Sequence
  Description of Artificial Sequence Synthetic primer
gacggagaatggctctgc

SEQ ID NO: 52 - DNA, 22 bp, Artificial Sequence
  Description of Artificial Sequence Synthetic primer
gccatgagataattgggatctt

SEQ ID NO: 53 - DNA, 24 bp, Artificial Sequence
  Description of Artificial Sequence Synthetic primer
caaagattgcaggtatcctatgaa

SEQ ID NO: 54 - DNA, 20 bp, Artificial Sequence
  Description of Artificial Sequence Synthetic primer
tggctcttcagaccttcctt

SEQ ID NO: 55 - DNA, 20 bp, Artificial Sequence
  Description of Artificial Sequence Synthetic primer
ccgagggacagtgtatccag

SEQ ID NO: 56 - DNA, 25 bp, Artificial Sequence
  Description of Artificial Sequence Synthetic primer
agatctgctgctcgaatagtatttt

SEQ ID NO: 57 - DNA, 19 bp, Artificial Sequence
  Description of Artificial Sequence Synthetic primer
tctaccgggcaatgaacaa

SEQ ID NO: 58 - DNA, 20 bp, Artificial Sequence
  Description of Artificial Sequence Synthetic primer
cccgacaaagtgcagataca

SEQ ID NO: 59 - DNA, 20 bp, Artificial Sequence
  Description of Artificial Sequence Synthetic primer
catgagtacctgcgaagcaa

SEQ ID NO: 60 - DNA, 18 bp, Artificial Sequence
  Description of Artificial Sequence Synthetic primer
ccccgtcataatggcact

SEQ ID NO: 61 - DNA, 22 bp, Artificial Sequence
  Description of Artificial Sequence Synthetic probe
cgcgcgcgcacgcgcgcgcgca

SEQ ID NO: 62 - DNA, 23 bp, Artificial Sequence
  Description of Artificial Sequence Synthetic oligonucleotide
  modified_base (3)..(21): a, c, t, g, unknown or other
aannnnnnnnnnnnnnnnnnntt

(19) United States
(12) Patent Application Publication
(10) Pub. No.: US 2018/0318285 A1
(43) Pub. Date: Nov. 8, 2018

(54) METHODS FOR TREATMENT OF AUTISM SPECTRUM DISORDERS

(71) Applicant: PRESIDENT AND FELLOWS OF HARVARD COLLEGE, Cambridge, MA (US)

(72) Inventors: Harrison Wren GABEL, Brookline, MA (US); Benyam KINDE, Boston, MA (US); Michael E. GREENBERG, Brookline, MA (US)

(73) Assignee: PRESIDENT AND FELLOWS OF HARVARD COLLEGE, Cambridge, MA (US)

(21) Appl. No.: 15/556,665

(22) PCT Filed: Mar. 9, 2016
(86) PCT No.: PCT/US16/21461

§ 371 Date: Sep. 8, 2017

Related U.S. Application Data
(60) Provisional application No. 62/130,769, filed on Mar. 10, 2015.

(51) Int. Cl. A61K 31/4745; A61P 25/00

(52) U.S. Cl. CPC A61K 31/4745; A61P 25/00

(57) ABSTRACT

We have determined that MeCP2 protein mediates modulation of long-gene expression in the brain and results in neurological dysfunction associated with autism spectrum disorders, including but not limited to, Fragile X Syndrome, Rett syndrome, and Angelman syndrome (AS). In particular, a lack of MeCP2 protein causes up-regulation of long gene expression in the brain which corresponds with the pathology of Rett syndrome and Fragile X Syndrome, while too much MeCP2 protein results in excessive repression of long gene expression in the brain and pathology related to MeCP2 duplication syndrome. Accordingly, embodiments of the invention are directed to methods for treatment of autism spectrum disorders. The methods involve administration, to a subject, agents that modulate the expression of long genes in the brain.

### CROSS REFERENCE TO RELATED APPLICATIONS

This Application claims the benefit under 35 U.S.C. 119(e) of U.S. Provisional Application Ser. No. 62/130,769 filed on Mar. 10, 2015, the contents of which are herein incorporated by reference in their entirety.

### GOVERNMENT SUPPORT

This invention was made with Government support under Grant No. 1RO1NS048276, awarded by the National Institutes of Health (NIH). The Government has certain rights in the invention.

### FIELD OF THE INVENTION

Embodiments of the invention are directed to methods for treatment of autism spectrum disorders. The methods involve modulation of the expression of long genes in the brain.

### BACKGROUND OF THE INVENTION

Recent evidence indicates that genetic mutations underlie many neurodevelopmental disorders, and thus a critical first step toward the rational design of therapeutics for these disorders is to understand the molecular function of the disease-causing genes. In females with Rett syndrome (RTT), mutations of the X-linked MECP2 gene lead to abnormal brain development, seizures, and severe motor dis-coordination in the first few years of life¹. MECP2 has high affinity for methylated DNA and has been proposed to function as a repressor of transcription². Although Mecp2 knockout (MeCP2 KO) mice faithfully recapitulate many aspects of RTT, in the absence of MeCP2 surprisingly small changes in gene expression have been observed in the brain³⁻⁹. In addition, across many studies there has been limited overlap in the specific genes that were identified as misregulated.

MeCP2 is highly expressed in neurons at a level similar to that of histones¹⁰, and chromatin immunoprecipitation analysis has revealed that MeCP2 binds broadly across the neuronal genome^{8,10,11}. These findings suggest that MeCP2 functions not as a promoter- or enhancer-specific transcription factor, but rather as a core component of chromatin. Because MeCP2 binds broadly across the genome rather than to discrete DNA regulatory elements, it has been challenging to determine how MeCP2 affects gene expression, and whether MeCP2 drives the induction or repression of transcription remains a subject of controversy. Furthermore, while it is known that MeCP2 displays a high degree of specificity for binding to methylated cytosine DNA in vitro², it is not well understood how MeCP2 functions with DNA methylation in vivo to regulate neuronal gene expression. Understanding how disruption of MeCP2 and other candidate autism genes result in neuropathologies will aid in the development of therapies for the treatment of this disorder.

### SUMMARY OF THE INVENTION

Embodiments of the invention are based, in part, on the discovery that modulation of long-gene expression in the brain results in neurological dysfunction associated with autism spectrum disorders, including but not limited to, Fragile X Syndrome, Rett syndrome, and Angelman syndrome (AS). In particular, we have elucidated the role that the MECP2 gene plays in Rett syndrome by determining that the MeCP2 protein modulates long gene expression, specifically long gene expression in the brain. We have discovered that MeCP2 normally, in healthy individuals, represses long genes (genes greater than 100 kilobases) by binding of MeCP2 to non-CpG methylated cytosines enriched in the brain and recruiting the NCoR co-repressor complex. We have further determined that in the absence of MeCP2 there is an increase in expression of long genes in the brain that specifically correlates with the severity and phenotypic onset of neuronal pathology in Rett syndrome. Significantly, our analysis indicates that long genes expressed in the brain include genes linked to autism spectrum disorders, and that the Fragile X syndrome protein, FMRP, also regulates long gene expression. Thus, we have discovered that a function of MeCP2 in the mammalian brain is to temper the expression of genes in a length-dependent manner and our analysis indicates that mutations in MeCP2 and other established autism genes cause neurological dysfunction by disrupting the expression of long genes in the brain.

Accordingly, embodiments of the invention are directed to the methods of treating autism spectrum disorders comprising administering an effective amount of an agent that modulates long gene expression in the brain. In one embodiment, the agent modulates expression of long genes in the brain by modulating the transcription of long genes. In another embodiment, the agent modulates expression of long genes in the brain by modulating the translation of long genes.

In certain aspects, for treatment of the autism spectrum disorder, the agent administered to the subject increases expression of long genes in the brain. In other aspects, for treatment of the autism spectrum disorder, the agent administered to the subject decreases expression of long genes in the brain. For example, in one embodiment, the autism spectrum disorder is MeCP2 duplication disorder and the agent increases the expression of long genes in the brain. In an alternative embodiment, the autism spectrum disorder is Rett syndrome and the agent decreases the expression of long genes in the brain. In another embodiment, the autism spectrum disorder is Fragile X syndrome and the agent decreases the expression of long genes in the brain. In still another embodiment, the autism spectrum disorder is caused by a mutation in topoisomerase and the agent increases expression of a long gene in the brain.

In certain embodiments, the agent is selected from the group consisting of a small molecule, a nucleic acid, a protein, a peptide, and an antibody. For example in one embodiment, the agent is an RNA interfering agent (RNAi). The agent may be administered by a route selected from the group consisting of topical administration, enteral administration, and parenteral administration.

In certain embodiments, the agent is administered using a chronic treatment regime, e.g. the agent is administered for the life of the patient, e.g. daily, weekly or monthly. In certain embodiments, the agent is formulated for delivery to the brain, e.g. formulated to cross the blood brain barrier, or formulated for intracranial injection.

Any agent known to up-regulate or down-regulate expression of long genes in the brain can be used in methods of the invention. In one embodiment, the agent is not an inhibitor of toposisomerase I. In another embodiment, the agent is not an inhibitor of toposisomerase II.

In one embodiment, the agent that increases expression of long genes in the brain is a DNA methyltransferase inhibitor, non-limiting examples include RG108, epigallocatachin-3-gallate, or 5-azacytosine.

In one embodiment, the agent that decreases expression of long genes in the brain and is selected from the group consisting of: a topoisomerase inhibitor, a nucleotide analog that inhibits transcriptional elongation, a BRD4 inhibitor that inhibits pro-elongation chromatin modifiers, an inhibitor of Dot1 that promotes elongation-associated chromatin modification, Alpha-Amanitin, a protein synthesis inhibitor, and a DNA intercalator that blocks RNA polymerases.

In one embodiment, the agent that decreases expression of long genes in the brain inhibits a protein that promotes elongation selected from the group consisting of: BRD4, Dot11, Ptefb, DSIF, SPt5p, Spt4p, PAF, Ccr4-Not, Sp3, ELL, P-TEFb, and. AFF4.

In one embodiment, the agent that increases expression of long genes in the brain activates a protein that promotes elongation selected from the group consisting of: BRD4, Dot11, Ptefb, DSIF, SPt5p, Spt4p, PAF, Ccr4, Not, Sp3, ELL, P-TEFb, and. AFF4.

In certain embodiments, the agent inhibits or activates proteins and complexes involved in translational elongation. In one embodiment, the agent is selected from the group consisting of: an agent selected from the group consisting of: Lactimidomycin, Diphthamide, Stm1p, 4EGI1, Orthoformimysin, e1F5A, Minocycline.

In another aspect, a method for treatment of Rett syndrome is provided. The method comprises administering to a subject an effective amount of a topoisomerase inhibitor, wherein the effective amount of the topoisomerase inhibitor decreases the expression of long genes in the brain. In still another aspect, a method for treatment of Fragile X syndrome is provided. The method comprises administering to a subject an effective amount of a topoisomerase inhibitor, wherein the effective amount of the topoisomerase inhibitor decreases the expression of long genes in the brain.

In certain embodiments of these aspects, the topoisomerase inhibitor is a topoisomerase I inhibitor selected from the group consisting of: Belotecan (CKD602), Camptothecin, 7-Ethyl-10-Hydroxy-CPT, 10-Hydroxy-CPT, Rubitecan (9-Nitro-CPT), 7-Ethyl-CPT, Topotecan, Irinotecan, Silatecan (DB67) and an indenoisoquinoline derivative.

In one embodiment, the topoisomerase inhibitor is:

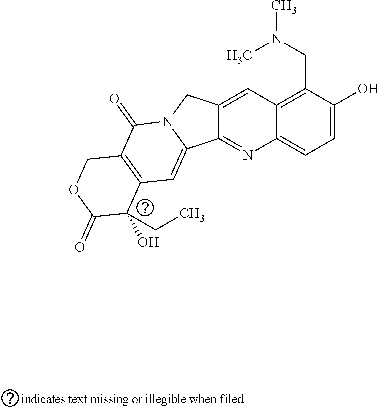 [SMILES: CC[C@@]1(O)C(=O)OCc2c1cc1n(c2=O)Cc2cc3c(CN(C)C)c(O)ccc3nc2-1]

In certain embodiments of these aspects, the topoisomerase inhibitor is a topoisomerase II inhibitor selected from the group consisting of: Doxorubicin; Etoposide; Amsacrine; ICRF-193, dexrazoxane (ICRF-187); Resveratrol; Epigallocatechin gallate; Genistein; Quercetin; and Myricetin.

### BRIEF DESCRIPTION OF THE DRAWINGS

This application file contains at least one drawing executed in color. Copies of this patent application publication with color drawings will be provided by the Office upon request and payment of the necessary fee.

FIGS. 1a to 1d are graphs that illustrate length-dependent gene misregulation is consistently detected in mouse models of RTT. FIG. 1a, Boxplots showing distributions of gene lengths (Refseq-annotated transcription start site to transcription termination site) for genes detected as misregulated in independent studies of brain regions from MeCP2 mutant mice (see methods for boxplot statistics). All genes, all genes in the genome; HYP, hypothalamus⁵; CB, cerebellum⁶; AMG, amygdala⁷; HC, hippocampus⁸; STR, striatum⁹; LVR, liver⁹. For HYP, CB, and AMG, genes were identified based on opposing changes in MeCP2 KO and MeCP2 OE mice⁵⁻⁷. For HC, STR, and LVR, alterations were assessed in MeCP2 KO alone^{8,9}. “MeCP2-induced” genes are down-regulated in MeCP2 KO and up-regulated in MeCP2 OE. “MeCP2-repressed” genes are up-regulated in MeCP2 KO and down-regulated in MeCP2 OE. FIG. 1b, Mean changes in expression for all genes binned according to length from microarray analysis of the MeCP2 KO hypothalamus⁵. FIG. 1c, Mean expression changes across five brain regions and liver of MeCP2 KO or MeCP2 OE mice for long genes (>100 kb) compared to the remaining genes in the genome (≤100 kb). FIG. 1d, Mean changes in expression for genes binned according to length in MeCP2 OE hypothalamus⁵. For FIG. 1b and FIG. 1d, the red line represents mean fold-change in MeCP2 mutant vs wild type for each bin and the red ribbon is standard error (SE) for genes within each bin and across all samples tested. Mean (black line) and two standard deviations (gray ribbon) are shown for Monte Carlo resampling of the data in which gene lengths were randomized with respect to fold-change 10,000 times. *, p<0.05; **, p<0.01; ***, p<1×10⁻¹⁰, n.s. p≥0.05 (two-tailed t-test, Bonferroni multiple testing correction). Comparison in FIG. 1a is each gene set vs all genes; comparison in c is genes >100 kb vs genes ≤100 kb. Note that the spike in mean fold-change at ˜1 kb that appears in FIG. 1b and FIG. 1d corresponds to misregulation of the olfactory receptor genes that occurs in MeCP2 mutants (see Discussion).

FIGS. 2a to 2c are graphs that depict length-dependent gene misregulation occurs in a human model of RTT. FIG. 2a-FIG. 2c, Mean changes in gene expression for genes binned according to length in human MECP2 null ES cells differentiated by Li and colleagues¹⁹ into neural progenitor cells (a), neurons cultured for 2 weeks (b), or neurons cultured for 4 weeks (c). For all plots, the red line represents mean fold-change in MECP2 null vs. wild type for each bin, the red ribbon is SE of genes within each bin and across samples tested. Mean (black line) and two standard deviations (gray ribbon) are shown for Monte Carlo resampling of the data in which gene lengths were randomized with respect to fold-change 10,000 times.

FIGS. 3a to 3f are graphs depicting mCH is enriched within long genes repressed by MeCP2. FIG. 3a, Mean changes in gene expression assessed by RNA-seq analysis of cortical tissue from MeCP2 KO compared to wild type mice. Fold-change values for genes binned according to gene length are shown (n=3 wild type, 3 MeCP2 KO). FIG. 3b, Mean changes in gene expression in cortical tissue of MeCP2 KO mice compared to wild type for genes binned according to mean fraction of cytosines methylated at CH dinucleotides (mCH/CH) within the gene body (transcription start site +3 kb, up to transcription termination site). FIG. 3c, Mean mCH/CH within gene bodies in cortical tissue for genes binned according to length. FIG. 3d, Mean changes in gene expression in cortical tissue of MeCP2 KO compared to wild type mice for high mCH genes (mCH/CH>0.020) and low mCH genes (mCH/CH<0.018), binned according to length. FIG. 3e, Mean changes in gene expression in cortical tissue of MeCP2 KO compared to wild type for long genes (>56 kb, longest 25% of genes) and short genes (<13 kb, shortest 25% of genes) binned according to gene-body mCH/CH levels. FIG. 3f, Mean changes in gene expression in the MeCP2 KO across three brain regions for all genes >100 kb compared to the subsets of genes >100 kb which land in the lowest (Low mCH) and highest (High mCH) quartiles of mCH/CH levels within their gene body. CTX, Cortex; HC, hippocampus; CB, cerebellum. In panels a through e, mean values for each bin are indicated as a line, and ribbon depicts SE for each bin. ***, p<1×10⁻¹⁰ two-tailed t-test, Bonferroni multiple testing correction.

FIGS. 4a to 4b are graphs depicting that interaction with the NCoR/SMRT histone deacetylase complex is required for length-dependent gene regulation by MeCP2. FIG. 4a, FIG. 4b, Mean changes in expression from microarray analysis of genes binned according to length in the cerebellum of MeCP2 KO (FIG. 4a) (n=5 wild type and 5 KO⁶) and MeCP2 R306C (FIG. 4b) mice (n=4 wild type, and 4 R306C). For each plot, the red line represents mean fold-change of each bin, and the red ribbon is SE for genes within the bin and across samples tested. Mean fold-change (black line) and two standard deviations (gray ribbon) are shown for Monte Carlo resampling of the data in which gene lengths were randomized with respect to fold-change 10,000 times.

FIGS. 5a to 5d are graphs depicting that long brain-specifically expressed genes are regulated by MeCP2 and FMRP. FIG. 5a, Cumulative distribution function of gene lengths for all genes in the genome, MeCP2-repressed genes identified in this study, SFARI autism candidate genes (http://sfari.org/), and genes encoding putative FMRP target mRNAs³¹ (p<1×10⁻¹⁵ for each geneset vs all genes, 2-sample Kolmogorov-Smirnov (KS) test). FIG. 5b, Overlap between MeCP2-repressed genes and autism spectrum disorder candidate loci or putative FMRP target mRNAs (p<5×10⁻⁵ for each overlap, hypergeometric test). Expected overlap for genes ≤100 kb and >100 kb was calculated by dividing the expected overlap for all genes (hypergeometric distribution) according to the distribution of all gene lengths in the genome. FIG. 5c, Mean expression of genes binned according to length in seven different neural and non-neural tissues from mouse. FIG. 5d, Mean expression of genes binned according to length in ten different human neural and non-neural tissues. In FIG. 5c and FIG. 5d mean expression for genes within each bin is indicated by the line, and the ribbon represents the SE of genes within each bin.

FIGS. 6a to 6d are graphs that depict Analysis of gene expression changes in MeCP2 mutant mice across multiple published datasets. FIG. 6a, Example scatter plots of fold-change in expression for the MeCP2 KO compared to wild type for the amygdala⁷ (left) which shows robust length-dependent misregulation, and the liver⁹ (right), which does not. Fold-change values for each gene (black points) and mean fold-change for 200 gene bins are shown (red line indicates mean, ribbon indicates SE for genes within each bin). Note that all genes near and above 1 megabase in length are up-regulated in the MeCP2 KO amygdala, while these genes are distributed above and below zero in the MeCP2 KO liver. FIG. 6b to FIG. 6d, Mean fold-change for genes binned according to length (top; 200 gene bins, 40 gene step), and the fraction of genes showing a positive change in expression for genes binned according to length (bottom; 100 gene bins, 50 genes step). FIG. 6b, Expression analysis of published microarray data from MeCP2 KO mice compared to wild type for five brain regions and liver⁵⁻⁹. c, Expression analysis of published microarray data from MeCP2 OE mice compared to wild type for three brain regions⁵⁻⁷. FIG. 6d, Expression analysis of published RNA-seq data from MeCP2 KO mice compared to wild type for purified cerebellar granule cells¹³. For all fold-change plots, the red line represents mean fold-change in MeCP2 mutant vs wild type for each bin, and the red ribbon is SE for each bin. Mean (black line) and two standard deviations (gray ribbon) are shown for Monte Carlo resampling in which gene lengths were randomized with respect to fold-change 10,000 times. The spike in mean fold-change at ˜1 kb that appears in several plots corresponds to misregulation of the olfactory receptor genes that occurs in MeCP2 mutants (see Example 1). Note that for completeness data from other figures have been re-presented here.

FIGS. 7a to 7c are graphs depicting timing and severity of gene expression changes in models of RTT parallels that of symptoms. FIG. 7a, Mean fold-change in gene expression versus gene length in the hippocampus of MeCP2 KO mice compared to wild type at four and nine weeks of age reveals increasing severity of length-dependent gene misregulation that parallels the onset of RTT-like symptoms in these mice⁸. FIG. 7b, Mean fold-change in gene expression versus gene length in hippocampal tissue of mice expressing truncated forms of MeCP2 that mimic human disease-causing alleles at four weeks of age. Re-expression of a longer truncated form of MeCP2 (G273X) in the MeCP2 KO normalizes expression of long genes more effectively than does expression of a shorter truncation of MeCP2 (R270X). This difference parallels the higher degree of phenotypic rescue observed in MeCP2 G273X-expressing mice compared to MeCP2 R270X-expressing mice⁸. FIG. 7c, Mean fold-change in gene expression versus gene length in hippocampal tissue of mice expressing truncated forms of MeCP2 at nine weeks of age. Consistent with the eventual onset of symptoms of these mouse strains, length-dependent gene misregulation is evident in both strains. Note that for completeness the same data for the MeCP2 KO is re-plotted across several panels.

FIGS. 8a to 8b are graphs depicting MeCP2 has high affinity for mCH in electrophoretic mobility shift assays. Recombinant MeCP2 protein containing the DNA-binding domain of MeCP2 (amino acids 81-170) was bound to ³²P-end-labeled oligonucleotides containing either a methylated cytosine in a CA context (FIG. 8a) or a CG context (FIG. 8b) and was exposed to increasing amounts of unlabeled competitor containing unmethylated, methylated, or hydroxymethylated cytosine in a CG or CA context. Full gels showing shifted and unshifted probe are displayed on the right, close-up views of the shifted bands are shown at the left. A mCA-containing oligonucleotide competes for MeCP2 binding with equal or higher efficacy to that of a symmetrically-methylated CG oligonucleotide. In contrast, hmCG-containing probes compete with similar efficacy to that of an unmethylated probe, while a hmCA-containing probe competes with high efficacy. The difference in affinity of MeCP2 for hmCA- and hmCG-containing probes may explain apparently incongruent results published on the affinity of MeCP2 for hydroxymethylated DNA^{13,26,27,28} (see Example 1).

FIGS. 9a to 9h are graphs depicting genomic analysis of mCG and hmCG in length-dependent gene regulation by MeCP2. FIG. 9a-FIG. 9c, Mean methylation of CG dinucleotides (mCG/CG) within gene bodies (transcription start site +3 kb, up to transcription termination site) in the cortex (FIG. 9a), hippocampus (FIG. 9b) and cerebellum (FIG. 9c) for genes binned according to length. FIG. 9d-FIG. 9f, Mean fold-change in gene expression in MeCP2 KO compared to wild type in the cortex (FIG. 9d), hippocampus (FIG. 9e), and cerebellum (FIG. 9f) for genes binned according to mCG levels (mCG/CG) within gene bodies. FIG. 9g, Mean hmCG levels (hmCG/CG) within gene bodies in the cortex for genes binned according to length. FIG. 9h, Mean fold-change in gene expression in MeCP2 KO compared to wild type in the cortex for genes binned according to hmCG levels (hmCG/CG) within gene bodies. In all panels, mean values for each bin are indicated as a line, and ribbon depicts SE for genes within each bin.

FIGS. 10a to 101 are graphs depicting genomic analysis of mCH in length-dependent gene regulation by MeCP2. FIG. 10a-FIG. 10c, Mean methylation at CH dinucleotides (mCH/CH) within gene bodies (transcription start site +3 kb, up to transcription termination site) in cortex (FIG. 10a), hippocampus (FIG. 10b), and cerebellum (FIG. 10c) for genes binned according to length. FIG. 10d-FIG. 10f, Mean changes in gene expression in cortex (FIG. 10d), hippocampus (FIG. 10e), and cerebellum (FIG. 10f) of MeCP2 KO compared to wild type mice for high mCH genes (top 25% mean gene body mCH/CH) and low mCH genes (bottom 66% mean gene body mCH/CH) binned according to length. FIG. 10g-FIG. 10i, Mean changes in gene expression in cortex (FIG. 10g), hippocampus (FIG. 10h), and cerebellum (FIG. 10i) of MeCP2 KO mice compared to wild type for genes binned according to mean gene body mCH/CH. FIG. 10j-FIG. 10l, Mean changes in gene expression in cortex (FIG. 10j), hippocampus (FIG. 10k), and cerebellum (FIG. 10l) of MeCP2 KO mice compared to wild type for long genes (top 25%) and short genes (bottom 25%) in each brain region binned according to mean gene body mCH/CH. A correlation between fold-change and mCH/CH is not observed in the hippocampus or cerebellum of the MeCP2 KO when all genes are analyzed together (FIG. 10h, FIG. 10i), but it is clearly present amongst the longest genes in the genome when analyzed alone (FIG. 10k, FIG. 10l). Inspection of average levels of mCH measured for all genes in the hippocampus and cerebellum indicates that they are lower than in the cortex (compare y-axis in FIG. 10a, FIG. 10b and FIG. 10c). This may explain why, in these brain regions, a correlation across all genes is not detected, while in long genes, where there is more mCH on average and the cumulative effect of mCH across the gene may be larger, a correlation is detected. In all panels, mean values for each bin are indicated as a line, ribbon depicts SE. Note that, for completeness, data from analysis of the cortex presented in FIG. 3 are re-presented here.

FIG. 11 is a graph depicting quantitative RT-PCR analysis of gene expression in the visual cortex of MeCP2 KO and MeCP2 R306C mice confirms up-regulation of long genes in this brain region. The expression of eighteen long genes (>100 kb) consistently misregulated across multiple brain regions in MeCP2 mutant mice (up-regulated across five brain regions in MeCP2 KO mice, and down-regulated across three brain regions in MeCP2 OE mice, see methods) was assessed by quantitative RT-PCR in the visual cortex of MeCP2 KO (n=4 WT, 6 KO) and MeCP2 R306C mice (n=4 WT, 4 R306C). A statistically significant number of genes show increased expression in the cortex of both MeCP2 KO (p<1×10⁻¹⁵) and MeCP2 R306C (p=1.69×10⁻⁶) mice compared to their respective wild-type littermate controls (Hotelling T² test for small sample size⁴⁰)

FIGS. 12a to 12d are graphs depicting that misregulation of long genes with brain-specific function in RTT, FXS and other ASDs. FIG. 12a, Cumulative distribution function (CDF) of gene lengths plotted exclusively for genes that are among the top 60% of expression levels in the brain (see Example 1). The extreme length of MeCP2-repressed genes, SFARI autism candidate genes (http://sfari.org/), and genes encoding FMRP target mRNAs³¹ compared to all genes, even when controlling for expression, indicates that the long length of these gene sets is not due to the high expression of long genes in the brain (p<1×10⁻¹⁵ for each geneset vs all expressed genes; 2-sample Kolmogorov-Smirnov (KS) test). FIG. 12b, The CDF of gene lengths for all genes compared to a second, independent set of FMRP targets identified by Brown and colleagues³² confirms the extreme length of genes encoding putative FMRP targets (p<1×10⁻¹⁵, KS-test). FIG. 12c, CDF of gene lengths exclusively for genes that are expressed at comparable levels in the brain and other somatic tissues (see Example 1). The extreme length of each gene set compared to all genes (p<1×10⁻¹⁵ for all datasets, KS-test), when filtering for genes that are expressed equivalently in all tissues, indicates that the regulation of long genes by MeCP2 and FMRP occurs independently of brain-specific expression. FIG. 12d, The CDF of mature mRNA lengths for MeCP2-repressed genes, FMRP target genes and SFARI autism candidates reveals that the mature transcripts derived from these genes are significantly longer than the transcriptome average (p<1×10⁻¹¹ for each geneset vs all genes, KS-test).

FIG. 13 is a Table showing gene ontology analysis of MeCP2-repressed genes and genes >100 kb Functional annotation clustering analysis of genes identified as MeCP2-repressed (see methods of Example 1, FIG. 5) and the longest genes in the genome (>100 kb) was performed using the David bioinformatics resource (David v6.7³⁹). The top fifteen enriched gene ontology terms with p<0.01 (Benjamini multiple testing correction) are listed for “Biological Process”, “Cellular Component”, and “Molecular Function” respectively.

FIG. 14 is Table listing primers for quantitative RT-PCR analysis.

FIG. 15 is a Table listing 466 MeCP2-repressed genes by gene name and gene ID, whose expression is robustly up-regulated in the absence of MeCP2 and down-regulated when MeCP2 is over-expressed.

FIGS. 16a to 16b are schematics and graphs. FIG. 16a, Boxplots of MeCP2 ChIP-seq read density within genes >100 kb plotted by quartile of mCA/CA in the cortex and cerebellum. FIG. 16d, Bar plots of the mean fold-change in expression for all genes >100 kb compared to subsets of genes >100 kb containing low mCA (bottom 50% mCA/CA) or high mCA (top 25% mCA/CA) within their gene body. Values shown for mice with the indicated Mecp2 genotypes (left) and human RTT brain (right). CTX, Cortex; HC, Hippocampus; CB, cerebellum; KO, MeCP2 Knockout; OE, MeCP2 overexpression; R306C, MeCP2 arginine 306 to cysteine missense mutation; ***, p<1×10⁻¹⁰, **, p<1×10⁻³; *, p<0.01; two-tailed t-test, Bonferroni correction. Error bars represent S.E.M. See FIG. 21 for sample size and other details.

FIGS. 17a to 17d are schematics and gels showing conditional knockout of Dnmt3a in vivo. FIG. 17a, Diagram of the Dnmt3a locus and Cre-dependent conditional knockout strategy for Dnmt3a²⁶. LoxP sites (green triangles) flank exon 17, which is removed following Cre-mediated recombination. Primers (purple arrows) were designed to flank exons 17 and 18. The wild-type (WT), floxed (FLX), and knockout (KO) allele are depicted. FIG. 17b, Representative PCR genotyping for tail DNA samples indicates presence or absence of the floxed (flx, ˜800 bp), wild-type (WT, ˜750 bp), and knockout (KO, ˜500 bp) alleles. Separate genotyping reaction for the Nestin-cre transgene (˜250 bp) is shown. FIG. 17c, Efficient excision of the floxed exon is detected in cerebellar DNA from conditional knockout (Dnmt3a^{flx/flx}; Nestin-Cre^{+/-}, Dnmt3a cKO) mice but not from and control animals (Dnmt3a^{flx/flx}, Control). FIG. 17d, Western blot analysis of Dnmt3a, MeCP2, and Gapdh (loading control) protein from the cerebellum of control and Dnmt3a cKO adult mice.

FIGS. 18a to 18d are box plots and graphs showing ChIP-seq analysis of MeCP2 binding in vivo. FIG. 18a, Boxplots of input-normalized read density within gene bodies (TSS+3 kb to TTS) for MeCP2 ChIP from the mouse frontal cortex plotted for genes according to quartile of mCA/CA, mCG/CG, hmCA/CA and hmCG/CG in the frontal cortex²⁴ for all genes and genes >100 kb. FIG. 18b, Similar analysis of MeCP2 ChIP from the mouse cortex (left) or cerebellum (right) plotted for genes according to quartile of mCA/CA or mCG/CG for all genes and genes >100 kb. MeCP2 ChIP-signal is correlated with mCA/CA levels from the frontal cortex, cortex, and cerebellum for all genes and this correlation is more prominent among genes >100 kb. mCG does not show as prominent a correlation with MeCP2 ChIP signal, and hmCG trends toward anti-correlation with MeCP2 ChIP. These results suggest that MeCP2 has a lower affinity for hmCG than mCG, suggesting that, in vivo, hmCG is associated with reduced MeCP2 occupancy (Supplementary Discussion). FIG. 18c, High resolution analysis of high-coverage bisulfite sequencing data from the frontal cortex showing a correlation between MeCP2 ChIP signal and mCA. Input-normalized ChIP signal plotted for mCA levels for 500 bp bins tiled across all genes. FIG. 18d, Aggregate plots of MeCP2 input-normalized ChIP signal (top) and relative methylation (log 2 enrichment in mC as compared to the flanking regions) for mCA, mCC, mCT, and mCG (bottom) are plotted around the 31,479 summits of MeCP2 ChIP enrichment identified using the MACS peak-calling algorithm (red) or 31,479 randomly selected control sites (gray, see Methods and Feng, J., Liu, T. & Zhang, Y. Using MACS to identify peaks from ChIP-Seq data. Current protocols in bioinformatics/editoral board, Andreas D. Baxevanis et al. Chapter 2, Unit 2 14, (2011).

FIGS. 19a to 19i are graphs depicting analysis of MeCP2 expressed genes and FMRP target genes. FIG. 19a, Mean fold-change in mRNA expression for examples of MeCP2-repressed genes across three different Mecp2 mutant genotypes (KO, OE, and R306C) and six brain regions. p-values for each gene are derived from the mean z-scores for fold-change across all datasets (see Methods of Examples). FIG. 19b, Gene expression and CA methylation data from the cerebellum for selected MeCP2-repressed genes from a (right), as well as examples of extremely long genes (>100 kb) that are not enriched for mCA and are not misregulated (left). Fold-changes in mRNA expression in Mecp2 mutants and the Dnmt3a cKO are shown (left axis), as well as mean mCA levels (gray; right axis). Red line indicates genomic median for gene body mCA/CA FIG. 19c, Boxplots of mCA levels in MeCP2-repressed genes compared to all genes. FIG. 19d, Mean fold-change for MeCP2-repressed genes in eight “training datasets” used to define these genes (see Methods), and nine “test datasets”: three Mecp2 mutant datasets not used to define MeCP2-repressed genes (CTX MeCP2 KO and CB MeCP2 R306C, generated in this study; HC MeCP2 KO 4wk, analyzed from Baker et al.⁸), and six datasets from brains of mouse models of neurological dysfunction generated using the same microarray platforms as the MeCP2 datasets (Geo accession # in order: GSE22115, GSE27088, GSE43051, GSE47706, GSE44855, GSE52584). Error bars are SEM of MeCP2-repressed gene expression across samples (n=4-8 microarrays per genotype per dataset); ** p<0.01, one-tailed t-test, Benjamini-Hochberg correction. Note that significance testing was not performed on training datasets. Brain regions indicated as in FIG. 1, (WB, whole brain). FIG. 19e, Cumulative distribution function (CDF) of gene lengths plotted exclusively for genes that are among the top 60% of expression levels in the brain (Supplementary Discussion). The extreme length of MeCP2-repressed genes and genes encoding FMRP target mRNAs³¹ when controlling for expression level indicates that the long length of these genesets is not a secondary effect of the preferential expression of long genes in the brain (p<1×10⁻¹⁵ for each geneset versus all expressed genes; 2-sample Kolmogorov-Smirnov (KS) test). FIG. 19f, The CDF of gene lengths for all genes compared to an independent set of FMRP targets identified by Brown and colleagues⁴⁵ (p<1×10⁻¹⁵, KS-test). FIG. 19g, CDF of gene lengths for genes expressed at similar levels in the brain and other somatic tissues (Example 2). The extreme length of each geneset (p<1×10⁻¹⁵, KS-test) when filtering for genes that are expressed in all tissues indicates that regulation of long genes by MeCP2 and FMRP is not dependent on brain-specific expression. FIG. 19h, CDF of mature mRNA lengths for MeCP2-repressed genes, and FMRP target genes (p<1×10⁻¹¹ for each geneset versus all genes, KS-test). FIG. 19i, Overlap of MeCP2-repressed genes and putative FMRP target mRNAs²⁹ (p<5×10⁻⁵, hypergeometric test). Expected overlap was calculated by dividing the expected overlap genome-wide (hypergeometric distribution) according to the distribution of all gene lengths in the genome. See Methods and FIG. 21.

FIGS. 20a to 20d are graphs and gels showing the consequences of long gene misregulation in neurons. FIG. 20a, Mean expression of genes binned according to length in human neural and non-neural tissues. Mean expression for genes within each bin (200 gene bins, 40 gene step) is indicated by the line; ribbon represents the S.E.M. of genes within each bin. FIG. 20b, Western blot analysis of MeCP2 from primary cortical neurons after control or MeCP2 shRNA knockdown (KD) and treatment with DMSO vehicle (−) or topotecan (+). FIG. 20c, Heatmap summary of nCounter analysis for the expression of selected MeCP2-repressed (MR) genes from primary neurons treated with control or MeCP2 shRNA and topotecan (n=3-4). Normalized log 2 fold-change relative to the DMSO-treated, control KD is shown. MeCP2 KD conditions are significantly different from control, (p=1e-4, repeated measures ANOVA across 8 genes). Newman-Keuls corrected, post-hoc comparisons: p<0.05 control KD, 0 nM drug versus MeCP2 KD, 0 nM drug; p>0.05, control KD, 0 nM drug versus MeCP2 KD, 50 nM drug; p<0.05 MeCP2 KD, 0 nM drug versus MeCP2 KD, 50 nM drug. FIG. 20d, Bioanalyzer profiles of 18S and 28S ribosomal RNA (top) and total RNA quantification (bottom) for treated neurons (n=3-5). Total RNA values normalized to DMSO-treated control KD, red dashed line. Two-way repeated measures ANOVA indicates a significant effect of KD (p<0.01) and drug treatment (p<0.05). Rescue assessed by one-tailed t-test, Bonferroni multiple testing correction, * p<0.05.

FIG. 21 is a Table of gene ontology analysis of MeCP2-repressed genes and genes >100 kb. Functional annotation clustering analysis of genes identified as MeCP2-repressed and the longest genes in the genome (>100 kb) was performed using the David bioinformatics resource (David v6.7)³⁹. The top fifteen enriched gene ontology terms with p<0.01 (Benjamini multiple testing correction) are listed for “Biological Process”, “Cellular Component”, and “Molecular Function”, respectively.

FIGS. 22a to 22b are graphs showing disruption of Dnmt3a in the brain leads to length-dependent up-regulation of genes containing high levels of mCA. FIG. 22a, Summary of genome-wide bisulfite-sequencing analysis of mCN (where N=G, A, T, or C) in control and Dnmt3a cKO cerebella (n=2 per genotype). Dashed line represents mean background non-conversion rate of the bisulfite-seq assay (see Methods). FIG. 22b, Mean fold-change in gene expression versus gene-body mCA for MeCP2 KO (left) or Dnmt3a cKO (right) cerebella. Long (top 25%, >60 kb) and short (bottom 25%, <14.9 kb) genes were binned according to gene-body mCA/CA levels. Lines represent mean fold-change in expression for each bin (200 gene bins, 40 gene step), and the ribbon is S.E.M. of genes within each bin. ***, p<0.005; two-tailed t-test, Bonferroni correction. Error bars represent S.E.M.

FIGS. 23a to 23d are graphs showing the timing and severity of gene expression changes in models of RTT. FIG. 23a, Mean fold-change in gene expression versus gene length in the hippocampus of MeCP2 KO mice compared to wild type at four and nine weeks of age reveals increasing magnitude of length-dependent gene misregulation that parallels the onset of RTT-like symptoms in these animals⁸. FIG. 23b, Mean fold-change in gene expression versus gene length in hippocampus of mice expressing truncated forms of MeCP2 mimicking human disease-causing alleles at four weeks of age. Re-expression of a longer truncated form of MeCP2 (G273X) in the MeCP2 KO normalizes expression of long genes more effectively than expression of a shorter truncation of MeCP2 (R270X), and parallels the higher degree of phenotypic rescue observed in MeCP2 G273X-expressing mice compared to MeCP2 R270X-expressing mice⁸. FIG. 23c, Mean fold-change in gene expression versus gene length in hippocampus of mice expressing truncated MeCP2 at nine weeks of age. Consistent with the eventual onset of symptoms of these mouse strains, length-dependent gene misregulation is evident in both strains. FIG. 23d, Changes in gene expression for genes binned by length in human MECP2 null ES cells differentiated into neural progenitor cells, neurons cultured for 2 weeks, or neurons cultured for 4 weeks¹⁹. In all plots, lines represent mean fold-change in expression for each bin (200 gene bins, 40 gene step), and the ribbon is S.E.M. of genes within each bin.

FIG. 24 is a graph behavior score versus days after implant in MeCP2 hemizygous mice, where the implant contains either vehicle (control: 50 mM tartaric acid) or Topotecan (25 μM).

FIG. 25 is a graph of percent survival versus days elapsed after treatment in MeCP2 hemizygous mice with an implant contains either vehicle (control: 50 mM tartaric acid) or Topotecan (25 μM).

### DETAILED DESCRIPTION OF THE INVENTION

Our data provides direct evidence that mutations in MeCP2 and other established autism genes cause neurological dysfunction by disrupting the expression of long genes in the brain. Accordingly, methods of treating autism spectrum disorders are provided that comprise administering an effective amount of an agent that modulates long gene expression in the brain.

As used herein the term “long gene” refers to a gene of greater than 100 kb, whose expression is either normally suppressed or up-regulated regulated within the brain of a healthy individual.

As used herein the term “modulate” refers to down regulation (inhibition/repression of expression) or up regulation (increased expression/removal of repression) of gene expression. Expression of a gene can be modulated by affecting transcription, translation, or post-translational processing. In one embodiment, a compound that modulates expression of a long gene, modulates transcription from the gene by either up-regulating or down-regulating transcription of a gene. In another embodiment, a compound that modulates expression of a long gene modulates mRNA translation of mRNA that is transcribed from the gene by either up-regulating or down-regulating translation. In still another embodiment, a compound that modulates expression of a long gene modulates post-translational modification of the protein encoded by the gene, for example to result in degradation of protein encoded by the gene or non-degradation of protein encoded by the gene, e.g. an agent the affects ubiquitin modification of a long gene protein.

To down regulate expression is to inhibit expression by at least 5%, at least 10%, at least 20%, at least 30%, at least 40%, at least 50%, at least 60%, at least 70%, at least 80%, at least 90%, at least 95%, at least 98%, or 100% (e.g. complete loss of expression) relative to an uninhibited control, e.g. a control not treated with the compound. To up-regulate expression is to increase expression by at least 5%, at least 10%, at least 20%, at least 30%, at least 40%, at least 50%, at least 60%, at least 70%, at least 80%, at least 90%, at least 95%, at least 98%, or 100% relative to a control not treated with an the compound. Expression can be measured, for example, by measuring the level of mRNA transcript, by measuring the level of encoded protein, or by monitoring post translational modification, e.g. by Western analysis quantitated by densitometry or by mass spectrometry. The effect of a compound on expression can also be monitored using in vitro reporter assays, for example by utilizing a vector or cell line comprising gene regulatory elements (e.g. promoter) operably linked to the gene and/or a measurable reporter gene, e.g. fluorescent reporter.

Agents that Modulate Expression of Long Genes

As used herein, the terms “compound” or “agent” are used interchangeably and refer to molecules and/or compositions that modulate expression of a long gene in the brain.

The compounds/agents include, but are not limited to, chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; nucleic acids; nucleic acid analogs and derivatives; extracts made from biological materials such as bacteria, plants, fungi, or animal cells or tissues; naturally occurring or synthetic compositions; peptides; aptamers; and antibodies, or fragments thereof.

A compound/agent can be a nucleic acid RNA or DNA, and can be either single or double stranded. Example nucleic acid compounds include, but are not limited to, a nucleic acid encoding a protein activator or inhibitor (e.g. transcriptional activators or inhibitors), oligonucleotides, nucleic acid analogues (e.g. peptide-nucleic acid (PNA), pseudo-complementary PNA (pc-PNA), locked nucleic acid (LNA) etc.), antisense molecules, ribozymes, small inhibitory or activating nucleic acid sequences (e.g. RNAi, shRNAi, siRNA, micro RNAi (mRNAi), antisense oligonucleotides etc.) A protein and/or peptide agent can be any protein that modulates gene expression or protein activity. Non-limiting examples include mutated proteins; therapeutic proteins and truncated proteins, e.g. wherein the protein is normally absent or expressed at lower levels in the target cell. Proteins can also be selected from genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. A compound or agent that increases expression of a gene or increases the activity of a protein encoded by a gene is also known as an activator or activating compound. A compound or agent that decreases expression of a gene or decreases the activity of a protein encoded by a gene is also known as an inhibitor or inhibiting compound.

The terms “polypeptide,” “peptide” and “protein” refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acids.

There are agents already developed and known in the art that modulate long gene expression. For example, topoisomerase is known to facilitate transcription of long genes, and topoisomerase inhibitors have been indicated to reduce expression of long gene in neurons' See for example King et al.¹². Interestingly, King et al. indicates that mutations in topoisomerase and chemicals that inhibit topoisomerases lead to down-regulation of long genes in neurons, and further indicate that this phenomenon is responsible for autism spectrum disorders and other neurodevelopmental disorders. For example, King et al. indicates that length-dependent impairment of gene transcription in neurons during critical periods of brain development, may be the unifying cause of pathology in individuals with autism spectrum disorders and other neurodevelopment disorders. However, this is in direct contrast to our discovery that, in fact, an increase in long gene expression in the brain is responsible for the neuropathology of autism spectrum disorders, including for example, Rett syndrome and Fragile X syndrome. Accordingly, compounds that down-regulate long gene expression in the brain are useful in methods of the invention for the treatment of autism spectrum disorders. In one embodiment, the autism spectrum disorder is Rett syndrome and the agent decreases the expression of long genes in the brain. In another embodiment, the autism spectrum disorder is Fragile X syndrome and the agent decreases the expression of long genes in the brain. In addition, in certain embodiments, the autism spectrum disorder is MeCP2 duplication syndrome or an autism spectrum disorder caused by a mutation in topoisomerase and the agent increases the expression of long genes in the brain.

In certain embodiments of the instant invention, the agent used to treat autism spectrum disorders is administered chronically, i.e. for the life of the patient.

In certain embodiments of the instant invention, the agent used in methods of the invention that down-regulates expression of long genes in the brain is not an inhibitor of topoisomerase 1. Inhibitors of topoisomerase are known in the art and include, for example, inhibitors of topoisomerase I or topoisomerase II. Topoisomerase I inhibitors include e.g. camptothecin derivatives such as Belotecan (CKD602), Camptothecin, 7-Ethyl-10-Hydroxy-CPT, 10-Hydroxy-CPT, Rubitecan (9-Nitro-CPT), 7-Ethyl-CPT, Topotecan, Irinotecan, Silatecan (DB67) and indenoisoquinoline derivatives, such as NSC706744, NSC725776, NSC724998 (See for example US 2013/0317018 for chemical structures, incorporated herein by reference in its entirety).

Thus, in certain embodiments, the agent for treatment of the autism spectrum disorder (e.g. Rett Syndrome, or Fragile X syndrome) is not a camptothecin derivative. In certain embodiments, the agent for treatment of the autism spectrum disorder (e.g. Rett Syndrome, or Fragile X syndrome) is not Belotecan (CKD602). In certain embodiments, the agent for treatment of the autism spectrum disorder (e.g. Rett Syndrome, or Fragile X syndrome) is not Camptothecin. In certain embodiments, the agent for treatment of the autism spectrum disorder (e.g. Rett Syndrome, or Fragile X syndrome) is not 7-Ethyl-10-Hydroxy-CPT. In certain embodiments, the agent for treatment of the autism spectrum disorder (e.g. Rett Syndrome, or Fragile X syndrome) is not 10-Hydroxy-CPT. In certain embodiments, the agent for treatment of the autism spectrum disorder (e.g. Rett Syndrome, or Fragile X syndrome) is not Rubitecan (9-Nitro-CPT), 7-Ethyl-CPT. In certain embodiments, the agent for treatment of the autism spectrum disorder (e.g. Rett Syndrome, or Fragile X syndrome) is not Topotecan. In certain embodiments, the agent for treatment of the autism spectrum disorder (e.g. Rett Syndrome, or Fragile X syndrome) is not Irinotecan. In certain embodiments, the agent for treatment of the autism spectrum disorder (e.g. Rett Syndrome, or Fragile X syndrome) is not Silatecan (DB67). In certain embodiments, the agent for treatment of the autism spectrum disorder (e.g. Rett Syndrome, or Fragile X syndrome) is not indenoisoquinoline.

In certain embodiments, the agent used in methods of the invention that down-regulates expression of long genes in the brain is not an inhibitor of topoisomerase II. Topoisomerase II inhibitors include, for example, Doxorubicin; Etoposide; acridine derivatives, such as Amsacrine; and podophyllotoxin derivatives, such as etoposide; and bisdioxopiperazine derivatives, such as ICRF-193, dexrazoxane (ICRF-187) (See for example US 2013/0317018 for chemical structures, incorporated herein by reference in its entirety). Other topoisomerase inhibitors include, Resveratrol (PMID: 20304553; PMID: 15796584), Epigallocatechin gallate (PMID: 18293940; PMID: 11594758; PMID: 11558576; PMID: 1313232) Genistein (PMID: 17458941), Daidzein (PMID: 17458941). Quercetin (PMID: 1313232; PMID: 16950806; PMID: 15312049), natural flavones related to quercetin that inhibit topoisomerase, such as acacetin, apigenin, kaempferol and morin (PMID: 8567688), Luteolin (PMID: 12027807; PMID: 16950806; PMID: 15312049); and Myricetin (PMID: 20025993).

Thus, in certain embodiments, the agent for treatment of the autism spectrum disorder (e.g. Rett Syndrome, or Fragile X syndrome) is not Doxorubicin. In certain embodiments, the agent for treatment of the autism spectrum disorder (e.g. Rett Syndrome, or Fragile X syndrome) is not Etoposide. In certain embodiments, the agent for treatment of the autism spectrum disorder (e.g. Rett Syndrome, or Fragile X syndrome) is not an acridine derivatives or a bisdioxopiperazine derivative. In certain embodiments, the agent for treatment of the autism spectrum disorder (e.g. Rett Syndrome, or Fragile X syndrome) is not Resveratrol (PMID: 20304553; PMID: 15796584). In certain embodiments, the agent for treatment of the autism spectrum disorder (e.g. Rett Syndrome, or Fragile X syndrome) is not Epigallocatechin gallate (PMID: 18293940; PMID: 11594758; PMID: 11558576; PMID: 1313232) Genistein (PMID: 17458941). In certain embodiments, the agent for treatment of the autism spectrum disorder (e.g. Rett Syndrome, or Fragile X syndrome) is not Daidzein (PMID: 17458941). In certain embodiments, the agent for treatment of the autism spectrum disorder (e.g. Rett Syndrome, or Fragile X syndrome) is not Quercetin (PMID: 1313232; PMID: 16950806; PMID: 15312049). In certain embodiments, the agent for treatment of the autism spectrum disorder (e.g. Rett Syndrome, or Fragile X syndrome) is not a natural flavones related to quercetin that inhibits topoisomerase, such as acacetin, apigenin, kaempferol and morin (PMID: 8567688), Luteolin (PMID: 12027807; PMID: 16950806; PMID: 15312049). In certain embodiments, the agent for treatment of the autism spectrum disorder (e.g. Rett Syndrome, or Fragile X syndrome) is not Myricetin (PMID: 20025993).

In some embodiments, the agent that increases expression of long genes in the brain is an activator of topoisomerase. In some embodiments, the agent that increases expression of long genes in the brain is a DNA methyltransferase inhibitor, non-limiting example of a DNA methyltransferase inhibitor include RG108, epigallocatachin-3-gallate, or 5-azacytosine, See for example Stresemann et al., Functional diversity of DNA methyltransferase inhibitors in human cancer cell lines Cancer Res. 2006 Mar. 1; 66(5):2794-800, incorporated by reference.

In one embodiment, the agent that decreases expression of long genes in the brain are small molecules that inhibit transcription of long genes in the brain. For example, in certain embodiments the inhibitor of long gene expression is a topoisomerase inhibitor (e.g. as described above), a nucleotide analog that inhibits transcriptional elongation, a BRD4 inhibitor that inhibits pro-elongation chromatin modifiers, an inhibitor of Dot1 that promotes elongation-associated chromatin modification, Alpha-Amantin, a protein synthesis inhibitor, or a DNA intercalator that blocks RNA polymerases. Such inhibitors are known to those of skill in the art. For example, any nucleotide analog that inhibits transcriptional elongation can be used in methods of the invention, examples include, but are not limited to 6-azauracil (6UA) (Sigma Aldich, Saint Louis Missori, USA) and MPA (mycophenolic acid)) (Sigma Aldich, Saint Louis Missori, USA), See also for example Malagon et al. Genetics. April 2006; 172(4): 2201-2209; and Mason et al. Molecular Cell, Volume 17, Issue 6, 831-840, 18 Mar. 2005, herein incorporated by reference in entirety.

Non-limiting examples of BRD4 inhibitors include (+)-JQ1, IBET762 and IBET151, See for example Helin and Dhanak, Chromatin proteins and modifications as drug targets, Nature, 502, Pages: 480-488 (24 Oct. 2013), for chemical structures.

Dot1 inhibitors are known to those in the art, non-limiting examples include EPZ-5676, See Blood. 2013 August 8; 122(6): 1017-1025. Alpha-Amanitin is described in Chafin et al. The Journal of Biological Chemistry, 270, 19114-19119, Aug. 11, 1995.

Non-limiting examples of DNA intercalators include Actinomycin D, Cisplatin; ET-743 (Trabectedin or Yondalis) (See e.g., Olivier Bensaude, Inhibiting eukaryotic transcription, which compound to choose? How to evaluate its activity? Transcription 2011 May-June; 2(3): 103-108); Triptolide (Bensaude, Transcription 2011 May-June; 2(3): 103-108); and TGT (Yuzenkova et al., Nucleic Acids Res. November 2013; 41(20): 9257-9265).

In certain embodiments, the agent inhibits or activates proteins and complexes involved in translational elongation. In one embodiment, the agent is selected from the group consisting of: Lactimidomycin (Larsen et al. Org. Lett., 2013, 15 (12), pp 2998-3001), eEF1A1 (eukaryotic translation elongation factor 1-alpha 1), Diphthamide (Free Radical Biology and Medicine Volume 67, February 2014, Pages 131-138), Stm1p (Van Dyke et al. Nucleic Acids Res. October 2009; 37(18): 6116-6125), 4EGI1 (a synthetic, biological molecule that inhibits e1F4E-e1F4G complex; Interlandi, Geneen. Focus Magazine. Harvard University. Feb. 9, 2007), Orthoformimysin (Mafioli et al. ACS Chem. Biol., 2013, 8 (9), pp 1939-1946), e1F5A (Saini et al. Nature. May 7, 2009; 459(7243): 118-121), Minocycline (or other tetracyline antibiotics that interferes with ribosomal translocation; Watabe et al, 2012).

### Screening of Agents

In addition, agents can be screened for their ability to modulate long gene expression in the brain.

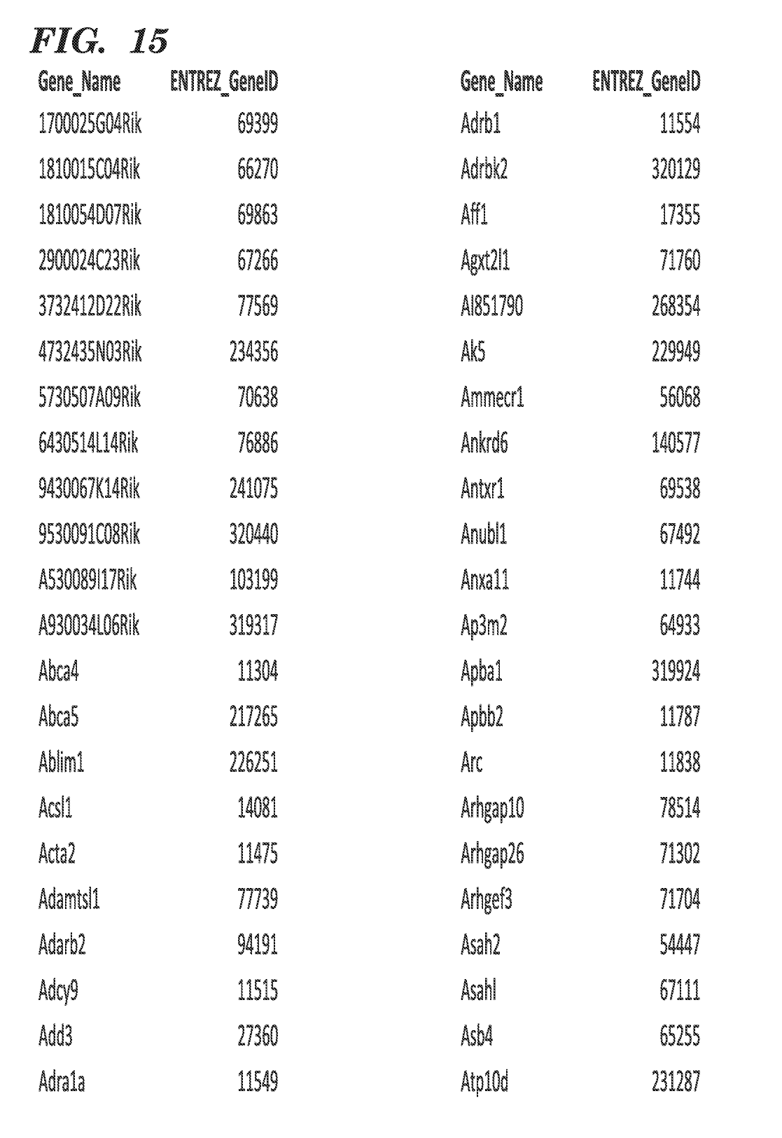

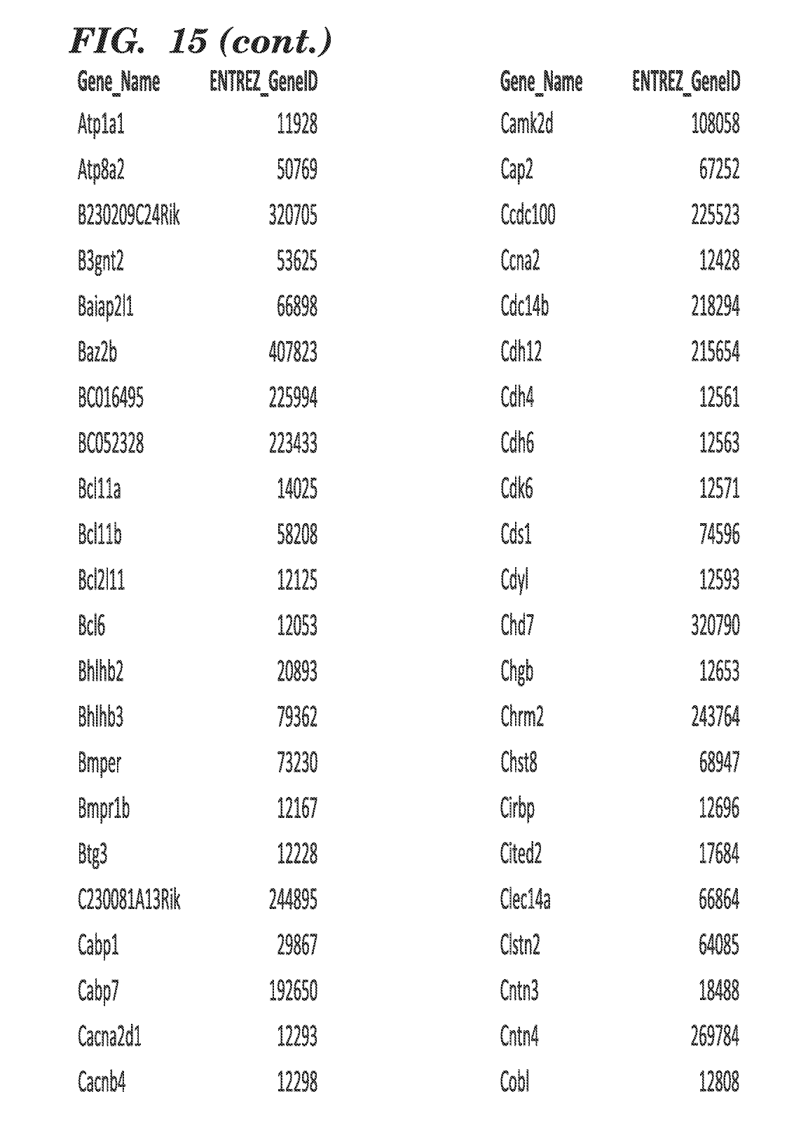

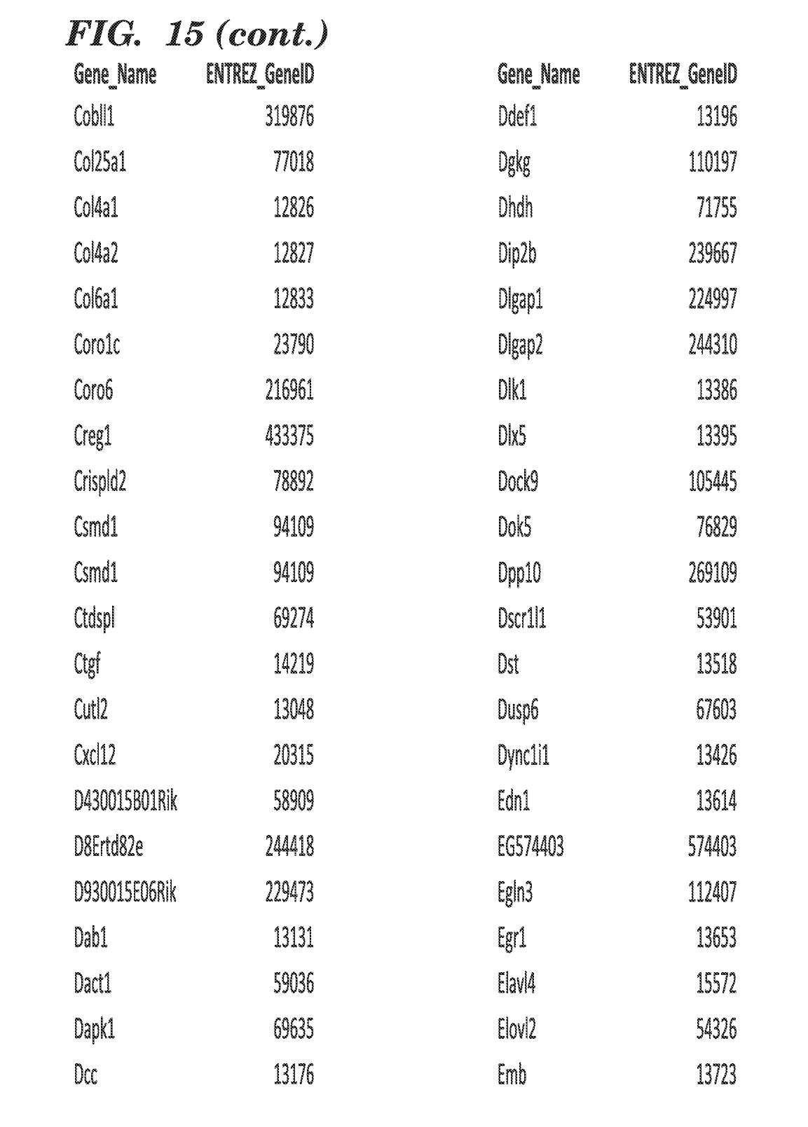

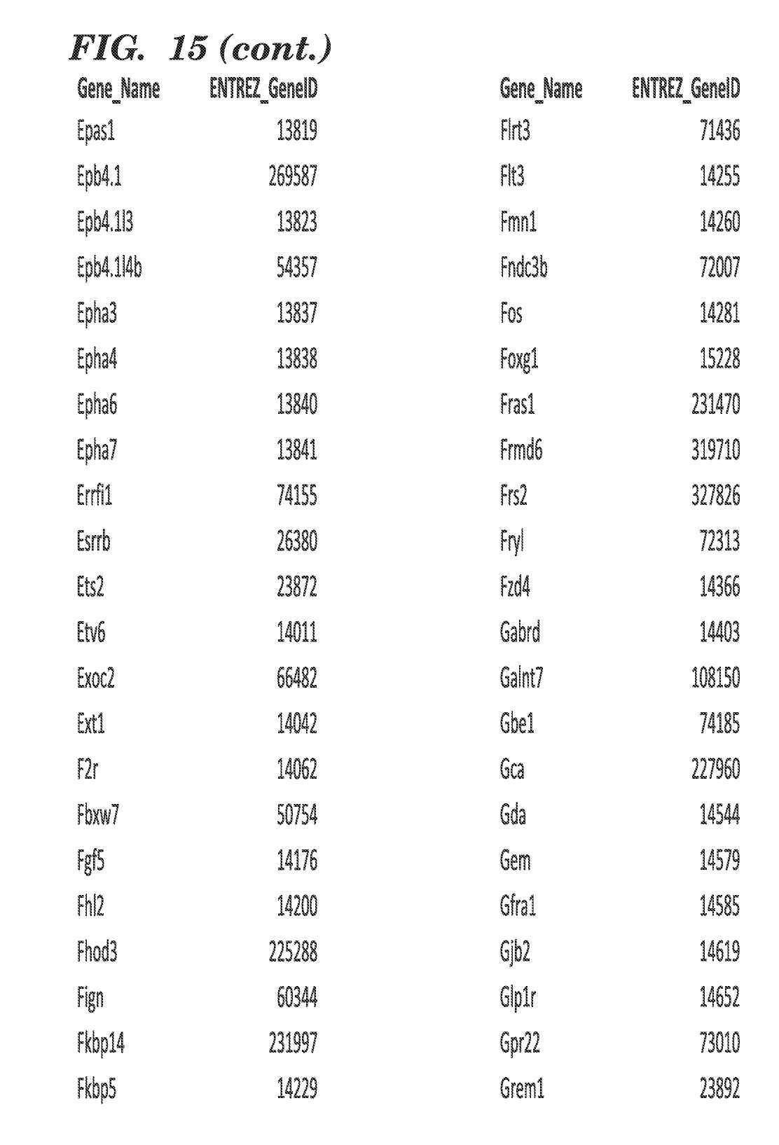

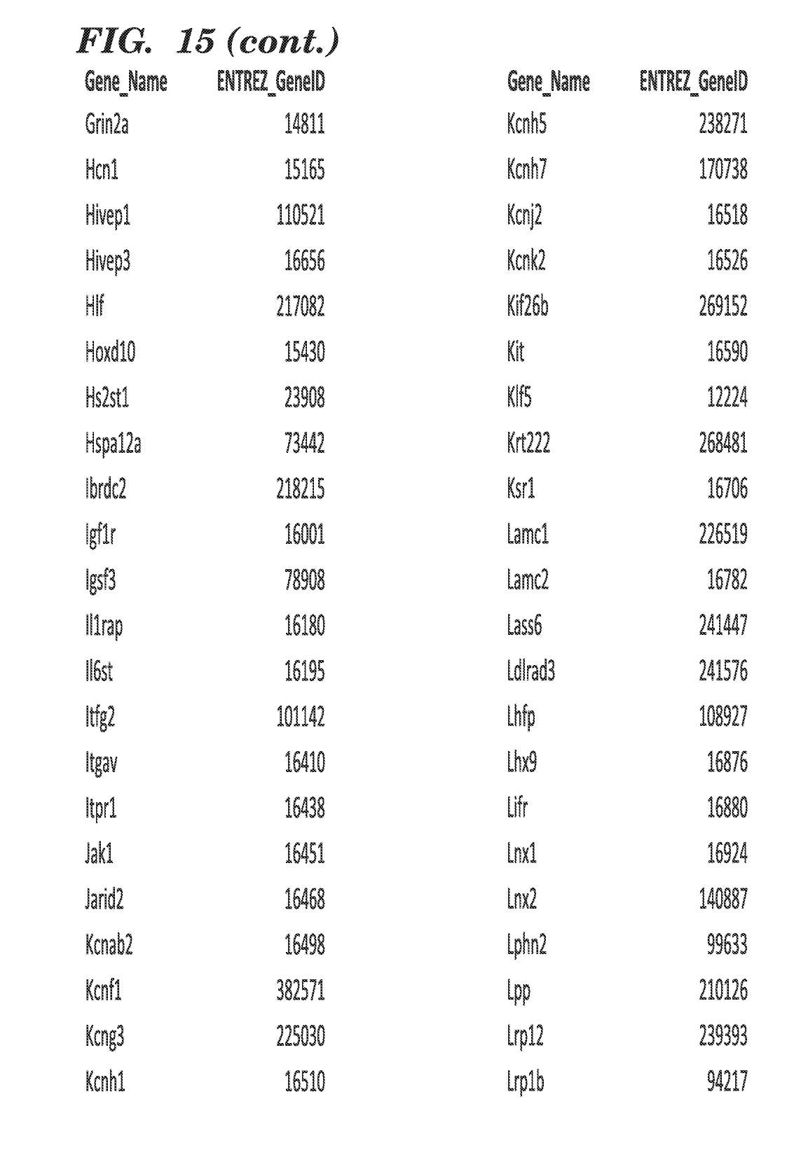

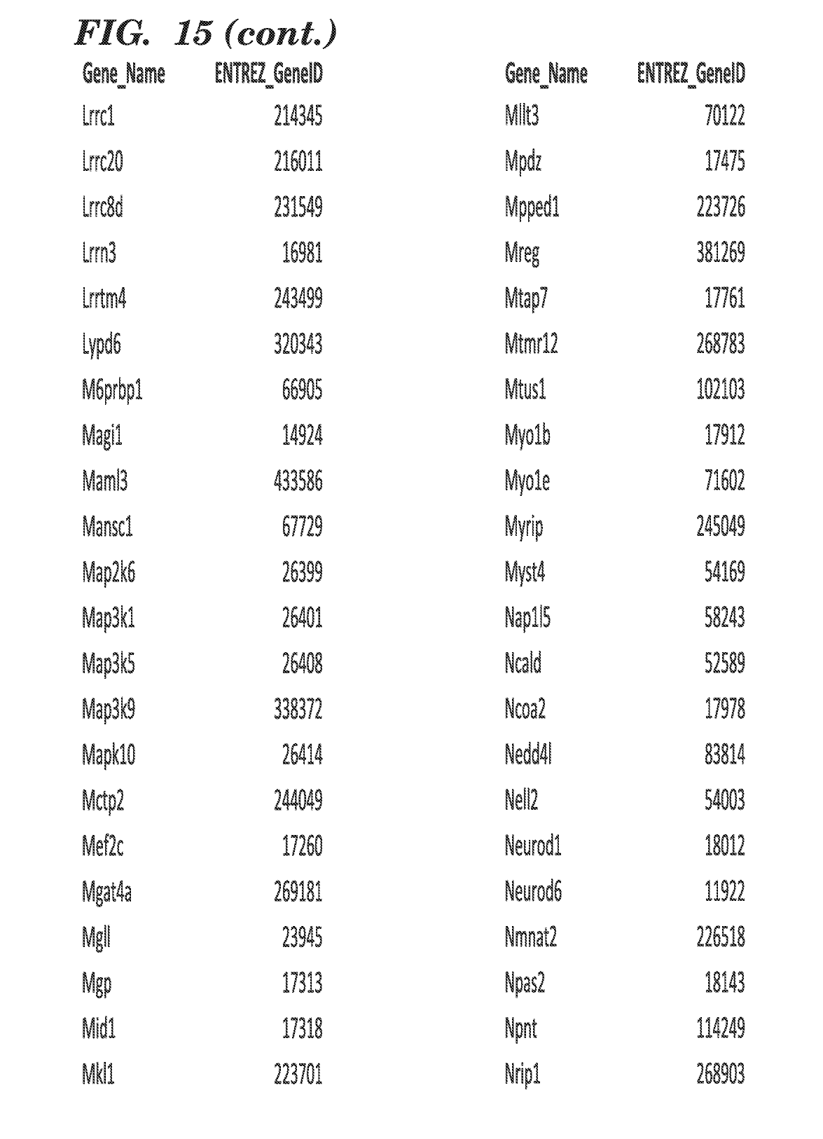

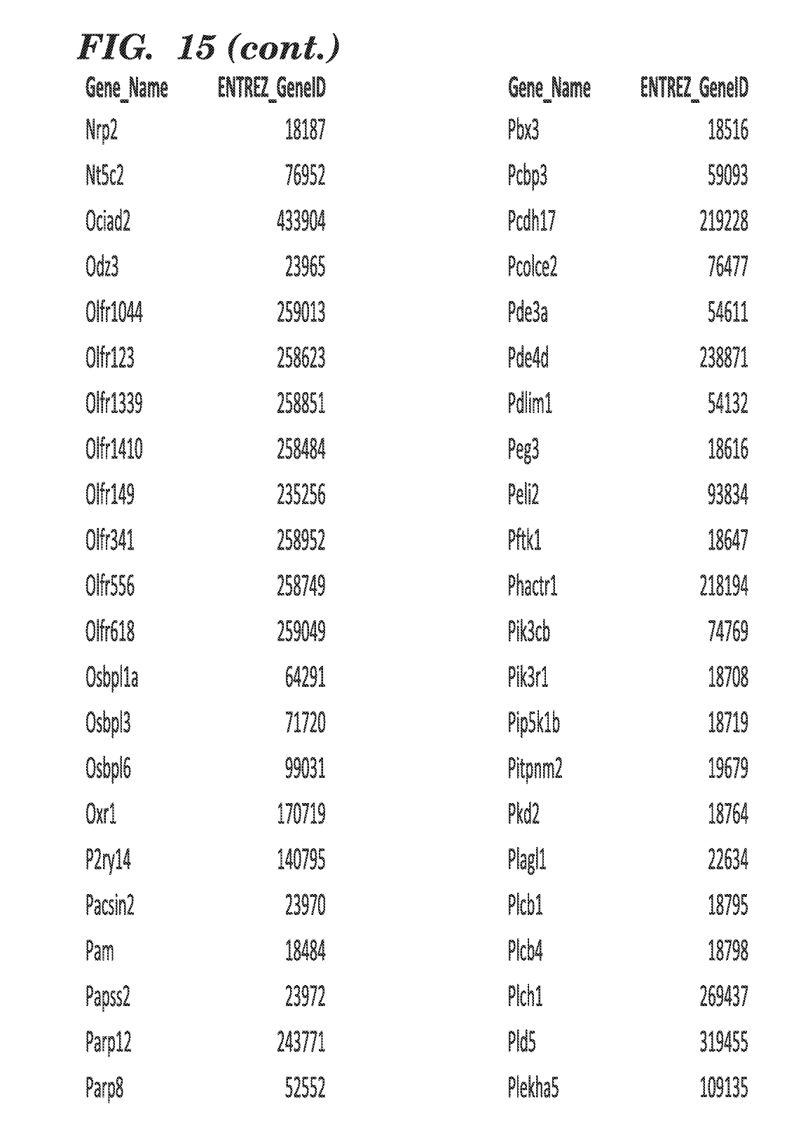

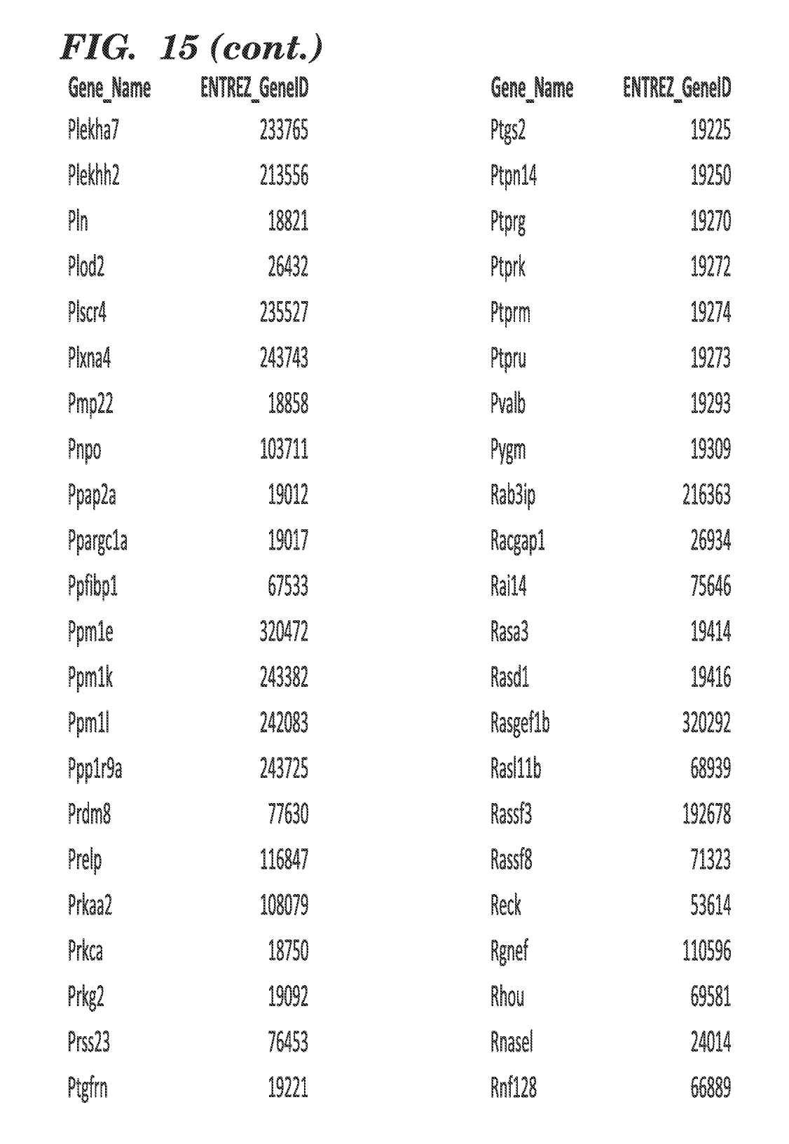

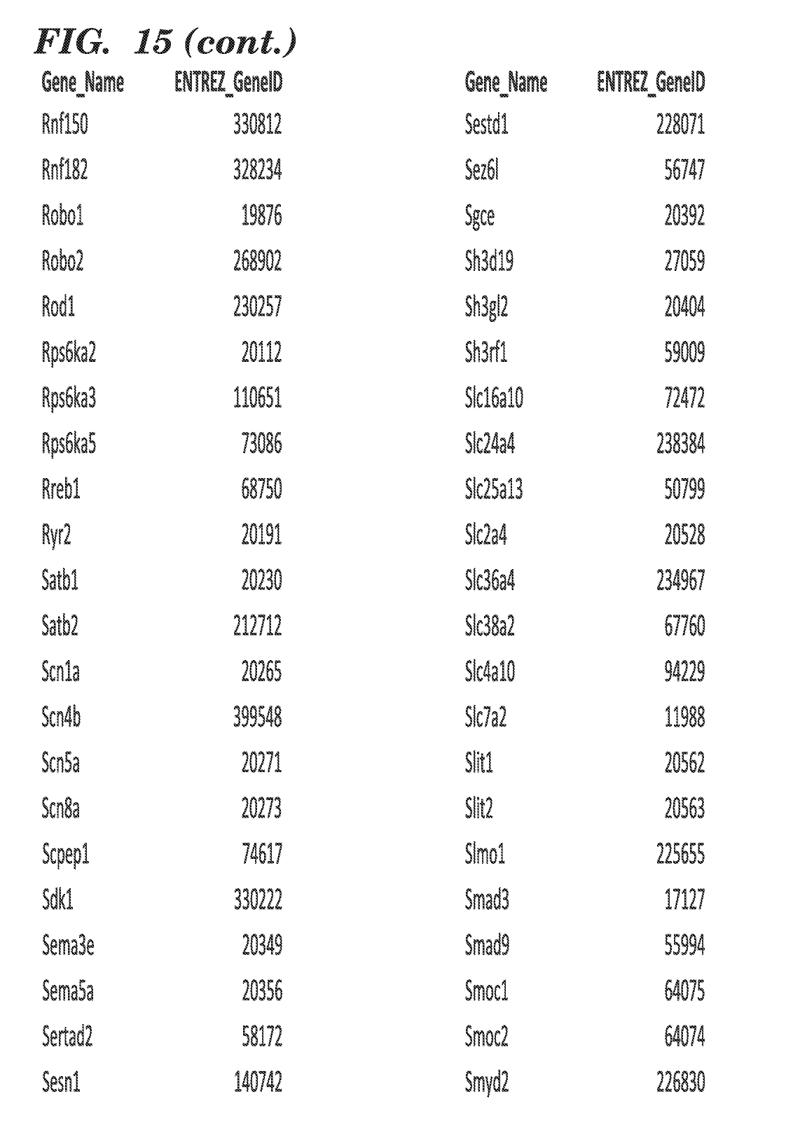

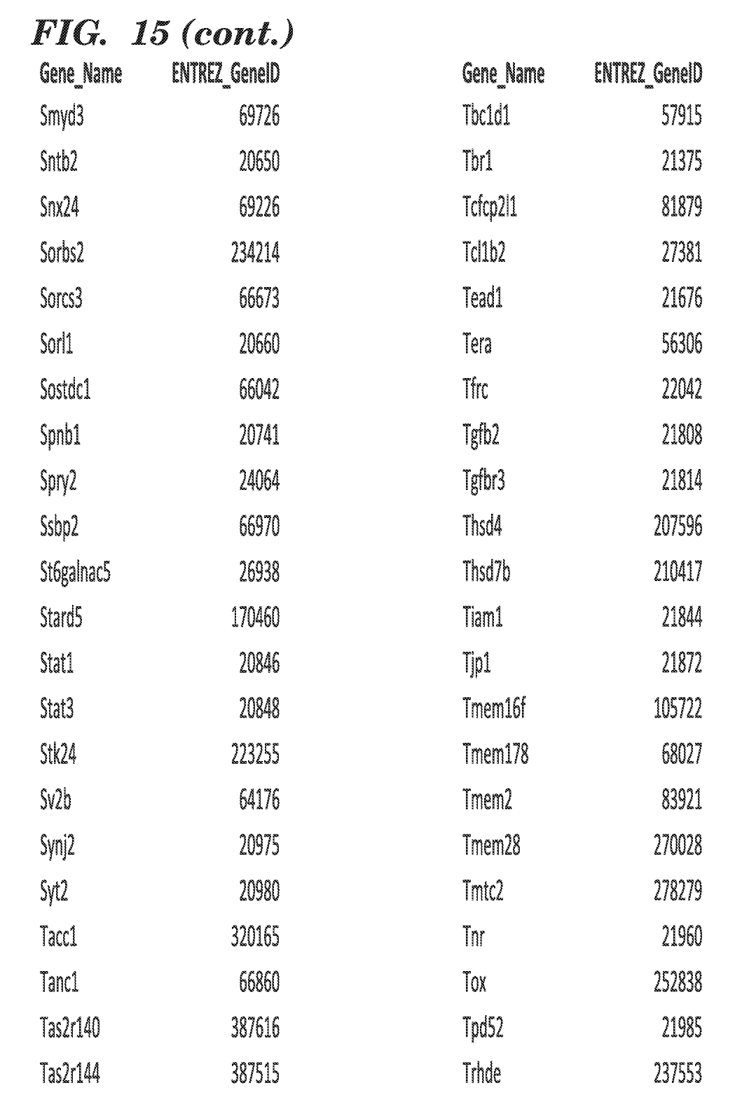

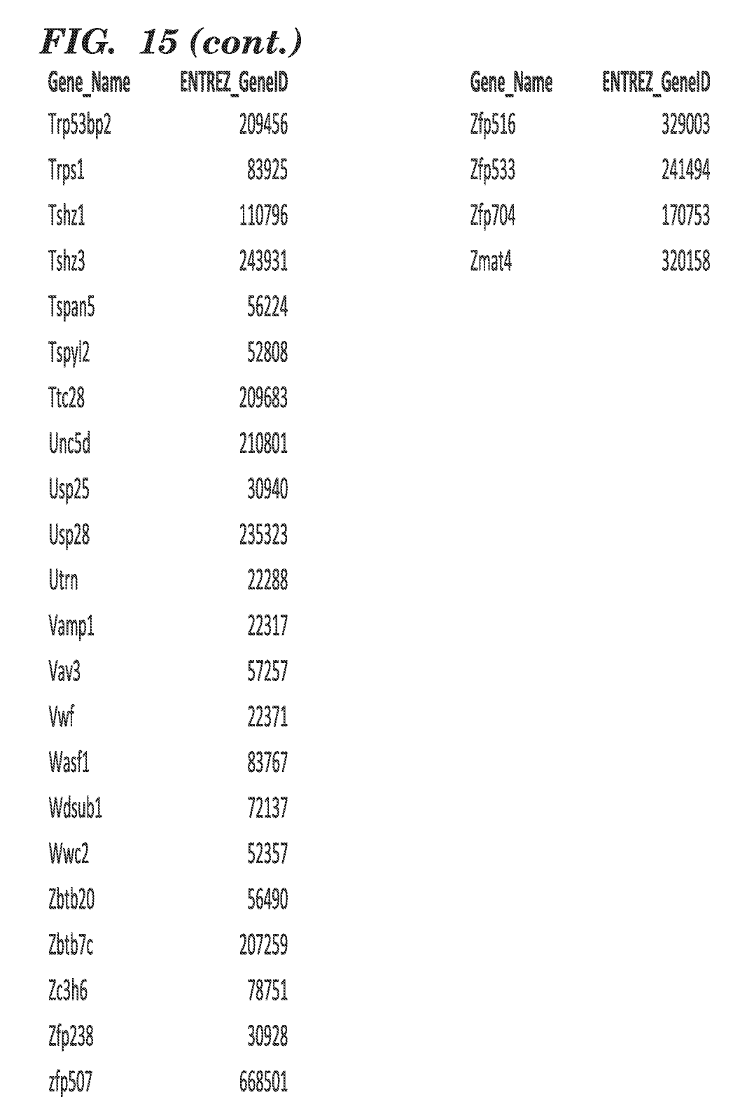

As used herein, the terms “test compound” or “test agent” refer to a compound or agent and/or compositions thereof that are to be screened for their ability to down-regulate or up-regulate a target gene that effects long gene expression. For example, test compounds can be assayed for their ability to inhibit or promote the activity of target genes involved in transcriptional elongation or translation elongation. Target genes can also be long genes of the brain (e.g. genes indicated in FIG. 15).

Proteins involved in transcriptional elongation and translational elongation are known to those in the art, for example proteins that promote elongation include BRD4, Dot11, Ptefb, DSIF (Wada et al., Genes & Dev. 1998. 12: 343-356); SPt5p (Anderson et al. May 27, 2011 J.B.C., 286, 18816-18824), Spt4p (Anderson et al. May 27, 2011 J.B.C., 286, 18816-18824); PAF (Gallard et al. (2009) Genome-Wide Analysis of Factors Affecting Transcription Elongation and DNA Repair: A New Role for PAF and Ccr4-Not in Transcription-Coupled Repair. PLoS Genet 5(2): e1000364.) Ccr4-Not; Sp3 (Valin and Gill, Cell Cycle 2013 Jun. 15; 12(12):1828-34), ELL (Lin et al. Mol. Cell, Volume 37, Issue 3, 12 Feb. 2010, Pages 429-437), P-TEFb (Lin et al. Mol. Cell, Volume 37, Issue 3, 12 Feb. 2010, Pages 429-437); and AFF4 (Lin et al. Mol. Cell, Volume 37, Issue 3, 12 Feb. 2010, Pages 429-437).

Various biochemical and molecular biology techniques or assays well known in the art that can be employed in a screen. For example, techniques are described in, e.g., Handbook of Drug Screening, Seethala et al. (eds.), Marcel Dekker (1st ed., 2001); High Throughput Screening: Methods and Protocols (Methods in Molecular Biology, 190), Janzen (ed.), Humana Press (1st ed., 2002); Current Protocols in Immunology, Coligan et al. (Ed.), John Wiley & Sons Inc (2002); Sambrook et al., Molecular Cloning: A Laboratory Manual, Cold Spring Harbor Press (3rd ed., 2001); and Brent et al., Current Protocols in Molecular Biology, John Wiley & Sons, Inc. (ringbound ed., 2003). Test agents are typically first screened in vitro for their ability to modulate gene expression (e.g. in brain tissue or neurons) and those test agents with modulatory effect are identified. Positive modulatory agents are then tested for efficacy in vivo animal models of autism spectrum disorders.

Test agents are first screened for their ability to modulate gene expression or protein activity of the target gene. Initially test agents can be screened for binding to a target gene or protein encoded by the target gene, or screened for modulating activity/function of a protein encoded by a gene. Binding assays are well known to those of skill in the art and include, for example, gel mobility shift assays, ELISA assay, co-immunoprecipitation, or e.g. FRET. The test agent can further tested to confirm to down-regulate or up-regulate expression of long gene expression.

In one embodiment, a test agent is assayed for the ability to inhibit or increase transcription of a target gene. Transcriptional assay are well known to those of skill in the art (see e.g. U.S. Pat. Nos. 7,319,933, 6,913,880). For example, modulation of expression of a gene can be examined in a cell-based system by transient or stable transfection of a reporter expression vector into cultured cell lines. Test compounds can be assayed for ability to inhibit or increase expression of a reporter gene (e.g., luciferase gene) under the control of a transcription regulatory element (e.g., promoter sequence) of a gene. An assay vector bearing the transcription regulatory element that is operably linked to the reporter gene can be transfected into any mammalian cell line for assays of promoter activity. Reporter genes typically encode polypeptides with an easily assayed enzymatic activity that is naturally absent from the host cell. Typical reporter polypeptides for eukaryotic promoters include, e.g., chloramphenicol acetyltransferase (CAT), firefly or Renilla luciferase, beta-galactosidase, beta-glucuronidase, alkaline phosphatase, and green fluorescent protein (GFP). Vectors expressing a reporter gene under the control of a transcription regulatory element of a gene can be prepared using routinely practiced techniques and methods of molecular biology (see, e.g., e.g., Samrbook et al., supra; Brent et al., supra).

In addition to a reporter gene, the vector can also comprise elements necessary for propagation or maintenance in the host cell, and elements such as polyadenylation sequences and transcriptional terminators. Exemplary assay vectors include pGL3 series of vectors (Promega, Madison, Wis.; U.S. Pat. No. 5,670,356), which include a polylinker sequence 5′ of a luciferase gene. General methods of cell culture, transfection, and reporter gene assay have been described in the art, e.g., Samrbook et al., supra; and Transfection Guide, Promega Corporation, Madison, Wis. (1998). Any readily transfectable mammalian cell line may be used to assay expression of the reporter gene from the vector, e.g., HCTl 16, HEK 293, MCF-7, and HepG2 cells. In certain embodiments, screened are performed in neuronal cells.

Alternatively, modulation of mRNA levels can be assessed using, e.g., biochemical techniques such as Northern hybridization or other hybridization assays, nuclease protection assay, reverse transcription (quantitative RT-PCR) techniques and the like. Such assays are well known to those in the art. In one embodiment, nuclear “run-on” (or “run-off”) transcription assays are used (see e.g. Methods in Molecular Biology, Volume: 49, Sep. 27, 1995, Page Range: 229-238). Arrays can also be used; arrays, and methods of analyzing mRNA using such arrays have been described previously, e.g. in EP0834575, EP0834576, WO96/31622, U.S. Pat. No. 5,837,832 or WO98/30883. WO97/10365 provides methods for monitoring of expression levels of a multiplicity of genes using high density oligonucleotide arrays.

In one embodiment the test agent is assayed for the ability to inhibit or increase translation of a target gene. Gene translation can be measured by quantitiation of protein expressed from a gene, for example by Western blotting, by an immunological detection of the protein, ELISA (enzyme-linked immunosorbent assay), Western blotting, radioimmunoassay (RIA) or other immunoassays and fluorescence-activated cell analysis (FACS) to detect protein.

In one embodiment, the modulating compound is an RNA interfering inhibitory or activating agent, for example a siRNA or a miRNA gene silencer or activator that decreases or increases respectively, the mRNA level of a gene identified herein. The modulating compound results in a decrease or increase, respectively, in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one embodiment, the mRNA levels are decreased or increased respectively by at least about 70%, about 80%, about 90%, about 95%, about 99%, about 100%.

As used herein, the term “RNAi” refers to any type of interfering RNA, including but are not limited to, siRNA, shRNA, endogenous microRNA and artificial microRNA; inhibitory or activating of gene expression.

As used herein an “siRNA” refers to a nucleic acid that forms a double stranded RNA, which double stranded RNA has the ability to reduce or inhibit expression of a gene or target gene when the siRNA is present or expressed in the same cell as the target gene, e.g. the long genes of the brain. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length). In one embodiment, the double stranded siRNA can contain a 3′ and/or 5′ overhang on each strand having a length of about 1, 2, 3, 4, or 5 nucleotides. In one embodiment, the siRNA is capable of promoting inhibitory RNA interference through degradation or specific post-transcriptional gene silencing (PTGS).

The term “complementary” or “complementarity” as used herein refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. A “complement” of a nucleic acid sequence as used herein refers to a nucleotide sequence whose nucleic acids show total complementarity to the nucleic acids of the nucleic acid sequence.

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNA are small RNAs naturally present in the genome which are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

Means for selecting nucleotide sequences (e.g. RNAi, siRNA, shRNA) that can serve as inhibitors or activators of target gene expression are well known and practiced by those of skill in the art. Many computer programs are available to design RNAi agents against a particular nucleic acid sequence. The targeted region of RNAi (e.g. siRNA etc.) can be selected from a given target gene sequence, e.g., a sequence of a (long gene identified herein in FIG. 15), beginning from about 25 to 50 nucleotides, from about 50 to 75 nucleotides, or from about 75 to 100 nucleotides downstream of the start codon. Nucleotide sequences can contain 5′ or 3′ UTRs and regions nearby the start codon. One method of designing a siRNA molecule of the present invention involves identifying the 23 nucleotide sequence motif AA(N19)TT (where N can be any nucleotide), and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search can be extended using the motif NA(N21), where N can be any nucleotide. In this situation, the 3′ end of the sense siRNA can be converted to TT to allow for the generation of a symmetric duplex with respect to the sequence composition of the sense and antisense 3′ overhangs. The antisense RNAi molecule can then be synthesized as the complement to nucleotide positions 1 to 21 of the 23 nucleotide sequence motif. The use of symmetric 3′ TT overhangs can be advantageous to ensure e.g. that the small interfering ribonucleoprotein particles (siRNPs) are formed with approximately equal ratios of sense and antisense target RNA-cleaving siRNPs (Elbashir et al. (2001) supra and Elbashir et al. 2001 supra).

In one embodiment, the RNAi agent targets at least 5 contiguous nucleotides in the identified target gene sequence. In one embodiment, the RNAi agent targets at least 6, 7, 8, 9 or 10 contiguous nucleotides in the identified target sequence. In one embodiment, the RNAi agent targets at least 11, 12, 13, 14, 15, 16, 17, 18 or 19 contiguous nucleotides in the identified target sequence.

In some embodiments, in order to increase nuclease resistance in an RNAi agent as disclosed herein, one can incorporate non-phosphodiester backbone linkages, as for example methylphosphonate, phosphorothioate or phosphorodithioate linkages or mixtures thereof, into one or more non-RNASE H-activating regions of the RNAi agents. Such non-activating regions may additionally include 2′-substituents and can also include chirally selected backbone linkages in order to increase binding affinity and duplex stability. Other functional groups may also be joined to the oligonucleoside sequence to instill a variety of desirable properties, such as to enhance uptake of the oligonucleoside sequence through cellular membranes, to enhance stability or to enhance the formation of hybrids with the target nucleic acid, or to promote cross-linking with the target (as with a psoralen photo-cross-linking substituent). See, for example, PCT Publication No. WO 92/02532 which is incorporated herein in by reference.

Agents in the form of a protein and/or peptide or fragment thereof can also be designed to modulate a gene expression. Such agents are intended to encompass proteins which are normally absent as well as proteins normally endogenously expressed within a cell, e.g. expressed at low levels. Examples of useful proteins are mutated proteins, genetically engineered proteins, peptides, synthetic peptides, recombinant proteins, chimeric proteins, antibodies, intrabodies, midibodies, minibodies, triabodies, humanized proteins, humanized antibodies, chimeric antibodies, modified proteins and fragments thereof. Agents also include antibodies (polyclonal or monoclonal), neutralizing antibodies, antibody fragments, peptides, proteins, peptide-mimetics, or hormones, or variants thereof that function to inactivate the nucleic acid and/or protein of the genes identified herein. Modulation of gene expression or protein activity can be direct or indirect. In one embodiment, a protein/peptide agent directly binds to a protein encoded by a gene identified herein, or directly binds to a nucleic acid of a gene identified herein.

The agent may function directly in the form in which it is administered. Alternatively, the agent can be modified or utilized intracellularly to produce something which modulates the gene, e.g. introduction of a nucleic acid sequence into the cell and its transcription resulting in the production of an inhibitor or activator of gene expression or protein activity.

The agent may comprise a vector. Many vectors useful for transferring exogenous genes into target mammalian cells are available, e.g. the vectors may be episomal, e.g., plasmids, virus derived vectors such cytomegalovirus, adenovirus, etc., or may be integrated into the target cell genome, through homologous recombination or random integration, e.g., retrovirus derived vectors such MMLV, HIV-1, ALV, etc. Many viral vectors are known in the art and can be used as carriers of a nucleic acid modulatory compound into the cell. For example, constructs containing the modulatory compound may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. Alternatively, the construct may be incorporated into vectors capable of episomal replication, e.g EPV and EBV vectors. The nucleic acid incorporated into the vector can be operatively linked to an expression control sequence when the expression control sequence controls and regulates the transcription and translation of that polynucleotide sequence.

The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence. In some examples, transcription of a nucleic acid modulatory compound is under the control of a promoter sequence (or other transcriptional regulatory sequence) which controls the expression of the nucleic acid in a cell-type in which expression is intended. It will also be understood that the modulatory nucleic acid can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene. The promoter sequence may be a “tissue-specific promoter,” which means a nucleic acid sequence that serves as a promoter, i.e., regulates expression of a selected nucleic acid sequence operably linked to the promoter, and which affects expression of the selected nucleic acid sequence in specific cells, e.g. pancreatic beta-cells, muscle, liver, or fat cells. The term also covers so-called “leaky” promoters, which regulate expression of a selected nucleic acid primarily in one tissue, but cause expression in other tissues as well.

In some embodiments, the modulatory compound used in methods of the invention is a small molecule. As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kD), preferably less than 3 kD, still more preferably less than 2 kD, and most preferably less than 1 kD. In some cases it is preferred that a small molecule have a molecular weight equal to or less than 700 Daltons.

Test agents can be small molecule compounds, e.g. methods for developing small molecule, polymeric and genome based libraries are described, for example, in Ding, et al. J Am. Chem. Soc. 124: 1594-1596 (2002) and Lynn, et al., J. Am. Chem. Soc. 123: 8155-8156 (2001). Commercially available compound libraries can be obtained from, e.g., ArQule, Pharmacopia, graffinity, Panvera, Vitas-M Lab, Biomol International and Oxford. These libraries can be screened using the screening devices and methods described herein. Chemical compound libraries such as those from NIH Roadmap, Molecular Libraries Screening Centers Network (MLSCN) can also be used. A comprehensive list of compound libraries can be found at www.broad.harvard.edu/chembio/platform/screening/compound_libraries/index.htm. A chemical library or compound library is a collection of stored chemicals usually used ultimately in high-throughput screening or industrial manufacture. The chemical library can consist in simple terms of a series of stored chemicals. Each chemical has associated information stored in some kind of database with information such as the chemical structure, purity, quantity, and physiochemical characteristics of the compound.

In one embodiment, the test agents include peptide libraries, e.g. combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins. The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

### Autism Spectrum Disorders

Methods are provided for the treatment of ASD Spectrum Disorders (ASDs). Autism spectrum disorders are also known as Pervasive Developmental Disorders (PDDs), cause severe and pervasive impairment in thinking, feeling, language, and the ability to relate to others. These disorders are usually first diagnosed in early childhood and range from a severe form, called autistic disorder, through pervasive development disorder not otherwise specified (PDD-NOS), to a much milder form, Asperger syndrome. They also include two rare disorders, Rett syndrome and childhood disintegrative disorder. Prevalence studies have been done in several states and also in the United Kingdom, Europe, and Asia. A recent study of a U.S. metropolitan area estimated that 3.4 of every 1,000 children 3-10 years old had ASD.

All children with ASD demonstrate deficits in 1) social interaction, 2) verbal and nonverbal communication, and 3) repetitive behaviors or interests. In addition, they will often have unusual responses to sensory experiences, such as certain sounds or the way objects look. Anxiety and hyperactivity may also be apparent. Each of these symptoms run the gamut from mild to severe. They will present in each individual child differently. For instance, a child may have little trouble learning to read but exhibit extremely poor social interaction. Each child will display communication, social, and behavioral patterns that are individual but fit into the overall diagnosis of an autism spectrum disorder. A skilled artisan is versed in diagnosis of autism spectrum disorders.

In social interactions and relationships, symptoms can include: significant problems developing nonverbal communication skills, such as eye-to-eye gazing, facial expressions, and body posture; failure to establish friendships with children the same age; lack of interest in sharing enjoyment, interests, or achievements with other people; lack of empathy. People with ASD can have difficulty understanding another person's feelings, such as pain or sorrow. Additionally, there is often an aversion to physical contact or signs of affection. In verbal and nonverbal communication, symptoms can include: delay in, or lack of, learning to talk. As many as 50% of people with ASD never speak and it is common for them to have problems taking steps to start a conversation. Also, people with ASD have difficulties continuing a conversation once it has begun. A repetitive use of language is can be present and patients will often repeat over and over a phrase they have heard previously (echolalia). Autistic individuals have difficulty understanding their listener's perspective. For example, a person with ASD may not understand that someone is using humor. They may interpret the communication word for word and fail to catch the implied meaning. People with ASD may show limited interest in activities or play and display an unusual focus on pieces. Younger children with ASD often focus on parts of toys, such as the wheels on a car, rather than playing with the entire toy or are preoccupied with certain topics. For example, older children and adults may be fascinated by train schedules, weather patterns, or license plates. A need for sameness and routines is often exhibited such as a need to always eat bread before salad or an insistance on driving the same route every day to school. People with ASD may also display typical behaviors such as body rocking and hand flapping.

Children with ASD do not follow the typical patterns of child development. In some children, hints of future problems may be apparent from birth. In most cases, the problems in communication and social skills become more noticeable as the child lags further behind other children the same age. Some other children start off well enough. Often times between 12 and 36 months old, the differences in the way they react to people and other unusual behaviors become apparent. Some parents report the change as being sudden, and that their children start to reject people, act strangely, and lose language and social skills they had previously acquired. In other cases, there is a plateau, or leveling, of progress so that the difference between the child with ASD and other children the same age becomes more noticeable.

ASD is defined by a certain set of behaviors that can range from the very mild to the severe. ASD has been associated with mental retardation (MR). It is said that between 75% and 90% of all autistics are mentally retarded. However, having ASD does not necessarily mean that one will have MR. ASD occurs at all IQ levels, from genius levels to the severely learning-disabled. Furthermore, there is a distinction between ASD and MR. People with MR generally show even skill development, whereas individuals with ASD typically show uneven skill development. Individuals with ASD may be very good at certain skills, such as music or mathematical calculation, yet perform poorly in other areas, especially social communication and social interaction.

Currently, there is no single test for ASD. In evaluating a child, clinicians rely on behavioral characteristics to make a diagnosis. Some of the characteristic behaviors of ASD can be apparent in the first few months of a child's life, or they can appear at any time during the early years. For the diagnosis, problems in at least one of the areas of communication, socialization, or restricted behavior must be present before the age of 3. The diagnosis requires a two-stage process. The first stage involves developmental screening during “well child” check-ups; the second stage entails a comprehensive evaluation by a multidisciplinary team.

In one embodiment, diagnosis is by the ASD Diagnostic Interview-Revised (ADI-R) (Lord C, et al., 1993, Infant Mental Health, 14:234-52). In another embodiment, diagnosis is by symptoms fitting an Autism Genetic Resource Exchange (AGRE) classification of ASD. Symptoms may be broad spectrum (patterns of impairment along the spectrum of pervasive developmental disorders, including PDD-NOS and Asperger's syndrome).

Several clinical methods of assessing the severity of ASD in totality as well as the severity of individual symptoms exist. These methods include, but are not limited to, the Austism Diagnostic Observation Schedule (ADOS), Childhood Autism Rating Scale (CARS), the Social Responsiveness Scale (SRS) and the ADI-R. The ADOS has recently been standardized specifically to allow for a severity metric (Gotham et al., Journal of Autism and Developmental Disorders 2009 39:693-705). Additionally, magnetoencephalography has been reported as a quantitative means of diagnosing ASD (Roberts et al., RSNA 2008; Roberts et al., International Journal of Psychophysiology 2008 68:149-60). Hand grip strength has also been correlated with CARS scores (Kern et al., Research in Autism Spectrum Disorders published online 2010). Repetitive behaviors can also be quantified by various means, including the Yale-Brown Obssessive Compulsive Scale (YBOCS) (US 2006/0105939 A1). The Autism Treatment Evaluation Checklist (ATEC) can also be used to quantify severity of impairments in speech, language, communication, sensory cognitive awareness, health, physical, and behavior, and social skills and demonstrate improvement in these metrics (US 2007/0254314 A1). Furthermore, correlations between expression of certain genes or biomarkers (including but not limited to neurexin-113, NBEA, FHR1, apolipoprotein B, transferrin, TNF-alpha converting enzyme, dedicator of cytokinesis protein 1 (DOCK 180), fibronectin 1, complement C1q, complement component 3 precursor protein, and complement component 4B proprotein) and ASD has been reported (US 2009/0197253 A1; US 2006/0194201 A1; U.S. Pat. No. 7,604,948). The specific autism spectrum disorders of Rett syndrome, Fragile X syndrome and Angleman syndrome, as well as others, are described in more detail below. These disorders can also be assessed by monitoring for genetic mutation in the subject.

Rett Syndrome (RTT)

In one embodiment the autism spectrum disorder to be treated using methods of the invention is Rett syndrome (RTT). RTT is a postnatal neurological disorder found in girls and is caused by an X-linked loss of function mutation of the MECP2 gene (Amir et al. Nature Genetics 23, 185-188 (1999), incorporated by reference in entirety). RTT causes problems in brain function responsible for cognitive, sensory, emotional, motor and autonomic function. Rett syndrome can effect learning, speech, sensory sensations, mood, movement, breathing, cardiac function, and even chewing, swallowing, and digestion.

Rett syndrome symptoms appear after an early period of apparently normal or near normal development until six to eighteen months of life, when there is a slowing down or stagnation of skills. A period of regression then follows when she loses communication skills and purposeful use of her hands. Soon, stereotyped hand movements such as handwashing, gait disturbances, and slowing of the normal rate of head growth become apparent. Other problems may include seizures and disorganized breathing patterns while she is awake. In the early years, there may be a period of isolation or withdrawal when she is irritable and cries inconsolably. Over time, motor problems may increase, but in general, irritability lessens and eye contact and communication improve.

Rett syndrome is confirmed with a simple blood test to identify the MECP2 mutation. However, since the MECP2 mutation is also seen in other disorders, the presence of the MECP2 mutation in itself is not enough for the diagnosis of Rett syndrome. Diagnosis requires either the presence of the mutation (a molecular diagnosis) or fulfillment of the diagnostic criteria (a clinical diagnosis, based on signs and symptoms that you can observe for autism spectrum disorders) or both.

Rett syndrome can present with a wide range of disability ranging from mild to severe. The course and severity of Rett syndrome is determined by the location, type and severity of the MECP-2 mutation. Therefore, two girls of the same age with the same mutation can appear quite different.

Fragile X Syndrome

In one embodiment the autism spectrum disorder to be treated using methods of the invention is Fragile X syndrome. Mutations in the FMR1 gene cause fragile X syndrome. The FMR1 gene encodes fragile X mental retardation 1 protein, or FMRP. Fragile X syndrome causes a range of developmental problems including learning disabilities and cognitive impairment. Usually, males are more severely affected by this disorder than females.

Affected individuals usually have delayed development of speech and language by age 2. Most males with fragile X syndrome have mild to moderate intellectual disability, while about one-third of affected females are intellectually disabled. Children with fragile X syndrome may also have anxiety and hyperactive behavior such as fidgeting or impulsive actions. They may have attention deficit disorder (ADD), which includes an impaired ability to maintain attention and difficulty focusing on specific tasks. About one-third of individuals with fragile X syndrome have features of autism spectrum disorders that affect communication and social interaction. Seizures occur in about 15 percent of males and about 5 percent of females with fragile X syndrome.

Most males and about half of females with fragile X syndrome have characteristic physical features that become more apparent with age. These features include a long and narrow face, large ears, a prominent jaw and forehead, unusually flexible fingers, flat feet, and in males, enlarged testicles (macroorchidism) after puberty.

Diagnosis of fragile-x syndrome is made by using the diagnosis methods for autism spectrum disorders and by genetic analysis for FMR1 mutation.

Angleman Syndrome

In one embodiment the autism spectrum disorder to be treated using methods of the invention is Angelman syndrome (AS). Angelman syndrome is a neuro-genetic disorder characterized by intellectual and developmental delay, sleep disturbance, seizures, jerky movements (especially hand-flapping), frequent laughter or smiling, and usually a happy demeanor. AS is caused by mutation of the E3 ubiquitin ligase Ube3A. AS can be caused by mutation on the maternally inherited chromosome 15 while the paternal copy, which may be of normal sequence, is imprinted and therefore silenced. It is estimated that 1/10,000 to 1/20,000 children present with AS.

Symptoms of Angelman syndrome can include; developmental delays such as a lack of crawling or babbling at 6 to 12 months, mental retardation, no speech or minimal speech, ataxia (inability to move, walk, or balance properly), a puppet-like gait with jerky movements, hyperactivity, trembling in the arms and legs, frequent smiling and laughter, bouts of inappropriate laughter, widely spaced teeth, a happy, excitable personality, epilepsy, an electroencephalographic abnormality with slowing and notched wave and spikes, seizures which usually begin at 2 to 3 years of age, stiff or jerky movements, seizures accompanied by myoclonus and atypical absence, partial seizures with eye deviation and vomiting, a small head which is noticeably flat in the back (microbrachyoephaly), crossed eyes (strabismus), thrusting of the tongue and suck/swallowing disorders, protruding tongue, excessive chewing/mouthing behaviors, hyperactive lower extremity deep tendon reflexes, wide-based gait with pronated or valgus-positioned ankles, increased sensitivity to heat, walking with the arms up in the air, fascination with water or crinkly items such as some papers or plastics, obesity in older children, constipation, a jutting lower jaw, light pigmentation of the hair, skin, and eyes (hypopigmentation), frequent drooling, prognathia, feeding problems and/or truncal hypotonia during infancy, and scoliosis. Symptoms are usually not evident at birth and are often first evident as developmental delays such as a failure to crawl or babble between the ages of 6 to 12 months as well as slowing head growth before the age of 12 months. Individuals with Angleman syndrome may also suffer from sleep disturbances including difficulty initiating and maintaining sleep, prolonged sleep latency, prolonged wakefulness after sleep onset, high number of night awakenings and reduced total sleep time, enuresis, bruxism, sleep terrors, somnambulism, nocturnal hyperkinesia, and snoring.

Severity of symptoms of AS has been measured clinically (Williams et al., American Journal of Medical Genetics 2005 140A; 413-8) and quantification of the severity of different symptoms is refined enough to allow segregation of patients based upon the particular genetic mechanism of their disease (Lossie et al., Journal of Medical Genetics 2001 38; 834-845; Ohtsuka et al., Brain and Development 2005 27; 95-100) and may include the extent of language ability, degree of independent mobility, frequency and severity of seizures, ability to comprehend language, acquisition of motor skills, growth parameters. Lossie et al. have developed a screening procedure for suspected Angelman syndrome patients that quantifies the severity of 22 distinct criteria. Other measurements of symptom severity include psychometric methods to distinguish the degree of developmental delay with respect to pyschomotoer developmental achievement, visual skills, social interactions based on non-verbal events, expressive language abilities, receptive language abilities, and speech impairment. The degree of gait and movement disturbances has been measured as well as attention ability and the extent of EEG abnormalities (Williams et al., American Journal of Medical Genetics 2005 140A; 413-8).

MeCP2 Duplication Syndrome

In one embodiment the autism spectrum disorder to be treated using methods of the invention is MeCP2 duplication syndrome. MECP2 duplication syndrome is a characterized by infantile hypotonia, severe mental retardation, poor speech development, progressive spasticity, recurrent respiratory infections (in ˜75% of affected individuals) and seizures (in ˜50%). MECP2 duplication syndrome is 100% penetrant in males. Occasionally females have been described with a MECP2 duplication and related clinical findings, often associated with concomitant X-chromosomal abnormalities that prevent inactivation of the duplicated region. Generalized tonic-clonic seizures are most often observed; atonic seizures and absence seizures have also been described. One third of affected males are never able to walk independently. Almost 50% of affected males die before age 25 years, presumably from complications of recurrent infection and/or neurologic deterioration. In addition to the core features, autistic behaviors and gastrointestinal dysfunction have been observed in several affected boys. Although interfamilial phenotypic variability is observed, severity is usually consistent within families.

Diagnosis is determined by identifying duplications in the MECP2 gene. Duplications of MECP2 ranging from 0.3 to 4 Mb are found in all affected males and are identified by a variety of test methods. In fewer than 5% of affected males routine G-banded cytogenetic analysis detects duplications of Xq28 (the chromosomal locus of MECP2) larger than approximately 8 Mb.

Other Autism Disorders Due to Loss of Function

In certain embodiments the autism spectrum disorder to be treated using methods of the invention is due to a loss of function mutation in topoisomerase, e.g. a loss of function mutation in TOP1 (Xu et al. Characterization of BTBD1 and BTBD2, two similar BTB-domain-containing Kelch-like proteins that interact with Topoisomerase IBMC Genomics. 2002; 3: 1), or other topoisomerase. In such embodiments, the agent to treat loss of function in topoisomerase is an agent that up-regulates the expression of long-genes in the brain.

In certain embodiments the autism spectrum disorder to be treated using methods of the invention is due to a loss of function mutation in CHD8 (Thomson et al., CHD8 is an ATP-Dependent Chromatin Remodeling Factor That Regulates β-Catenin Target Genes, Mol Cell Biol. June 2008; 28(12): 3894-3904. March, 2008).

In certain embodiments the autism spectrum disorder to be treated using methods of the invention is due to a loss of function mutation in MBD5 (Hodge et al. Disruption of MBD5 contributes to a spectrum of psychopathology and neurodevelopmental abnormalities Molecular Psychiatry 19, 368-379 March, 2014).

In certain embodiments, agents that modulate long gene expression in the brain are used to treat Schizophrenia and cognitive impairment due to disruption of Top3B (a Loss-of-function of TOP3B) (Stoll et al. Deletion of TOP3β, a component of FMRP-containing mRNPs, contributes to neurodevelopmental disorders Nature Neuroscience 16, 1228-1237, 2013). In such embodiments, the agent to treat Schizophrenia and cognitive impairment due to disruption of Top3B is an agent that down-regulates the expression of long genes in the brain.

### Treatment of ASDs

Methods are provided for treatment of autism spectrum disorders ASDs comprising administering to a subject an effective amount of an agent that modulate the expression of long genes in the brain.

In some embodiments, the methods of the invention further comprise selecting a subject identified as being in need of treatment. As used herein, the phrase “subject in need of treatment” refers to a subject who is diagnosed with or identified as suffering from, having or at risk for developing, ASD. A subject in need can be identified using any method known in the art used for diagnosis of an ASD, including for example those described herein and including genetic analysis.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, at least one symptom of the ASD are alleviated by at least 5%, at least 10%, at least 20%, at least 30%, at least 40%, at least 50%, at least 60%, at least 70%, at least 80%, at least 90%. The term treatment is not intended to include cure of the disorder, but rather ameliorate, inhibit or decrease symptoms of the disorder. In certain embodiments, the agent is administered for the life of the patient in order to effect long term amelioration of the disease or disorder.

In some embodiments, a goal of treatment of ASDs is to reduce repetitive behaviors, increase social interaction, reduce anxiety, reduce hyperactivity, increase empathy, and/or to improve speech e.g. by at least 5%, at least 10%, at least 20%, at least 30%, at least 40%, at least 50%, at least 60%, at least 70%, at least 80%, at least 90%. Severity of symptoms can be measured by means well known to clinicians, See, for example, the heading “Autism Spectrum Disorder” including the subheadings “Fragile X syndrome”, “Angleman syndrome” and “Rett Syndrome” etc. herein.

In some embodiments, a goal of treatment of ASDs is to reduce seizure activity, e.g. by at least 5%, at least 10%, at least 20%, at least 30%, at least 40%, at least 50%, at least 60%, at least 70%, at least 80%, at least 90%. Severity of symptoms can be measured by means well known to clinicians, See, for example, the heading “Autism Spectrum Disorder” including the subheadings “Fragile X syndrome”, “Angleman syndrome” and “Rett Syndrome” etc. herein.

Delaying the onset of ASD in a subject refers to delay of onset of at least one symptom of the syndrome or disorder, or combinations thereof, for at least 1 week, at least 2 weeks, at least 1 month, at least 2 months, at least 6 months, at least 1 year, at least 2 years, at least 5 years, at least 10 years, at least 20 years, at least 30 years, at least 40 years or more, and can include the entire lifespan of the subject.

As used herein, the term “subject”, “individual” and “patient” are used interchangeably and means a human or animal. Usually the animal is a vertebrate such as a primate, rodent, domestic animal or game animal. In certain embodiments, the subject is a mammal, e.g., a primate, e.g., a human.

Preferably, the subject is a mammal. The mammal can be a human, non-human primate, mouse, rat, dog, cat, horse, or cow, but are not limited to these examples. Mammals other than humans can be advantageously used as subjects that represent animal models, e.g. animal models of Fragile X syndrome or Retts syndrome, or other ASD. In addition, the methods described herein can be used to treat domesticated animals and/or pets. A subject can be male or female. A subject can be one who has been previously diagnosed with or identified as suffering from an autism spectrum disorder. A subject can also be one who is not yet suffering from an autism spectrum disorder, but is at risk of developing an ASD.

### Pharmaceutical Compositions

For administration to a subject, the agents can be provided in pharmaceutically acceptable compositions. These pharmaceutically acceptable compositions comprise a therapeutically-effective amount of one or more of agents, formulated together with one or more pharmaceutically acceptable carriers (additives) and/or diluents. As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “effective amount” as used herein means that amount of a compound, material, or composition comprising an agent of the present invention which is effective for producing the desired therapeutic effect (i.e. of symptom amelioration) at a reasonable benefit/risk ratio applicable to any medical treatment. For example, an amount of a compound administered to a subject that is sufficient to produce a statistically significant, measurable change in at least one symptom of ASD.

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents. In one embodiment a therapeutically effective amount reduces at least one symptom of ASD by at least 5%, at least 10%, at least 20%, at least 30%, at least 40%, at least 50%, at least 60%, at least 70%, at least 80%, at least 90%. Thus, e.g. a therapeutically effective amount of a topoisomerase inhibitor (e.g. Topoisomerase I inhibitor or Topoisomerase II inhibitor) that reduces long gene expression in the brain, reduces at least one symptom of Rett syndrome or Fragile X syndrome, by at least 5%, at least 10%, at least 20%, at least 30%, at least 40%, at least 50%, at least 60%, at least 70%, at least 80%, or by at least 90%.

The therapeutically effective dose can be estimated initially from a suitable cell culture assays, then a dose may be formulated in animal models to achieve a circulating plasma concentration range that includes the EC50 as determined in cell culture.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

In certain embodiments, the agents are formulated for administration to the brain, e.g. formulated as to cross the blood brain barrier. For example, formulation of agents with exosomes have been shown to cross the blood brain barrier. siRNAs, antisense oligonucleotides, chemotherapeutic agents and proteins formulated with exosomes were delivered to neurons after injecting them systemically (Alvarez-Erviti L, et al. (2011) Delivery of siRNA to the mouse brain by systemic injection of targeted exosomes Nat Biotechnol April 29(4):341-5; Andaloussi S, et al. (2012) Exosome-mediated delivery of siRNA in vitro and in vivo Nat Protoc December 7 (12):2112-26; Andaloussi S, et al. (2013). Extracellular vesicles: biology and emerging therapeutic opportunities Nat Rev Drug Discov May; 12(5):347-57; and Andaloussi S, Lakhal S, Mäger I, Wood M J. (2013). Exosomes for targeted siRNA delivery across biological barriers. Adv Drug Deliv Rev. March 65(3):391-7).

Agents can also be formulated with lipophilic molecules or peptides that allow it to better sneak through the Blood Brain Barrier. Such pro-drugs can be designed using more lipophillic elements or peptides that can be removed by either enzyme degradation or some other mechanism to release the drug into its active form. Agents can also be formulated in nanoparticles, where the agent is bound (in or on) to a nanoparticle capable of traversing the Blood Brain Barrier. Studies have shown that overcoating of nanoparticles with polysorbate 80 yielded doxorubicin concentrations in the brain of up to 6 μg/g after Intravenus injection of 5 mg/kg as compared to no detectable increase in an injection of the drug alone or the uncoated nanoparticle (EL Andaloussi S, et al. (2013) Extracellular vesicles: biology and emerging therapeutic opportunities Nat Rev Drug Discov. 2013 May; 12(5):347-57; and Dadparvar, M., Wagner, S., Wien, S., Kufleitner, J., Worek, F., von Briesen, H., & Kreuter, J. (2011). HI 6 human serum albumin nanoparticles, development and transport over an in vitro blood-brain barrier model Toxicology Letters, 206(1), 60-66).

Exemplary modes of administration that can be used in methods of the invention include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

Methods of delivering RNAi interfering (RNAi) agents (e.g., an siRNA), other nucleic acid modulators, or vectors containing modulatory nucleic acids, to the target cells (e.g. neuronal cells) can include, for example directly contacting the cell with a composition comprising a modulatory nucleic acid, or local or systemic injection of a composition containing the modulatory nucleic acid. In one embodiment, nucleic acid agents (e.g. RNAi, siRNA, or other nucleic acid) are injected directly into any blood vessel, such as vein, artery, venule or arteriole, via, e.g., hydrodynamic injection or catheterization. In some embodiments modulatory nucleic acids can delivered locally to specific organs or delivered by systemic administration, wherein the nucleic acid is complexed with, or alternatively contained within a carrier. Example carriers for modulatory nucleic acid compounds include, but are not limited to, peptide carriers, viral vectors, gene therapy reagents, and/or liposome carrier complexes and the like.

The compound/agents described herein for treatment of ASD can be administered to a subject in combination with another pharmaceutically active agent. Exemplary pharmaceutically active compound include, but are not limited to, those found in Harrison's Principles of Internal Medicine, 13^{th} Edition, Eds. T. R. Harrison et al. McGraw-Hill N.Y., N.Y.; Physicians Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference. In some embodiments, pharmaceutically active agent include those agents known in the art for treatment of seizures, for example, Tegretol or Carbatrol (carbamazepine), Zarontin (ethosuximide), Felbatol, Gabitril, Keppra, Lamictal, Lyrica, Neurontin (Gabapentin), Dilantin (Phenytoin), Topamax, Trileptal, Depakene, Depakote (valproate, valproic acid), Zonegran, Valium and similar tranquilizers such as Klonopin or Tranxene, etc.

The compounds and the additional pharmaceutically active agent (e.g. anti-seizure medication) can be administrated to the subject in the same pharmaceutical composition or in different pharmaceutical compositions (at the same time or at different times). When administrated at different times, compound of the invention and the pharmaceutically active agent can be administered within 5 minutes, 10 minutes, 20 minutes, 60 minutes, 2 hours, 3 hours, 4, hours, 8 hours, 12 hours, 24 hours of administration of the other. When the modulatory compound, and the pharmaceutically active agent are administered in different pharmaceutical compositions, routes of administration can be different.

The amount of compound which can be combined with a carrier material to produce a single dosage form will generally be that amount of the compound which produces a therapeutic effect. Generally out of one hundred percent, this amount will range from about 0.1% to 99% of compound, preferably from about 5% to about 70%, most preferably from 10% to about 30%.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage may be determined by a physician and adjusted, as necessary, to suit observed effects of the treatment. Generally, the compositions are administered so that a modulatory agent/compound is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg etc. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg etc.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the agents. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more. The pharmaceutical compositions can be administered during infancy (between 0 to about 1 year of life), childhood (the period of life between infancy and puberty) and during puberty (between about 8 years of life to 18 years of life). The pharmaceutical compositions can also be administered to treat adults (greater than about 18 years of life).

In certain embodiments, the agent is administered using a chronic treatment regime, e.g. the agent is administered for the life of the patient, e.g. daily, weekly or monthly.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean ±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. For example, a decrease by at least 10% as compared to a reference level, a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount, e.g. increase of at least 10% as compared to a reference level, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) above or below normal or control values. The term refers to statistical evidence that there is a difference. The decision is often made using the p-value.

As used herein, the term “IC50” refers to the concentration of an inhibitor that produces 50% of the maximal inhibition of activity or expression measurable using the same assay in the absence of the inhibitor. The IC50 can be as measured in vitro or in vivo. The IC50 can be determined by measuring activity using a conventional in vitro assay (e.g. protein activity assay, or gene expression assay).

As used herein, the term “EC50,” refers to the concentration of an activator that produces 50% of maximal activation of measurable activity or expression using the same assay in the absence of the activator. Stated differently, the “EC50” is the concentration of activator that gives 50% activation, when 100% activation is set at the amount of activity that does not increase with the addition of more activator. The EC50 can be as measured in vitro or in vivo.

To the extent not already indicated, it will be understood by those of ordinary skill in the art that any one of the various embodiments herein described and illustrated may be further modified to incorporate features shown in any of the other embodiments disclosed herein.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%, when used to describe degrees Celsius is ±1 degree.

In one respect, the present invention relates to the herein described compositions, methods, and respective component(s) thereof, as essential to the invention, yet open to the inclusion of unspecified elements, essential or not (“comprising”). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Some embodiments of the present invention may be defined in any of the following numbered paragraphs:
Paragraph 1 A method for treating an autism spectrum disorder comprising administering to a subject an effective amount of an agent that modulates the expression of long genes in the brain.
Paragraph 2 The method of paragraph 1, wherein the agent modulates expression of long genes in the brain by modulating the transcription of long genes.
Paragraph 3 The method of paragraph 1, wherein the agent modulates expression of long genes in the brain by modulating the translation of long genes.
Paragraph 4 The method of any of paragraphs 1-3, wherein the agent increases the expression of long genes in the brain.
Paragraph 5 The method of any of paragraphs 1-3, wherein the agent decreases the expression of long genes in the brain.
Paragraph 6 The method of paragraph 1, wherein the autism spectrum disorder is MeCP2 duplication syndrome and the agent increases the expression of long genes in the brain.
Paragraph 7 The method of paragraph 1, wherein the autism spectrum disorder is Rett syndrome and the agent decreases the expression of long genes in the brain.
Paragraph 8 The method of paragraph 1, wherein the autism spectrum disorder is Fragile X syndrome and the agent decreases the expression of long genes in the brain.
Paragraph 9 The method of any of paragraphs 1-8, wherein the subject is a human subject.
Paragraph 10 The method of any of paragraphs 1-9, wherein the agent is selected from the group consisting of a small molecule, a nucleic acid, a protein, a peptide, and an antibody.
Paragraph 11 The method of any of paragraphs 1-10, wherein the agent is an RNA interfering agent (RNAi).
Paragraph 12 The method of any of paragraphs 1-11, wherein the agent is administered by a route selected from the group consisting of topical administration, enteral administration, and parenteral administration.
Paragraph 13 The method of any of paragraphs 1-12, wherein the agent is administered in a dose ranging from about 0.1 mg/kg to about 1000 mg/kg.
Paragraph 14 The method of any of paragraphs 1-13, wherein the agent is administered daily.
Paragraph 15 The method of any of paragraphs 1-14, wherein the agent is formulated for delivery to the brain.
Paragraph 16 The method of any of paragraphs 1-15, wherein the agent is not an inhibitor of toposisomerase I.
Paragraph 17 The method of any of paragraphs 1-15, wherein the agent is not an inhibitor of toposisomerase II.
Paragraph 18 The method of paragraph 1, wherein the autism spectrum disorder is caused by a mutation in topoisomerase and the agent increases expression of a long gene in the brain.
Paragraph 19 The method of any of paragraphs 4, 6, or 18 wherein the agent that increases expression of long genes in the brain is a DNA methyltransferase inhibitor.
Paragraph 20 The method of any of paragraphs 5, 7, or 8 wherein the agent that decreases expression of long genes in the brain and is selected from the group consisting of: a topoisomerase inhibitor, a nucleotide analog that inhibits transcriptional elongation, a BRD4 inhibitor that inhibits pro-elongation chromatin modifiers, an inhibitor of Dot1 that promotes elongation-associated chromatin modification, Alpha-Amanitin, a protein synthesis inhibitor, and a DNA intercalator that blocks RNA polymerases.
Paragraph 21 The method of any of paragraphs 5, 7, or 8 wherein the agent that decreases expression of long genes in the brain inhibits a protein that promotes elongation selected from the group consisting of: BRD4, Dot11, Ptefb, DSIF, SPt5p, Spt4p, PAF, Ccr4-Not, Sp3, ELL, P-TEFb, and. AFF4.
Paragraph 22 The method of any of paragraphs 4 or 6 wherein the agent that increases expression of long genes in the brain activates a protein that promotes elongation selected from the group consisting of: BRD4, Dot11, Ptefb, DSIF, SPt5p, Spt4p, PAF, Ccr4, Not, Sp3, ELL, P-TEFb, and. AFF4.
Paragraph 23 The method of any of paragraphs 1-20, wherein the agent inhibits a protein involved in translational elongation and is selected from the group consisting of: Lactimidomycin, Diphthamide, Stm1p, 4EGI1, Orthoformimysin, e1F5A, Minocycline.
Paragraph 24 The method of any of paragraphs 1-20, wherein the agent activates a protein involved in translational elongation and is selected from the group consisting of: Lactimidomycin, Diphthamide, Stm1p, 4EGI1, Orthoformimysin, e1F5A, Minocycline.
Paragraph 25 A method for treatment of Rett syndrome comprising administering to a subject an effective amount of a topoisomerase inhibitor, wherein the effective amount of the topoisomerase inhibitor decreases the expression of long genes in the brain.
Paragraph 26 A method for treatment of Fragile X syndrome comprising administering to a subject an effective amount of a topoisomerase inhibitor, wherein the effective amount of the topoisomerase inhibitor decreases the expression of long genes in the brain.
Paragraph 27 The method of any of paragraphs 25-26, wherein the topoisomerase inhibitor is a topoisomerase I inhibitor selected from the group consisting of: Belotecan (CKD602), Camptothecin, 7-Ethyl-10-Hydroxy-CPT, 10-Hydroxy-CPT, Rubitecan (9-Nitro-CPT), 7-Ethyl-CPT, Topotecan, Irinotecan, Silatecan (DB67) and an indenoisoquinoline derivative.
Paragraph 28 The method of any of paragraphs 25-26, wherein the topoisomerase inhibitor is a topoisomerase II inhibitor selected from the group consisting of: Doxorubicin; Etoposide; Amsacrine; ICRF-193, dexrazoxane (ICRF-187); Resveratrol; Epigallocatechin gallate; Genistein; Quercetin; and Myricetin.
Paragraph 29 The method of any of paragraphs 25-28, wherein the subject is a human subject.
Paragraph 30 The method of any of paragraphs 25-29, wherein the agent is administered by a route selected from the group consisting of topical administration, enteral administration, and parenteral administration.
Paragraph 31 The method of any of paragraphs 25-30, wherein the agent is administered in a dose ranging from about 0.1 mg/kg to about 1000 mg/kg.
Paragraph 32 The method of any of paragraphs 25-31, wherein the agent is administered daily.
Paragraph 33 The method of any of paragraphs 25-32, wherein the agent is formulated for delivery to the brain.
Paragraph 34 The method of any of paragraphs 25-33, wherein the agent is not an inhibitor of toposisomerase I.
Paragraph 35 The method of any of paragraphs 25-33, wherein the agent is not an inhibitor of toposisomerase II.

Embodiments of the invention will be further illustrated by the following non-limiting examples.

### EXAMPLES

### Example 1: Length Dependent Gene Misregulation in Autism Spectrum Disorders

Gene Length-Dependent Misregulation in RTT Models

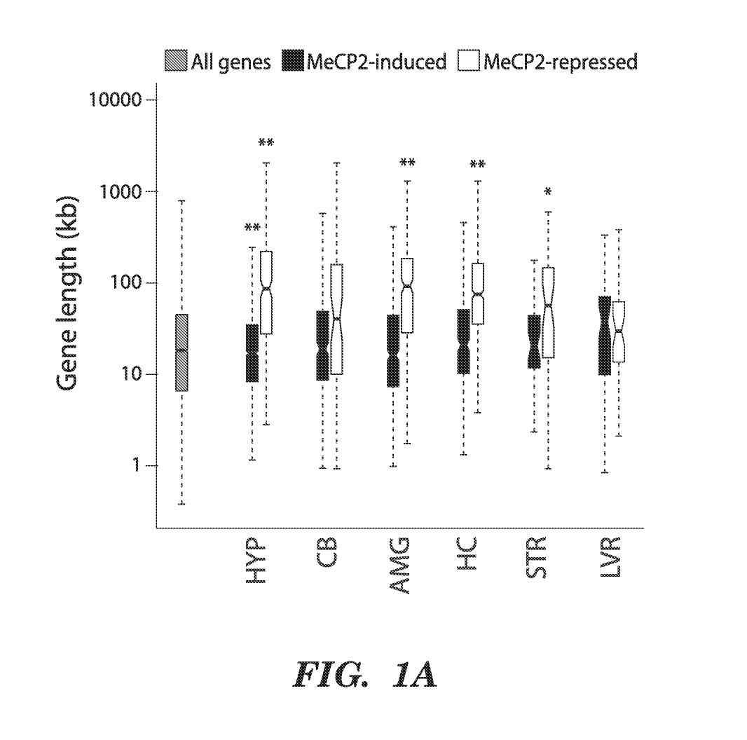

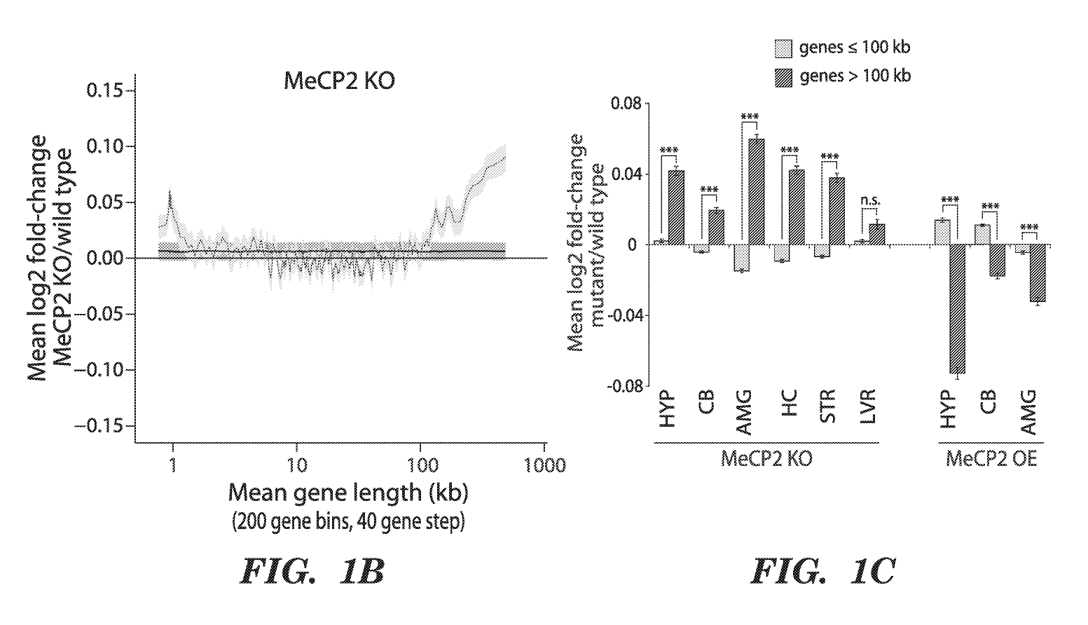

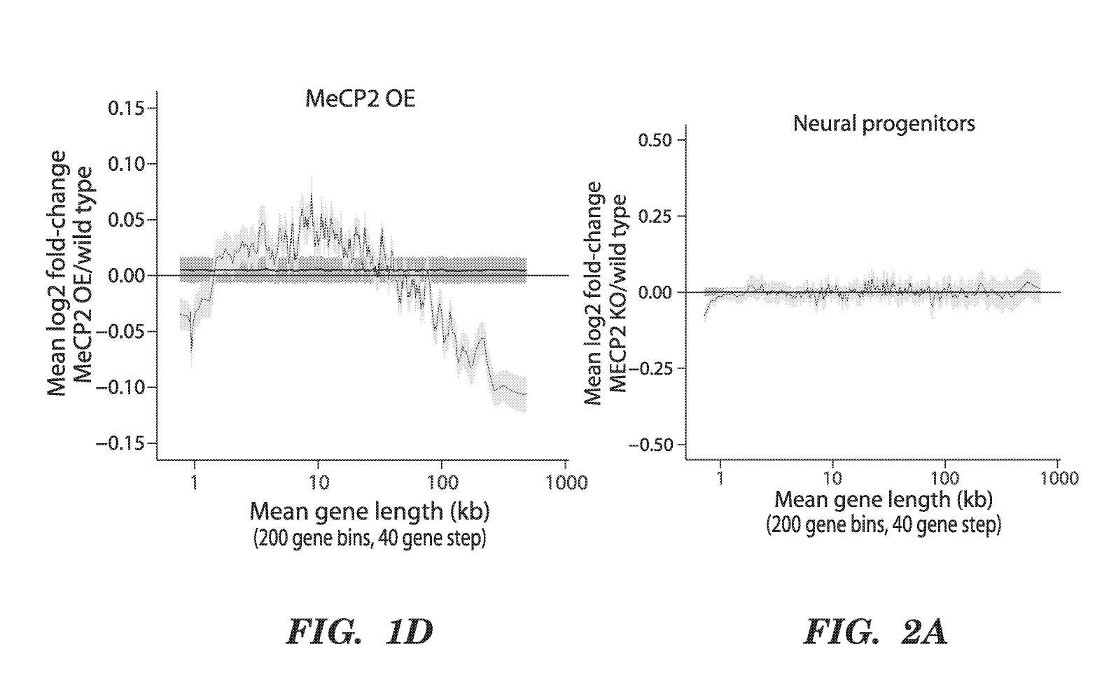

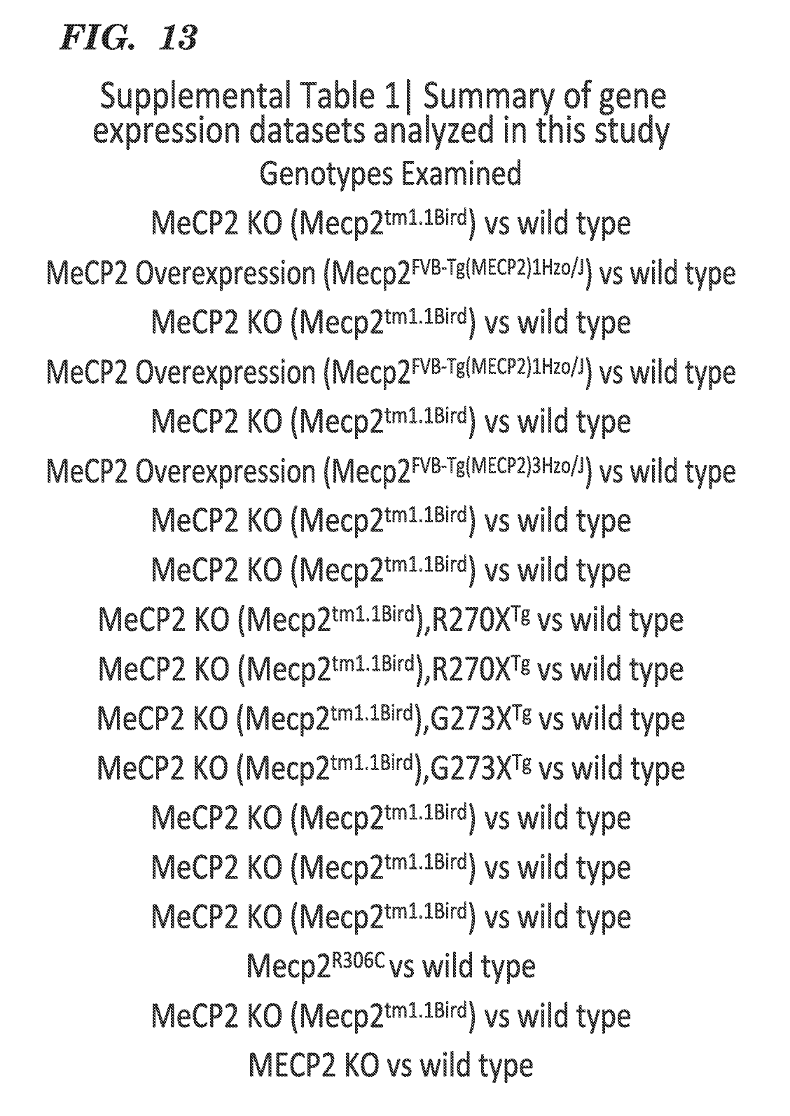

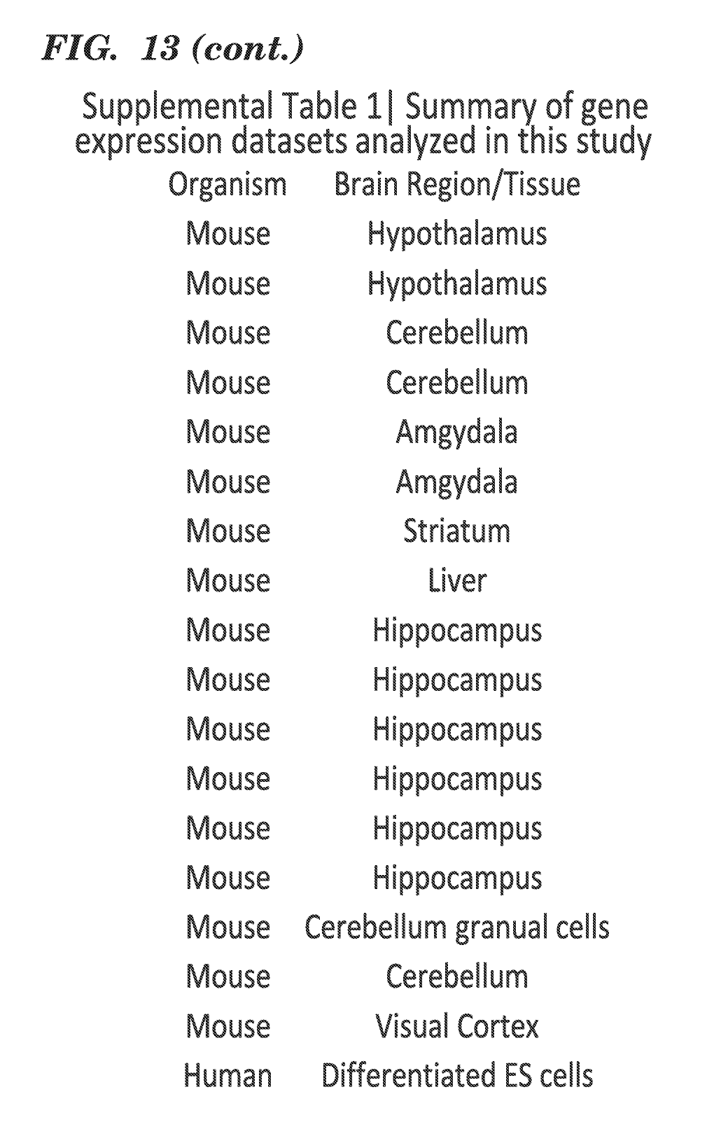

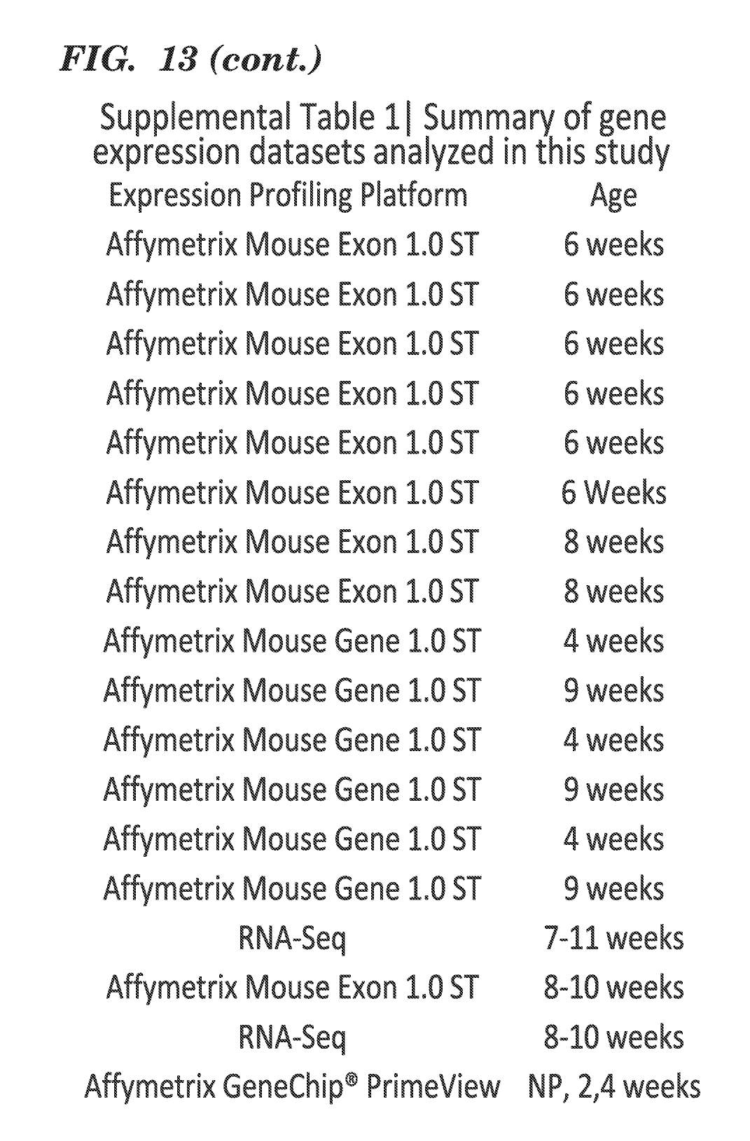

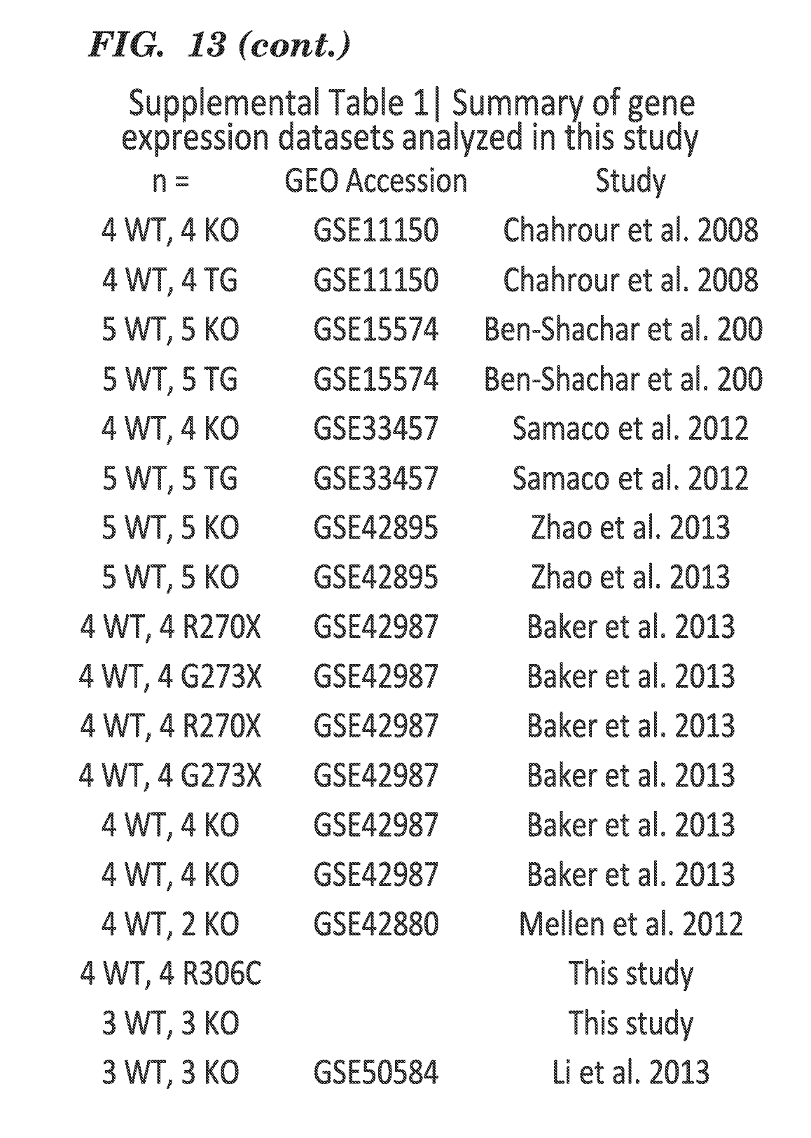

To search for features of chromatin biology or gene structure that are regulated by MeCP2, we asked if genes that are misregulated when MeCP2 function is disrupted have anything in common with respect to MeCP2 binding, DNA methylation, histone modifications, mRNA expression, sequence composition, or gene length. This analysis revealed that genes that are consistently up-regulated in the MeCP2 KO relative to wild-type brains are significantly longer than the genome-wide distribution of gene lengths (FIG. 1a). The extreme length of the genes that are up-regulated in MeCP2 KO brains is apparent in genesets from distinct brain regions in multiple studies performed by different laboratories⁵⁻⁹ (See FIG. 13 for details).

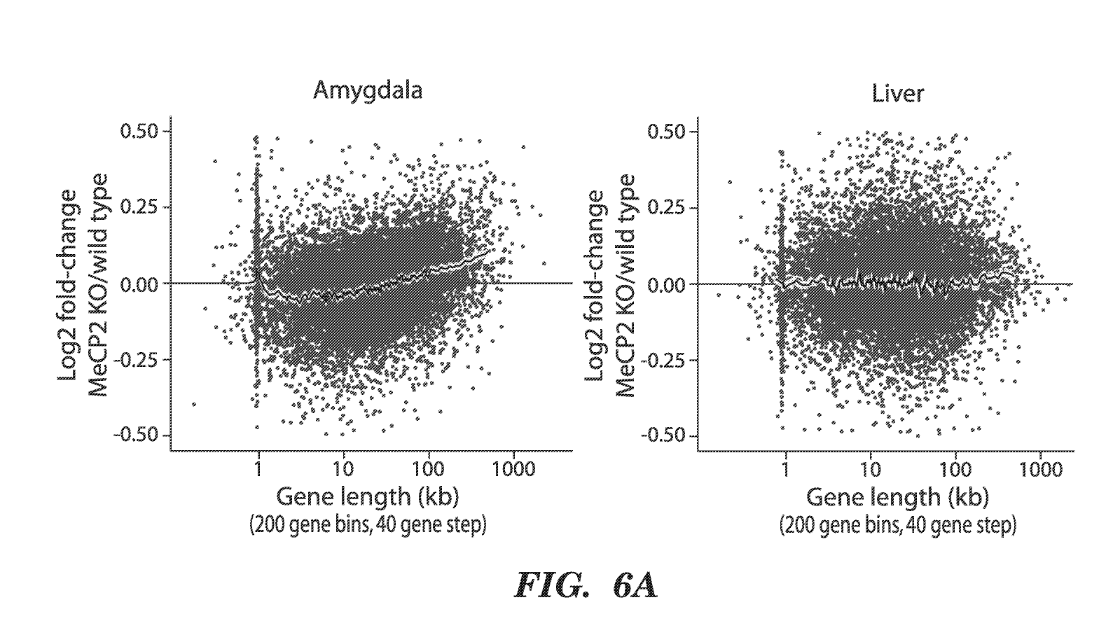

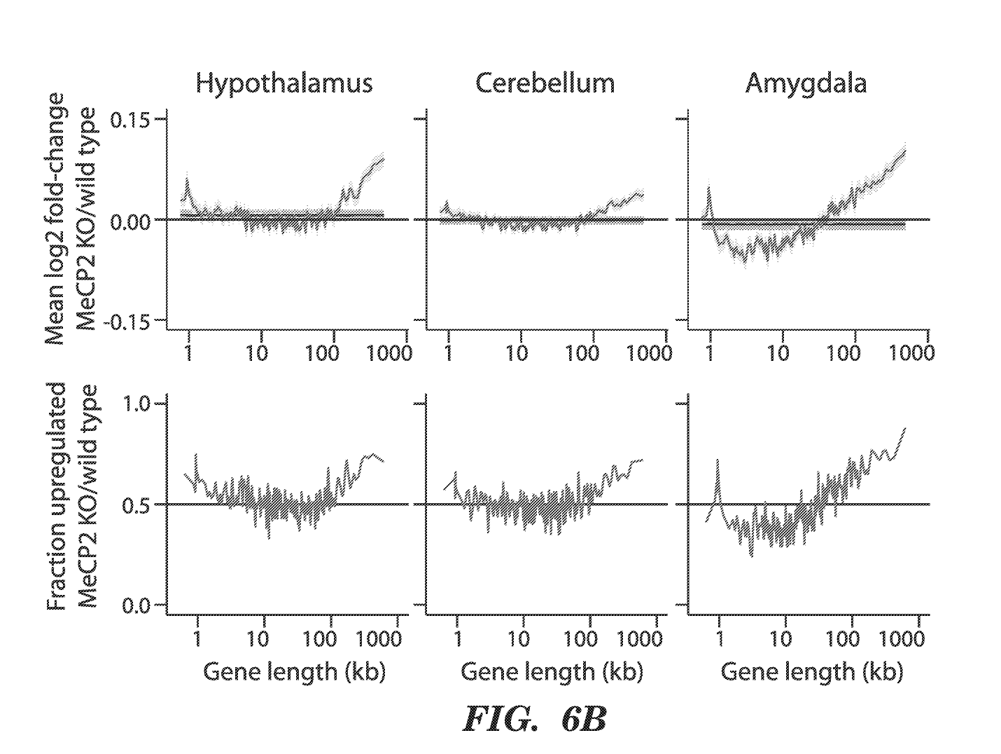

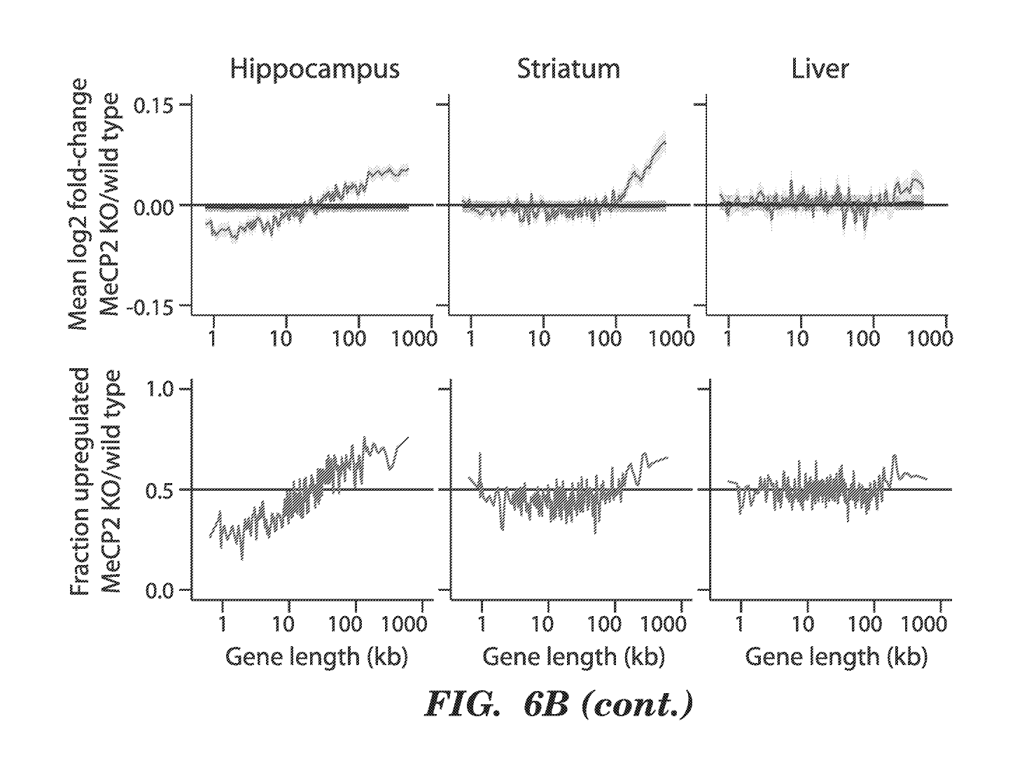

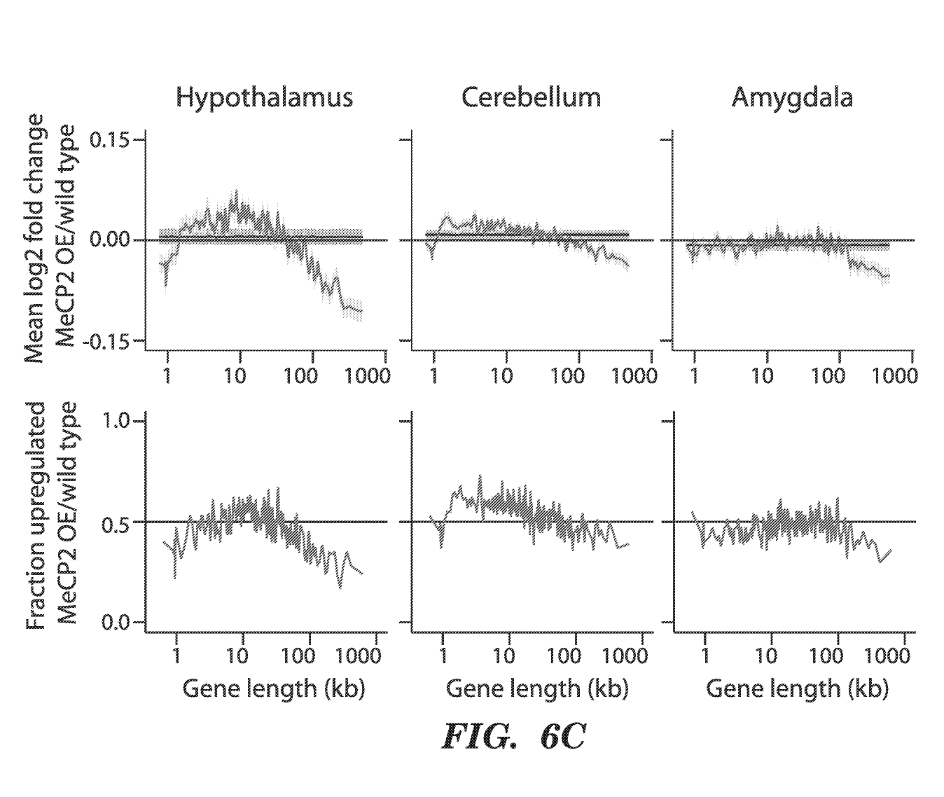

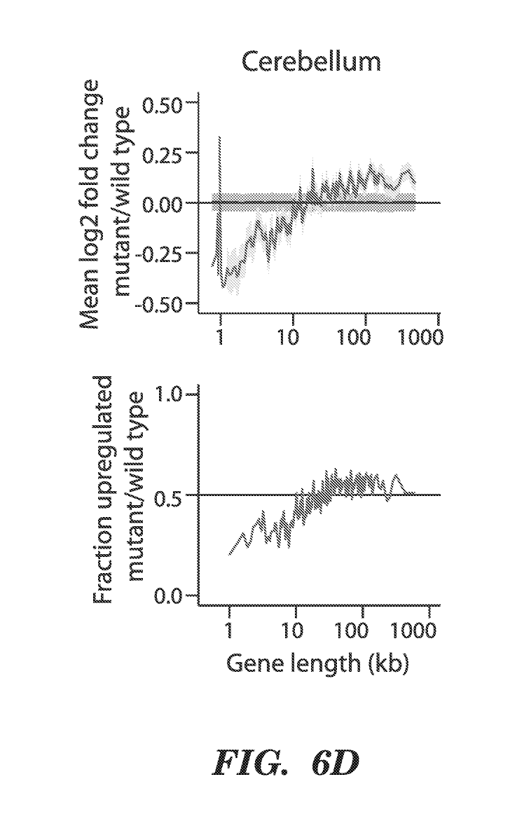

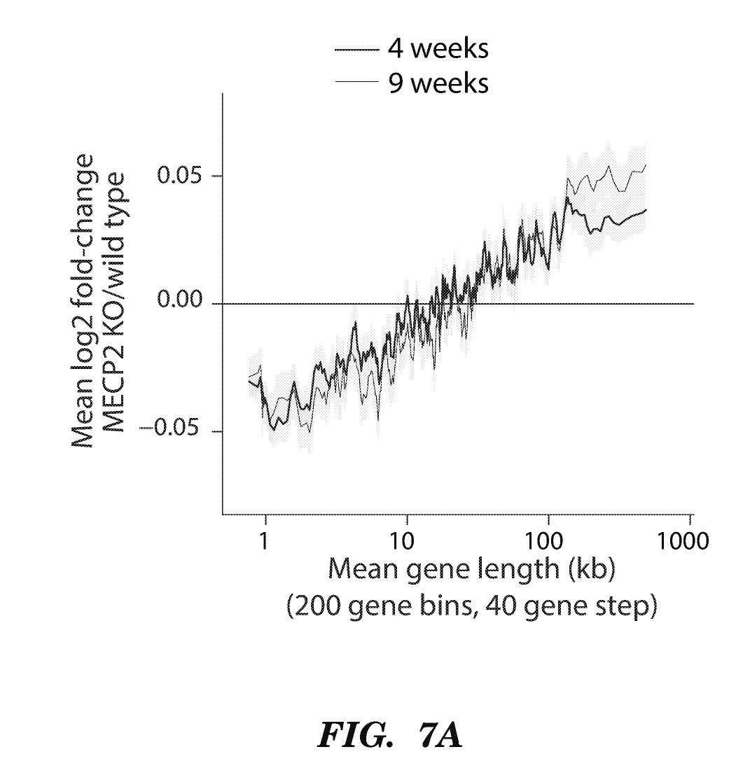

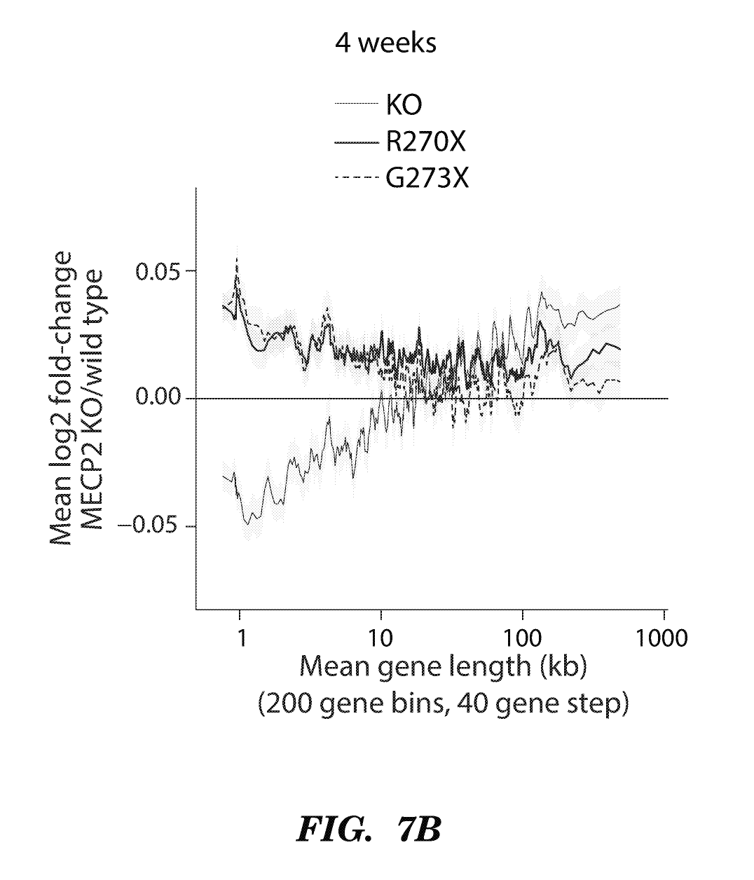

The long lengths of the genes that are up-regulated in the MeCP2 KO raised the possibility that gene length might directly correlate with the extent of transcriptional misregulation that occurs in the absence of MeCP2. Given the relatively subtle differences in gene expression observed when wild-type and MeCP2 KO mice were compared in previous studies, the investigators conducting these studies needed to set thresholds of statistical significance to identify misregulated genes. Thus, a low signal to noise ratio in these experiments may have led to a high false-negative rate of discovery, possibly obscuring detection of a genome-wide length-dependent effect on gene expression in MeCP2 mutant mice. To investigate this possibility, we interrogated published microarray datasets of gene expression (FIG. 13) and plotted the average mRNA fold-change (MeCP2 KO compared to wild type) versus gene length¹². This analysis revealed a widespread length-dependent misregulation of gene expression in MeCP2 KO brains, with the longest genes in the genome displaying the highest level of up-regulation and short genes showing a relative reduction or no change in gene expression (FIG. 1b, FIG. 1c and FIG. 6). Consistent with previous studies demonstrating a relatively modest misregulation of gene expression in MeCP2 KO mice, the magnitude of the up-regulation of long gene expression when MeCP2 function is disrupted is relatively small, but it is robust, occurring in five independent MeCP2 KO microarray datasets derived from several different brain regions (FIG. 1c). Importantly, this length-dependent effect is not an artifact of hybridization-based gene profiling methods, as similar results were obtained when high-throughput RNA-sequencing (RNA-seq) data¹³ was analyzed (FIG. 6c, see below). We conclude from these studies that, relative to the genomic average, loss of MeCP2 results in the up-regulation of long genes in the brain. Thus, a function of MeCP2 may be to constrain transcription in a length-dependent manner in neurons.

To investigate whether these gene expression changes are due to a direct effect of MeCP2, or instead reflect a secondary effect on cell health in Mecp2 mutant mice, we examined gene expression changes in transgenic mice overexpressing MeCP2 (MeCP2 OE). Additional copies of MECP2 have been shown to cause neurological impairment in humans and transgenic mice¹⁴⁻¹⁷. However, like the MeCP2 loss-of-function phenotype, the nature of the gene expression changes in the MeCP2 OE brain is poorly understood. We reasoned that if the observed increase in long gene expression in MeCP2 KO brains reflects a generalized reduction in cell health, gene expression changes in MeCP2 OE samples should be similar to, or uncorrelated with, those observed in the MeCP2 KO. Remarkably, we observed a specific, consistent down-regulation of long genes in microarray datasets generated from three distinct brain regions of MeCP2 OE mice⁵⁻⁷ (FIG. 1c,d). This is the opposite of the effect on long gene expression observed in MeCP2 KO samples. Taken together, these findings indicate that MeCP2 restrains neuronal gene expression in a length-dependent manner.

We next investigated if the length-dependent changes in gene expression correlate with RTT pathology. Using available microarray datasets we assessed the degree to which the length-dependent changes in gene expression observed when MeCP2 function is perturbed track with cellular dysfunction and pathology in various RTT model systems. To begin, we investigated whether the length-dependent gene expression changes observed upon MeCP2 loss are restricted to neural tissues. MeCP2 protein levels are highly enriched in the brain^{2,10,13}, and selective MeCP2 loss in neurons has been shown to cause RTT phenotypes¹⁸ We considered the possibility that MeCP2's ability to restrain long gene expression might be brain-specific, and that this specificity might explain why mutations in MeCP2 principally lead to neuronal dysfunction. While we found that the length-dependent gene misregulation occurs in the absence of MeCP2 in all brain regions tested, we observed little or no length-dependent increase in gene expression in the liver of MeCP2-deficient mice⁹ (FIG. 1a,c). These findings are consistent with the possibility that length-dependent gene misregulation underlies neuronal dysfunction in RTT.

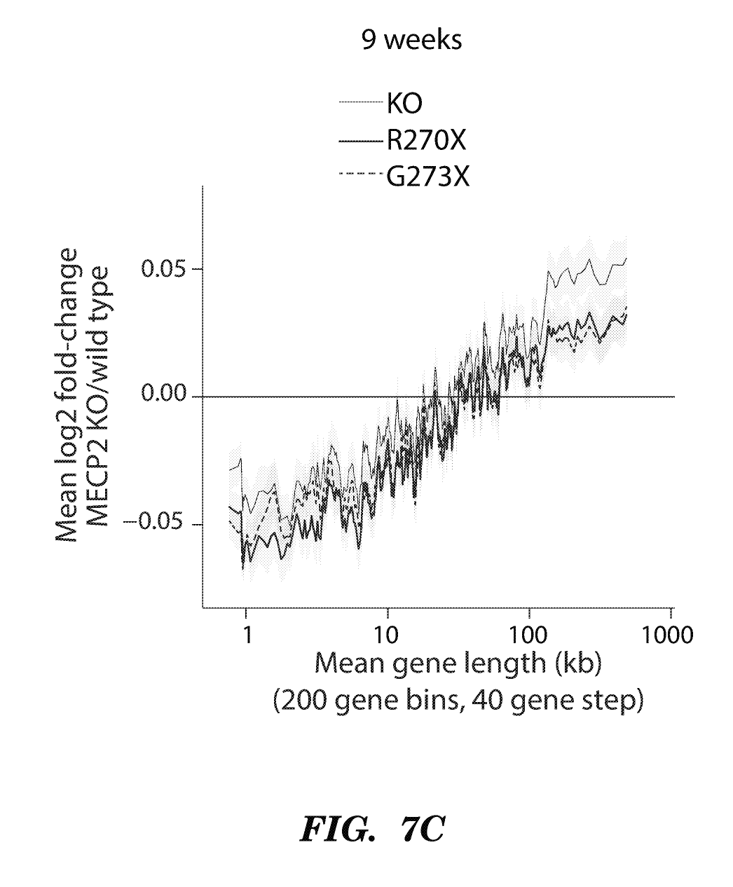

RTT is a progressive disorder, with the onset of symptoms occurring in the postnatal period, just as MeCP2 levels are rising dramatically and synapses are maturing¹⁰. We hypothesized that if the effect of MeCP2 loss on length-dependent gene expression is relevant to RTT then we should observe an increase in the magnitude of length-dependent gene expression changes as MeCP2 KO mice mature and RTT progresses. Consistent with this prediction, we find that misregulation of long gene expression in the hippocampi of MeCP2 KO mice is more dramatic at nine weeks of age than at four weeks of age⁸ (FIG. 7a). Notably, while the length-dependent changes in gene expression are less prominent at four weeks of age than at nine weeks, the changes are still detectable in the hippocampi of MeCP2 KO animals at four weeks of age. This is prior to the appearance of the severe neurological symptoms that occur as RTT progresses, and supports the idea that these gene expression changes are due directly to the loss of MeCP2.

To determine if the magnitude of length-dependent gene misregulation correlates with the severity of RTT phenotypes, we took advantage of published microarray datasets obtained from allele-specific mouse models of RTT. Baker and colleagues' have recently characterized two disease-causing MECP2 truncations, MeCP2-R270X and MeCP2-G273X, by expressing these mutant forms of MeCP2 in MeCP2 KO mice. While both of the MeCP2 mutant proteins are capable of partially rescuing the MeCP2 KO phenotype, the R270X mice still show severe, early-onset RTT, while the G273X animals exhibit more moderate symptoms with later onset. Consistent with the idea that the magnitude of the changes in long gene expression correlate with the severity of RTT pathology, analysis of microarray-based gene expression datasets obtained from the hippocampi of G273X mice early in development indicate less up-regulation of long genes than do samples from R270X mice of the same age (FIG. 7b, FIG. 7c). The more subtle length-dependent misregulation of long gene expression in G273X mice correlates with the slightly delayed kinetics of symptom onset and death in these mice compared to the R270X and KO mice⁸.

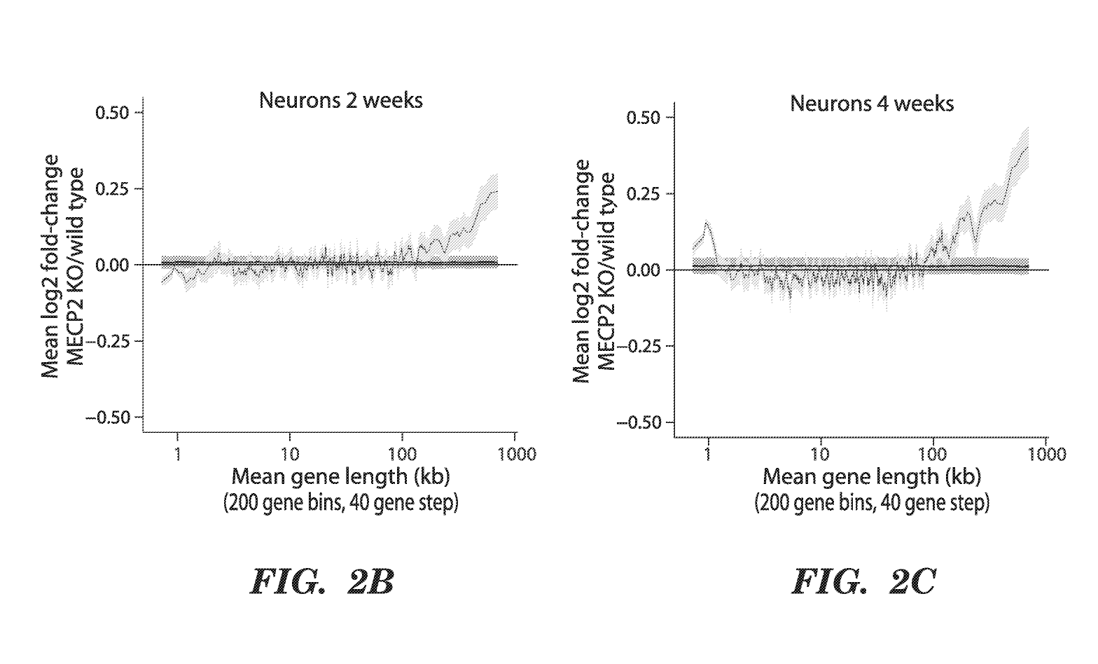

We next asked if long genes are up-regulated when MeCP2 function is disrupted in human neurons. Li and colleagues recently compared the patterns of gene expression in wild-type and MECP2-deficient neurons derived from human embryonic stem cells¹⁹. Upon differentiation, MECP2-deficient human neurons displayed progressive cellular dysfunction compared to control neurons, exhibiting reduced dendritic complexity and a near-absence of detectable neuronal activity. We analyzed the published microarray expression data generated from these MECP2 mutant and control cells, including neural progenitor cells and cultures differentiated into neurons for two or four weeks. We observe no difference in the expression of long genes when wild-type and MECP2 mutant neural progenitors were compared, as might be expected since MECP2 expression is low in these cells (FIG. 2a). By contrast, when neural progenitors were differentiated into neurons we observe a prominent length-dependent misregulation of gene expression in MECP2-deficient human neurons relative to wild-type neurons that becomes more severe between two and four weeks in culture (FIG. 2b,c). Notably, the length-dependent effects we detect here are independent of the reduction in mRNA observed by Li and colleagues in these cells¹⁹. These findings suggest that a conserved function of MeCP2 is to restrain long gene expression in neurons and that an increase in long gene expression, when MeCP2 function is disrupted in the brain, may contribute to RTT.

Non-CpG Methylation and NCoR Function with MeCP2 in Long Gene Repression

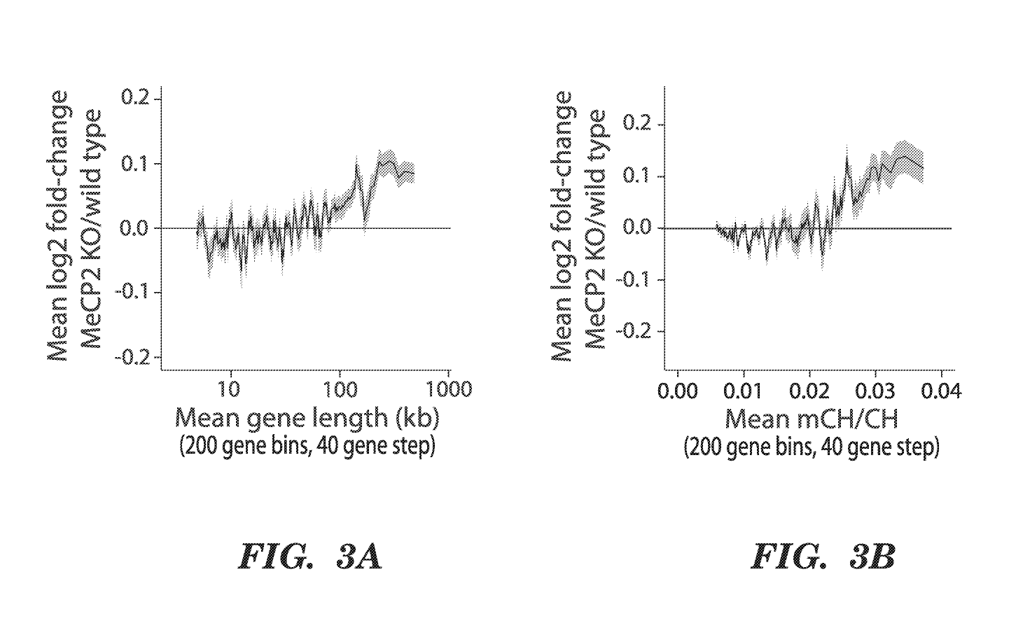

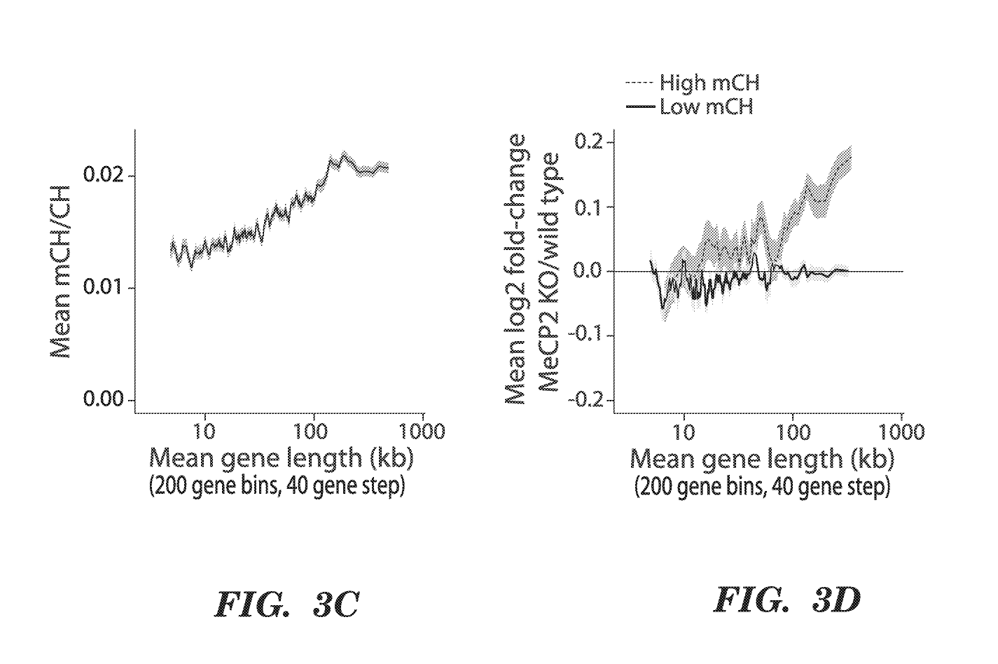

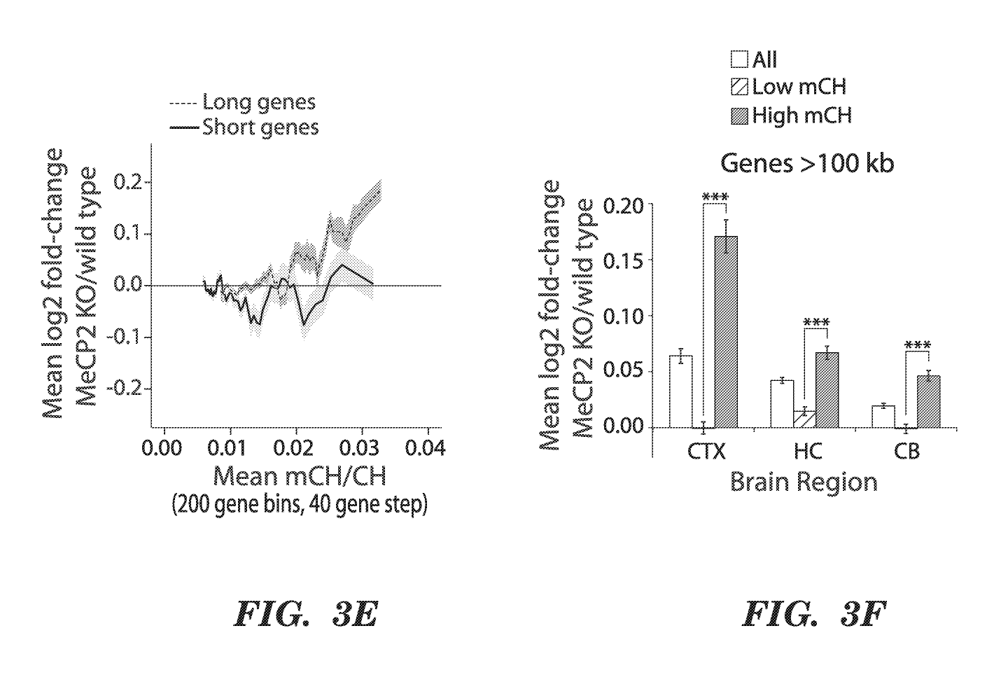

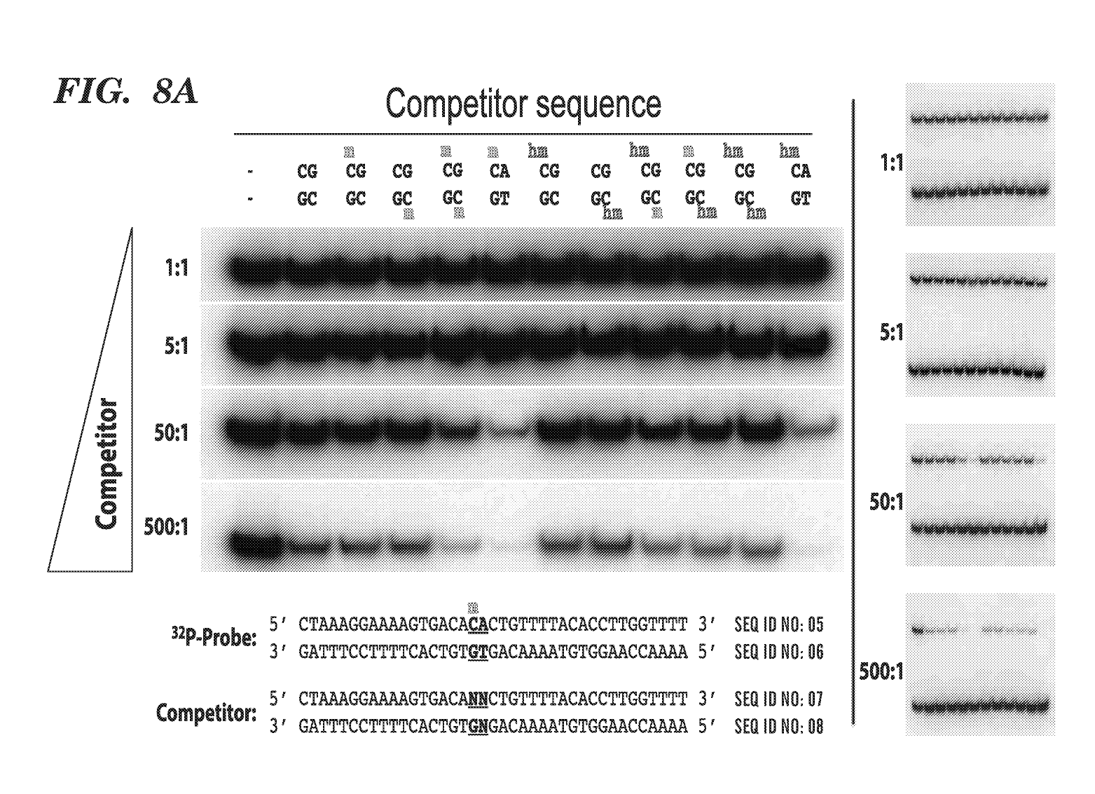

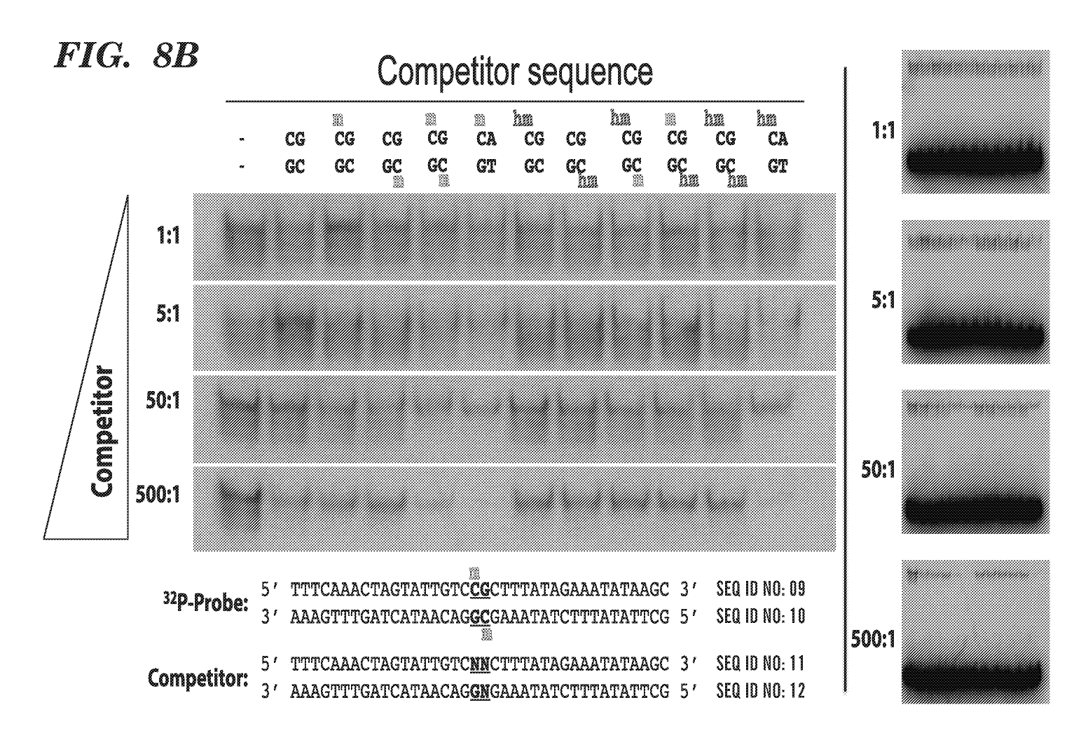

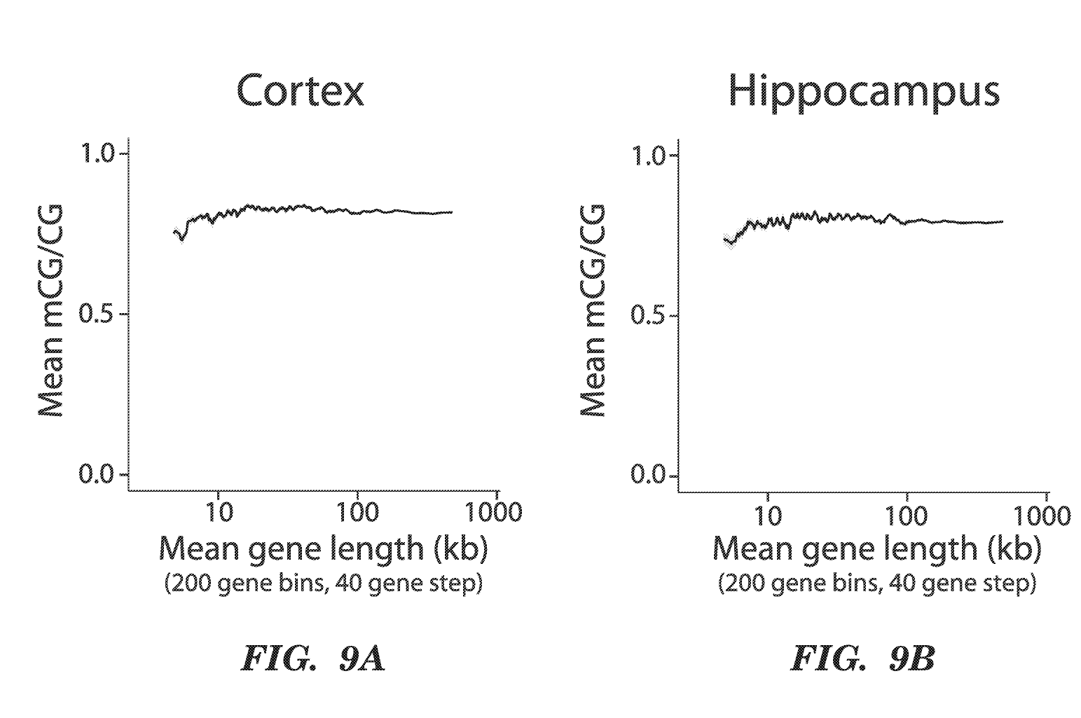

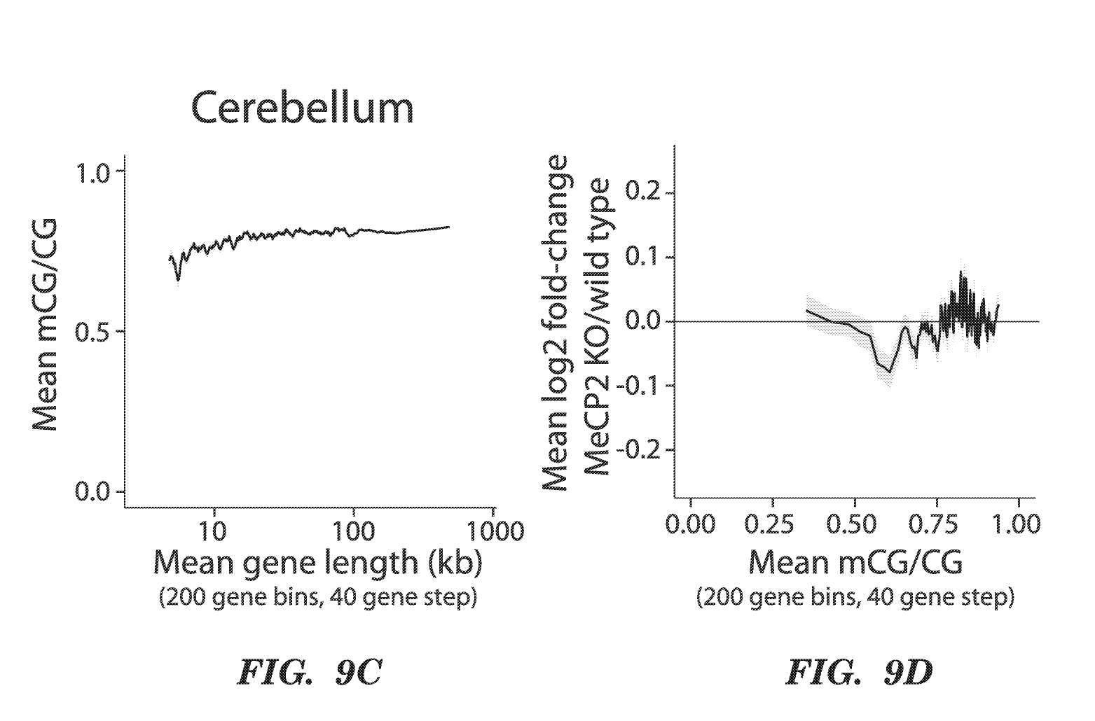

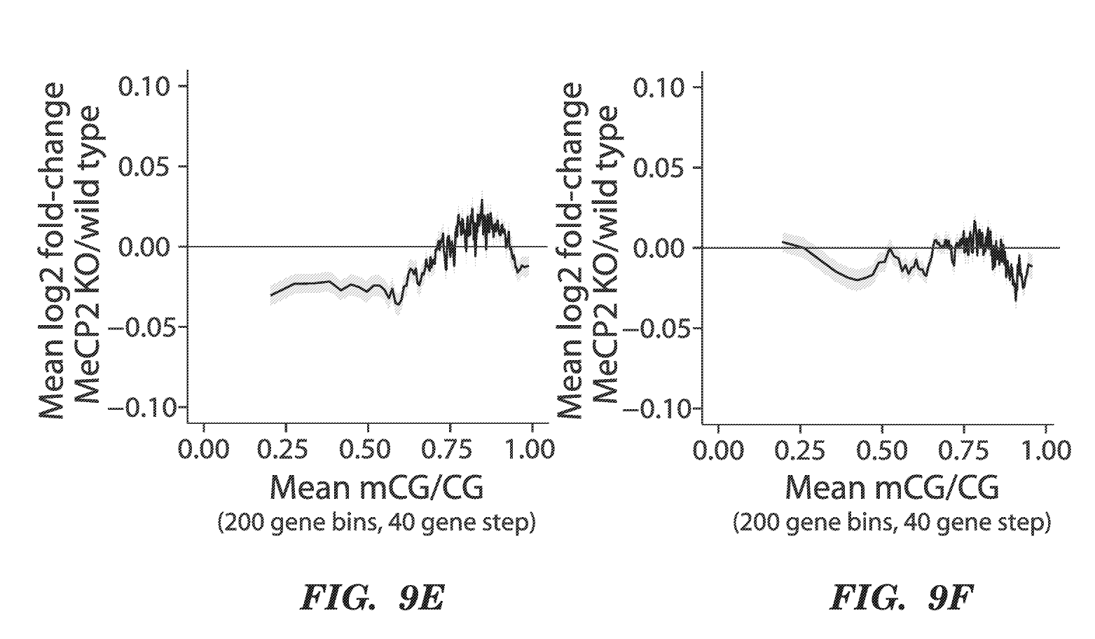

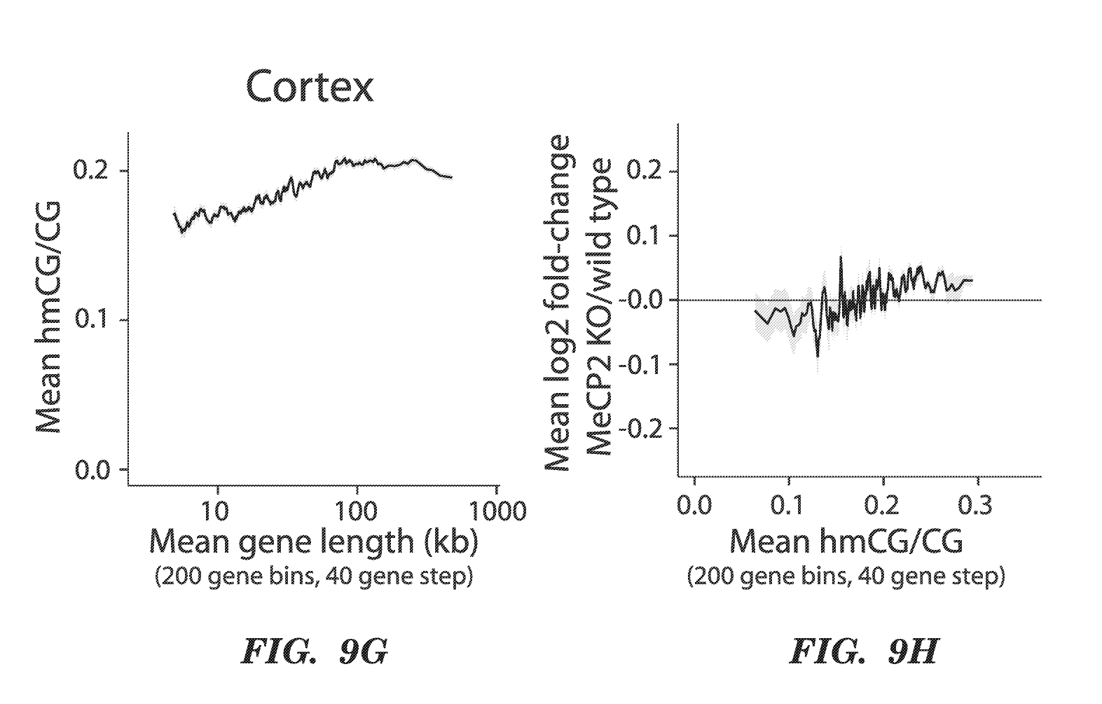

We next investigated the mechanism by which MeCP2 mediates length-dependent gene repression. MeCP2 was initially identified based on its ability to bind methylated cytosines in the context of a CpG dinucleotide²⁰ (mCG). However, to date, it is not well understood how MeCP2 binding to methylated cytosines affects gene expression in vivo. In addition to binding mCG, MeCP2 has recently been suggested to bind to two additional forms of DNA methylation that occur in the brain: hydroxymethylcytosine (hmC)¹³ and methylated cytosines followed by a nucleotide other than guanine (mCH, where H=A or T or C)²¹. Notably, the abundance of hmC and mCH dinucleotides increases significantly across the neuronal genome at the same time during the postnatal period that the level of MeCP2 protein increases dramatically^{10,22-25.} To examine the affinity of MeCP2 for these brain-enriched forms of methylation, we performed an electrophoretic mobility shift assay (EMSA) using the methyl-DNA binding domain (MBD) of MeCP2. In agreement with several studies²⁶⁻²⁸, we find that the MeCP2 MBD does not exhibit high affinity for hmCG. In contrast, we find that the MeCP2 MBD binds mCH with comparable or higher affinity to that of mCG (FIG. 8a to FIG. 8b, Example 1, and Data not shown). Taken together, these data suggest that in addition to binding to mCG, MeCP2 might bind to mCH within the transcribed regions of genes and regulate gene expression in a length-dependent manner. We investigated this possibility by performing RNA-seq on cortical tissue of wild-type and MeCP2 KO mice (FIG. 3a) and comparing this to single-basepair-resolution DNA methylation and hydroxymethylation data from the mouse cortex²⁵ to determine if there is a correlation between the degree of misregulation of gene expression and the levels of mCG, hmCG, and/or mCH (see methods) within the transcribed region of genes. Strikingly, we find a correlation between the levels of mCH, but not mCG or hmCG, within the transcribed region of a gene and the up-regulation of gene expression in the MeCP2 KO compared to wild-type cortex (FIG. 3b, FIG. 9a to FIG. 9h). When we examined gene-body methylation levels across the genes, according to length, we detect higher average levels of mCH in the longest genes in the genome compared to shorter genes, while mCG and hmCG do not show this trend (FIG. 3c, FIG. 9a to FIG. 9h). This suggests that mCH, present at high density in the transcribed region of long genes, may provide high-affinity binding sites for MeCP2, which then functions to temper long gene transcription.

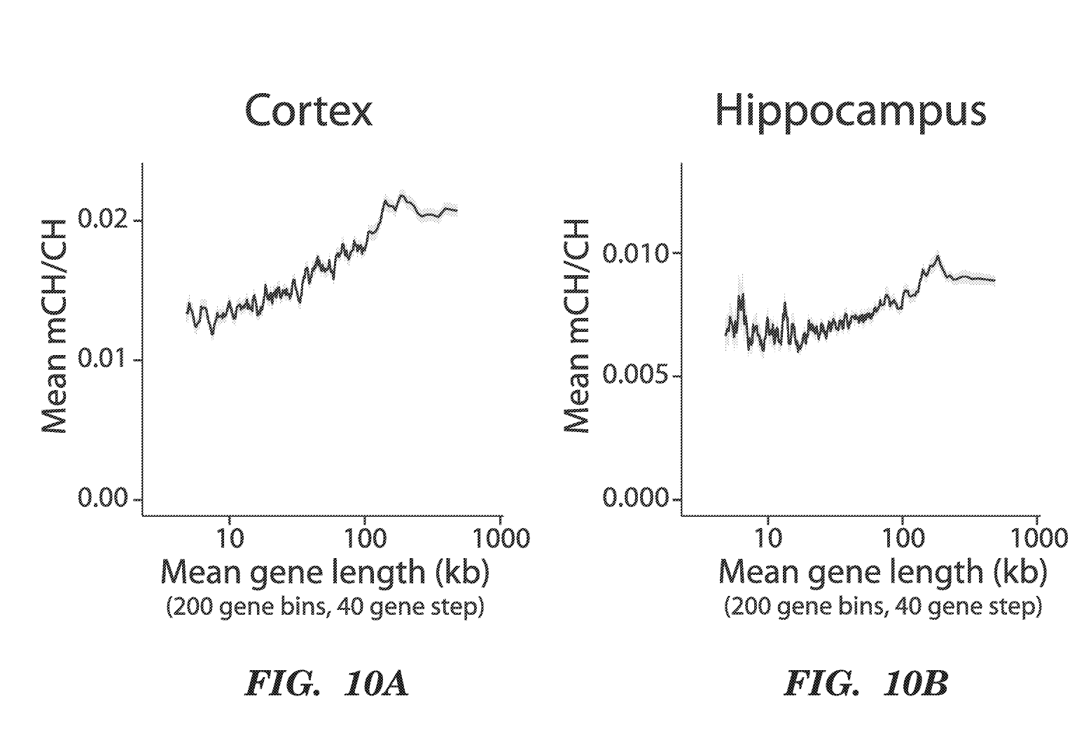

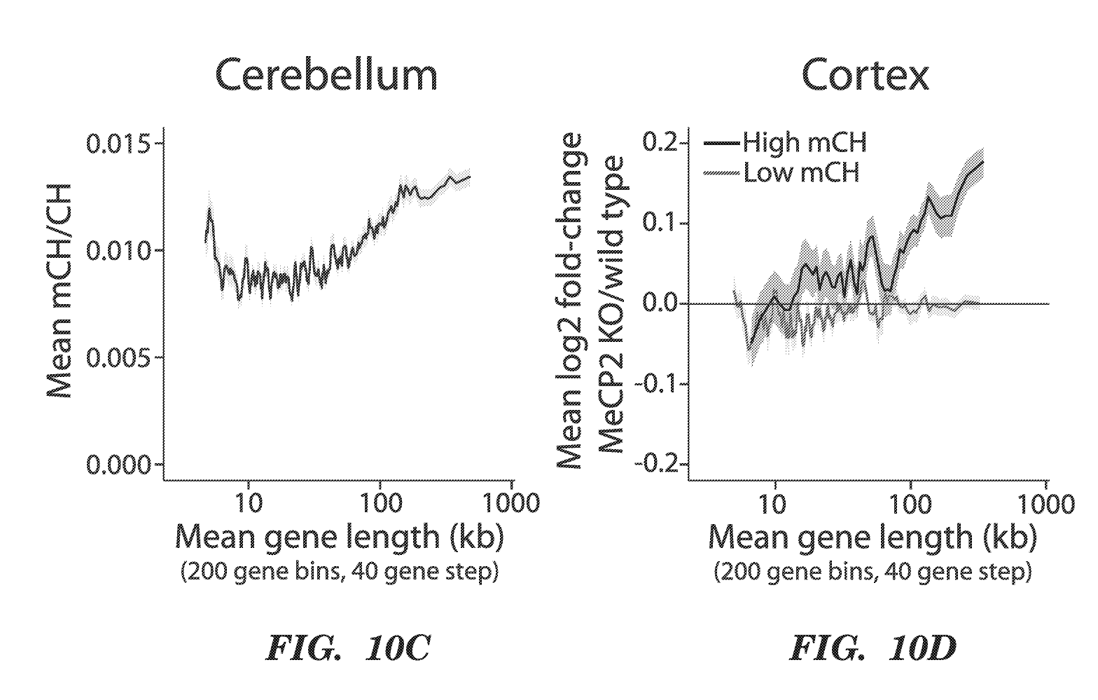

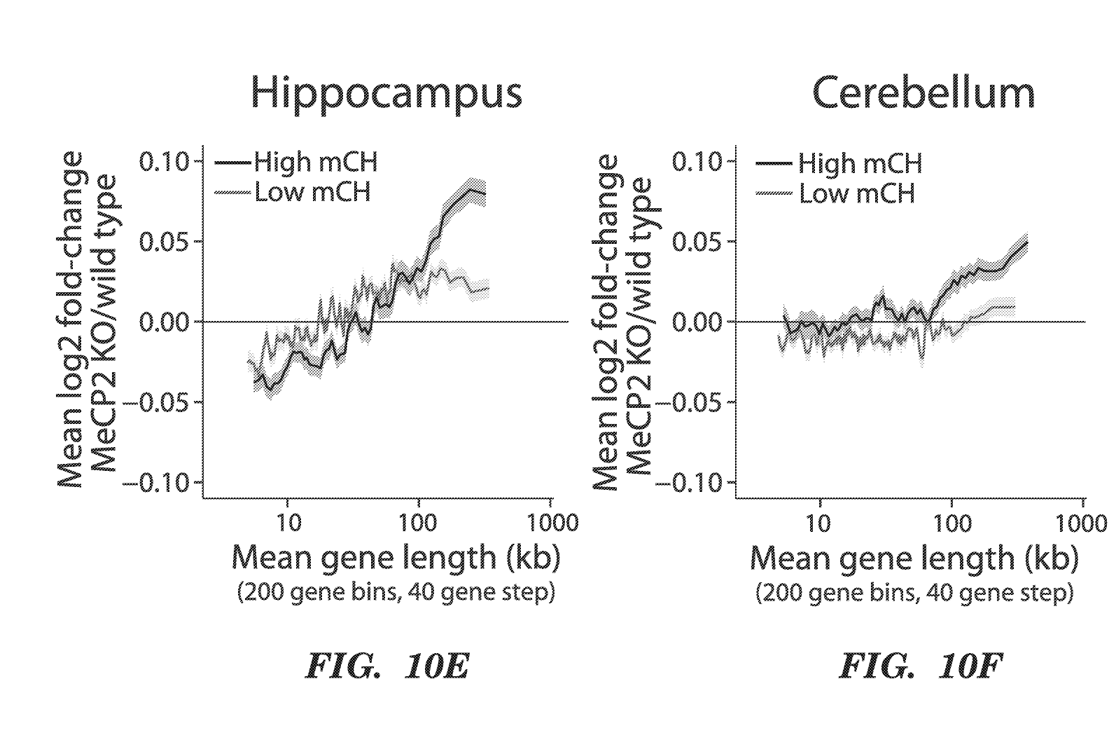

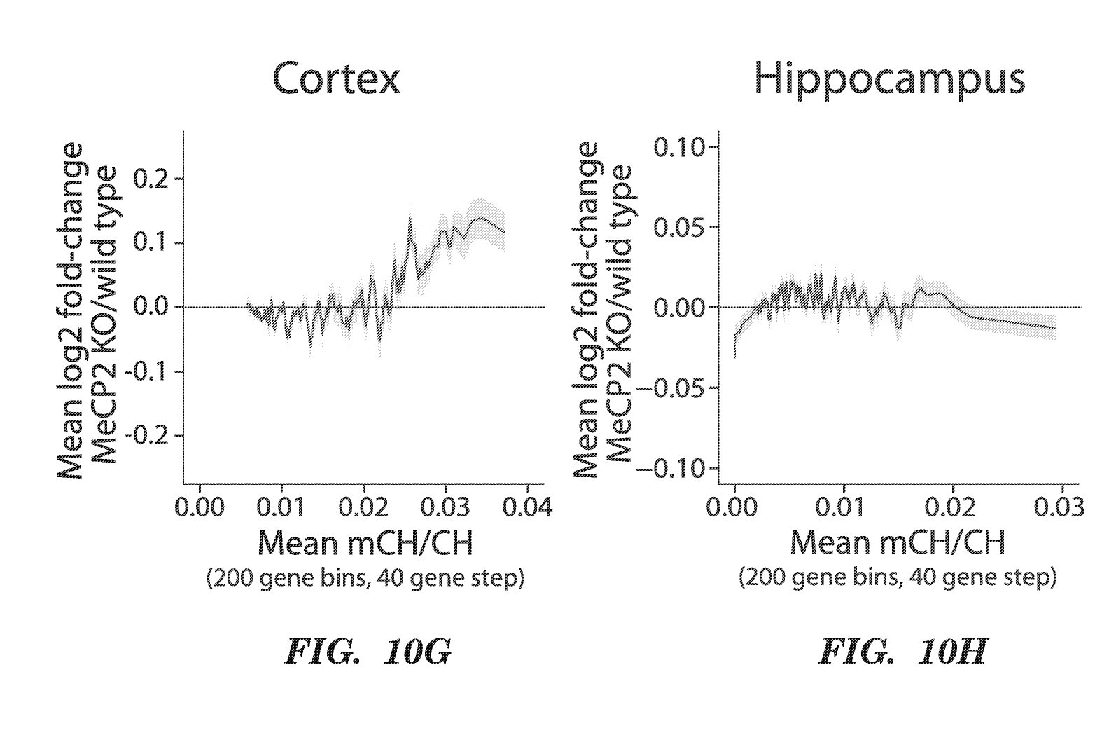

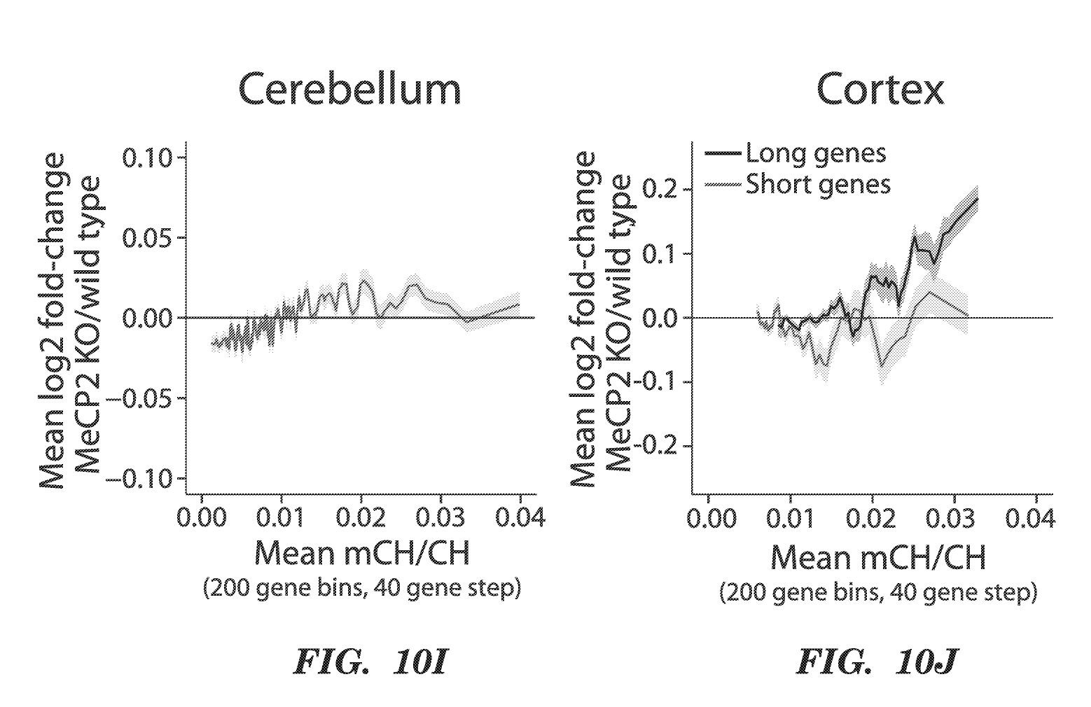

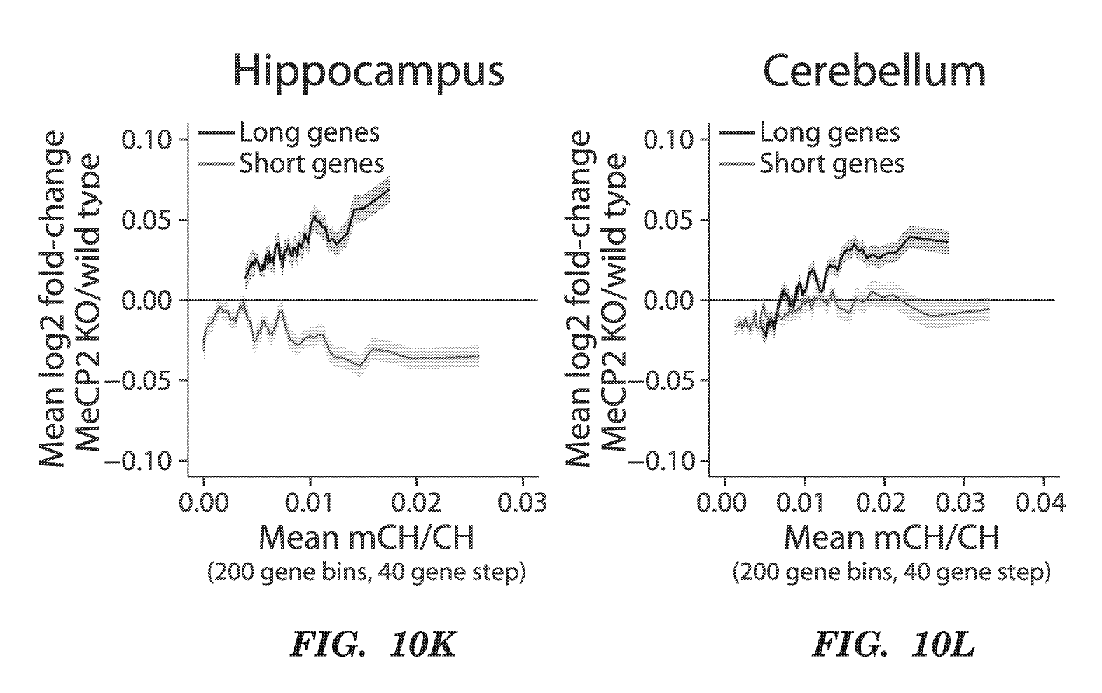

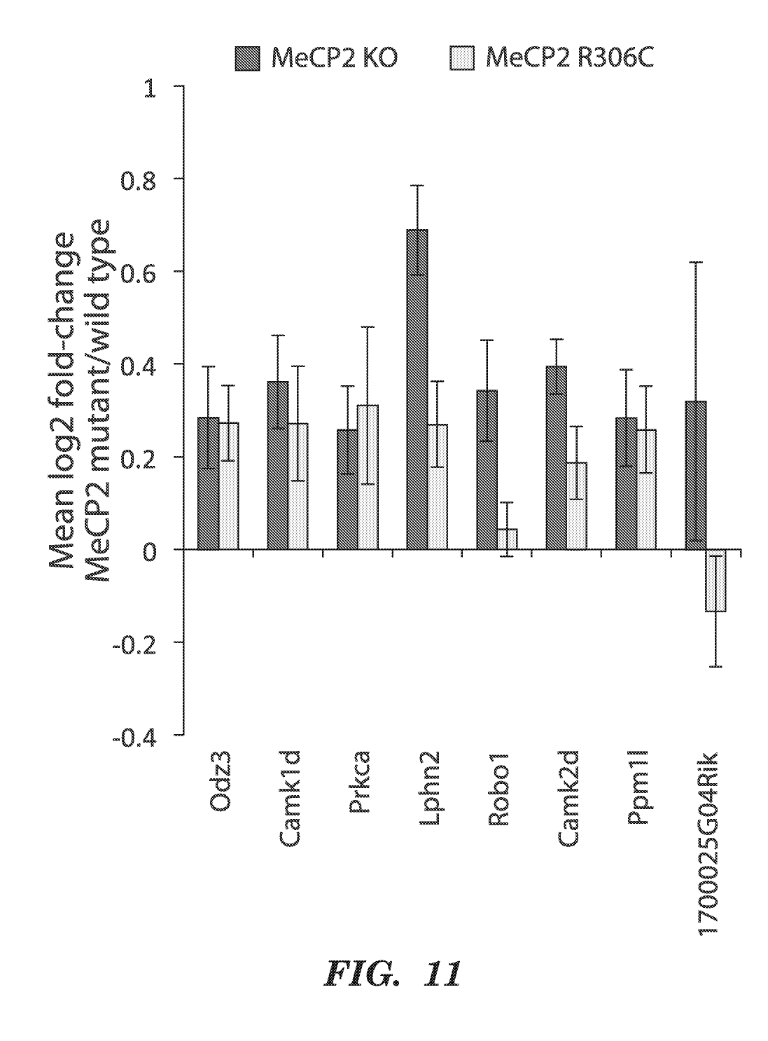

To directly test if the presence of mCH within the transcribed region of a gene is required for length-dependent gene regulation by MeCP2, we asked if the subset of genes in the genome that have low mCH levels are subject to length-dependent gene regulation by MeCP2. Consistent with a requirement for mCH in long gene regulation, we find that long genes with relatively low average levels of mCH across the length of the gene are not misregulated in the MeCP2 KO cortex (FIG. 3d). In addition, we find that the shortest genes in the genome are not up-regulated in the MeCP2 KO, even when the average levels of mCH within their gene bodies is relatively high (FIG. 3e). This suggests that a minimum gene length is required for mCH to facilitate MeCP2-dependent gene repression. To test if mCH is required for length-dependent gene repression by MeCP2 in multiple brain regions, we examined gene expression changes in the MeCP2 KO hippocampus⁸, comparing it to recently published basepair-resolution DNA methylation from this brain region²¹. In addition, we assessed DNA methylation in the cerebellum, performing high-throughput bisulfite sequencing of DNA isolated from the cerebellum of wild type mice and comparing this data to gene expression analysis previously performed on MeCP2 KO and wild-type cerebellum⁶. Consistent with our findings in the cortex, we find that long genes have higher levels of mCH within their gene bodies in the hippocampus and the cerebellum (FIG. 10b, FIG. 10c) and that up-regulation of mRNA expression in the MeCP2 KO does not occur in long genes that have low levels of mCH (FIG. 3f, FIG. 10e, FIG. 10f). Furthermore, we find that in both the hippocampus and cerebellum only long genes display a correlation between mCH and up-regulation of gene expression in the MeCP2 KO (FIG. 10g-FIG. 10l). Together, these results suggest that the extent of gene regulation by MeCP2 may be determined by the number of mCH marks occurring within the gene. Thus, long genes with a high density of mCH in their gene body are most likely to come under control of MeCP2 and therefore become misregulated when MeCP2 is mutated.

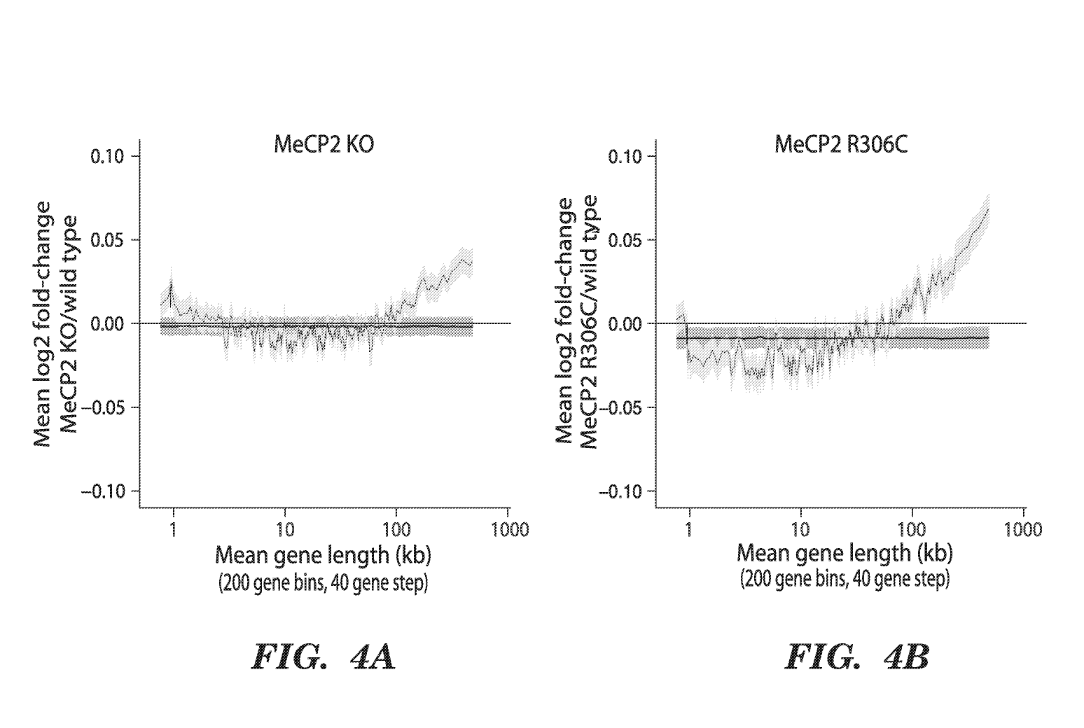

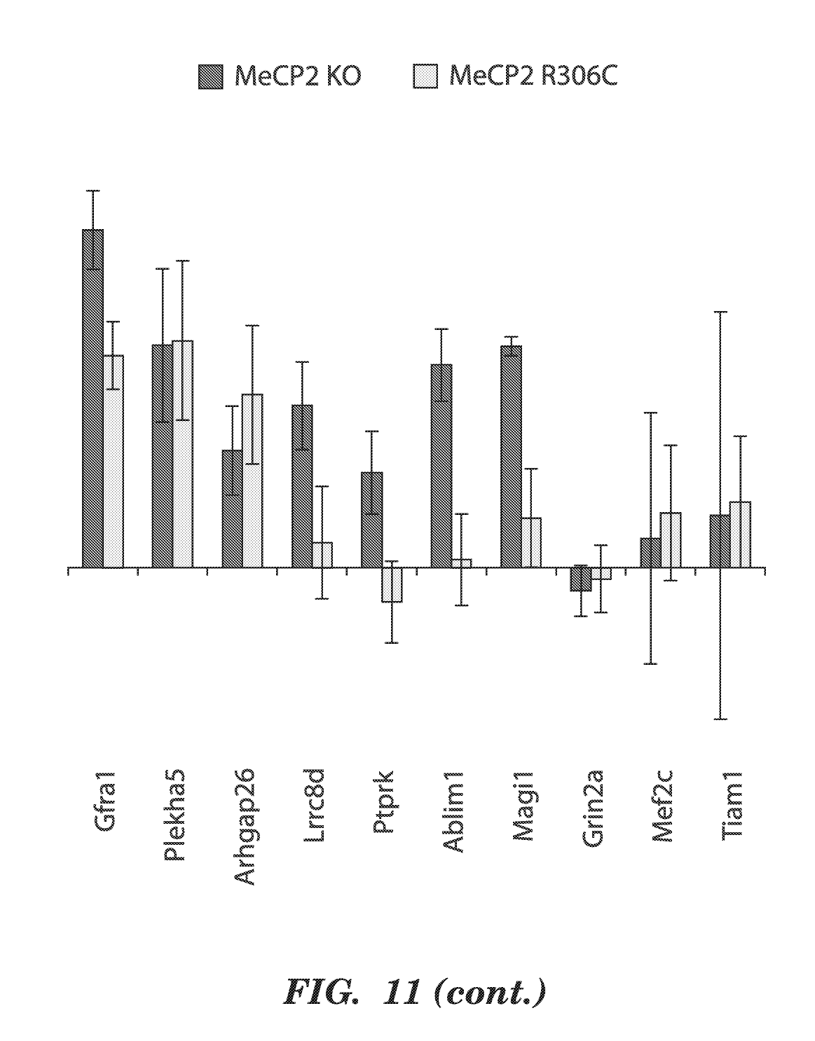

When bound to methylated DNA, MeCP2 is thought to repress transcription through recruitment of transcriptional co-repressor complexes². We therefore asked if abrogation of the repressor activity of MeCP2 affects long gene expression in the brain. Recent analysis has implicated the NCoR/SMRT co-repressor complex as a critical binding partner of MeCP2²⁹. Mutation of arginine 306 to cysteine (R306C) in the C-terminal region of the MeCP2 transcriptional repression domain is a common mutation that leads to RTT. The R306C mutation abolishes the interaction between MeCP2 and the NCoR complex and disrupts MeCP2-dependent transcriptional repression in vitro, but it does not alter MeCP2 protein levels or disrupt interaction between MeCP2 and other protein interactors. Furthermore, transgenic mice carrying a mutation that mimics this patient mutation (MeCP2 R306C) exhibit Rett-like phenotypes²⁹. To determine if NCoR co-repressor binding to MeCP2 is required for MeCP2 regulation of long gene expression, we performed microarray analysis of RNA isolated from the cerebellum of wild-type and MeCP2 R306C mice. Strikingly, we observe a length-dependent increase in mRNA transcribed from long genes in the MeCP2 R306C mutant as compared to wild-type mice (FIG. 4a,b). This finding was corroborated by quantitative RT-PCR analysis of RNA isolated from visual cortices of MeCP2 R306C mutant and wild-type mice (FIG. 11). These observations suggest that the MeCP2-NCoR interaction is required for length-dependent regulation of gene expression and that MeCP2 functions through NCoR to temper long gene expression in the brain.

Long Brain-Specific Genes Affected in RTT and Fragile X Syndrome

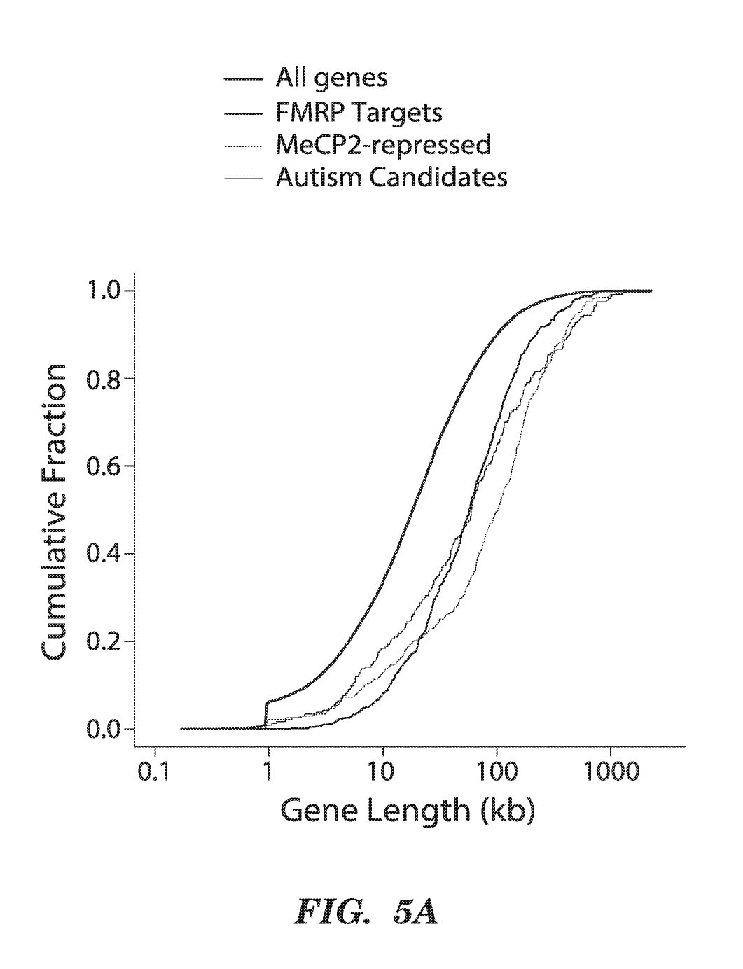

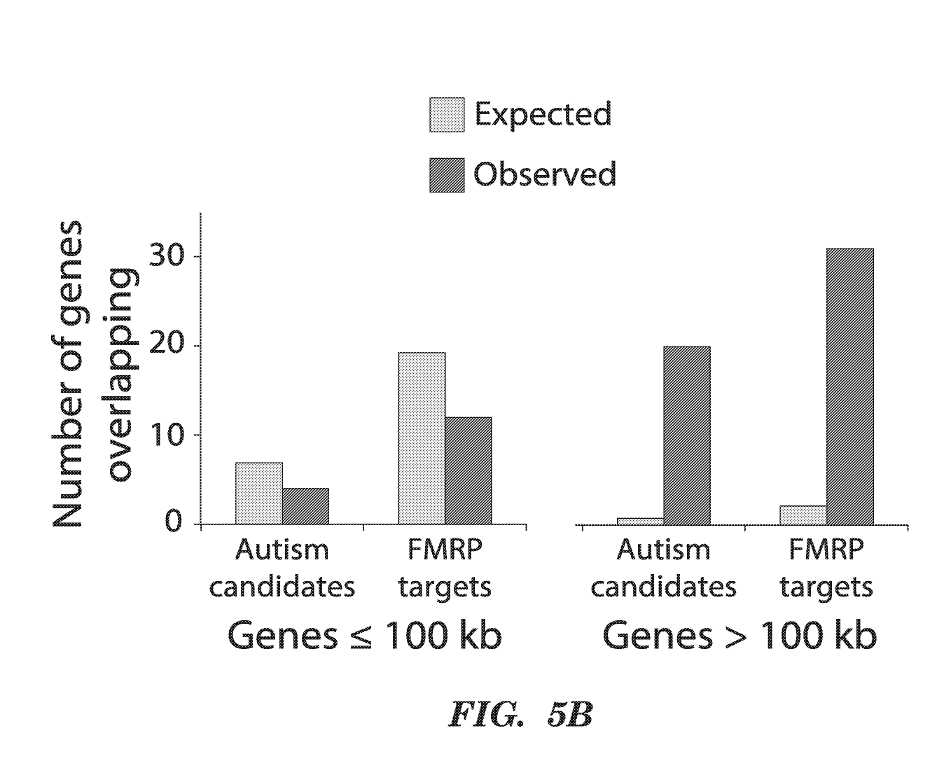

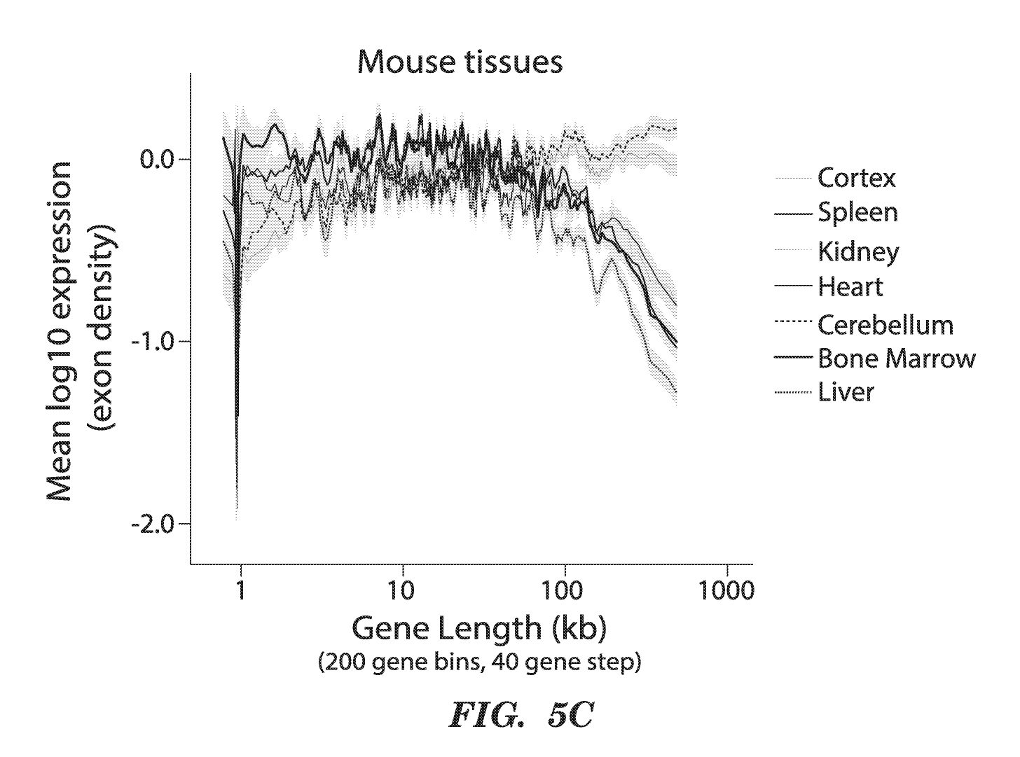

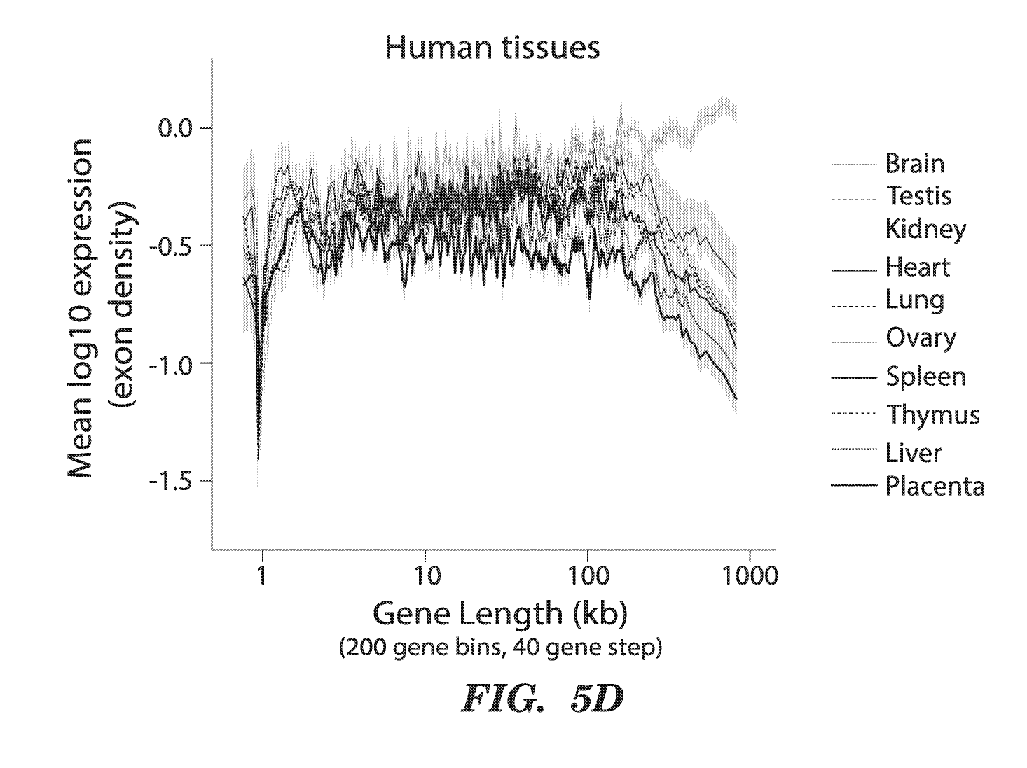

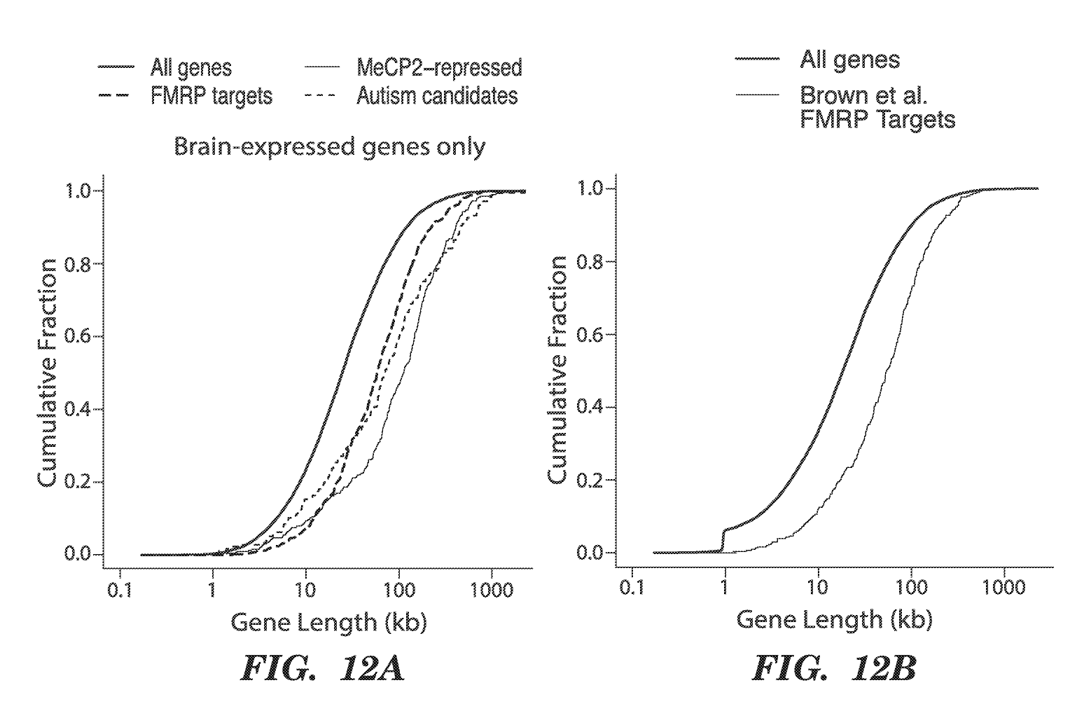

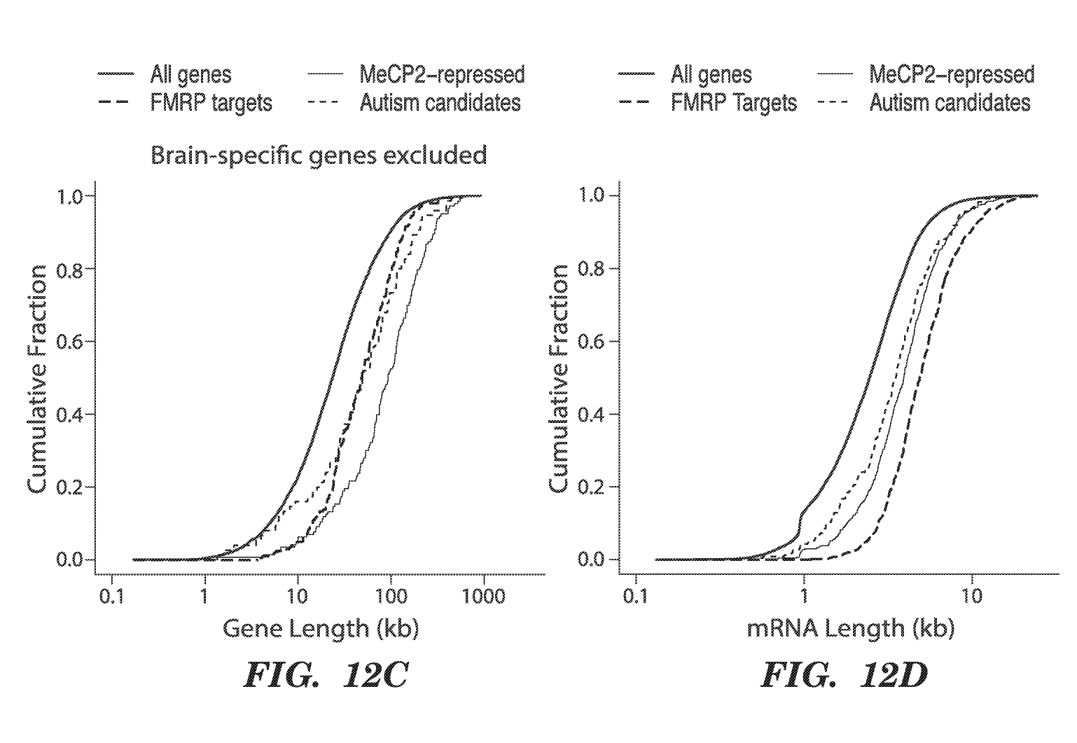

To begin to understand how the misregulation of long gene expression contributes to RTT, we identified the specific long genes that are up-regulated when MeCP2 function is perturbed. We analyzed the data from eight different microarray studies across multiple brain regions to identify 466 MeCP2-repressed genes whose expression is robustly up-regulated in the absence of MeCP2 and down-regulated when MeCP2 is over-expressed (see methods, FIG. 15). Several striking findings are evident from this analysis. First, we find that MeCP2-repressed genes are exceptionally long (FIG. 5a, FIG. 12a, FIG. 12c, FIG. 12d). Second, we note that the MeCP2-repressed genes are enriched for genes that, by gene ontology analysis, have neuronal functions (e.g. post-synaptic density, axonogenesis, voltage-gated cation channel activity; FIG. 13). Third, we find that this set of MeCP2-repressed genes are significantly enriched for genes that have been suggested to be mutated or misregulated in autism spectrum disorders (ASD) (e.g. Cntn4, Mef2C, Sema5a, Chd7; FIG. 5b). This raises the possibility that RTT results from a relatively subtle, yet widespread over-expression of autism genes and genes that have specific functions in the nervous system, while the MeCP2 duplication syndrome may be due to excessive repression of these long genes in the brain. Thus, the precise regulation of long gene expression in neurons may be critical for normal brain function, and the under- or over-expression of long genes or the disruption of the cellular mechanisms that control long gene expression may contribute to autism. Consistent with these findings, King and colleagues recently reported that many genes that have been implicated in autism are exceptionally long¹².

To explore the possibility that disruption of proteins that specifically regulate long gene expression underlies ASDs in general, we asked if a similar misregulation of gene expression might occur in a prominent ASD, Fragile X syndrome (FXS). FXS occurs due to the inactivation of FMRP, a protein that is thought to repress translation of mRNAs in neurons by restricting the rate of ribosome translocation along the mRNA. Like MeCP2, loss of FMRP results in small but widespread changes in gene expression³⁰. We asked if the disruption of FMRP function might preferentially affect the translation of long mRNAs. Strikingly, examination of the gene-length distribution of reported FMRP target mRNAs^{31,32} demonstrated that these FMRP target mRNAs and that the genes encoding them are significantly longer than the genome average (FIG. 5a, FIG. 12a-FIG. 12d, Example 1). Moreover, a comparison of the RTT and FXS datasets revealed a significant overlap between MeCP2-repressed genes and genes encoding FMRP target mRNAs, with this high degree of overlap occurring due to the enrichment of these genesets among genes over 100 kb in length (FIG. 5b). We propose that the up-regulation of long gene function, either through the increased gene transcription (MeCP2) or mRNA translation (FMRP), represents a common axis of pathology in RTT and FXS.

A question that remained to be addressed is why the misregulation of long genes in RTT, FXS and other autism spectrum disorders would lead specifically to neuronal dysfunction. It has been noted that many genes with neuronal function are very long^{33,34}. We considered the possibility that long genes overall might be enriched for functions in the nervous system relative to other tissues. If so, the expression or function of proteins such as MeCP2 and FMRP may have evolved to regulate the expression of long genes specifically in the nervous system. To test this idea, we performed gene ontology analysis of all genes in the genome >100 kb. We find that the longest genes in the genome are highly enriched for neuronal annotations (FIG. 13). Moreover, by examining tissue-specific gene expression datasets we find that long genes are preferentially expressed in the brain: analysis of raw mRNA expression levels in mouse cerebellum and cortex compared to five non-neural somatic tissues³⁵ revealed strikingly high expression of the longest genes in the genome specifically in the brain (FIG. 5c). This brain enrichment is conserved in humans, as analysis of RNA-seq data from ten human tissues (Gray, Harmin et al. in press) revealed robust brain-specific expression of long genes (FIG. 5d). Thus, in mammals long genes, as a population, appear to be expressed and function preferentially in the brain. Notably, while long genes tend to have brain-specific function and expression, brain-specific expression is not a prerequisite for regulation of long genes by MeCP2 or FMRP. Analysis of the subset of genes that are expressed at comparable levels in both brain and non-brain tissues reveals that even these commonly-expressed MeCP2-repressed genes and FMRP targets are extremely long (FIG. 12d, Example 1). These findings suggest that the inappropriate up-regulation or down-regulation of long gene expression preferentially affects genes that function in the brain and contributes to the pathology of RTT, FXS, and other ASDs.

Discussion

Our analysis of gene expression defects in MeCP2 mutant mice suggests that a major role for MeCP2 in the mammalian brain is to temper the transcription of genes in a length-dependent manner. In RTT, loss of this length-dependent gene regulation would lead to a modest but widespread increase in the expression of long genes relative to short genes. Because long genes encode proteins that play important roles in synaptic function and other aspects of neuronal physiology, an imbalance in the expression of these genes may contribute to the cellular and circuit-level defects that occur in RTT.

While it has been known for some time that MeCP2 binds mCG-containing DNA, whether MeCP2 binds mCH and how it exerts its repressive effects in vivo remained largely unexplored. By integrating MeCP2 KO and OE expression datasets with genome-wide bisulfate analysis from the brain, we have obtained evidence that MeCP2 tempers long gene expression in part by binding to mCH within the transcribed region of long genes. Our analysis indicates that the longest genes in the genome tend to have higher mCH density within their gene bodies compared to shorter genes and suggests that the higher the number of MeCP2 molecules bound to mCH in gene bodies, the stronger the MeCP2-dependent repression of gene expression will be. High affinity binding of MeCP2 to mCH within long genes may keep MeCP2 from being removed from the DNA, creating more repressive chromatin structure. Alternatively, binding to mCH may activate the recruitment of the NCoR co-repressor by MeCP2 to alter histone modifications. These chromatin alterations in gene bodies may impede transcriptional elongation, leading to reduced gene expression. The high expression of MeCP2 together with the enrichment of mCH specifically in neurons may have evolved to provide an additional level of regulation for long transcripts that are preferentially expressed in neurons, thus facilitating the fine-tuning of transcription for these critical brain-specific genes. Our study has uncovered evidence of gene-body-mediated regulation of transcription by mCH and MeCP2. Future investigation may reveal additional roles for MeCP2 with mCG, mCH and hmCG in gene regulation within genes or at gene regulatory sites across the genome.

While the mechanism by which MeCP2 constrains gene transcription remains to be fully elucidated, our analysis of MeCP2 R306C mutant mice suggests that the interaction of MeCP2 with the NCoR/SMRT co-repressor complex is required for repression of long gene transcription. The NCoR/SMRT complex contains HDAC3, a histone deacetylase, raising the possibility that MeCP2-NCoR-mediated histone deacetylation may create a repressive chromatin environment within the body of a gene. Interestingly, MeCP2 becomes newly phosphorylated in response to neuronal stimulation, at sites such as threonine 308, whose phosphorylation perturbs the interaction of MeCP2 with NCoR³⁶. This raises the possibility that regulation of the MeCP2-NCoR interaction through phosphorylation of MeCP2 T308 might relieve length-dependent repression of gene expression. This reversal of gene body repression may allow activity-dependent gene transcription to occur rapidly and effectively, facilitating neuronal transcriptional responses to external stimuli. Future experimentation will be required to uncover how the activity-dependent phosphorylation of MeCP2 affects the ability of MeCP2-NCoR to regulate long gene transcription.

A recent study by King and colleagues noted that many candidate autism genes are very long, and showed that inhibition of topoisomerases leads to down-regulation of long genes in neurons¹². Based on the recent detection of a single de novo mutation in topoisomerase genes in individuals with autism, the authors proposed that long gene down-regulation may underlie ASDs. Strikingly, our independent investigation, focused on understanding the molecular etiology of RTT, has uncovered a role for MeCP2 in tempering long gene expression in the brain. This to our knowledge represents the first direct evidence that mutation of a known ASD gene leads to widespread misregulation of long gene transcripts. Our additional observation that FMRP targets are unusually long provides further support that loss of long gene repression may contribute to neurodevelopmental disorders. Finally, we have shown that over-expression of MeCP2 leads to the widespread inhibition of long gene expression. Because MECP2 overexpression leads to neurological dysfunction in human patients and mice, this suggests that a precise set-point of expression for the longest genes in the genome may be critical for proper neuronal function. Taken together, our study implicates mutations in regulators of long gene expression as a major underlying mechanism of pathology in ASDs.

Our finding that up-regulation of long gene transcripts occurs in RTT, together with the finding that inhibition of topoisomerases leads to the selective down-regulation of long genes in neurons¹², suggests that pharmacological interventions (e.g. topoisomerase inhibition) that temper the expression of long genes to restore them to appropriate levels in individuals with RTT could ameliorate aspects of the cellular dysfunction that occur in this neurological disorder.

Olfactory Receptor Misregulation in MeCP2 Mutants

A notable exception to the length-dependent alterations in gene expression that we observe in MeCP2 mutants is a distinct population of very short genes, approximately 1 kb in length, that display up-regulation in the MeCP2 KO and down-regulation in the MeCP2 OE in most datasets. This altered population is visible as a spike in mean fold-change vs length plots for both mouse brain regions and human cells (e.g. FIG. 1a,b; FIG. 2c, FIG. 6). Inspection of the genes at this length revealed that this spike reflects changes in the expression of the olfactory receptor genes. Several hundred highly paralogous olfactory receptor transcripts of nearly uniform length are present in mice and humans. They occur in several large clusters in the genome and are highly repressed in all cell types except for the neurons of the olfactory system⁴¹ (this is visible as a downward spike in expression in FIG. 5c, FIG. 5d). While the very low expression of these genes leads to a high degree of noise in their measured expression levels (observable as a large spread of fold-change values in FIG. 6a and FIG. 6b), the consistent change of the population average across multiple datasets suggests that MeCP2 is required to maintain full repression of these genes. Unlike the length-dependent regulation by MeCP2 that we observe, the regulation of the olfactory receptor genes by MeCP2 is likely to occur independently of mCH, as recent basepair-resolution analysis of DNA methylation in the brain detected little or no mCH across the large genomic domains containing the olfactory receptors genes²⁵. It is unclear what the functional consequences in the brain will be as a result of olfactory receptor misregulation in MeCP2 mutants, as even upon derepression in the MeCP2 KO the levels of these transcripts are extremely low. Future studies of the olfactory neurons in the MeCP2 KO may uncover an important role for MeCP2 in the repression of olfactory receptors.

Affinity of MeCP2 for methylcytosine and hydroxymethylcytosine

The recent discovery that mCH and hmCG build up postnatally in the brain to high levels²²⁻²⁵ suggests that these forms of DNA methylation may play a unique and important role in the maturation and function of neurons. The build up of MeCP2 levels in neurons parallels the increase of hmCG and mCH¹⁰, suggesting that MeCP2 may work in conjunction with these marks. The affinity of MeCP2 for hmC and mCH can provide clues to how they might affect MeCP2 binding or function in vivo. Several studies have assessed the affinity of MeCP2 for hmC or mC in vitro^{21,26-28,42,43,} but there has been limited work explicitly assessing the relative affinity of MeCP2 for all possible forms of methylation (unmethylated DNA, mCG, hmCG, mCH and hmCH) in parallel and within an otherwise identical DNA sequence context.

To directly compare the relative affinity of the methyl-DNA binding domain (MBD) of MeCP2 for each form of DNA methylation we have performed EMSA analysis using competitor oligonucleotides in which the central dinucleotide is altered, while the rest of the oligonucleotide sequence and the position of the methylation site(s) are kept constant. Using unlabeled oligonucleotides to compete for binding against a mCG or mCA radiolabeled probe, we find that the relative affinity of a MeCP2 MBD fragment (amino acids 81-170) for mCA is substantially higher than its affinity for unmethylated DNA, and this affinity is comparable to or higher than the affinity of MeCP2 for symmetrically-methylated CG (FIG. 8a, FIG. 8b). These results are largely consistent with the recent study by Guo and colleagues, which detected a strong affinity of MeCP2 for mCH that is comparable to that of mCG²¹.

In contrast to mC, we find that the dinucleotide context of hmC dramatically alters the relative affinity of the MeCP2 MBD in EMSA assays. We observe that probes containing hydroxymethylation at one or both cytosines in the CG context compete for binding of MeCP2 with similar efficacy to that of an unmethylated oligonucleotide (FIG. 8a,b). This suggests that the binding affinity of MeCP2 to hmCG is similar to that of unmethylated DNA. Strikingly, an oligonucleotide containing hmCA competes for binding with a high efficacy that is comparable to that of mCG and mCA, suggesting that conversion of mCA to hmCA does not substantially reduce the affinity of MeCP2 for this methylated dinucleotide.

These results may resolve seemingly incongruent findings from several previous studies examining the affinity of MeCP2 for hmC. Mellen and colleagues¹³ recently observed a high affinity of MeCP2 for hmC-containing DNA that was comparable to the affinity of MeCP2 for mC-containing DNA, while multiple other studies have noted reduced affinity of MeCP2 for hmCG compared to mCG²⁶⁻²⁸. Notably, Mellen et al. used probes that incorporated hmC throughout the DNA sequence and therefore contained many hmCH sites, while the other studies were performed with probes in which only defined hmCG sites were present. Thus, in agreement with our results, the high relative affinity of MeCP2 for hmC observed by Mellen et al. may stem from the presence of hmCH in their DNA probes, while the lower relative affinity detected for hmC in other studies may have resulted from the presence of hmCG alone.

The differential affinity of MeCP2 for hmC, depending on the dinucleotide context, may have important implications for the binding and function of MeCP2 with hmC across the genome. Recent genome-wide, basepair-resolution analysis of hydroxymethylation in the brain indicates that while hmCG is present at substantial levels, hmCH is exceedingly rare²⁵. Thus the primary effect of the conversion of mC to hmC in the neuronal genome may be to reduce the affinity of MeCP2 binding at mCG sites, while conversion of a small number of mCH sites to hmCH sites may not substantially alter the binding of MeCP2 at these locations. Future analysis may uncover how these differing affinities of MeCP2 for hmCG and hmCH affect MeCP2-dependent gene regulation in vivo.

Brain-Specific Expression of Long Genes and Regulation by MeCP2 and FMRP

Our finding that long genes in general are expressed more highly in the brain than in other tissues raised the possibility that the long length of FMRP targets, and MeCP2-repressed genes is not due to a primary effect of length in determining regulation but instead occurs as a secondary consequence of the longer average length of genes that are expressed in the brain. Therefore, to control for expression in the brain, we first filtered the genome for genes that are robustly expressed in the brain, calculating the average expression (exon density) of all genes across the cortex and cerebellum and selecting only genes that lie in the top 60% of expression values. We then reexamined the length distribution of each geneset (FIG. 12a). This analysis confirms that putative FMRP targets, MeCP2-repressed genes and autism candidate genes are not composed of extremely long genes solely as a result of the high expression of long genes in the brain.

In addition to raw expression levels, the finding that long genes as a population are specifically expressed in the brain also raised the possibility that MeCP2 or FMRP primarily target brain-specific genes for repression and that the up-regulation of many long genes that we observed in the MeCP2 KO is only a secondary effect of the de-repression of these genes because they are brain-specific. To examine this possibility directly, we filtered the genome for genes that are comparably expressed in the brain and other somatic tissues, selecting only genes that have expression in the mouse brain (average exon density of cortex and cerebellum) that is within two-fold of their average expression in non-brain tissues (average exon density of all other tissues). Examination of the MeCP2-repressed genes, FMRP target genes, and autism candidate genes that are within this subset of genes with comparable neural and non-neural expression revealed that they are extremely long compared to all genes that meet this criterion (FIG. 12d). This further supports the conclusion that gene length, not brain-specific expression, is an underlying determinant for regulation by MeCP2 or FMRP.

Recent studies have shown that FMRP binds to target mRNAs and stalls translation^{30,31}. It is therefore likely that the relative long length of genes encoding FMRP targets reflects targeting of long mRNA transcripts. To assess the length of FMRP target mRNA directly, we examined the length of the mature transcripts for FMRP targets. We find that FMRP target mRNAs are extremely long compared to the transcriptome average (FIG. 12e). Furthermore, similar results were observed when controlling for expression in the brain (data not shown). These findings are consistent with FMRP binding throughout the coding sequence of mRNAs to impede ribosomes³¹ and suggest that mRNA length contributes directly to the level of regulation by FMRP. Notably, while proteome-wide analysis of translational control by FMRP has not been performed, Darnell and colleagues³¹ did assess the level of repression by FMRP for several target mRNAs, measuring the level of ribosome stalling on these mRNAs in vitro. Consistent with a role for length in determining regulation by FMRP, they reported that the degree of ribosome stalling on FMRP mRNA targets is correlated with mRNA length. Together with our observation that FMRP target mRNAs are exceedingly long relative to the transcriptome average, these results point to mRNA length as a major determinant in translational regulation by FMRP.

### Methods of Examples 1-4

Analysis of Published MeCP2-Regulated Gene Lists

To search for unique characteristics of genes found to be misregulated in MeCP2 mutant mice we interrogated the list of genes found to be significantly activated or repressed by MeCP2 in the cerebellum of MeCP2 KO and MeCP2 OE mice⁶. Using published datasets for the mouse cerebellum from ENCODE and other sources, these genes were assessed for epigenetic marks at promoters and gene bodies including histone acetylation and methylation as measured by ChIP-seq analysis, as well as DNA methylation and hydroxymethylation as measured by affinity purification methods¹³. In addition, we interrogated sequence attributes of genes including dinucleotide frequencies, exon number, repeat density within genes and gene length. To determine if the misregulated genes were exceptional with respect to any epigenetic marks or sequence attributes, they were compared to several sets of control genes, selected to be matched for gene expression levels (data not shown). While no obvious epigenetic differences were apparent from this analysis, we detected the extreme length of genes (measured as Refseq total basepairs from transcription start site to transcription termination site) repressed by MeCP2 (up-regulated in the MeCP2 KO and down-regulated in the MeCP2 OE). Subsequent analysis of multiple published gene lists from several brain regions revealed the consistent, extreme length of the genes identified as repressed by MeCP2 in each brain region. These findings are presented in FIG. 1a as boxplots where each plot depicts the median (line), the 2nd through 3rd quartiles (box), 1.5× the interquaratile range (whiskers), and 1.58× the interquartile range/√# genes) (notches). The notches on each box approximate a 95% confidence interval for the median value.

To analyze gene expression genome-wide with respect to gene length, the raw hybridization data in CEL files from multiple MeCP2 KO and MeCP2 OE gene expression studies was downloaded from GEO (http://www.ncbi.nlm.nih.gov/geo/; study details, sample numbers and genotypes provided in FIG. 13) and analyzed for expression at the gene level using the GeneSpring software suite (Agilent Technologies) with RMA summarization of “Core” probesets. To facilitate unambiguous analysis of individual genes, expression values for transcript cluster IDs were filtered to include only transcript clusters that map to single Refseq genes, and expression values for genes with multiple transcript clusters were derived by taking the average log 2 expression or fold-change value across all transcript clusters corresponding to each gene. To facilitate comparison between microarray platforms, throughout this study we present analysis only for genes represented on all microarray platforms; this corresponds to 14,168 genes for mouse, 17,989 genes for human. While this represents a subset of genes in each genome we have obtained similar results for length-dependent changes in gene expression for expanded gene sets covered by individual platforms (data not shown). In addition, similar results were obtained using the Affymetrix Power Tools pipeline with PLIER as an alternative summarization method. For consistency, microarray data for gene expression in human cells was presented using a comparable array summarization scheme as the mouse microarray data (RMA). Similar qualitative results showing length-dependent gene misregulation were obtained from gene expression values generated by Li and colleagues using a normalization scheme that included spike-controls¹⁹ (These summarized transcript expression values were downloaded directly from GEO). However, with this normalization procedure, the absolute values of fold-change of all genes across the entire genome were downshifted in MECP2 null neurons relative to wild type.

To quantify the relationship between fold-change and gene length, we sorted genes by the lengths of their immature transcripts (RefSeq annotated transcriptional start site through transcriptional end site) and employed a sliding window containing 200 consecutive genes in steps of 40 genes. The log-fold-change values for the 200 genes within each length bin were averaged and plotted; displayed standard errors (SEs) for a bin were calculated by propagating the SE deduced from the bin's log-fold-change values and the mean SE of the individual genes reflecting their sample variability. Null distributions displayed on fold-change plots were constructed for each bin from 10,000 random samples of 200 genes selected without regard to transcript length.

RNA Sequencing and Analysis

Total RNA was prepared from visual cortex of wild-type and MeCP2 KO mice at 8-9 weeks of age. Brain samples were dissected on ice in HBSS and immediately frozen in liquid nitrogen. To extract RNA, the tissue was thawed in trizol (Ambion), homogenized, extracted with chloroform, and further purified on RNeasy Columns (Qiagen) using on-column DNAse treatment to remove residual DNA, as specified in the manufacturers instructions. High-throughput sequencing of total RNA was performed as a service by BGI America. Briefly, ERCC control RNAs (Ambion) were added to samples, and total RNA was depleted of ribosomal RNA using the ribozero rRNA removal kit (Epicentre), heat-fragmented to 200-700 bp in length and cloned using Uricil-N-Glycosylase-based strand specific cloning. cDNA fragments were sequenced using an Illumina HiSeq 2000, typically yielding 20M-40M usable 50-bp single-end reads per sample (see FIG. 13 for details). After filtering out adapter and low quality reads, reads were mapped using BWA³⁷ [to the mm9 genome augmented by an additional set of splicing targets (˜3M sequences of length≤98 bp representing all possible mm9 sequences that could cross at least one exon-exon junction based on the RefSeq annotation). Samples were normalized based on uniquely mapped reads that fell outside of rRNA and noncoding genes in order to avoid skewing by spikes in incompletely depleted ribosomal and transfer RNA. Normalization of each sample was referred to an in-house standard of 10M 35-bp reads. Gene expression within exons was quantified as “Density,” defined as read coverage of the exons, equal to the total number of read bases per total number of feature bases multiplied by the overall normalization coefficient. Units of Density are always proportional to RPKM (Density=0.35×RPKM).

Average read Density within a gene's exons was taken as a proxy for gene expression (for genes with multiple annotated transcripts, exonic loci were unioned together). For a given set of samples, a quantile distribution (QD) was constructed from all samples' sorted expression levels, and values from the QD were reassigned to each gene according to its rank in each sample. Within each subset of samples corresponding to wild type (WT), knockout (KO), etc., each gene was assigned its mean log QD value and a standard error (SE) over its values for this subset in order to quantify its sample-to-sample variability within the subset. (Precisely zero expression levels were ignored in constructing the QD.) The log of the fold-change (FC) between subsets for each gene, e.g., log (KO/WT), was set to the difference of the means of the KO and WT log-values for the gene, along with a propagated SE of the log values (variance equal to the sum of KO and WT variances). For consistency the RNA-seq analysis in this study is presented for the common set of genes covered by microarray analyses in previous studies (see above). Similar results were obtained for larger sets of genes defined by all Refseq genes.

Electromobility Shift Assays

Oligonucleotide probes (Integrated DNA Technologies) were 5′-³²P-end-labeled by T4 polynucleotide kinase (New England Biolabs) with [γ-³²P]ATP (Perkin Elmer) under conditions recommended by the enzyme supplier. 5′-³²P-end-labeled upper strands were purified over NucAway Spin Columns (Ambion) and annealed to equal molar concentration of the appropriate unlabeled complement strand in 10 mM Tris, pH 8.0, 50 mM NaCl, 1 mM EDTA at 95 C for 5 minutes, followed by slow cooling to room temperature. Similarly, unlabeled competitors were annealed. Double-strandedness of probes and competitors was verified by native gel electrophoresis.

Each binding reaction was incubated with 180 ng of the MeCP2 MBD (AA 81-170; Abnova), 50 fmol of 5′-32P-end-labed probe with 1, 5, 50, or 500-fold excess of an unlabeled competitor in the presence of 1 ug of pdIdC (Sigma), 1× Tris-borate-EDTA (TBE) buffer, 1 mM DTT, 20 mM HEPES, pH 7.5, 0.5 mM EDTA, 0.2% Tween-20, 30 mM KCl, and 1× Orange DNA loading dye (Thermo Scientific) in a 10 ul reaction volume for 10 minutes at room temperature. Each reaction was loaded on a 10% non-denaturing polyacrylamide (37.5:1, acrylamide/bis-acrylamide) gel in 1×TBE buffer and electrophoresed for 30 minutes at 240V on ice. Gels were then dried on Whatman filter paper on a gel drier at 80° C. for 1 hour. For imaging, dried gels were exposed to film overnight (Kodak X-Omat XB film) at −80 C.

Whole Genome Bisulfite Sequencing and Analysis

For bisulfite sequencing analysis of the cerebellum, cerebelli from four, eight-week old mice were dissected and genomic DNA extracted. Starting with 25 ng of genomic DNA, 0.25 ng of unmethylated lambda DNA was added and libraries were generated using the Ovation Ultralow Methyl-Seq Library System (Nugen). Bisulfite treatment was performed using the EpiTect bisulfite conversion kit (Qiagen) following manufacturer instructions. Libraries were constructed using TruSeq reagents (Illumina) and sequenced on a Hiseq 2500 (Illumina). Reads were mapped to the mm9 genome using BS seeker″, allowing up to four mismatches. Duplicate reads were removed and only uniquely mapping reads were kept (See FIG. 13 for details). For analysis of published bisulfite sequencing datasets^{21,25} short read files were downloaded from GEO mapped and analyzed as described above (See FIG. 13 for details). Methylation levels in all datasets were calculated as # of cytosine base calls/(# of cytosine+# of thymine base calls) within mapped reads at genomic sites where the reference genome encodes cytosine. For hydroxymethylation analysis, the same approach was applied to TAB-seq data from cortical tissue²⁵. To examine the effects of gene body methylation independently of promoters, only genes greater than 4.5 kb that contained sequenced CGs and CHs were used in our analysis, and methylation levels within regions of the transcription start site +3 kb to transcription end site were calculated by taking the average methylation levels for all reads mapping within this region. Comparison to gene expression was performed using corresponding microarray expression values for the hippocampus and the cerebellum or RNA-seq from the cortex. To facilitate fold-change analysis of RNA-seq data, the genes analyzed were filtered for minimal (non-zero) expression values.

Gene Expression Analysis of MeCP2 R306C Mice

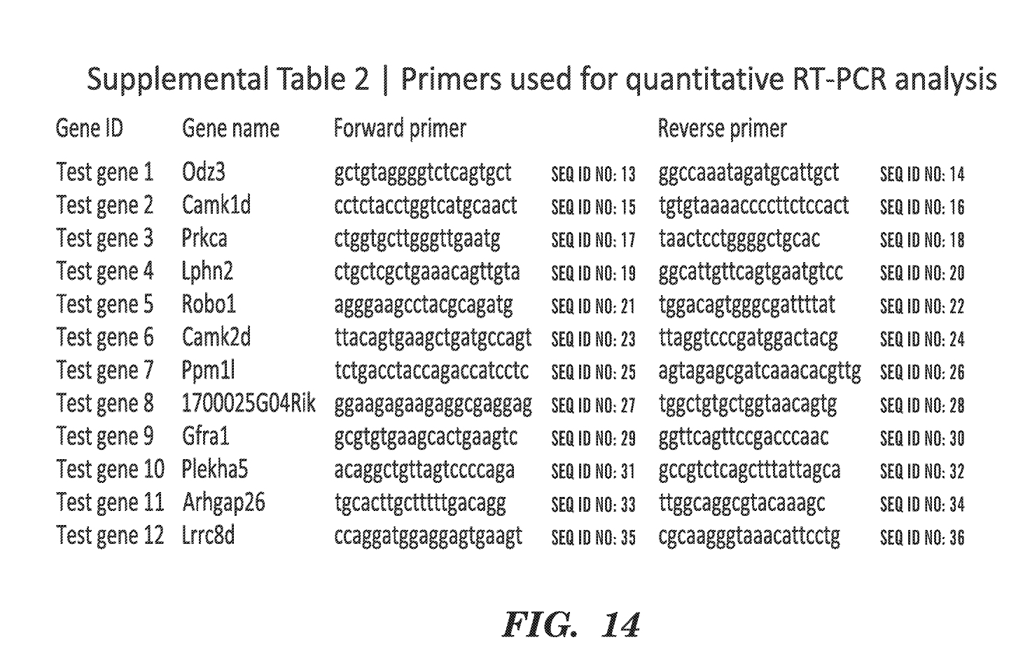

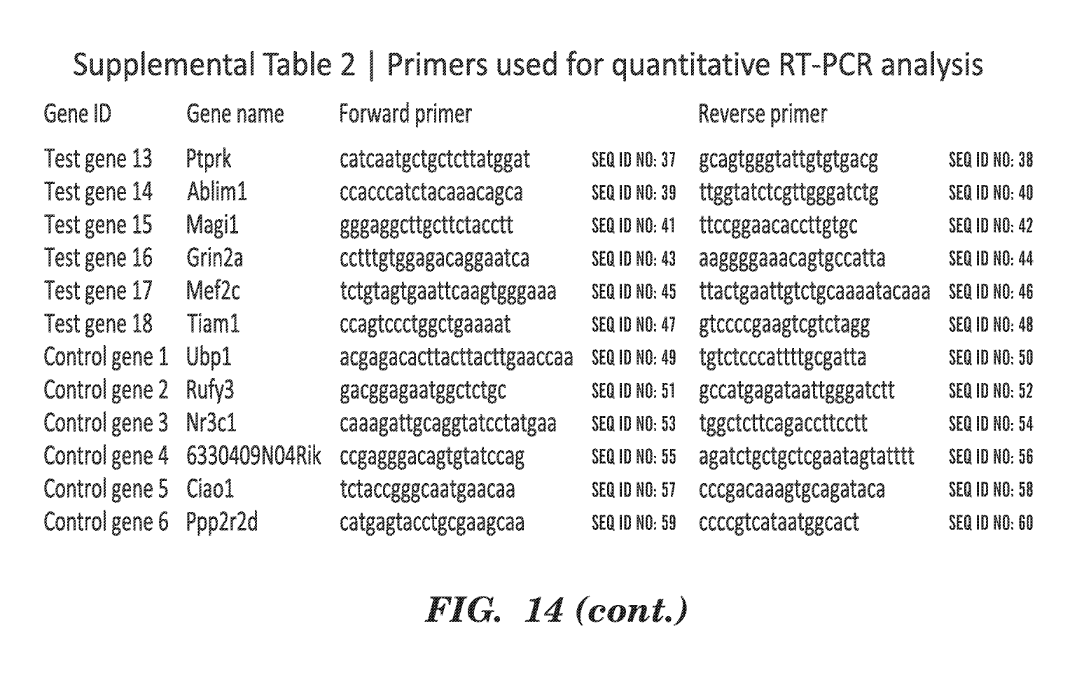

Consistent with nomenclature from past descriptions of RTT missense mutations, the R306C nomenclature refers to the mouse MeCP2 isoform 2 (MeCP2_e2; NCBI Reference Sequence NP_034918). For gene expression analysis brain regions were dissected from male Mecp2^{R306C}/y mice²⁹ and wild type littermates at 8-10 weeks of age and RNA was isolated as described above. Microarray analysis of cerebellar RNA was performed using the Affymetrix Mouse Exon 1.0 ST array platform. Analysis was performed in the Dana Farber microarray core facility following manufacturers recommendations. Analysis of hybridization data was performed as described above. For reverse transcription-quantitative PCR, genes were selected for analysis in the visual cortex based on consistent up-regulation in the MeCP2 KO (log 2 fold-change greater than zero) and down-regulation in the MeCP2 OE (log 2 fold-change less than zero) across eight published microarray datasets in five brain regions (hypothalamus, cerebellum, amygdala, striatum, hippocampus). Genes with this profile and high average fold-changes across all analyses were selected for qPCR assessment in the visual cortex. cDNA was generated from 500 ng of visual cortex total RNA (High-Capacity cDNA Reverse Transcription Kit, Applied Biosystems), and quantitative PCR was performed using transcript-specific primers (designed with the universal probe library design center, Roche, FIG. 14 and SYBR green detection on the Lightcycler 480 platform (Roche), and relative transcript levels and fold-changes were calculated by normalizing qPCR signal within each sample to six genes that do not show evidence of altered expression across published microarray data sets (See FIG. 14). Similar results were obtained by analyzing raw Cp values for test transcripts without normalization to control genes (data not shown).

Identification and Analysis of MeCP2-Repressed Genes

To facilitate identification of genes repressed by MeCP2 in the context of extremely small changes in gene expression, we analyzed the 14,168 common genes quantified across eight published microarray datasets in five brain regions (hypothalamus, cerebellum, amygdala, striatum, hippocampus) applying the lowest possible threshold for fold-change (fold-change >0 in the MeCP2 KO, fold-change <0 in the MeCP2 OE) but demanding consistent misregulation in the appropriate direction (at least 7 out of 8 datasets). Genes meeting these criteria were then filtered for minimum average change in gene expression (>7.5%), yielding 466 MeCP2-repressed genes (FIG. 15). While the analysis presented here utilizes these 466 genes identified on the criteria described above, similar results for gene length, enriched overlap with autism candidate and FMRP target genes, and enrichment for neuronal annotations were obtained with gene lists generated using alternative criteria (e.g. up in MeCP2 KO, down in MeCP2 OE in 8 out 8 datasets without minimum expression threshold). For gene ontology analysis, genes were input into the DAVID v6.7 bioinformatics resource³⁹ (http://david.abcc.ncifcrf.gov/, using the 14,168 genes covered in our analysis as background. Overlap of MeCP2-repressed genes with autism candidates and FMRP target genes was performed by mapping all SFARI genes (http://sfari.org/), and putative FMRP target lists^{31,32} to the 14,168 genes used for identification of MeCP-repressed genes and determining overlapping genes (FIG. 15). Overlap of autism candidates shown in this study is for all genes in the SFARI database, but a significant degree of overlap is observed for subsets of genes within the database that are classified as higher-confidence autism candidates (data not shown). Data processing, plotting, and statistical analysis were performed using available packages and custom scripts in R.

Brain Specific Expression of Long Genes

To assess expression of long genes across neural and non-neural tissues, RNA-Seq datasets for seven mouse tissues dissected from eight week old mice³⁵ and ten human tissues (Gray, Harmin et al., in press) were mapped and quantified as described above. Similar results of brain specific long gene expression were obtained for microarray data from the wild type samples of the five brain regions analyzed in MeCP2 mutant studies compared to the wild-type liver (data not shown).

Analysis of Dnmt3a^{flx/flx}; Nestin-Cre^{+/-} Mice

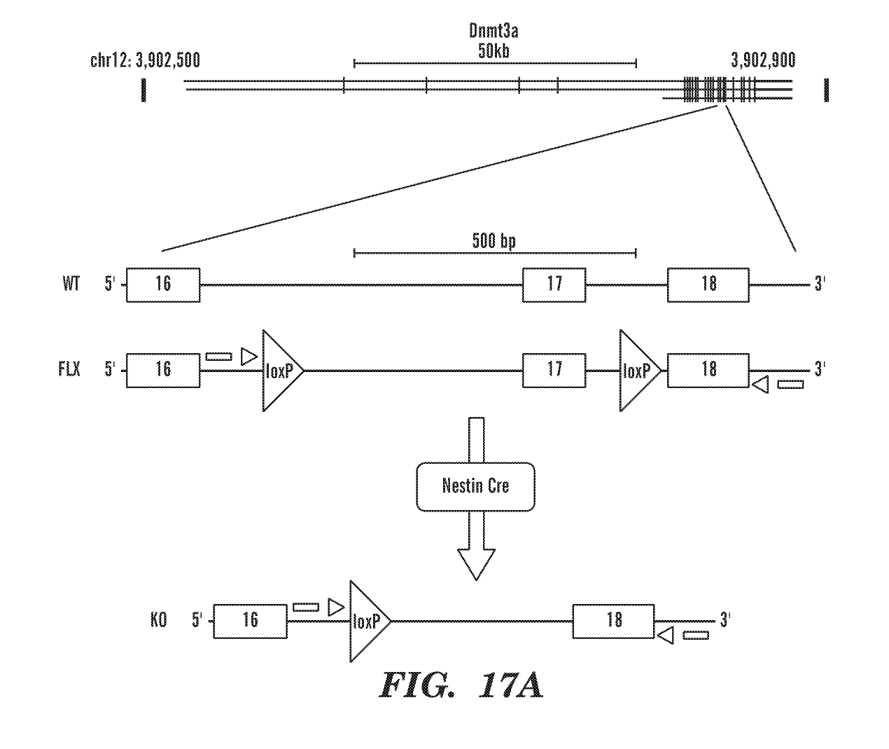

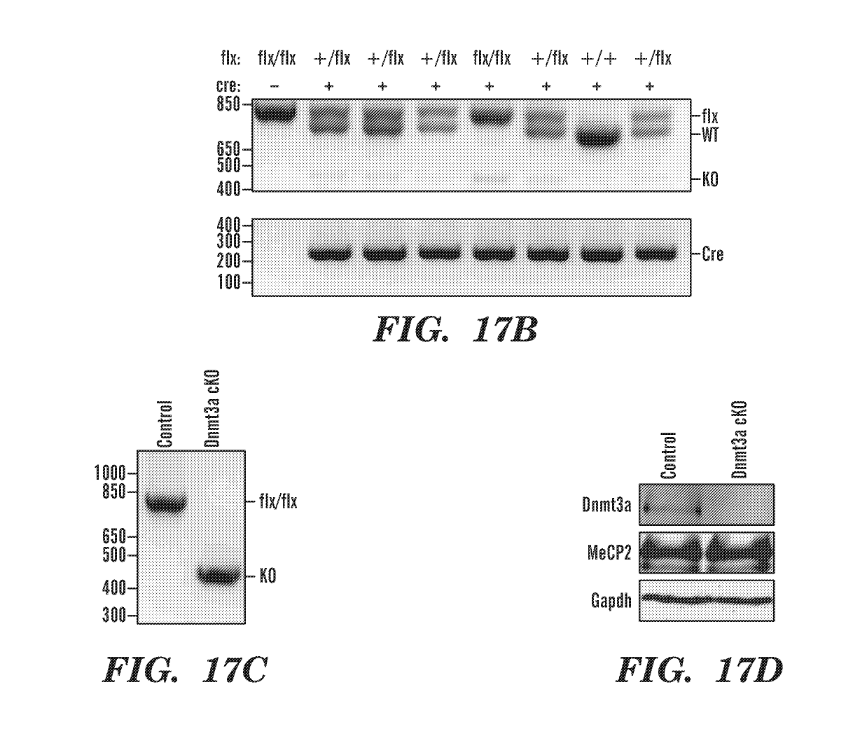

Female Dnmt3a^{flx/flx} mice⁴⁵ (kindly provided by M. Goodell) were bred to male Nestin-Cre^{+/-} mice⁴⁶ to generate Dnmt3a^{flx/+}; Nestin-Cre^{+/-} animals. To ensure expression of the imprinted Nestin-Cre transgene, male Dnmt3a^{flx/+} Tg(Nes-cre)1Kln/J animals were bred to Dnmt3a^{flx/flx} females to generate Dnmt3a^{flx/flx} Tg(Nes-cre)1K1n/J conditional knockout mice (“Dnmt3a cKO”) and Dnmt3a^{flx/flx} control animals (“Control”). For western blot, DNA methylation and gene expression analyses, cerebella were dissected from 10-11-week-old animals. Proteins were resolved by SDS-PAGE and immunoblotted using the following antibodies: Dnmt3a (abcam, ab13888), MeCP2 (custom antisera⁴⁴) and Gapdh (Sigma Aldrich, #G9545-25UL). Genotyping for the Dnmt3a locus was performed by PCR with primers flanking both loxP sites (F: 5′-GCAGCAGTCCCAGGTAGAAG-3′ (SEQ ID NO:1), R: 5′-ATTTTTCATCTTACTTCTGTGGCATC-3′ (SEQ ID NO:2),) on DNA derived from tails. The presence of the cre allele was detected using primers to this transgene (F:5′-GCAAGTTGAATAACCGGAAATGGTT-3′ (SEQ ID NO:3), R:5′-AGGGTGTTATAAGCAATCCCCAGAA-3′(SEQ ID NO:4)). This genotyping scheme allows for simultaneous assessment of the presence of the floxed allele and the relative level of loxP recombination that has occurred in the sample. Brain-specific recombination was confirmed by PCR of tail DNA compared to cerebellar DNA (see FIG. 17). For gene expression analysis RNA was extracted and analyzed as described above for MeCP2 R306C cerebellum samples.

Neuronal Cell Culture and Topotecan treatment

Primary cortical neurons were prepared from E16.5 mouse embryos and cultured as described by Kim et al. For lentiviral-mediated shRNA knockdown, virus was prepared as described in Tiscornia et al.⁴⁸ using the MeCP2 shRNA and control shRNA plasmids previously validated in Zhou et al.⁴⁹. Virus was concentrated and titrated using the GFP signal expressed from IRES GFP in the virus. After one day in vitro (DIV), cells were infected with lentivirus (knockdown or control) at an MOI of ˜5, such that >90% of cells were infected. On DIV 4 cells were fed (neurobasal media with AraC, 2 μM final concentration) and subsequently treated with various dilutions of topotecan in DMSO (0.05% DMSO final concentration). At DIV 10, cells were collected in trizol for RNA analysis, or protein gel loading buffer for protein. RNA samples were processed and analyzed using the Nanostring nCounter assay as described above, with the exception that 6 control genes were used for normalization. Western blot analysis to confirm knockdown of MeCP2 was performed as described in Chen et al.⁴⁴. Mean values shown in Extended Data FIG. 9 (n=3-5) are derived from separate cultures obtained from independent litters of mice (independent biological replicates), dissected on separate days, cultured and collected independently.

Gene Expression Analysis of MeCP2 R306C Mice

Consistent with nomenclature from past descriptions of RTT missense mutations, the R306C nomenclature refers to the mouse MeCP2 isoform 2 (MeCP2_e2; NCBI Reference Sequence NP_034918). For gene expression analysis brain regions were dissected from male Mecp2^{R306C}/y mice 29 and wild type littermates at 8-10 weeks of age and RNA was isolated as described above. Animals were preselected based on genotype before collection to insure that paired samples were taken within litters, but collection was randomized and the experimenter was uninformed of genotype during collection, sample processing, and analysis. Microarray analysis of cerebellar RNA was performed using the Affymetrix Mouse Exon 1.0 ST array platform. Analysis was performed in the Dana Farber microarray core facility following manufacturer's recommendations. Analysis of hybridization data was performed as described above. Sample size (4 per genotype) was determined based on previous detection of length-dependent gene expression effects from datasets that used similar sample sizes (see FIG. 6 and FIG. 13).

Validation of Microarray and RNA-Seq Findings.

For reverse transcription-quantitative PCR expression analysis candidate genes were selected for analysis in the visual cortex based on consistent up-regulation in the MeCP2 KO (log 2 fold-change greater than zero) and down-regulation in the MeCP2 OE (log 2 fold-change less than zero) across eight published microarray datasets in five brain regions (hypothalamus, cerebellum, amygdala, striatum, hippocampus). For Nanostring nCounter validation genes were selected based on the above criteria and evidence of up-regulation in the visual cortex RNA-seq analysis. Genes with this profile were selected for qPCR assessment in the visual cortex. cDNA was generated from 500 ng of visual cortex total RNA (High-Capacity cDNA Reverse Transcription Kit, Applied Biosystems), and quantitative PCR was performed using transcript-specific primers (designed with the universal probe library design center, Roche, Supplementary Table 2) and SYBR green detection on the Lightcycler 480 platform (Roche). Relative transcript levels and fold-changes were calculated by normalizing qPCR signal within each sample to six genes that do not show evidence of altered expression across published microarray data sets (data not shown). Similar results were obtained by analyzing raw Cp values for test transcripts without normalization to control genes (data not shown).

For non-amplification-based gene expression analysis, Nanostring nCounter reporter CodeSets were designed to detect candidate MeCP2-repressed genes in 250 ng of total RNA extracted from MeCP2 KO and R306C mice. Samples were processed at Nanostring Technologies, Inc. following the nCounter Gene Expression protocol. Briefly, total RNA was incubated at 65° C. with reporter and capture probes in hybridization buffer overnight, and captured probes were purified and analyzed on the nCounter Digital Analyzer. The number of molecules of a given transcript was determined by normalizing detected transcript counts to the geometric mean of ERCC control RNA sequences and a set of control genes that do not show evidence of altered expression across published microarray data sets. Hotelling T2 test for small sample size⁵⁰ was used to calculate significance in order to incorporate variance across both samples and genes. Significant differences between wild-type and MeCP2 KO or MeCP2 R306C samples (p<0.01) were also detected by paired two-tailed t-test comparing the paired mean values for each gene (averaged across samples within each genotype) between genotypes

MeCP2 Chromatin Immunoprecipitation Analysis

MeCP2 ChIP analysis was performed on cortex and cerebella dissected from 8-week-old wild-type male mice as previously described^{11,51.} To facilitate direct comparison of MeCP2 ChIP to published frontal cortex DNA methylation and hydroxymethylation data24, we also performed MeCP2 ChIP analysis using the same brain region at the same developmental stage (frontal cortex isolated from 6-week-old mice). ChIP DNA was cloned into libraries and sequenced on the Illumina HiSeq 2000 or Hiseq 2500 platform to generate 49 or 50 bp single-end reads. Reads were mapped to mouse genome mm9 using BWA33 and custom perl scripts were employed to quantify read density (reads/kb) for each gene. Normalized read density values were calculated as reads/kb in each genomic feature (e.g. gene), normalized to the total number of reads sequenced for each sample, and divided by the reads/kb in that feature for the input DNA that was isolated prior to the ChIP and sequenced in parallel. As with the methylation analysis, gene bodies were defined as +3000 bp to the predicted transcription termination site in the Refseq gene model. To ensure sufficient coverage and accurate assessment of density in gene bodies, only genes greater than 4500 bp in total length with at least one read in the input sample were included in the analysis.

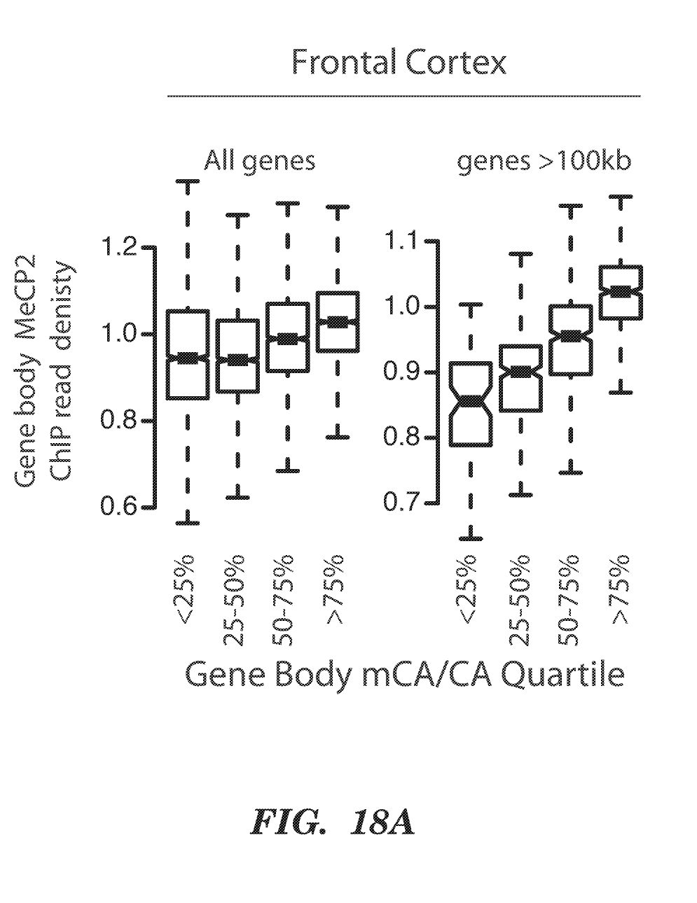

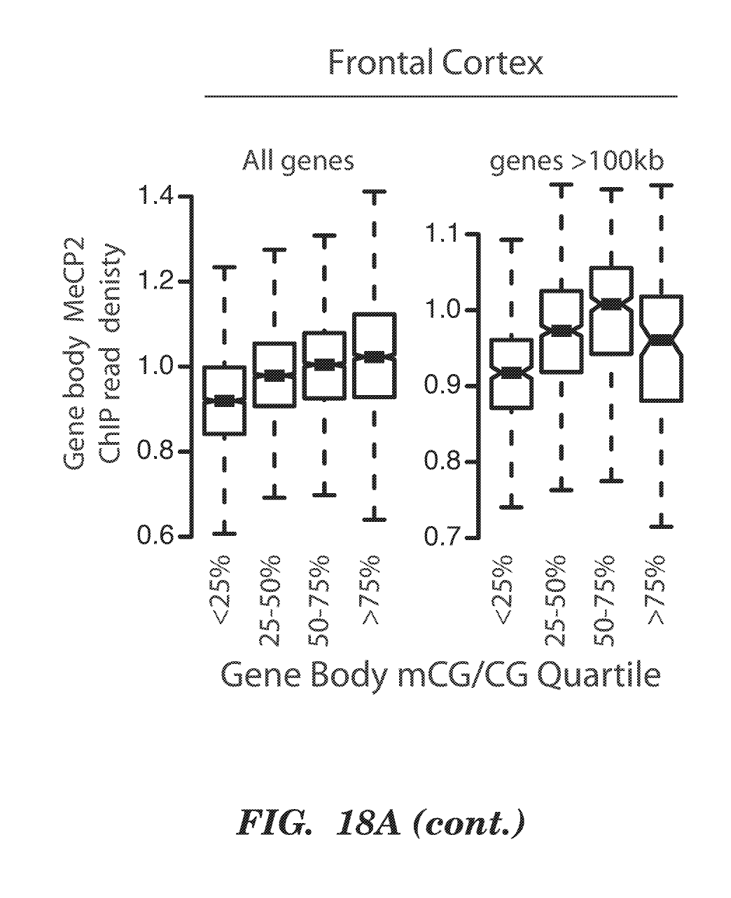

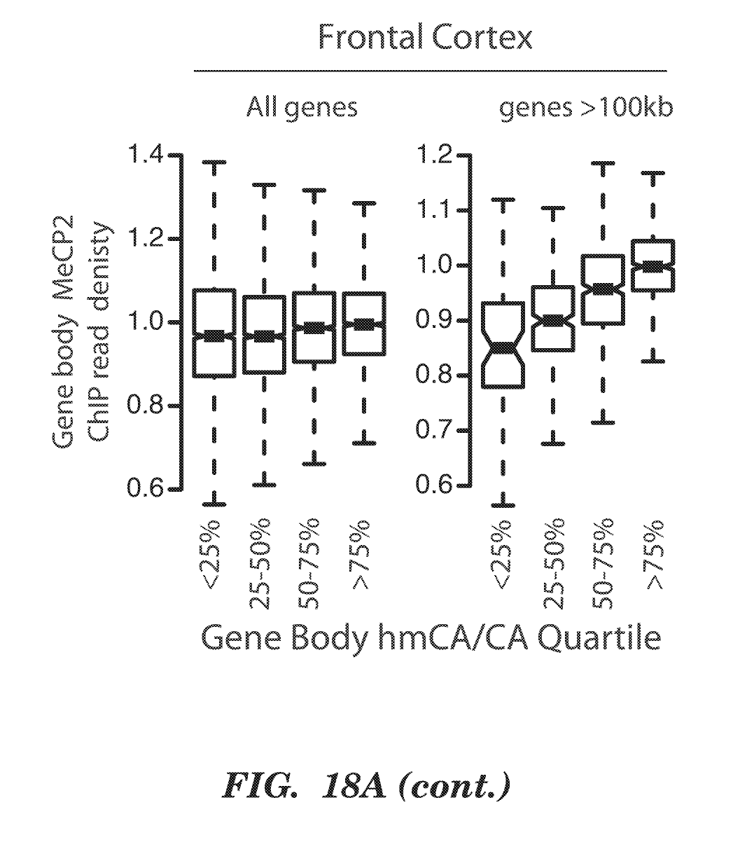

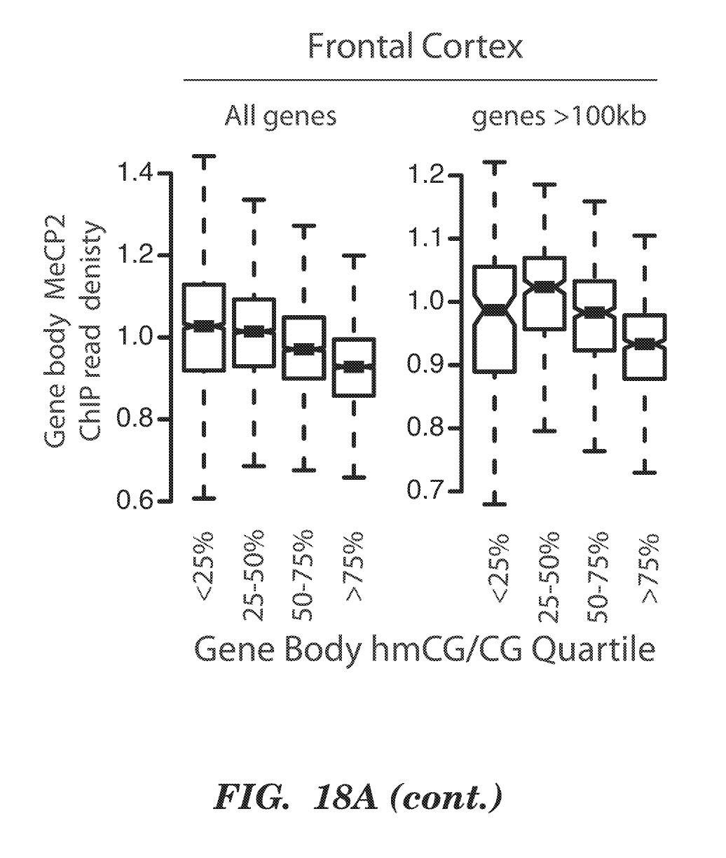

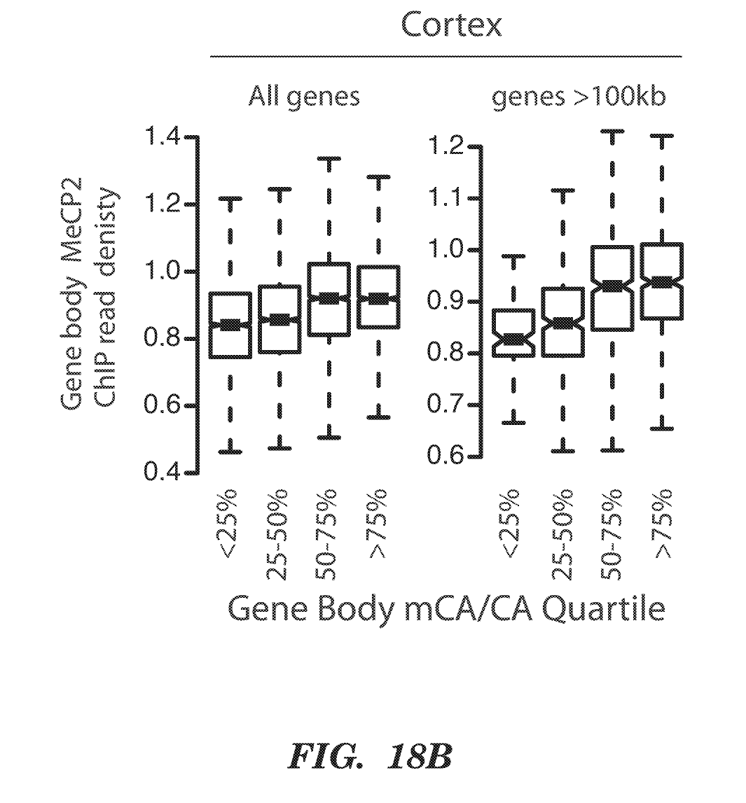

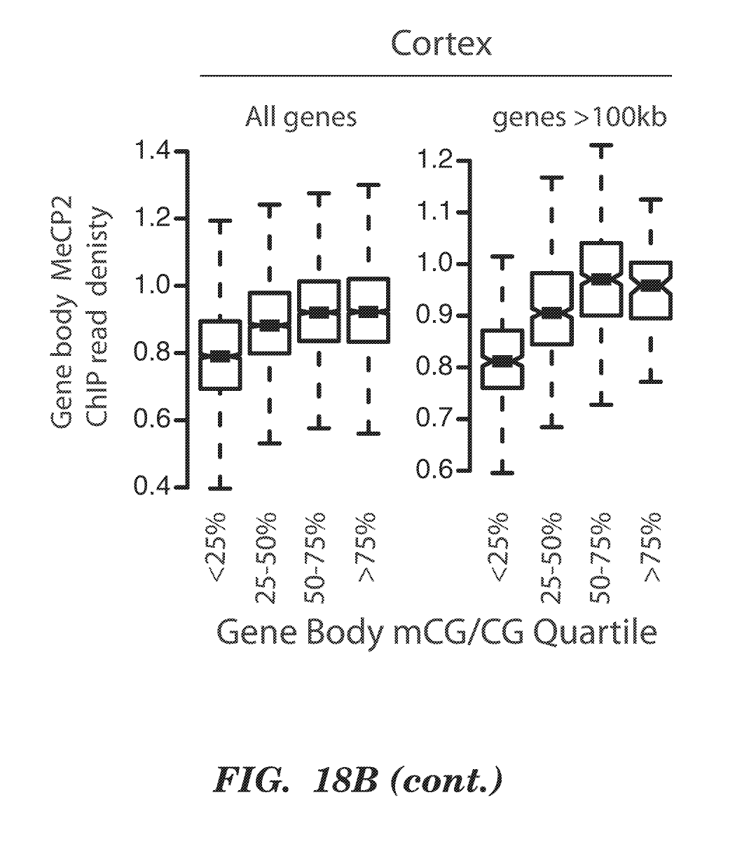

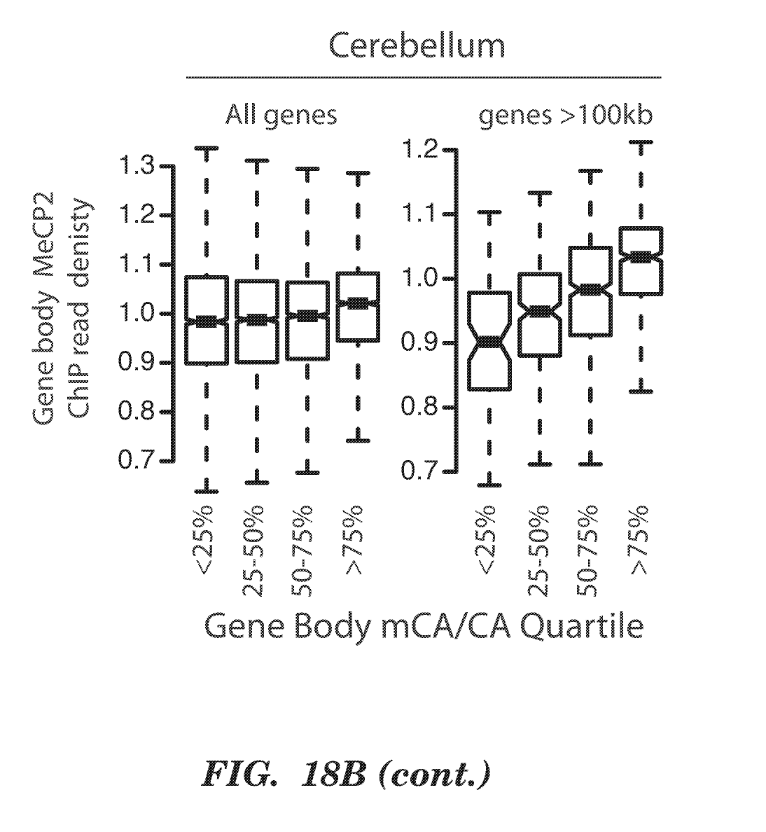

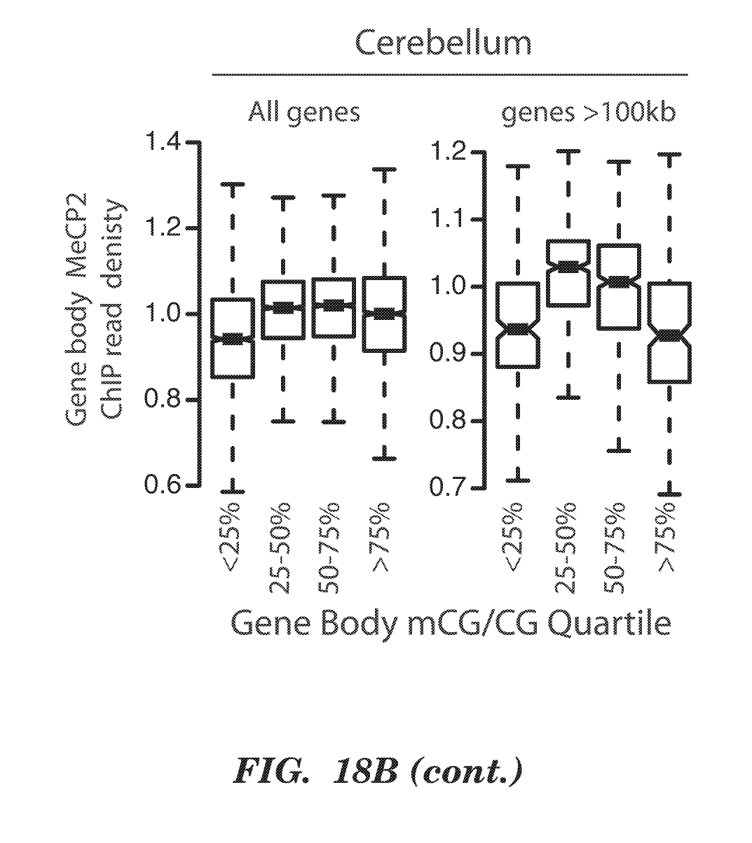

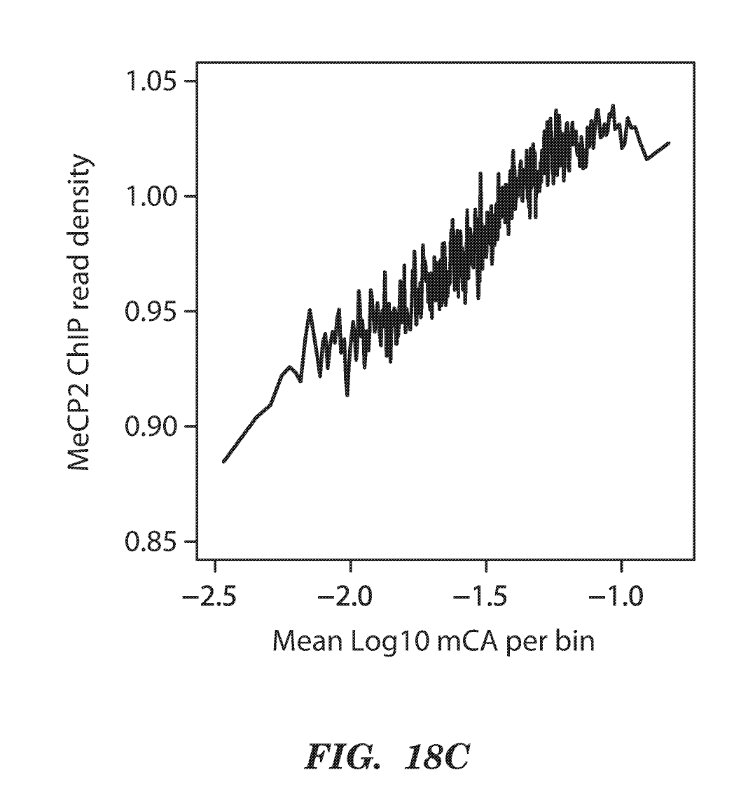

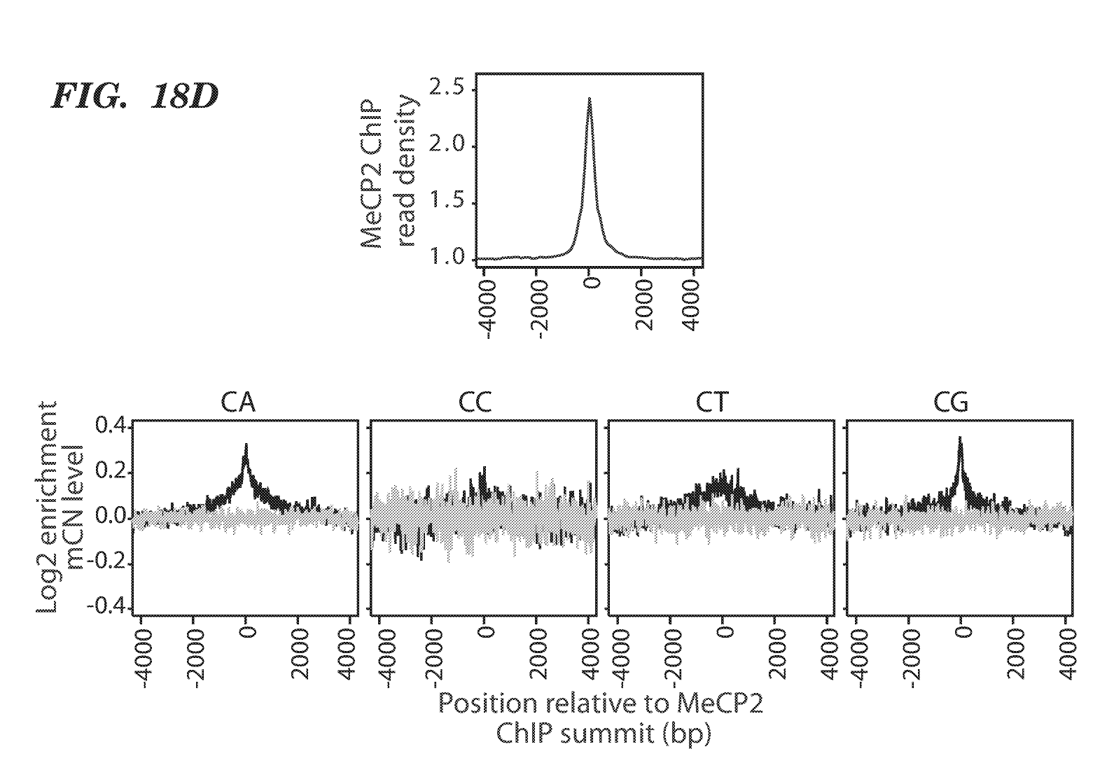

To explore the relationship between MeCP2 binding and mCA at high resolution, we also quantified the MeCP2 ChIP signal from the frontal cortex in 500 bp bins tiled for all genes in the genome and compared it to mCA levels derived from high-coverage DNA methylation analysis of this brain region (FIG. 18)²⁵. In addition, we employed the MACS40 algorithm to identify sites of MeCP2 ChIP enrichment, or “summits”, across the genome and looked for evidence of mCN at these sites. Due to the broad binding of MeCP2 across the genome, MeCP2 ChIP yields numerous sites of modest local enrichment (˜2-fold), not isolated, highly-enriched peaks (>10-fold) that are characteristic of transcription factors. Thus, to define MeCP2 summits, we utilized a low threshold of MeCP2 ChIP over input enrichment (>1-fold) and a low stringency p-value threshold (p<0.2), which yielded 31,479 summits of MeCP2 ChIP signal. Aggregate plots across all 31,479 MeCP2 summits were generated using the annotatePeaks.pl program in the Hypergeometric Optimization of Motif EnRichment (HOMER)41 software. Input-normalized MeCP2 ChIP signal was calculated as the ratio of MeCP2 ChIP/Input read coverage. Log 2 enrichment of mCN under MeCP2 summits was determined by calculating the level of methyl-cytosine (# non-converted cytosines sequenced)/(# converted and non-converted cytosines sequenced) occurring at CA, CC, CT, or CG positions in the genome, normalized to the flanking region (mean of −4 kb to −3 kb and 3 kb to 4 kb region relative to the MeCP2 summit). The average value for the ChIP signal or relative mCN was then calculated for windows (100 bp for ChIP, 10 bp for mCN) tiled across each summit location and averaged across all of the 31,479 summits of MeCP2 ChIP enrichment identified using the MACS peak-calling algorithm40 (red) and 31,479 randomly selected control sites (gray).

Regulatory Approval

All animal experiments were performed in accordance with regulations and procedures approved by the Harvard Medical Area Standing Committee on Animals (HMA IACUC).

### References for Examples and Methods

1 Chahrour, M. & Zoghbi, H. Y. The story of Rett syndrome: from clinic to neurobiology. Neuron 56, 422-437, doi:10.1016/j.neuron.2007.10.001 (2007).
2 Guy, J., Cheval, H., Selfridge, J. & Bird, A. The role of MeCP2 in the brain. Annual review of cell and developmental biology 27, 631-652, doi:10.1146/annurev-cellbio-092910-154121 (2011).
3 Tudor, M., Akbarian, S., Chen, R. Z. & Jaenisch, R. Transcriptional profiling of a mouse model for Rett syndrome reveals subtle transcriptional changes in the brain. Proceedings of the National Academy of Sciences of the United States of America 99, 15536-15541, doi:10.1073/pnas.242566899 (2002).
4 Jordan, C., Li, H. H., Kwan, H. C. & Francke, U. Cerebellar gene expression profiles of mouse models for Rett syndrome reveal novel MeCP2 targets. BMC medical genetics 8, 36, doi:10.1186/1471-2350-8-36 (2007).
5 Chahrour, M. et al. MeCP2, a key contributor to neurological disease, activates and represses transcription. Science 320, 1224-1229, doi:10.1126/science.1153252 (2008).
6 Ben-Shachar, S., Chahrour, M., Thaller, C., Shaw, C. A. & Zoghbi, H. Y. Mouse models of MeCP2 disorders share gene expression changes in the cerebellum and hypothalamus. Human molecular genetics 18, 2431-2442, doi:10.1093/hmg/ddp181 (2009).
7 Samaco, R. C. et al. Crh and Oprm1 mediate anxiety-related behavior and social approach in a mouse model of MECP2 duplication syndrome. Nature genetics 44, 206-211, doi:10.1038/ng.1066 (2012).
8 Baker, S. A. et al. An AT-hook domain in MeCP2 determines the clinical course of Rett syndrome and related disorders. Cell 152, 984-996, doi:10.1016/j.cell.2013.01.038 (2013).
9 Zhao, Y. T., Goffin, D., Johnson, B. S. & Zhou, Z. Loss of MeCP2 function is associated with distinct gene expression changes in the striatum. Neurobiology of disease 59, 257-266, doi:10.1016/j.nbd.2013.08.001 (2013).
10 Skene, P. J. et al. Neuronal MeCP2 is expressed at near histone-octamer levels and globally alters the chromatin state. Molecular cell 37, 457-468, doi:10.1016/j.molcel.2010.01.030 (2010).
11 Cohen, S. et al. Genome-wide activity-dependent MeCP2 phosphorylation regulates nervous system development and function. Neuron 72, 72-85, doi:10.1016/j.neuron.2011.08.022 (2011).
12 King, I. F. et al. Topoisomerases facilitate transcription of long genes linked to autism. Nature 501, 58-62, doi:10.1038/nature12504 (2013).
13 Mellen, M., Ayata, P., Dewell, S., Kriaucionis, S. & Heintz, N. MeCP2 binds to 5hmC enriched within active genes and accessible chromatin in the nervous system. Cell 151, 1417-1430, doi:10.1016/j.cell.2012.11.022 (2012).
14 Meins, M. et al. Submicroscopic duplication in Xq28 causes increased expression of the MECP2 gene in a boy with severe mental retardation and features of Rett syndrome. Journal of medical genetics 42, e12, doi:10.1136/jmg.2004.023804 (2005).
15 Van Esch, H. et al. Duplication of the MECP2 region is a frequent cause of severe mental retardation and progressive neurological symptoms in males. American journal of human genetics 77, 442-453, doi:10.1086/444549 (2005).
16 Sanlaville, D., Schluth-Bolard, C. & Turleau, C. Distal Xq duplication and functional Xq disomy. Orphanet journal of rare diseases 4, 4, doi:10.1186/1750-1172-4-4 (2009).
17 Collins, A. L. et al. Mild overexpression of MeCP2 causes a progressive neurological disorder in mice. Human molecular genetics 13, 2679-2689, doi:10.1093/hmg/ddh282 (2004).
18 Chen, R. Z., Akbarian, S., Tudor, M. & Jaenisch, R. Deficiency of methyl-CpG binding protein-2 in CNS neurons results in a Rett-like phenotype in mice. Nature genetics 27, 327-331, doi:10.1038/85906 (2001).
19 Li, Y. et al. Global transcriptional and translational repression in human-embryonic-stem-cell-derived Rett syndrome neurons. Cell stem cell 13, 446-458, doi:10.1016/j.stem.2013.09.001 (2013).
20 Lewis, J. D. et al. Purification, sequence, and cellular localization of a novel chromosomal protein that binds to methylated DNA. Cell 69, 905-914 (1992).
21 Guo, J. U. et al. Distribution, recognition and regulation of non-CpG methylation in the adult mammalian brain. Nature neuroscience 17, 215-222, doi:10.1038/nn.3607 (2014).
22 Kriaucionis, S. & Heintz, N. The nuclear DNA base 5-hydroxymethylcytosine is present in Purkinje neurons and the brain. Science 324, 929-930, doi:10.1126/science.1169786 (2009).
23 Szulwach, K. E. et al. 5-hmC-mediated epigenetic dynamics during postnatal neurodevelopment and aging. Nature neuroscience 14, 1607-1616, doi:10.1038/nn.2959 (2011).
24 Xie, W. et al. Base-resolution analyses of sequence and parent-of-origin dependent DNA methylation in the mouse genome. Cell 148, 816-831, doi:10.1016/j.cell.2011.12.035 (2012).
25 Lister, R. et al. Global epigenomic reconfiguration during mammalian brain development. Science 341, 1237905, doi:10.1126/science.1237905 (2013).
26 Valinluck, V. et al. Oxidative damage to methyl-CpG sequences inhibits the binding of the methyl-CpG binding domain (MBD) of methyl-CpG binding protein 2 (MeCP2). Nucleic acids research 32, 4100-4108, doi:10.1093/nar/gkh739 (2004).
27 Hashimoto, H. et al. Recognition and potential mechanisms for replication and erasure of cytosine hydroxymethylation. Nucleic acids research 40, 4841-4849, doi:10.1093/nar/gks155 (2012).
28 Spruijt, C. G. et al. Dynamic readers for 5-(hydroxy)methylcytosine and its oxidized derivatives. Cell 152, 1146-1159, doi:10.1016/j.cell.2013.02.004 (2013).
29 Lyst, M. J. et al. Rett syndrome mutations abolish the interaction of MeCP2 with the NCoR/SMRT co-repressor. Nature neuroscience 16, 898-902, doi:10.1038/nn.3434 (2013).
30 Darnell, J. C. & Klann, E. The translation of translational control by FMRP: therapeutic targets for FXS. Nature neuroscience 16, 1530-1536, doi:10.1038/nn.3379 (2013).
31 Darnell, J. C. et al. FMRP stalls ribosomal translocation on mRNAs linked to synaptic function and autism. Cell 146, 247-261, doi:10.1016/j.cell.2011.06.013 (2011).
32 Brown, V. et al. Microarray identification of FMRP-associated brain mRNAs and altered mRNA translational profiles in fragile X syndrome. Cell 107, 477-487 (2001).
33 Raychaudhuri, S. et al. Accurately assessing the risk of schizophrenia conferred by rare copy-number variation affecting genes with brain function. PLoS genetics 6, e1001097, doi:10.1371/journal.pgen.1001097 (2010).
34 Polymenidou, M. et al. Long pre-mRNA depletion and RNA missplicing contribute to neuronal vulnerability from loss of TDP-43. Nature neuroscience 14, 459-468, doi:10.1038/nn.2779 (2011).
35 Mortazavi, A., Williams, B. A., McCue, K., Schaeffer, L. & Wold, B. Mapping and quantifying mammalian transcriptomes by RNA-Seq. Nature methods 5, 621-628, doi:10.1038/nmeth.1226 (2008).
36 Ebert, D. H. et al. Activity-dependent phosphorylation of MeCP2 threonine 308 regulates interaction with NCoR. Nature 499, 341-345, doi:10.1038/nature12348 (2013).
37 Li, H. & Durbin, R. Fast and accurate short read alignment with Burrows-Wheeler transform. Bioinformatics 25, 1754-1760, doi:10.1093/bioinformatics/btp324 (2009).
38 Chen, P. Y., Cokus, S. J. & Pellegrini, M. BS Seeker: precise mapping for bisulfate sequencing. BMC bioinformatics 11, 203, doi:10.1186/1471-2105-11-203 (2010).
39 Huang da, W., Sherman, B. T. & Lempicki, R. A. Systematic and integrative analysis of large gene lists using DAVID bioinformatics resources. Nature protocols 4, 44-57, doi:10.1038/nprot.2008.211 (2009).
40 Wu, Y., Genton, M. G. & Stefanski, L. A. A multivariate two-sample mean test for small sample size and missing data. Biometrics 62, 877-885, doi:10.1111/j.1541-0420.2006.00533.x (2006).
41 Magklara, A. & Lomvardas, S. Stochastic gene expression in mammals: lessons from olfaction. Trends in cell biology 23, 449-456, doi:10.1016/j.tcb.2013.04.005 (2013).
42 Meehan, R. R., Lewis, J. D. & Bird, A. P. Characterization of MeCP2, a vertebrate DNA binding protein with affinity for methylated DNA. Nucleic acids research 20, 5085-5092 (1992).
43 Nan, X., Meehan, R. R. & Bird, A. Dissection of the methyl-CpG binding domain from the chromosomal protein MeCP2. Nucleic acids research 21, 4886-4892 (1993).
44. Chen, W. G. et al. Derepression of BDNF transcription involves calcium-dependent phosphorylation of MeCP2. Science 302, 885-889, doi:10.1126/science.1086446 (2003).
45. Kaneda, M. et al. Essential role for de novo DNA methyltransferase Dnmt3a in paternal and maternal imprinting. Nature 429, 900-903, doi:10.1038/nature02633 (2004).
46. Tronche, F. et al. Disruption of the glucocorticoid receptor gene in the nervous system results in reduced anxiety. Nature genetics 23, 99-103, doi:10.1038/12703 (1999).
47. Kim, T. K. et al. Widespread transcription at neuronal activity-regulated enhancers. Nature 465, 182-187, doi:10.1038/nature09033 (2010).
48 Tiscornia, G., Singer, O. & Verma, I. M. Production and purification of lentiviral vectors. Nature protocols 1, 241-245, doi:10.1038/nprot.2006.37 (2006).
49 Zhou, Z. et al. Brain-specific phosphorylation of MeCP2 regulates activity-dependent Bdnf transcription, dendritic growth, and spine maturation. Neuron 52, 255-269, doi:10.1016/j.neuron.2006.09.037 (2006).
50. Wu, Y., Genton, M. G. & Stefanski, L. A. A multivariate two-sample mean test for small sample size and missing data. Biometrics 62, 877-885, doi:10.1111/j.1541-0420.2006.00533.x (2006).
51. Ebert, D. H. et al. Activity-dependent phosphorylation of MeCP2 threonine 308 regulates interaction with NCoR. Nature 499, 341-345, doi:10.1038/nature12348 (2013).
52. Yazdani, M. et al. Disease modeling using embryonic stem cells: MeCP2 regulates nuclear size and RNA synthesis in neurons. Stem cells 30, 2128-2139, doi:10.1002/stem.1180 (2012).
53. Hashimoto, H. et al. Recognition and potential mechanisms for replication and erasure of cytosine hydroxymethylation. Nucleic acids research 40, 4841-4849, doi:10.1093/nar/gks155 (2012).
54. Magklara, A. & Lomvardas, S. Stochastic gene expression in mammals: lessons from olfaction. Trends in cell biology 23, 449-456, doi:10.1016/j.tcb.2013.04.005 (2013).
55. Meehan, R. R., Lewis, J. D. & Bird, A. P. Characterization of MeCP2, a vertebrate DNA binding protein with affinity for methylated DNA. Nucleic acids research 20, 5085-5092 (1992).
56. Nan, X., Meehan, R. R. & Bird, A. Dissection of the methyl-CpG binding domain from the chromosomal protein MeCP2. Nucleic acids research 21, 4886-4892 (1993).
57. Nguyen, S., Meletis, K., Fu, D., Jhaveri, S. & Jaenisch, R. Ablation of de novo DNA methyltransferase Dnmt3a in the nervous system leads to neuromuscular defects and shortened lifespan. Developmental dynamics: an official publication of the American Association of Anatomists 236, 1663-1676, doi:10.1002/dvdy.21176 (2007).
58. Guy, J., Hendrich, B., Holmes, M., Martin, J. E. & Bird, A. A mouse Mecp2-null mutation causes neurological symptoms that mimic Rett syndrome. Nature genetics 27, 322-326, doi:10.1038/85899 (2001).
59. Darnell, J. C. & Klann, E. The translation of translational control by FMRP: therapeutic targets for FXS. Nature neuroscience 16, 1530-1536, doi:10.1038/nn.3379 (2013)
60. Polymenidou, M. et al. Long pre-mRNA depletion and RNA missplicing contribute to neuronal vulnerability from loss of TDP-43. Nature neuroscience 14, 459-468, doi:10.1038/nn.2779 (2011).
61. Raychaudhuri, S. et al. Accurately assessing the risk of schizophrenia conferred by rare copy-number variation affecting genes with brain function. PLoS genetics 6, e1001097, doi:10.1371/journal.pgen.1001097 (2010).
62. Khrapunov, S. et al. Unusual characteristics of the DNA binding domain of epigenetic regulatory protein MeCP2 determine its binding specificity. Biochemistry 53, 3379-3391, doi:10.1021/bi500424z (2014).

### Example 2

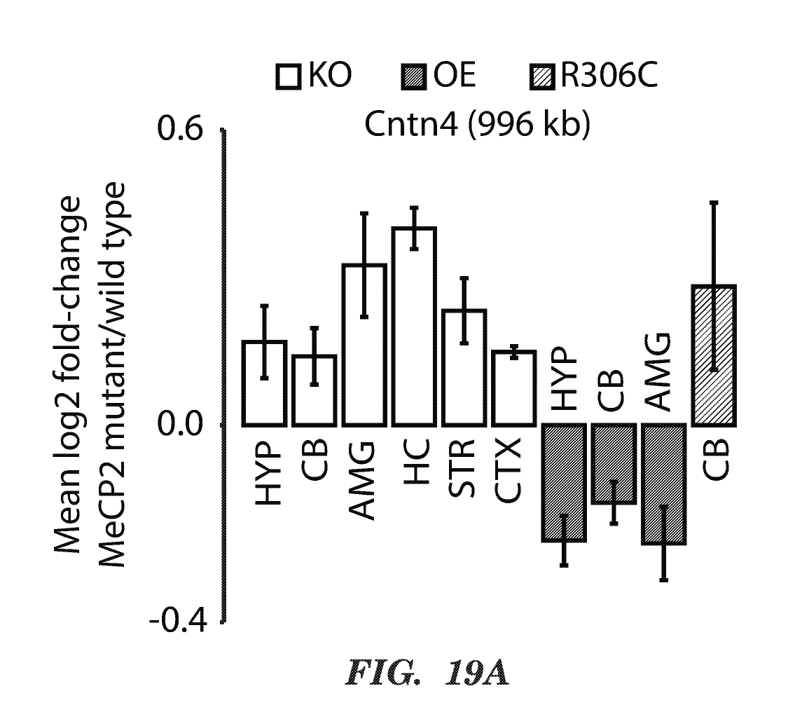

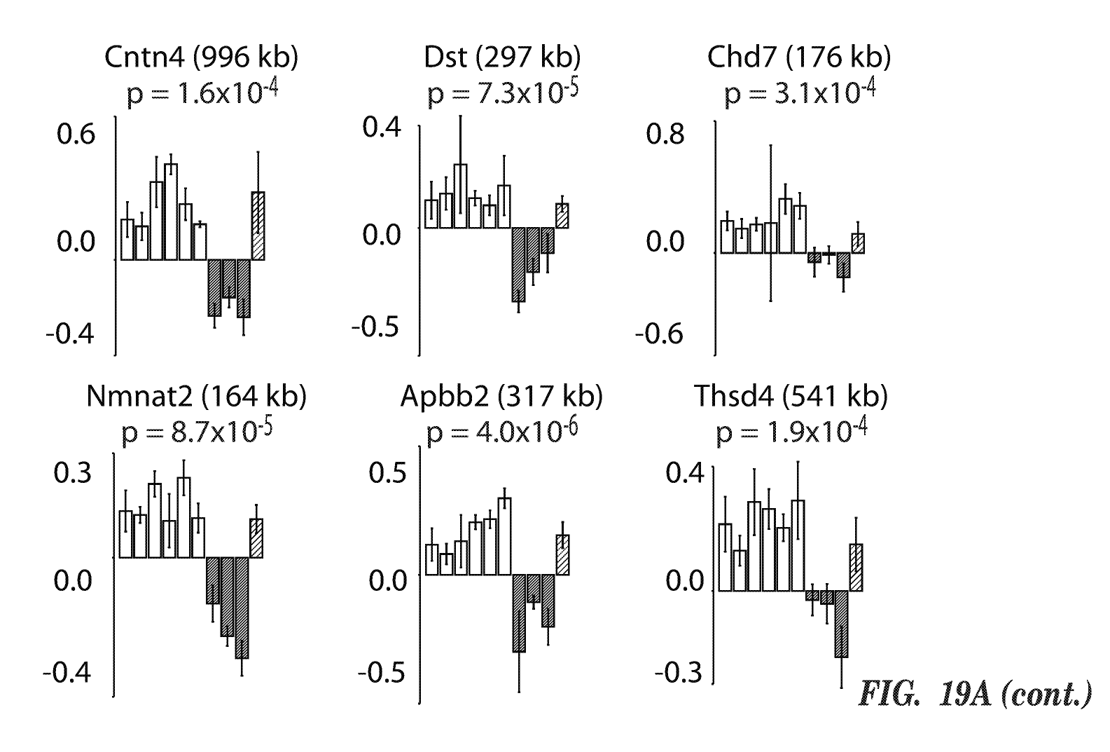

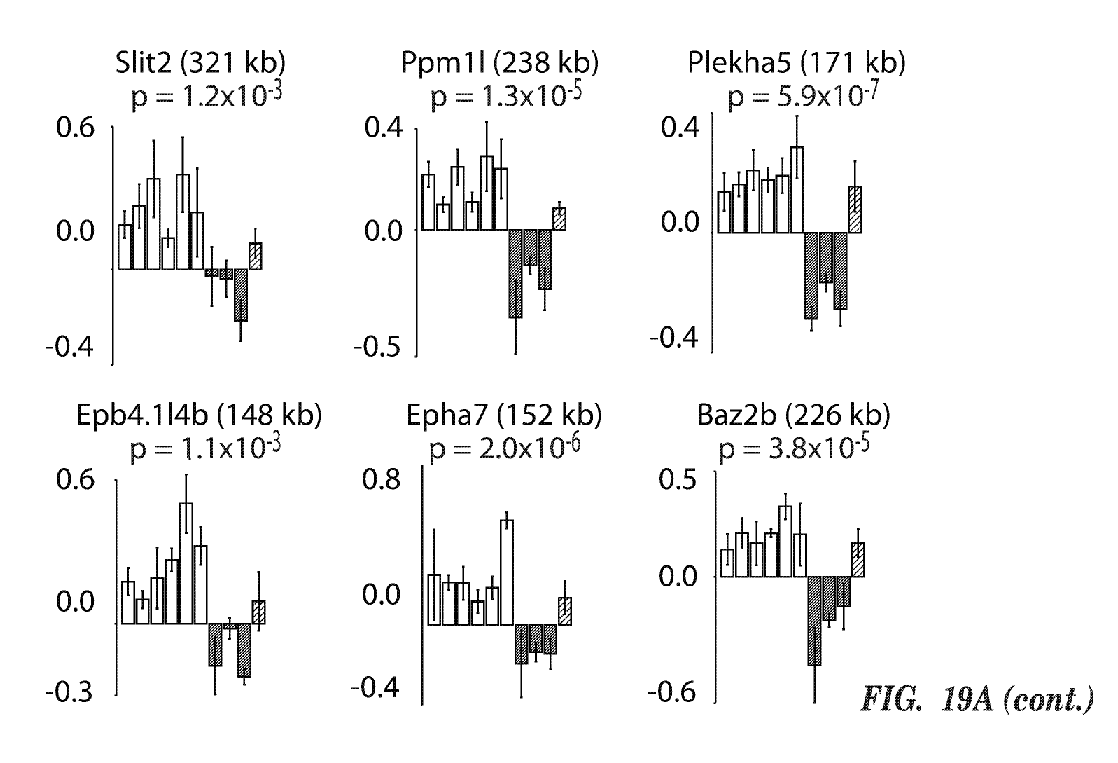

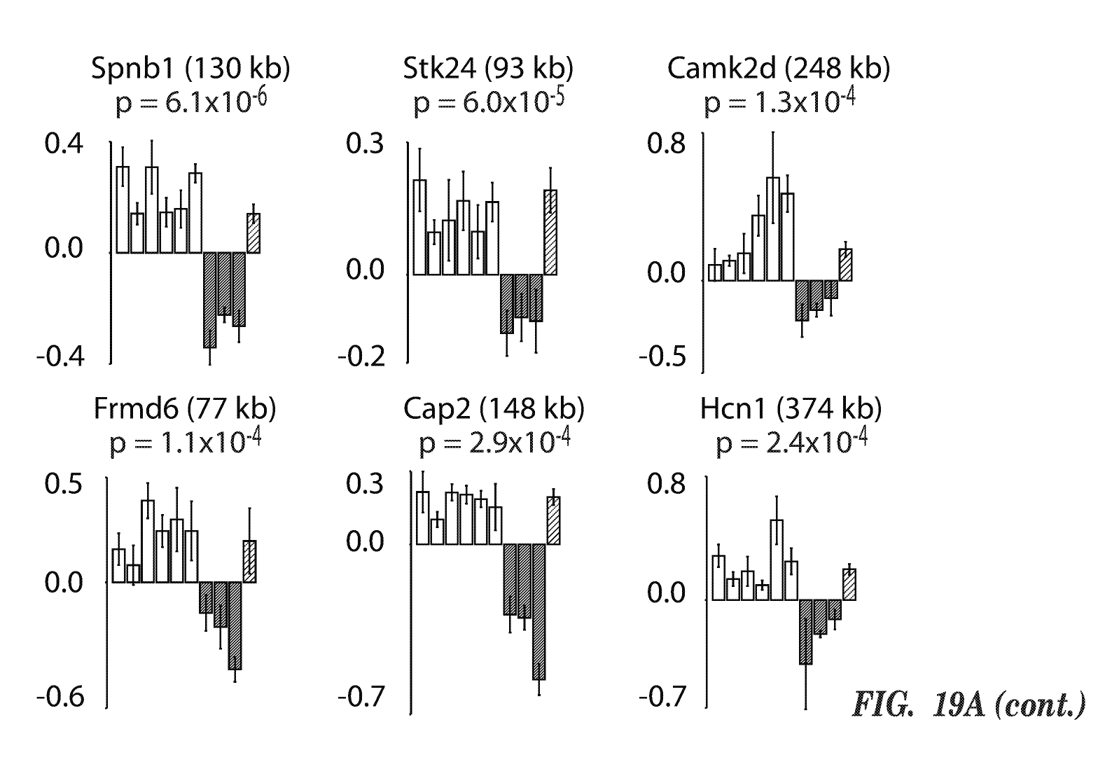

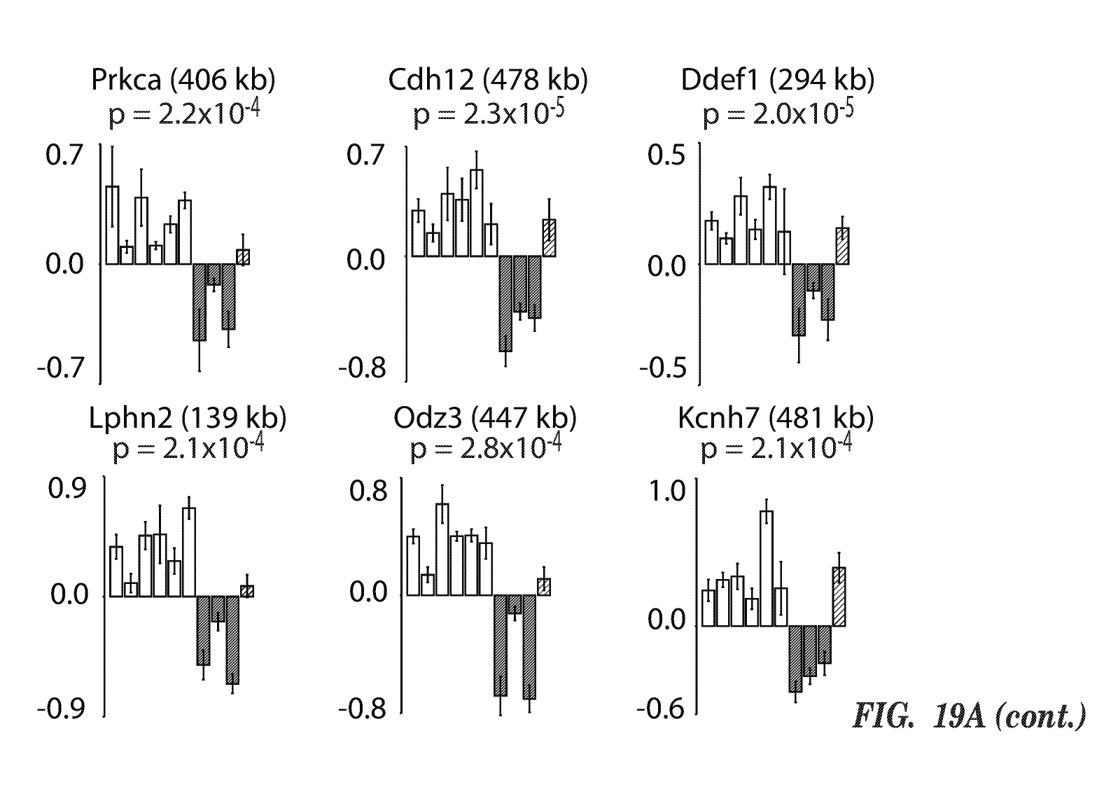

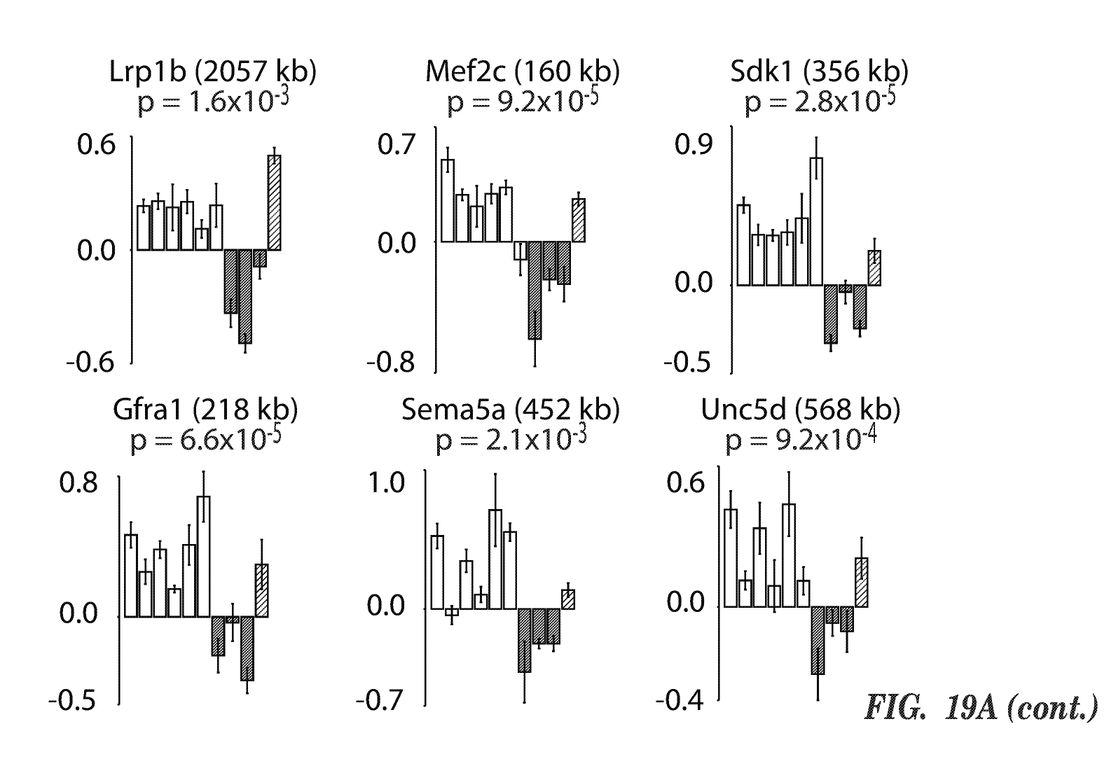

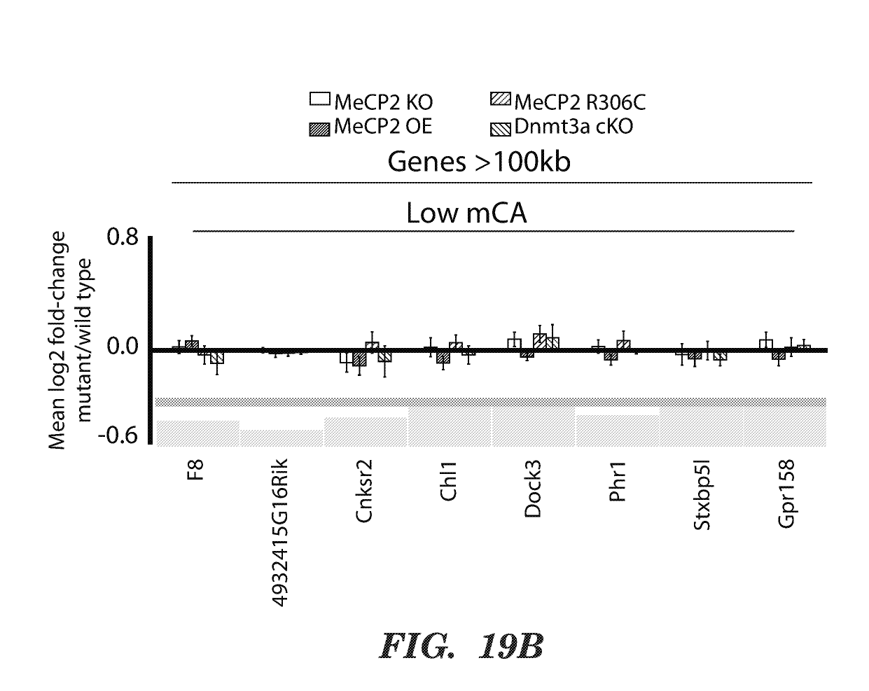

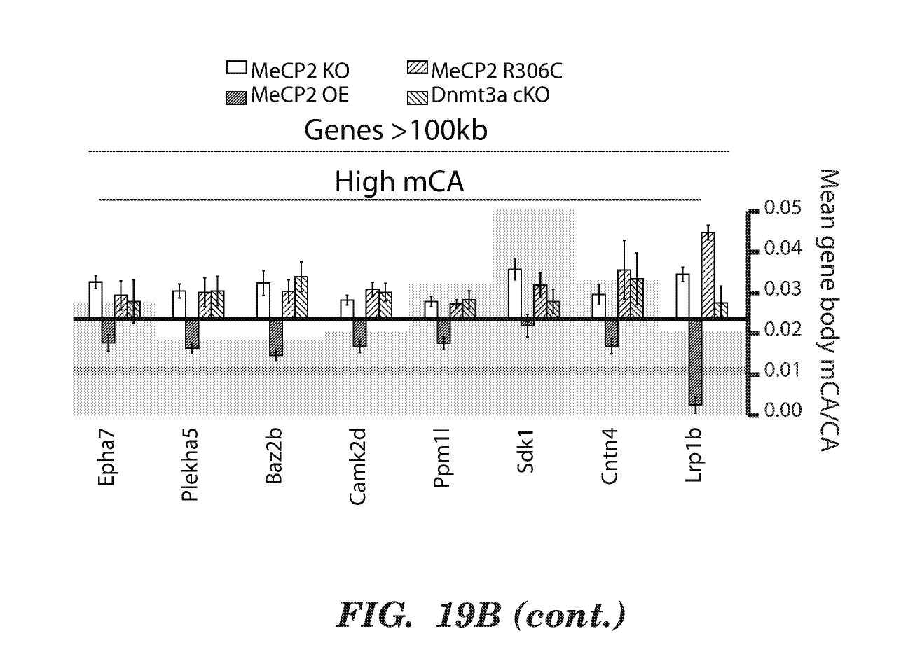

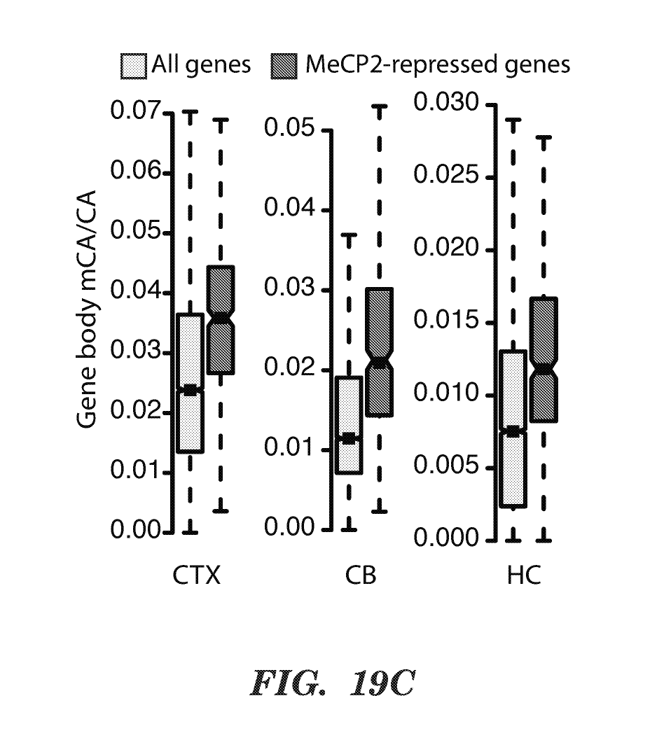

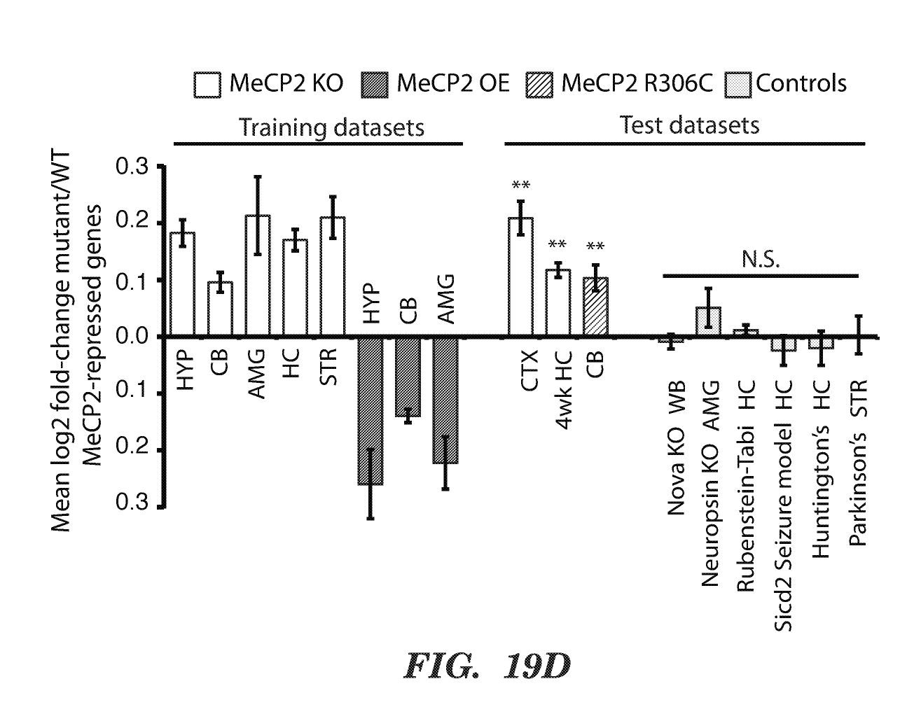

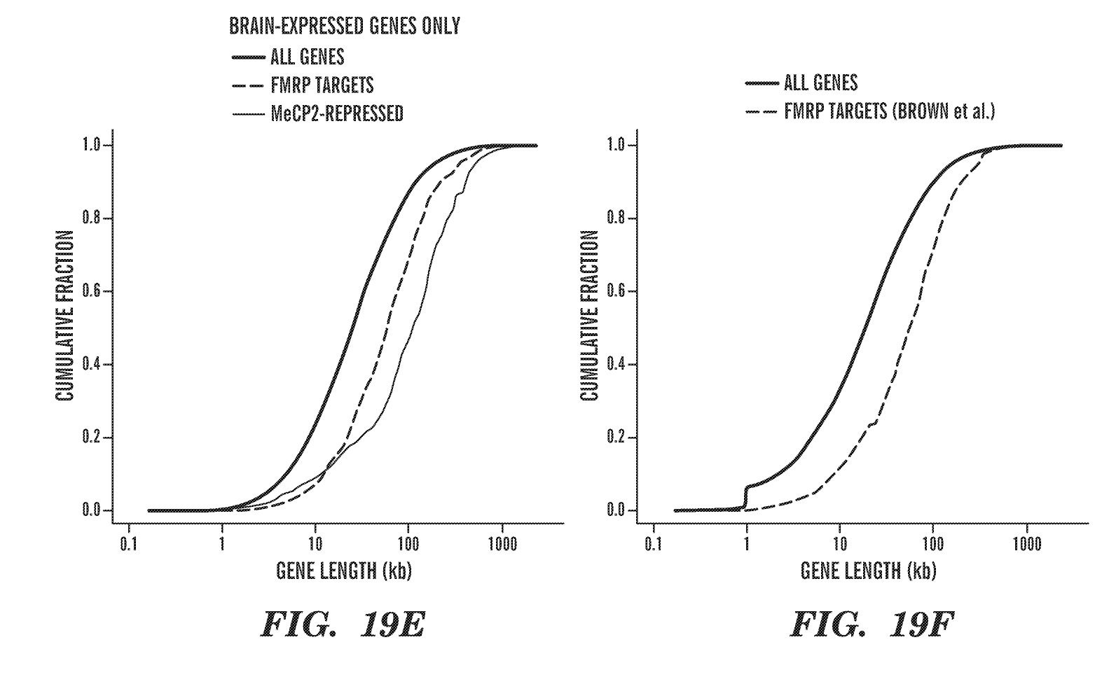

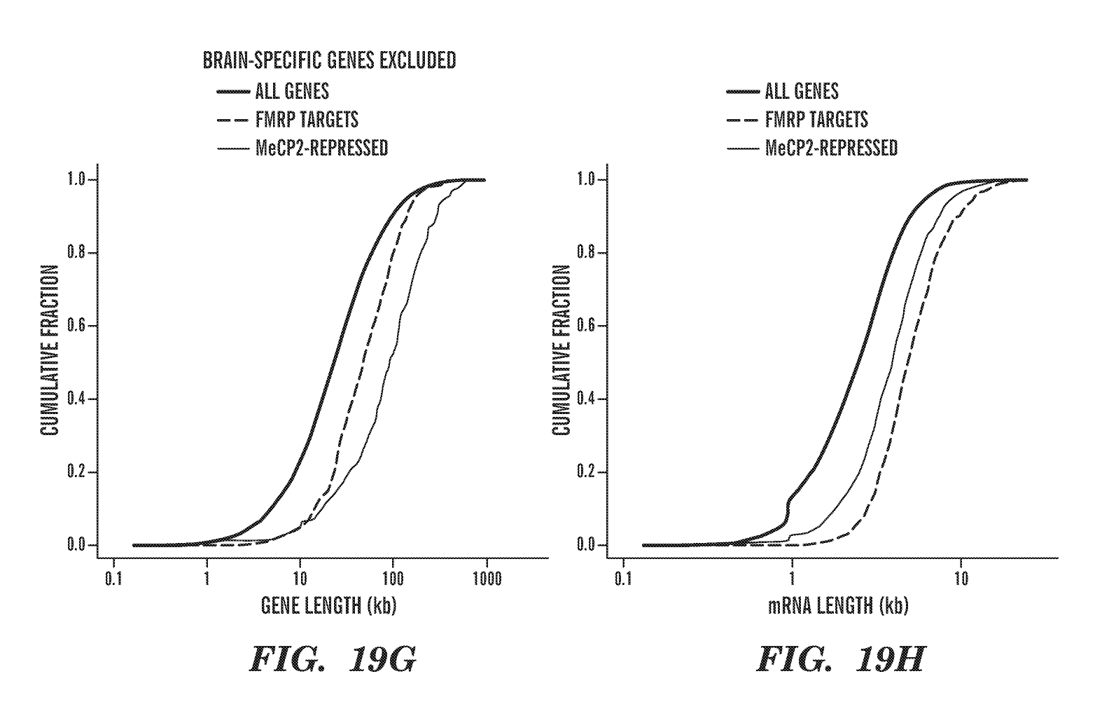

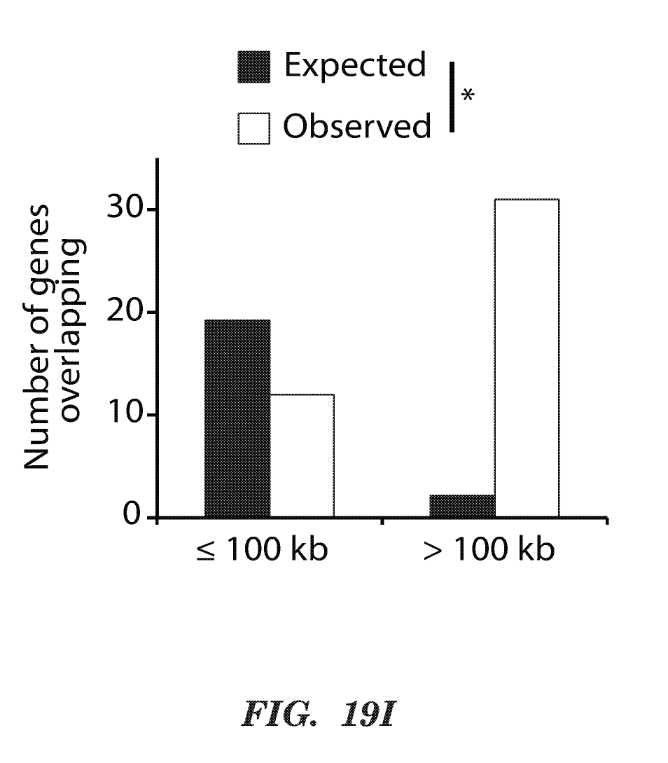

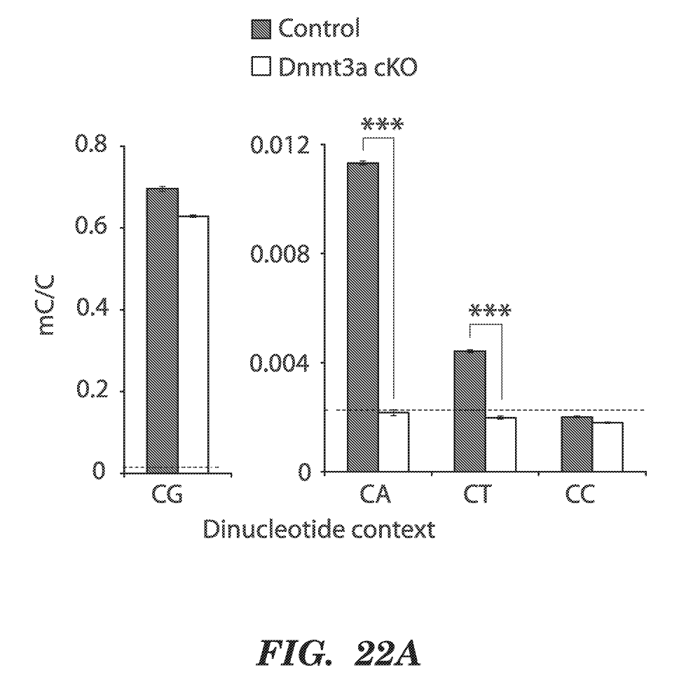

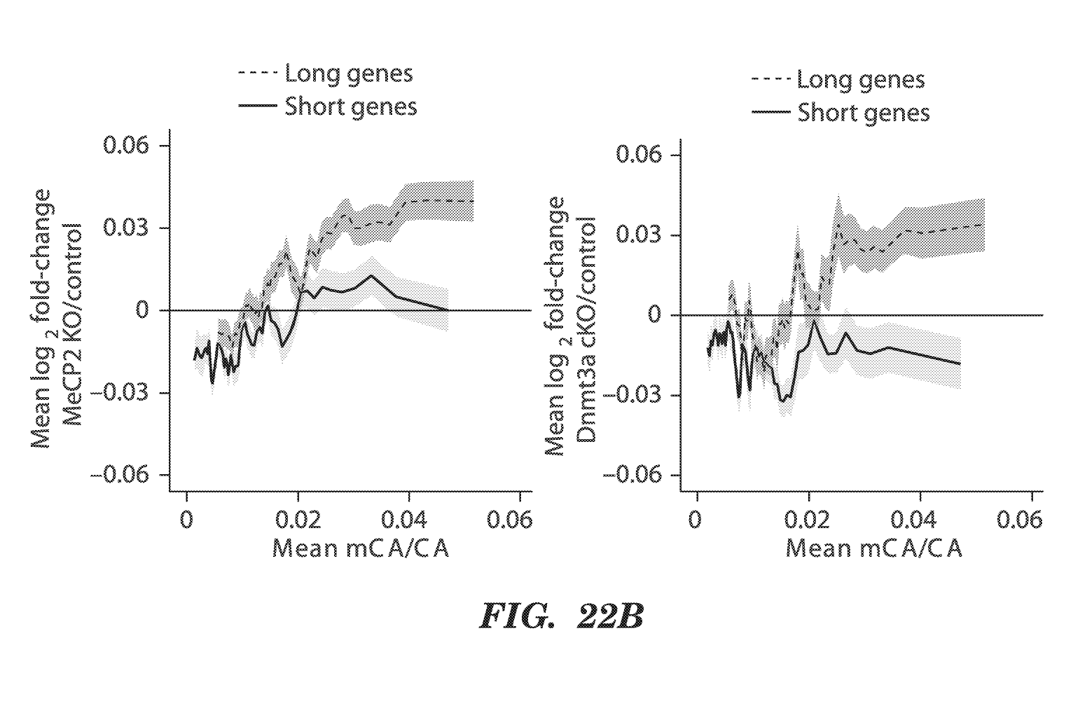

To further test if MeCP2 tempers long gene transcription by binding to mCA within genes we asked if elimination of mCA in the brain has an effect on gene expression that is similar to that observed in the MeCP2 KO. Recent evidence suggests that Dnmt3a is the enzyme that catalyzes the deposition of mCA in maturing neurons^{21,25}. We therefore conditionally disrupted the Dnmt3a gene¹¹ in the brain to block the accumulation of mCA (Nestin-Cre; Dnmt3a^{flx/flx} mice, designated Dnmt3a cKO, FIG. 17). Bisulfite sequencing of cerebellum DNA indicated that methylation of DNA at CA, but not CG, is eliminated from the genome in the Dnmt3a cKO (FIG. 22a). Microarray analysis of cerebella from Dnmt3a cKO mice revealed a length- and mCA-dependent up-regulation of gene expression that is similar to the gene misregulation detected in MeCP2 KO mice (FIGS. 19a to 19i, FIG. 22b). While the deletion of Dnmt3a also leads to a decrease in methylation at CT and CC, given that MeCP2 selectively binds to mCA in vitro, we conclude that reduction of mCA within gene bodies in the Dnmt3a cKO likely disrupts length-dependent gene repression by MeCP2. Taken together, these findings support a model in which Dnmt3a catalyzes the methylation of CA in the neuronal genome. MeCP2 then binds to these sites within the transcribed regions of genes to restrain transcription in a length-dependent manner.

Length-Dependent Gene Misregulation in Hypomorphic MeCP2 Mutants and Human RTT Models

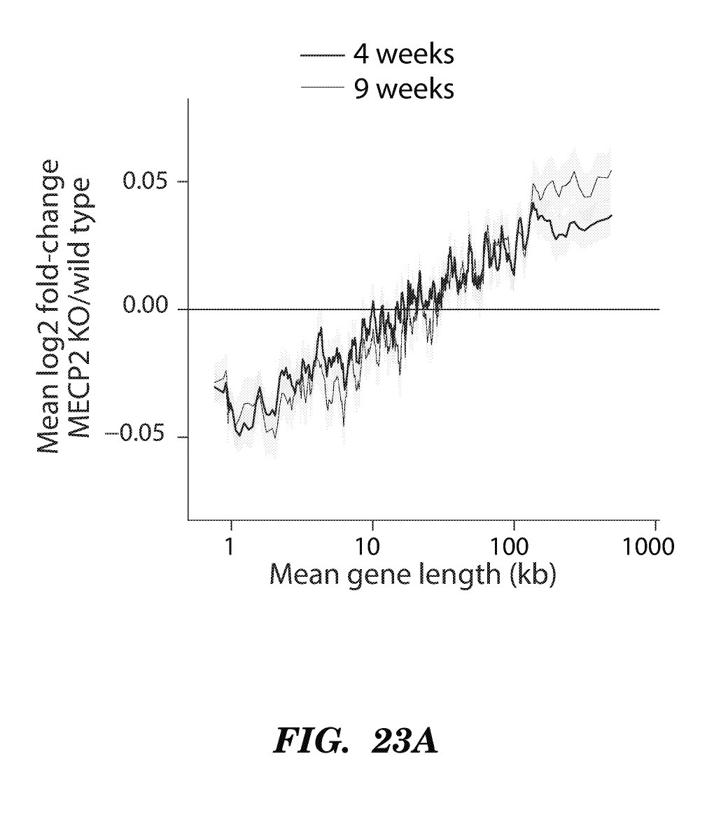

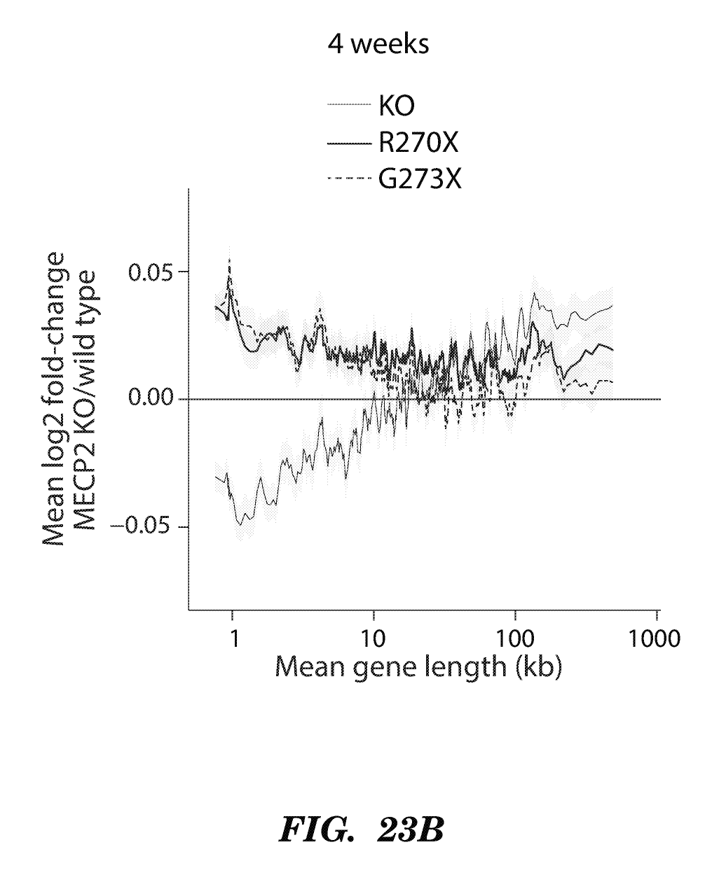

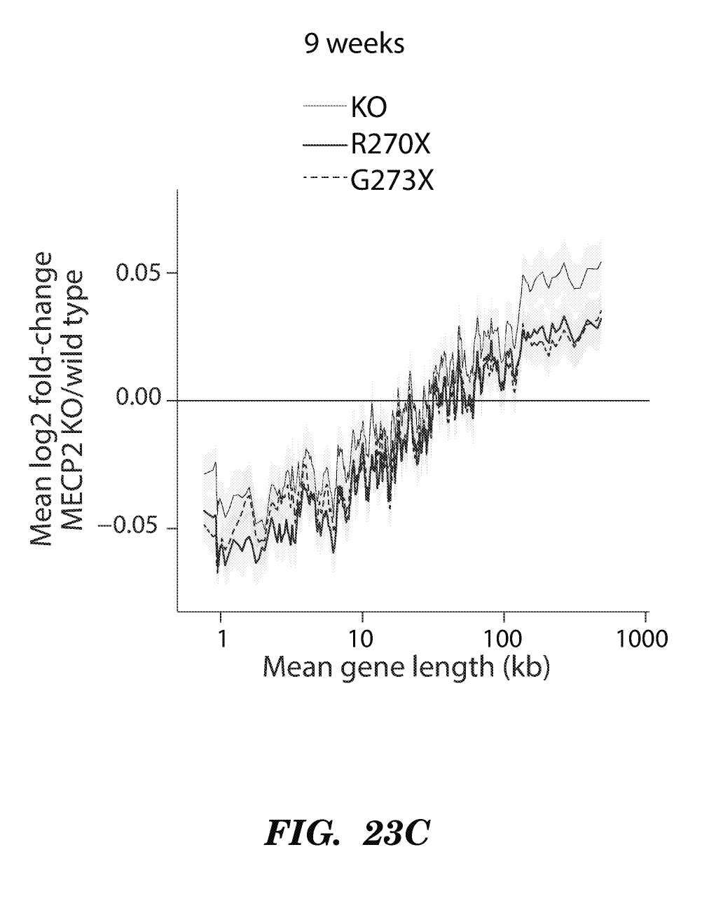

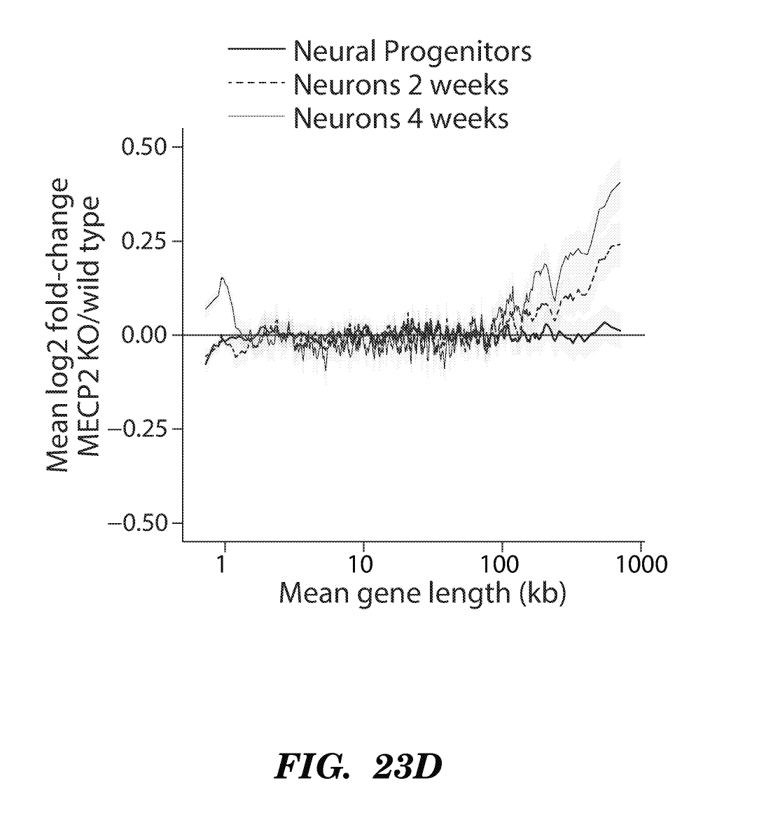

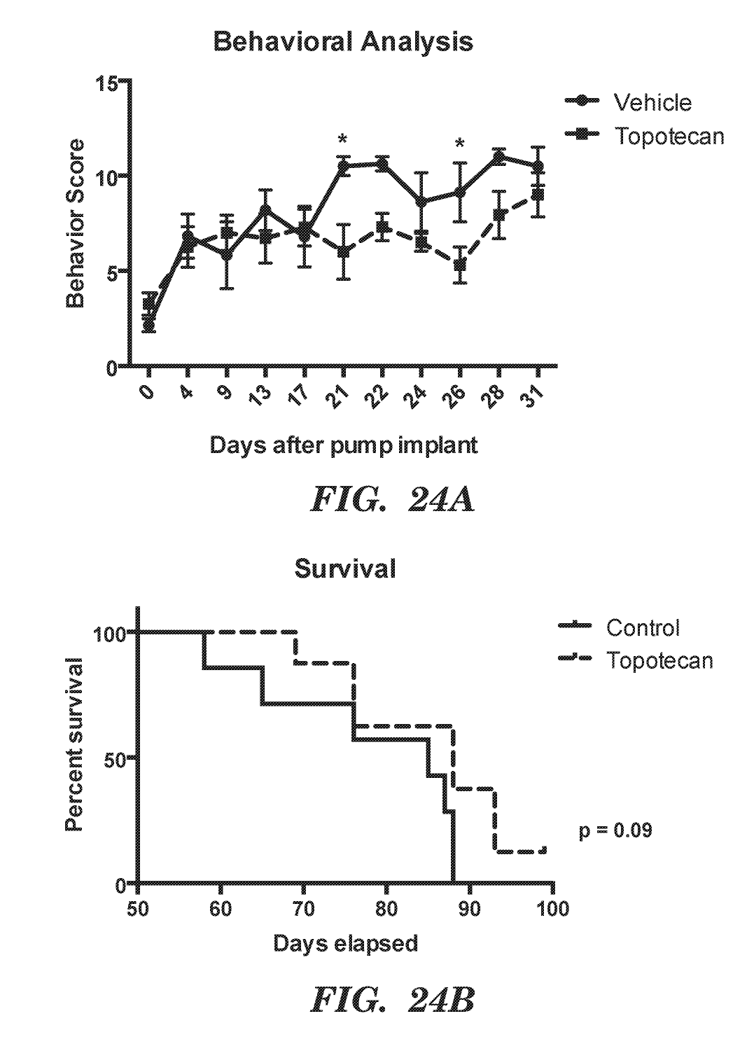

Baker and colleagues⁸ recently characterized two disease-causing MECP2 truncations, MeCP2-R270X and MeCP2-G273X, by expressing these mutant forms of MeCP2 in MeCP2 KO mice. While both of the MeCP2 mutant proteins are capable of partially rescuing the MeCP2 KO phenotype, the R270X mice still show severe, early-onset RTT, while the G273X animals exhibit more moderate symptoms with later onset. Consistent with the idea that the magnitude of the changes in long gene expression correlate with the severity of RTT pathology, we observe a trend toward less up-regulation of long genes in the hippocampus of G273X mice early in development (4 weeks) than in the brains of R270X mice of the same age (FIG. 23b, FIG. 23c). The more subtle length-dependent misregulation of long gene expression in G273X mice correlates with the delayed kinetics of symptom onset and death in these animals⁸.

Li, Jaenisch and colleagues recently reported that upon differentiation from ES cells, MECP2-deficient human neurons display progressive cellular dysfunction compared to control neurons, exhibiting reduced dendritic complexity, reduced ribosomal RNA levels, and a near-absence of detectable neuronal activity¹⁹. We analyzed microarray expression data from this study to determine if length-dependent gene misregulation occurs in this human model of RTT cellular dysfunction. Analysis of MECP2 null or wild-type neural progenitor cells¹⁹ revealed no difference in the expression of long genes, as might be expected, since MECP2 expression is low in these cells (FIG. 23d). By contrast, when neural progenitors are differentiated into neurons, we observe a prominent length-dependent misregulation of gene expression in MECP2-deficient human neurons relative to wild-type neurons that becomes more severe between two and four weeks in culture (FIG. 23d). Notably, the length-dependent increase in long gene expression relative to shorter genes is detected independently of an overall reduction in the total RNA (ribosomal and mRNA) content that occurs as the health of cultured human neurons declines due to the absence of MeCP2¹⁹: this length-dependent effect can be observed in gene expression data that is either normalized to spike-in controls¹⁹ (see Methods), or processed without these controls (FIG. 23d).

Olfactory Receptor Misregulation in MeCP2 Mutants

A notable exception to the length-dependent alterations in gene expression that we observe in Mecp2 mutants is a distinct population of very short genes, approximately 1 kb in length, that display up-regulation in the MeCP2 KO and down-regulation in the MeCP2 OE in some datasets. This altered population is visible as a spike in mean fold-change vs length plots for both mouse brain regions and human cells (FIGS. 6a to 6d). Inspection of the genes at this length revealed that the spike corresponds to the olfactory receptor genes. Several hundred highly paralogous olfactory receptor transcripts of nearly uniform length are present in mice and humans. They occur in several large clusters in the genome and are highly repressed in all cell types except for the neurons of the olfactory system⁵⁴. This is visible as a downward spike in expression in FIG. 4b. The very low expression of these genes leads to a high degree of noise in their measured expression levels (observable as a large spread of fold-change values in FIG. 6a), and it is possible that this spike is an artifact of this low expression and high variance. However, the change of the population average in some datasets suggests that MeCP2 may be required to maintain full repression of these genes. Unlike the length-dependent regulation by MeCP2 that we observe, the regulation of the olfactory receptor genes by MeCP2 is likely to occur independently of mCA, as recent basepair-resolution analysis of DNA methylation in the brain detected little or no mCA across the large genomic domains containing the olfactory receptors genes²⁵. It is unclear what functional consequences in the brain could result from olfactory receptor misregulation in Mecp2 mutants, as even upon derepression in the MeCP2 KO the levels of these transcripts would be extremely low.

To characterize how the misregulation of long gene expression contributes to RTT pathology, we identified a representative set of genes that is consistently misregulated in multiple gene expression datasets when MeCP2 function is perturbed. Combined analysis of microarray studies across multiple brain regions identified 466 MeCP2-repressed genes whose expression is consistently up-regulated in MeCP2 KO mice and down-regulated in MeCP2 OE mice (FIG. 15). Consistent with the conclusion that MeCP2-repressed genes are targets of gene-length- and mCA-dependent repression, these genes are exceptionally long and are enriched for mCA (FIG. 5a, FIG. 19a to 19i). Disruption of the expression of this geneset is specific to RTT, as these genes were not misregulated in datasets obtained from six other mouse models of neurological dysfunction (FIGS. 19a to 19i).

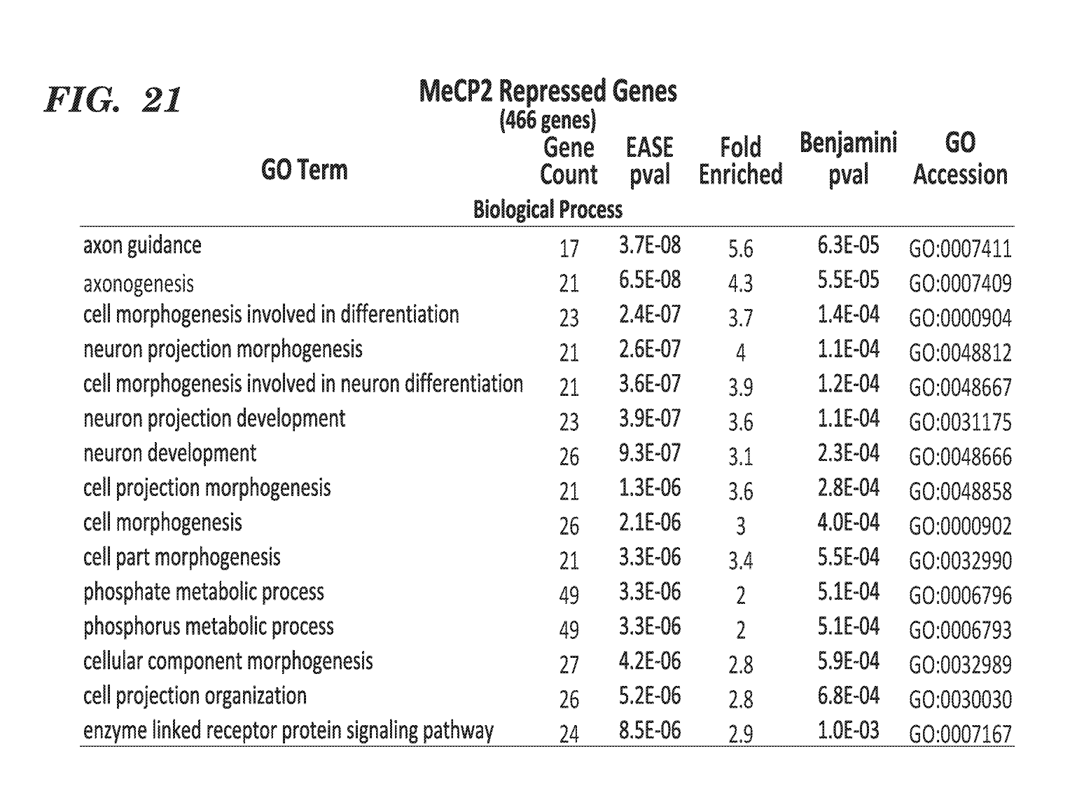

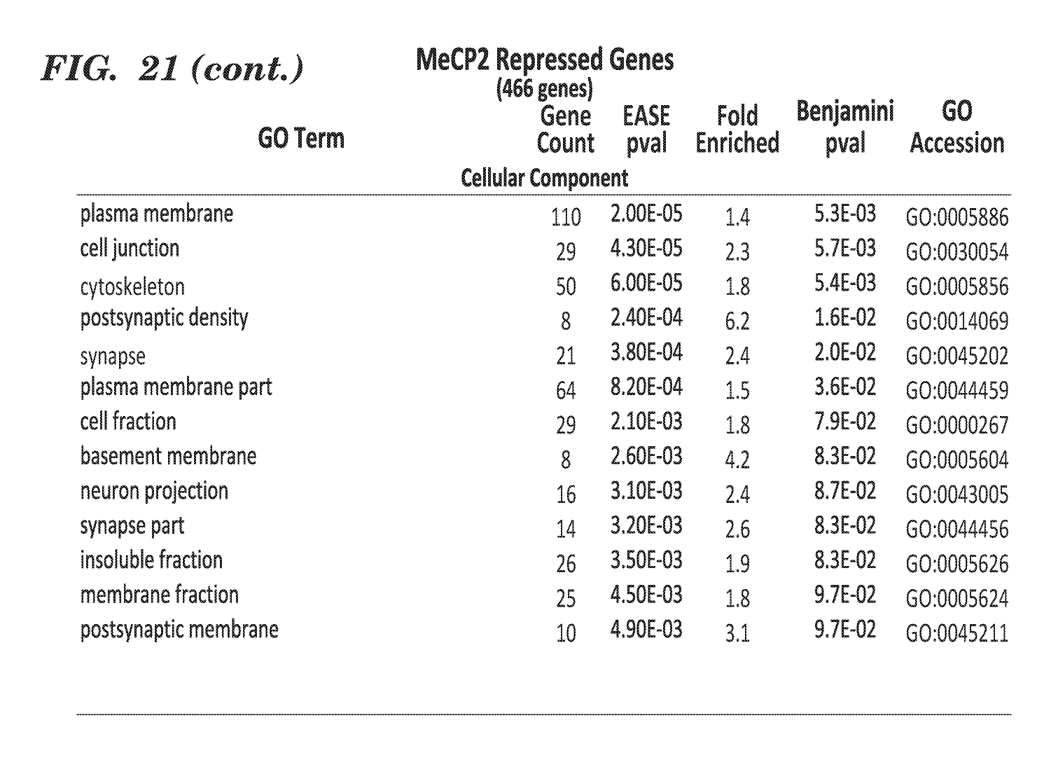

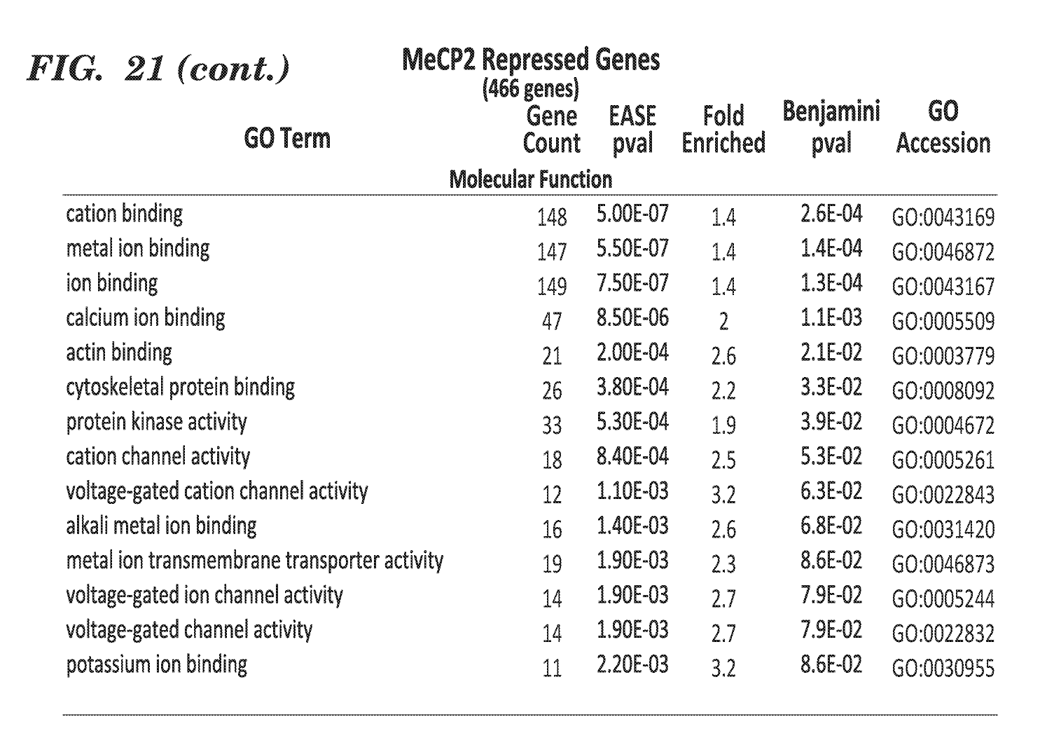

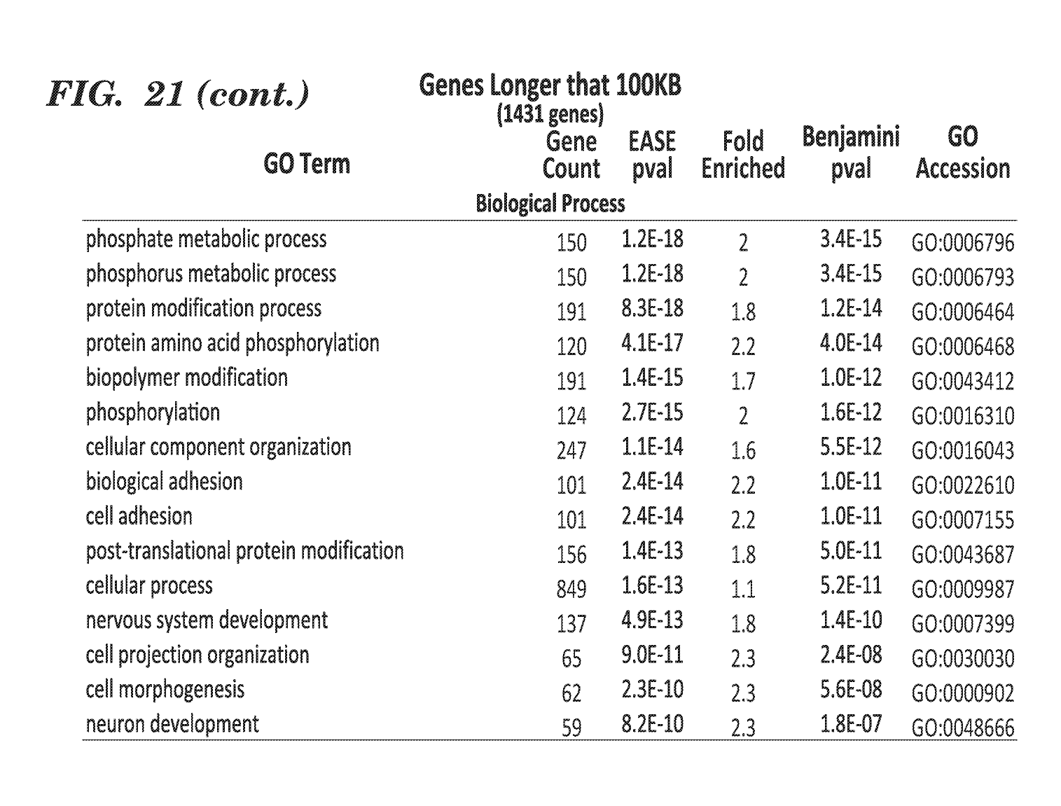

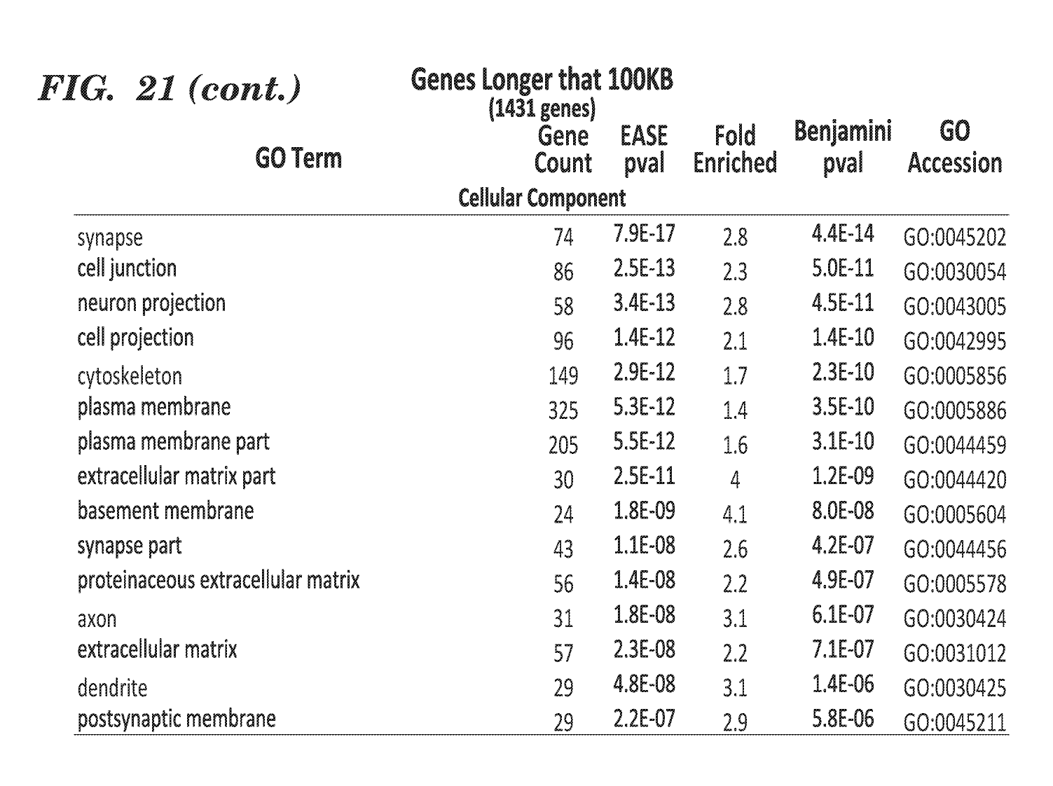

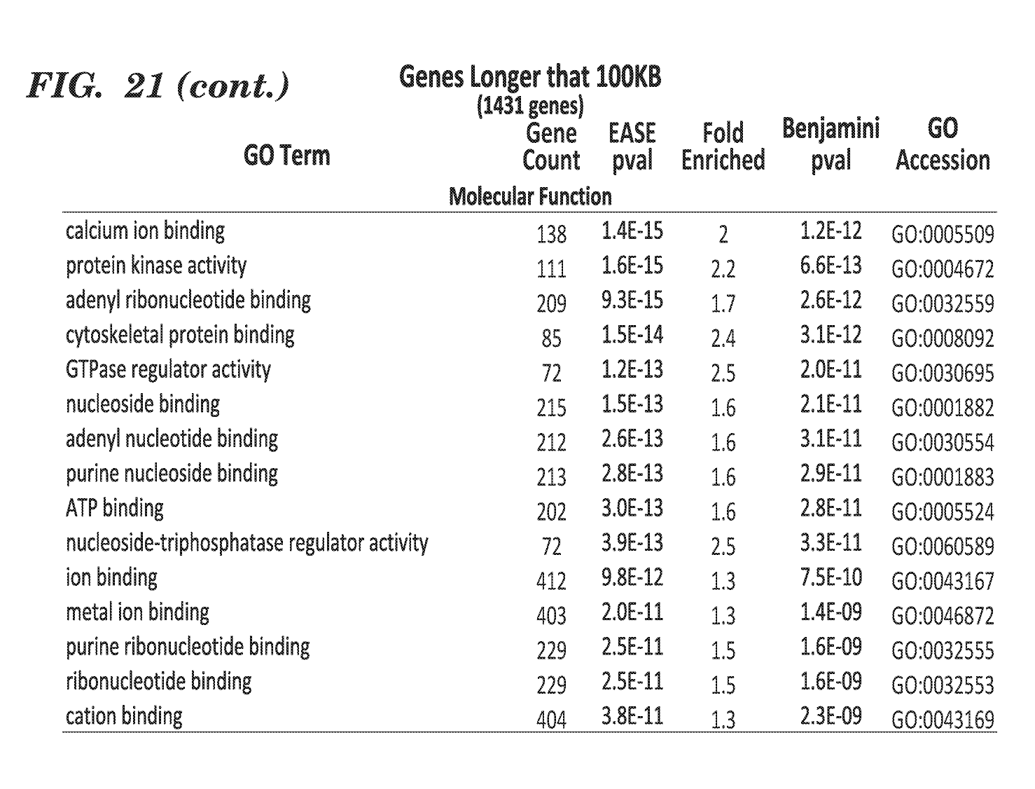

We examined the functional annotations of the 466 MeCP2-repressed genes to gain insight into how their disruption might contribute to RTT pathology. Many of these MeCP2-repressed genes encode proteins that modulate neuronal physiology (e.g. calcium/calmodulin-dependent kinase Camk2d and the voltage-gated potassium channel Kcnh7). In addition, multiple genes involved in axon guidance and synapse formation were identified, including Epha7, Sdk1 and Cntn4 (FIGS. 19a to 19i). Consistent with these observations, gene ontology analysis of MeCP2-repressed genes indicates that they are enriched for annotated neuronal functions (e.g. post-synaptic density, axonogenesis, voltage-gated cation channel activity; FIG. 21). These findings suggest that RTT results from a subtle, yet widespread over-expression of long genes that have specific functions in the nervous system.

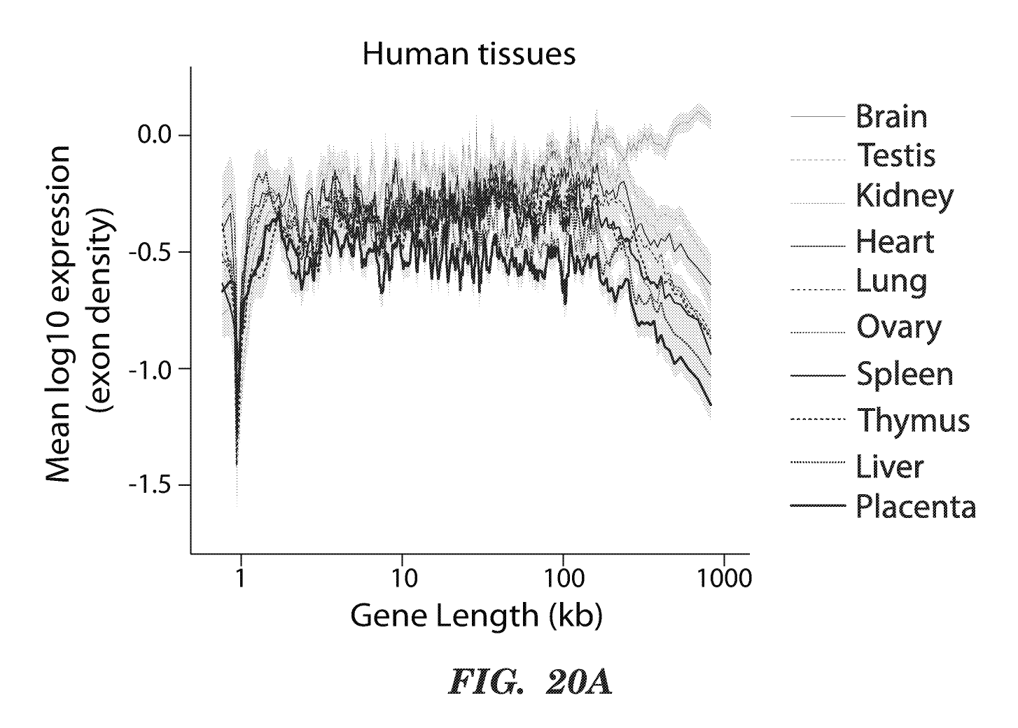

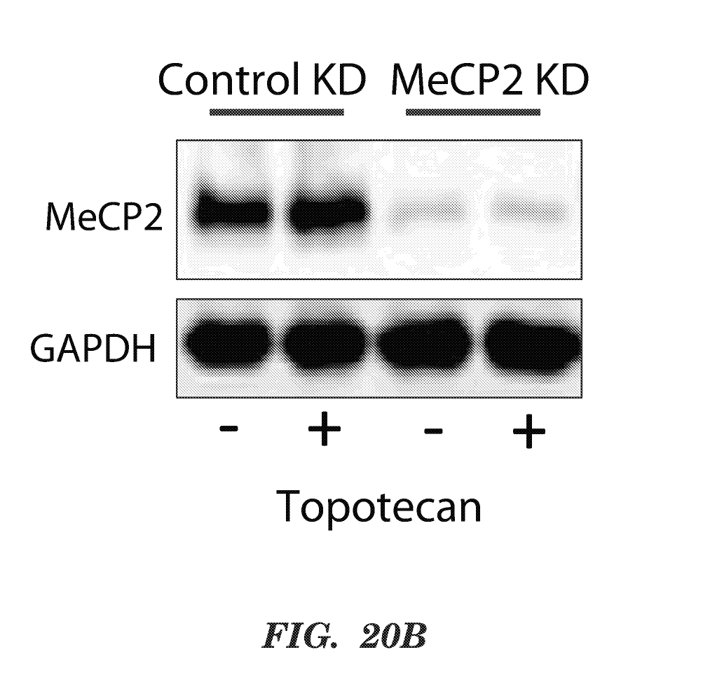

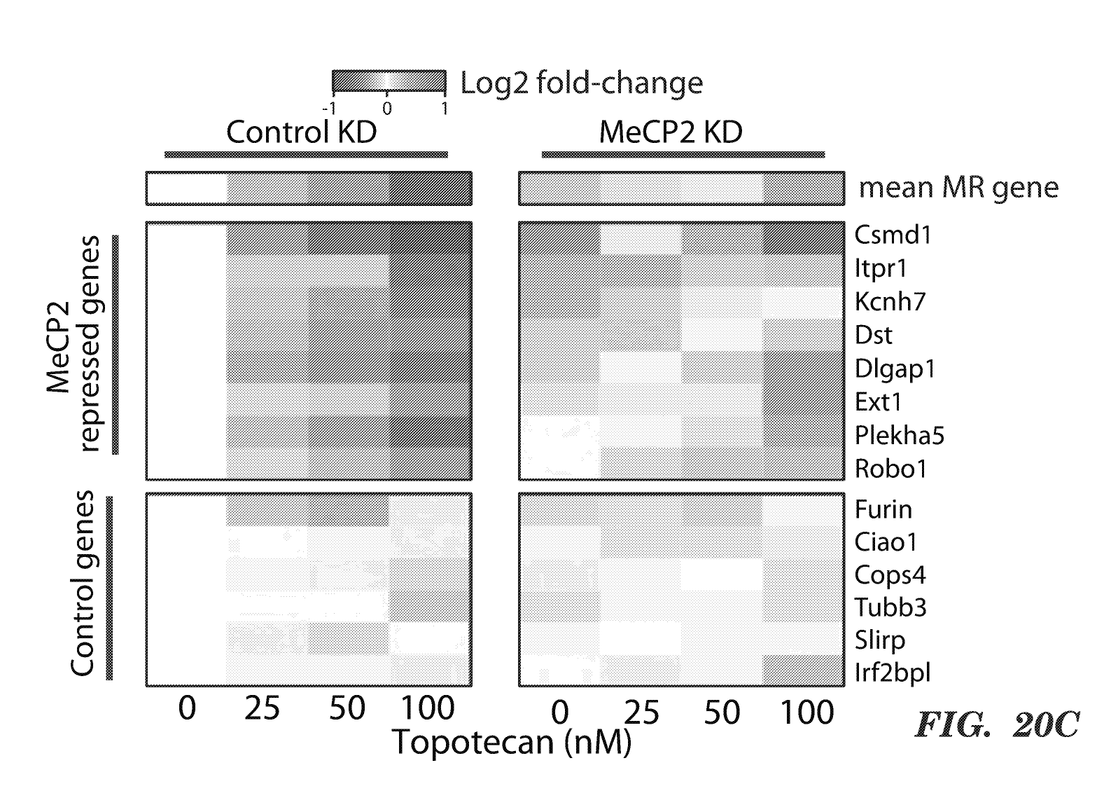

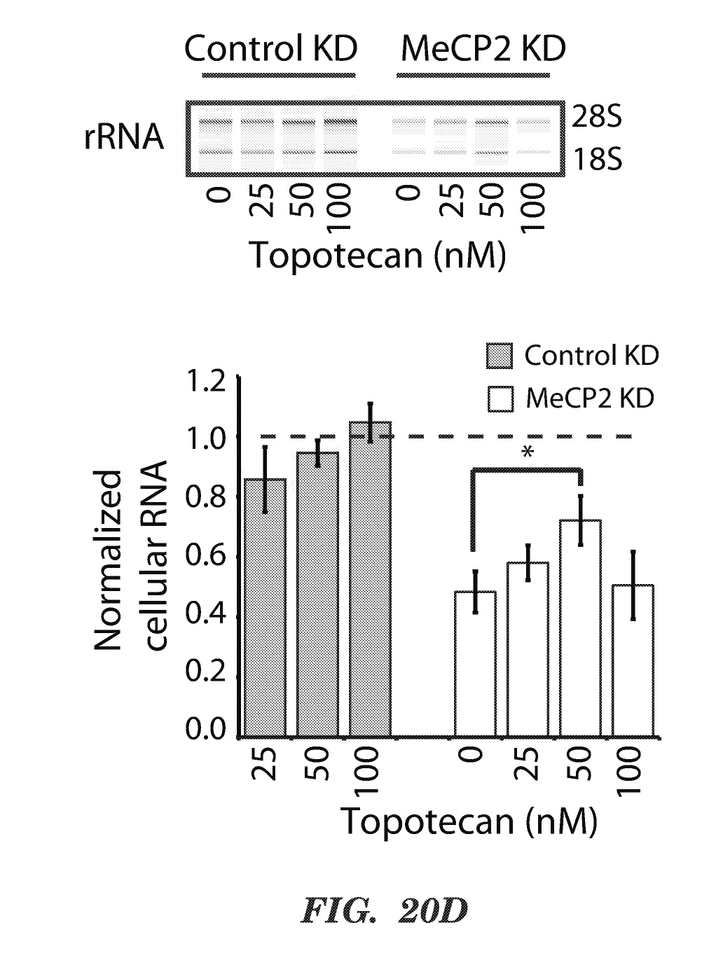

We next considered why the misregulation of long genes as a population in RTT leads specifically to neuronal dysfunction. Many genes with neuronal function are very long^{60,61}, raising the possibility that long genes as a population might be enriched for functions in the nervous system relative to other tissues. If so, the high level of mCA and MeCP2 in neurons may have evolved to temper the expression of long genes specifically in the brain. Indeed, gene ontology analysis of all genes in the genome above 100 kb indicates that the longest genes in the genome are enriched for neuronal annotations (FIG. 21). Moreover, by examining tissue-specific gene expression datasets, we find that long genes as a population are preferentially expressed in mouse and human brain relative to other tissues (FIG. 5c, FIGS. 20a to 20d). We note that, while long genes typically have brain-specific function and expression, brain-specific expression is not a prerequisite for regulation of long genes by MeCP2 in neurons: some long genes are ubiquitously expressed but selectively repressed by MeCP2 in the brain. (FIGS. 19a to 19i).

Affinity of MeCP2 for Methylcytosine and Hydroxymethylcytosine

The recent appreciation that mCH and hmCG build up postnatally in the brain to high levels²²⁻²⁵ suggests that these forms of DNA methylation may play a unique and important role in the maturation and function of neurons. We and others have noted that the build-up of MeCP2 levels in neurons parallels the increase of hmCG and mCH¹⁰, suggesting that MeCP2 may work in concert with these marks. The affinity of MeCP2 for hmC and mCH can provide clues to how they might affect MeCP2 binding in vivo. Several studies have assessed the affinity of MeCP2 for hmC or mC in vitro^{21,26-28,55,56,62} but there has been limited work explicitly assessing the relative affinity of MeCP2 for all possible forms of methylation (unmethylated DNA, mCG, hmCG, mCH and hmCH) within an otherwise identical DNA sequence context.

To compare directly the relative affinity of the methyl binding domain (MBD) of MeCP2 for each form of DNA methylation we have performed EMSA analysis using competitor oligonucleotides in which the central dinucleotide is altered, while the rest of the oligonucleotide sequence and the position of the methylation site(s) are kept constant. Using unlabeled oligonucleotides to compete for binding against a mCG or mCA radiolabeled probe, we find that the relative affinity of two MeCP2 MBD fragments (amino acids 81-170 and 78-162) for mCA is comparable to that of symmetrically methylated CG (data not shown, electrophoretic mobility shift assays for mCG, mCA and hmCA, and FIG. 8). These results are largely consistent with the recent study by Song and colleagues which detected a strong affinity of MeCP2 for mCH that is comparable to mCG²¹. However, the design of our binding assays allows for the assessment of cytosine methylation occurring in the CG, CA, CT, and CC dinucleotide context. In this way, we uniquely identify mCA, and not mCT or mCC, as the high-affinity, mCH binding substrate for MeCP2 in vitro.

In contrast to mC, we find that the MeCP2 MBD has dramatically different affinities for hmCG and hmCA dinucleotides in EMSA assays. We observe that probes containing hydroxymethylation at one or both cytosines in the CG context compete for binding of MeCP2 with similar efficacy to that of an unmethylated oligonucleotide (data not shown, electrophoretic mobility shift assays for mCG, mCA and hmCA, and FIG. 8). This suggests that the binding affinity of MeCP2 to hmCG is similar to unmethylated DNA. Strikingly, an oligonucleotide containing hmCA competes for binding with a high efficacy that is comparable to that of mCG and mCA, suggesting that conversion of mCA to hmCA does not substantially reduce the affinity of MeCP2 for this methylated dinucleotide.

Our results provide an explanation for seemingly incongruent findings from several previous studies examining the affinity of MeCP2 for hmC. Mellen and colleagues¹³ recently observed high affinity of MeCP2 for hmC-containing DNA that was comparable to the affinity of MeCP2 for mC-containing DNA, whereas other studies have noted reduced affinity of MeCP2 for hmCG compared to mCG^{26-28,62}. Notably, Mellen et al. used probes that incorporated hmC throughout the DNA sequence and therefore contained many hmCA sites, while the other studies were performed with probes in which only defined hmCG sites were present. Thus, given our results, the high relative affinity of MeCP2 for hmC observed by Mellen et al. may stem from the presence of hmCA in their DNA probes, while the lower relative affinity detected for hmC in other studies likely resulted from the presence of hmCG alone.

The differential affinity of MeCP2 for hmC depending on the dinucleotide context may have important implications for the binding and function of MeCP2 with hmC across the genome. Recent genome-wide basepair-resolution analysis of hydroxymethylation in the brain indicates that while hmCG is present at appreciable levels, hmCA is exceedingly rare and/or may not be detectable due to limitations of TAB-seq analysis²⁵. Thus, the primary effect of the conversion of mC to hmC in the neuronal genome may be to reduce the affinity of MeCP2 binding at mCG sites, while conversion of a small number of mCA sites to hmCA sites may not substantially alter the binding of MeCP2 at these locations. If hmCA does occur at functionally relevant levels in the genome, our analysis in combination with a previous study suggests that hmCA may in fact serve as a repressive mark: Lister and colleagues²⁵ noted that unlike hmCG, which is correlated with gene expression, the limited hmCH signal that can be detected in genes (while difficult to distinguish from background in the TAB-seq method) is inversely correlated with gene expression levels. This suggests that hmCH may contribute to transcriptional repression. Consistent with this possibility we find that genes that contain high levels of hmCA signal are up-regulated when MeCP2 is lost (see FIG. 9, and data not shown). Together, this suggests a possible model in which upon binding of MeCP2 to hmCA, genes are repressed in a length-dependent manner. Because of the ambiguity created by the low levels of hmCA, however, and the possibility that the hmCA signal detected by current technologies is incomplete definitive conclusions about the existence hmCA and its function with MeCP2 in vivo will require additional studies.

Genomic Analysis of DNA Methylation and MeCP2-Dependent Gene Regulation

To investigate the potential role for DNA methylation or hydroxymethylation in the regulation of gene expression by MeCP2, we assessed whether there is a correlation between the degree of misregulation of gene expression upon the disruption of MeCP2 function (determined by our RNA-seq analysis, or by previous microarray studies) and the levels of mC and hmC associated with genes (assayed by genome-wide bisulfite sequencing or Tet-assisted bisulfite sequencing²⁵). While we assessed methylation at gene regulatory elements as well as gene bodies, our analysis of data obtained from the mouse cortex revealed a correlation between the density of mCA, but not mCG or hmCG, within the transcribed region of genes and the degree to which the genes are up-regulated in the MeCP2 KO compared to wild type mice (FIGS. 9a to 9h and FIG. 10a to 10l). A similar effect was present but less robust, or not apparent, for analysis of gene-body mCA for all genes in the cerebellum and hippocampus respectively. The more subtle nature of this effect in the analysis of all genes in these brain regions may be due to the lower levels of mCA detected there (FIG. 10a to 10l). The requirement of gene-body mCA for MeCP2-dependent repression of long genes specifically is apparent across all brain regions however, as mCA-associated up-regulation is detected for long genes but not short genes in the cortex, hippocampus and cerebellum of MeCP2 KO mice (FIGS. 10a to 10l).

As discussed above, relative to mCA, the level of hmCA in neurons appears to be extremely low and may reflect background signal in the assay used to detect it. However, as with mCA, we also find that in the cortex the density of hmCA signal within gene bodies correlates with the degree of gene up-regulation, raising the possibility that both hmCA and mCA play a role in the repression of long gene expression (FIGS. 9a to 9h). Thus, if technical limitations of current assays underestimate the level of hmCA in neurons, when bound by MeCP2 hmCA would have a similar repressive function as mCA. However, given the current data indicating that the level of hmCA is very low in neurons, we have focused our analysis and discussion on the role of mCA in the regulation of long gene expression.

Elimination of mCA in the Brain by Conditional Deletion of Dnmt3a

Our model predicts that mCA is critical for gene repression by MeCP2 and that decreasing the density of mCA across the transcribed regions of long genes should lead to a length- and mCA-dependent up-regulation of gene expression. To test this prediction, we sought to decrease mCA in neurons by disrupting the function of the DNA methyltransferase that catalyzes the addition of the methyl group to CA dinucleotides in neurons. Dnmt3a is highly expressed in the brain at the time during the postnatal period when the density of mCH across the neuronal genome increases dramatically²⁵, and shRNA knockdown studies of Dnmt3a suggest that it is required for methylation of CH, but not CG, sites within neuronal DNA²¹. To test the role of mCA in gene regulation directly, we mated the Dnmt3a conditional knockout mouse⁴⁵ with a Nestin-cre mouse line⁴⁶, removing Dnmt3a specifically in the brain before high levels of mCA have accumulated (designated Dnmt3a cKO mice). We confirmed, by PCR and western blotting, that excision of the Dnmt3a gene occurs in the cerebellum of Dnmt3a cKO mice, ablating Dnmt3a protein expression (FIGS. 17a to 17d). Bisulfite sequencing of DNA from the cerebellum indicated that methylation of DNA at CH (primarily in the form of mCA) is eliminated from the genome in Dnmt3a cKO compared to control mice (FIG. 22a), but little effect on CG sites is observed. Since CG methylation occurs on both DNA strands, it can be catalyzed by the maintenance methyltransferase, Dnmt1, and does not require the activity of a de novo demethylase, Dnmt3a. This characteristic likely explains how methylation of CG, but not CH, is maintained in the brain in the absence of Dnmt3a.

Given that methylation at CA dinucleotides is significantly decreased in Dnmt3a cKO mice, we next compared the gene expression profiles of wild-type and Dnmt3a cKO mice to determine if eliminating mCA from the transcribed region of long genes leads to their up-regulation. Strikingly, we observe a clear length- and mCA-dependent up-regulation of gene expression in the cerebellum of Dnmt3a cKO mice relative to control mice that is similar in magnitude and scope to the misregulation that we observe in MeCP2 KO mice (FIG. 22b). In addition to this genome-wide analysis, the mCA-dependence of gene up-regulation in MeCP2 KO and Dnmt3a cKO mice can be observed at single genes. For example, Ppm11, a 238 kb gene that contains high levels of mCA, is up-regulated in the MeCP2 KO, MeCP2 R306C and Dnmt3a cKO and is down-regulated in the MeCP2 OE, while Cnksr2, a similarly long gene (221 kb) containing low mCA levels, is largely unaffected in these mutants (for this and additional examples see FIGS. 19a to 19i).

Notably, MeCP2 appears to serve primarily as a reader rather than a writer of DNA methylation, as methyl-sensitive restriction digest, bisulfate sequencing, and affinity-based analysis of hmC and mC in the MeCP2 KO brain did not reveal detectable changes in global methylation patterns (data not shown). Taken together, these findings suggest that Dnmt3a catalyzes the methylation of CA in the neurons and MeCP2 serves specifically as a reader of this mark, binding to these sites within the transcribed regions of genes to restrain their transcription in a length-dependent manner. Consistent with this model, the phenotypes that we observe and that have been previously reported for mice lacking Dnmt3a in the brain, (including neurological deficits and premature death) show similarities to those seen in the MeCP2 KO (data not shown)^{57,58}.

Identification and Analysis of MeCP2-Repressed Genes

To begin to understand how the misregulation of long gene expression contributes to RTT pathology, we identified a representative set of genes that are consistently misregulated when MeCP2 function is perturbed. We analyzed the data from eight different microarray studies across multiple brain regions to identify 466 MeCP2-repressed genes whose expression is consistently increased in the absence of MeCP2 and down-regulated when MeCP2 is over-expressed (FIG. 15). This number of reproducibly misregulated genes is at least 15-fold higher than would be detected by chance (p<1.5×10⁻⁶, see Methods), providing further support for the conclusion that a substantial number of genes are reproducibly misregulated in the Mecp2 mutants. Consistent with the conclusion that these genes are targets of gene-length and mCA-dependent repression, we found that MeCP2-repressed genes are exceptionally long and are enriched for mCA but not for mCG or hmCG (FIG. 5a, FIGS. 19a to 19i, data not shown). Furthermore, this geneset represents a predictive signature of gene misregulation in the absence of MeCP2, since it was found to be significantly up-regulated in multiple MeCP2 mutant brain samples that were not used to define the original geneset (see “test dataset” analysis FIGS. 19a to 19i). Importantly, this same geneset was not found to be consistently misregulated in datasets obtained from multiple mouse models of neurological dysfunction due to disruption of genes other than Mecp2 (FIGS. 19a to 19i). We note that while these MeCP2-repressed genes are a useful representative set of MeCP2 regulated genes, the low signal-to-noise in MeCP2 mutant gene expression data and the continuous nature of the length-dependent effect across the genome suggest that a much broader set of genes is affected in the absence of MeCP2 that would not be captured with the criteria used to define MeCP2-repressed genes (see Methods). Nevertheless, we believe that detailed analysis of the 466 representative genes helps to define important functional characteristics of the population of genes that are up-regulated when MeCP2 function is disrupted.

Brain-Specific Expression of Long Genes and Regulation by MeCP2 and FMRP

Our finding that long genes in general are expressed more highly in the brain than in other tissues raised the possibility that the long length of FMRP targets, and MeCP2-repressed genes is not due to a primary effect of length in determining regulation by these proteins but instead occurs as a secondary consequence of the longer average length of genes that are expressed in the brain. Therefore to control for expression in the brain we first filtered the genome for genes that are robustly expressed in the cortex and cerebellum, calculating the average expression (exon density) of all genes across the cortex and cerebellum and selecting only genes that lie in the top 60% of expression values. We then reexamined the length distribution of each gene list (FIGS. 19a to 19i). This analysis confirms that putative FMRP targets, and MeCP2-repressed genes are not composed of extremely long genes solely as a result of the high expression of long genes in the brain.

In addition to raw expression levels, the finding that long genes as a population are specifically expressed in the brain also raised the possibility that MeCP2 or FMRP primarily target brain-specific genes for repression and that the up-regulation of many long genes that we observed in the MeCP2 KO is only a secondary effect of the de-repression of these brain-specific genes (which tend to be long). To examine this possibility directly, we filtered the genome for genes that are comparably expressed in the brain and other somatic tissues, selecting only genes that have expression in the mouse brain (average exon density of cortex and cerebellum) that is within two-fold of their average expression in non-brain tissues (average exon density of all other tissues). Examination of the MeCP2-repressed genes and FMRP target genes that are within this subset of genes with comparable neural and non-neural expression revealed that they are also extremely long (FIGS. 19a to 19i). This strongly suggests that gene length, not brain-specific expression, is an underlying determinant for regulation by MeCP2 or FMRP.

Recent studies suggest that FMRP binds to its target mRNAs and stalls translation^{31,30}. It is therefore likely that the relatively long length of genes encoding FMRP targets reflects targeting of long mature mRNA. To assess the length of FMRP target mRNA directly we examined the length of the mature transcripts for FMRP targets. We find that FMRP target mRNAs are extremely long compared to the transcriptome average (FIGS. 19a to 19i), even when controlling for minimal expression of mRNAs in the brain (data not shown). These findings are consistent with FMRP binding throughout the coding sequence of mRNAs to impede ribosomes³¹ and suggests that mRNA length contributes directly to the level of regulation by FMRP. Notably, while proteome-wide analysis of translational control by FMRP has not been performed, Darnell and colleagues³¹ did assess the level of repression by FMRP for several target mRNAs, measuring the level of ribosome stalling on these mRNAs in vitro. Consistent with a role for length in determining regulation by FMRP, they reported that the degree of ribosome stalling on FMRP mRNA targets was correlated with mRNA length. Together with our observation that FMRP target mRNAs are exceedingly long relative to the transcriptome average, these results point to mRNA length as a major determinant in translational regulation by FMRP.

### Example 3 MecP2 Binds mCA in the Brain

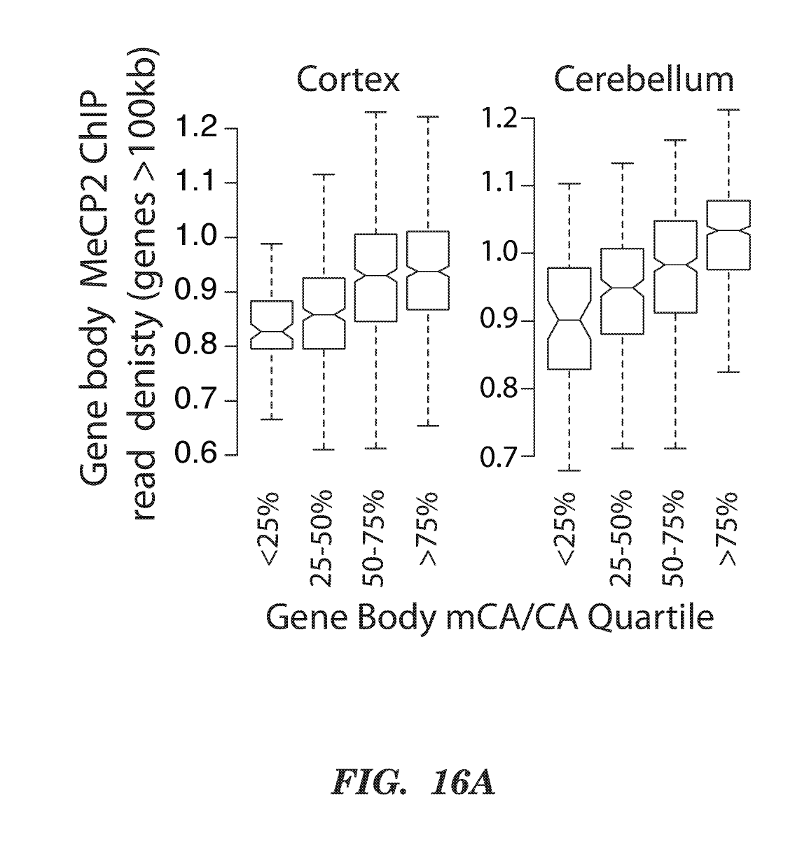

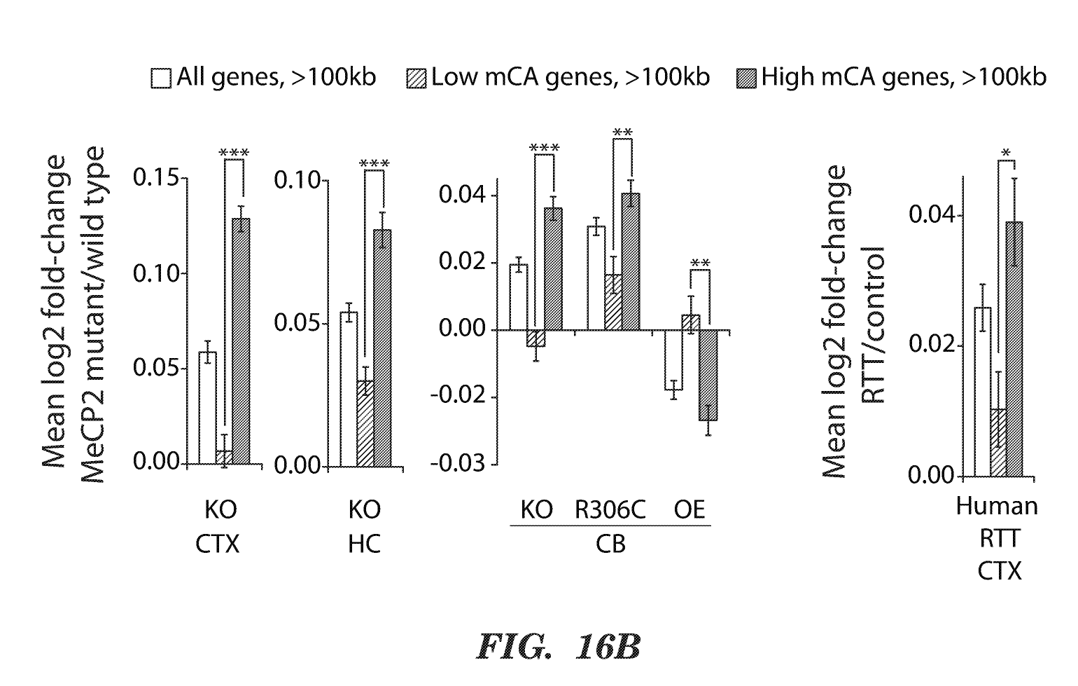

To examine if MeCP2 binds mCA in the brain, we performed chromatin immunoprecipitation sequencing analysis (ChIP-seq) of MeCP2, comparing the MeCP2 binding profile across the genome to base-pair resolution DNA methylation data (see Methods)²⁵. As previously reported^{10,11}, we find that MeCP2 binds broadly across the genome. Nevertheless, within the context of this broad binding, we detect a relative enrichment of MeCP2 at gene bodies that have a high level of mCA (level=(h)mCN/CN within the gene, see Methods), and a depletion of MeCP2 binding at gene bodies where the level of hmCG is high (FIG. 18a to 18d). Notably, long genes (>100 kb) display a strong relationship between mCA levels and MeCP2 ChIP-seq read density (FIG. 16a, FIG. 18a to 18d). Higher resolution analysis of MeCP2 ChIP and mCA levels in the frontal cortex revealed increased mCA under sites of local MeCP2 enrichment in the genome, supporting the conclusion that MeCP2 binds to mCA in vivo (FIG. 18a to 18d). We note that genes containing the highest level of hmCA are also enriched for the MeCP2 ChIP signal (FIG. 18a to 18d). Therefore, if due to limitations of the methods of analysis the amount of hmCA within gene bodies is being underestimated, some of the effects of MeCP2 deletion that are being attributed to MeCP2 binding to mCA might be due to MeCP2 binding to hmCA (see Example 2).

### Example 4 Autism Spectrum Disorders

To explore if disruption of proteins that regulate long gene expression may broadly contribute to autism spectrum disorders (ASDs), we asked if a similar misregulation of gene expression occurs in a prominent ASD, Fragile X syndrome (FXS). FXS is caused by inactivation of FMRP, a protein that represses mRNA translation in neurons²⁹. Strikingly, we find that FMRP target mRNAs and the genes that encode them are significantly longer than the genome average31 (FIG. 18a to 18d, FIGS. 19a to 19i, Example 2,). Moreover, we detect significant overlap between MeCP2-repressed genes and genes encoding FMRP target mRNAs (FIGS. 19a to 19i). These results suggest that up-regulation of long gene function, either through increased transcription (RTT) or mRNA translation (FXS), may represent a common cause of pathology in neurodevelopmental disorders.

A recent study demonstrated that pharmacological inhibition of topoisomerases leads to the broad down-regulation of long genes in neurons¹², suggesting that topoisomerase inhibitors might reverse the up-regulation of long gene expression observed in the absence of MeCP2. To test this, we knocked-down MeCP2 expression in cultured cortical neurons with RNAi and treated these cells with the topoisomerase inhibitor topotecan. We found that MeCP2 knockdown leads to the up-regulation of long genes and that exposure of MeCP2-deficient neurons to topotecan results in a dose-dependent reversal of long gene misregulation (FIGS. 20a to 20d).

The disruption of MeCP2 function in both mouse and human neurons leads to an overall reduction in cell health that can be measured as a decrease in the level of ribosomal RNA and cell size^{15,30}. Strikingly, we found that the concentration of topotecan that most effectively reverses overexpression of long genes (50 nM) partially reverses the decreased ribosomal RNA content observed in neurons lacking MeCP2 (FIGS. 20a to 20d). This result suggests that the rebalancing of long gene expression improves cell health in MeCP2 knockdown neurons, leading to increased cellular rRNA content. Taken together, these data suggest that rebalancing long gene expression in neurons lacking MeCP2 may attenuate the cellular dysfunction observed in these cells. We find that long genes are misregulated in RTT, and that this misregulation can be reversed by topotecan treatment.

### Example 5 In Vivo

In Vivo Topotecan Treatment:

MeCP2 hemizygous male mice at 8 weeks of age (Jackson Labs) were separated into two equal groups based weight. Using standard stereotaxic techniques, cannula (Alzet, Brain Infusion Kit 3) were guided to the right lateral ventricle (from bregma; A/P 1 mm, LV 1 mm) and connected to an osmotic pump (Alzet, 1004, 28-day release) that was implanted subcutaneously. The osmotic pumps had been previously loaded with either vehicle (50 mM tartaric acid) or 25 μM topotecan and primed at 37° C. for approximately 2 days prior to implantation. All mice survived surgery and the following postoperative day. Survival monitoring began on postoperative day 2 and behavioral scoring began on postoperative day 4. MeCP2 behavior was scored as previously reported by Guy et al. Science 2005. Briefly, 6 behavioral domains are monitored to capture the progression of the MeCP2 phenotype: locomotor activity, gait, hindlimb clasp, tremor, breathing, and overall condition. Each behavioral domain is scored as 0 (absence), 1 (mild to moderate), or 2 (severe), and summed to give the total behavioral score. Survival statistics: p=0.09; Mantel-Cox. Behavioral statistics: * p<0.05; 2-way ANOVA, Fisher's LSD. N=7-8.

All references described herein and throughout the specification are incorporated by reference in their entirety.

1. A method for treating an autism spectrum disorder comprising administering to a subject an effective amount of an agent that modulates the expression of long genes in the brain.

2. The method of claim 1, wherein the agent modulates expression of long genes in the brain by modulating the transcription of long genes.

3. The method of claim 1, wherein the agent modulates expression of long genes in the brain by modulating the translation of long genes.

4. The method of claim 1, wherein the agent increases the expression of long genes in the brain.

5. The method of claim 1, wherein the agent decreases the expression of long genes in the brain.

6. The method of claim 1, wherein the autism spectrum disorder is MeCP2 duplication syndrome and the agent increases the expression of long genes in the brain.

7. The method of claim 1, wherein the autism spectrum disorder is Rett syndrome and the agent decreases the expression of long genes in the brain.

8. The method of claim 1, wherein the autism spectrum disorder is Fragile X syndrome and the agent decreases the expression of long genes in the brain.

9. (canceled)

10. The method of claim 1, wherein the agent is selected from the group consisting of a small molecule, a nucleic acid, a protein, a peptide, an RNA interfering agent (RNAi), and an antibody.

11. (canceled)

12. The method of claim 1, wherein the agent is administered by a route selected from the group consisting of topical administration, enteral administration, and parenteral administration.

13. (canceled)

14. (canceled)

15. The method of claim 1, wherein the agent is formulated for delivery to the brain.

16. (canceled)

17. (canceled)

18. The method of claim 1, wherein the autism spectrum disorder is caused by a mutation in topoisomerase and the agent increases expression of a long gene in the brain.

19. The method of claim 1, wherein the agent that increases expression of long genes in the brain is a DNA methyltransferase inhibitor.

20. The method of claim 1, wherein the agent that decreases expression of long genes in the brain and is selected from the group consisting of: a topoisomerase inhibitor, a nucleotide analog that inhibits transcriptional elongation, a BRD4 inhibitor that inhibits pro-elongation chromatin modifiers, an inhibitor of Dot1 that promotes elongation-associated chromatin modification, Alpha-Amanitin, a protein synthesis inhibitor, and a DNA intercalator that blocks RNA polymerases.

21. The method of claim 1, wherein the agent that decreases expression of long genes in the brain inhibits a protein that promotes elongation selected from the group consisting of: BRD4, Dot11, Ptefb, DSIF, SPt5p, Spt4p, PAF, Ccr4-Not, Sp3, ELL, P-TEFb, and AFF4.

22. The method of claim 1, wherein the agent that increases expression of long genes in the brain activates a protein that promotes elongation selected from the group consisting of: BRD4, Dot11, Ptefb, DSIF, SPt5p, Spt4p, PAF, Ccr4, Not, Sp3, ELL, P-TEFb, and AFF4.

23. The method of claim 1, wherein the agent inhibits a protein involved in translational elongation and is selected from the group consisting of: Lactimidomycin, Diphthamide, Stm1p, 4EGI1, Orthoformimysin, e1F5A, Minocycline.

24. The method of claim 1, wherein the agent activates a protein involved in translational elongation and is selected from the group consisting of: Lactimidomycin, Diphthamide, Stm1p, 4EGI1, Orthoformimysin, e1F5A, Minocycline.

25. A method for treatment of Rett syndrome comprising administering to a subject an effective amount of a topoisomerase inhibitor, wherein the effective amount of the topoisomerase inhibitor decreases the expression of long genes in the brain, thereby treating Rhett syndrome.

26. A method for treatment of Fragile X syndrome comprising administering to a subject an effective amount of a topoisomerase inhibitor, wherein the effective amount of the topoisomerase inhibitor decreases the expression of long genes in the brain, thereby treating Fragile X syndrome.

27.-35. (canceled)
